SEQUENCE LISTING (1342 sequences)

SEQ ID NO: 1 - DNA, 22 bp, Artificial Sequence
  source: /note=“Description of Artificial Sequence Synthetic primer”
aaaaactgcttgccttcttctt

SEQ ID NO: 2 - DNA, 23 bp, Artificial Sequence
  source: /note=“Description of Artificial Sequence Synthetic primer”
tctatgggttctcacaactcaac

SEQ ID NO: 3 - DNA, 21 bp, Artificial Sequence
  source: /note=“Description of Artificial Sequence Synthetic primer”
tggacaaaaataccatcatca

SEQ ID NO: 4 - DNA, 21 bp, Artificial Sequence
  source: /note=“Description of Artificial Sequence Synthetic primer”
agatcatcctgaacataaggt

SEQ ID NO: 5 - DNA, 23 bp, Artificial Sequence
  source: /note=“Description of Artificial Sequence Synthetic primer”
catctaaatacatgaaaaaggag

SEQ ID NO: 6 - DNA, 22 bp, Artificial Sequence
  source: /note=“Description of Artificial Sequence Synthetic primer”
tcaagtatccaggacttgttcg

SEQ ID NO: 7 - DNA, 22 bp, Artificial Sequence
  source: /note=“Description of Artificial Sequence Synthetic primer”
ggacccaagatctgattctagc

SEQ ID NO: 8 - DNA, 20 bp, Artificial Sequence
  source: /note=“Description of Artificial Sequence Synthetic primer”
agggtgagctgttctcagga

SEQ ID NO: 9 - DNA, 23 bp, Artificial Sequence
  source: /note=“Description of Artificial Sequence Synthetic primer”
tccccagactaattatggaaaaa

SEQ ID NO: 10 - DNA, 22 bp, Artificial Sequence
  source: /note=“Description of Artificial Sequence Synthetic primer”
tcactttactgttcaccaaacg

SEQ ID NO: 11 - DNA, 22 bp, Artificial Sequence
  source: /note=“Description of Artificial Sequence Synthetic primer”
ggattttagggcactaggaagg

SEQ ID NO: 12 - DNA, 23 bp, Artificial Sequence
  source: /note=“Description of Artificial Sequence Synthetic primer”
gagagtttttaaagagtgtcgtt

SEQ ID NO: 13 - DNA, 23 bp, Artificial Sequence
  source: /note=“Description of Artificial Sequence Synthetic primer”
tgtatttgcctaaaagtaagagg

SEQ ID NO: 14 - DNA, 20 bp, Artificial Sequence
  source: /note=“Description of Artificial Sequence Synthetic primer”
ggcagagttctcttgacgtg

SEQ ID NO: 15 - DNA, 24 bp, Artificial Sequence
  source: /note=“Description of Artificial Sequence Synthetic primer”
cagctaaaggaaaactattaatgc

SEQ ID NO: 16 - DNA, 22 bp, Artificial Sequence
  source: /note=“Description of Artificial Sequence Synthetic primer”
tctctttgtctgttagggtttt

SEQ ID NO: 17 - DNA, 22 bp, Artificial Sequence
  source: /note=“Description of Artificial Sequence Synthetic primer”
tcatctgtgaaatagggacacc

SEQ ID NO: 18 - DNA, 22 bp, Artificial Sequence
  source: /note=“Description of Artificial Sequence Synthetic primer”
gctcttaaaactcatcccaagc

SEQ ID NO: 19 - DNA, 23 bp, Artificial Sequence
  source: /note=“Description of Artificial Sequence Synthetic primer”
agaaattattcaggacacagaga

SEQ ID NO: 20 - DNA, 23 bp, Artificial Sequence
  source: /note=“Description of Artificial Sequence Synthetic primer”
tcctgacaagacagttatcatct

SEQ ID NO: 21 - DNA, 23 bp, Artificial Sequence
  source: /note=“Description of Artificial Sequence Synthetic primer”
gagaagaatgattagaccttgct

SEQ ID NO: 22 - DNA, 22 bp, Artificial Sequence
  source: /note=“Description of Artificial Sequence Synthetic primer”
acaagagtacacgagagaaaaa

SEQ ID NO: 23 - DNA, 22 bp, Artificial Sequence
  source: /note=“Description of Artificial Sequence Synthetic primer”
tgatgtggaatagtttaggtga

SEQ ID NO: 24 - DNA, 23 bp, Artificial Sequence
  source: /note=“Description of Artificial Sequence Synthetic primer”
tccaaaaggtaattccaatatgc

SEQ ID NO: 25 - DNA, 20 bp, Artificial Sequence
  source: /note=“Description of Artificial Sequence Synthetic primer”
ggatatgccgcttttcctct

SEQ ID NO: 26 - DNA, 23 bp, Artificial Sequence
  source: /note=“Description of Artificial Sequence Synthetic primer”
gctaagtaaataatttggcagtt

SEQ ID NO: 27 - DNA, 23 bp, Artificial Sequence
  source: /note=“Description of Artificial Sequence Synthetic primer”
tcacagtgtttctcatagtttta

SEQ ID NO: 28 - DNA, 22 bp, Artificial Sequence
  source: /note=“Description of Artificial Sequence Synthetic primer”
cagcagctagtgttgcactaat

SEQ ID NO: 29 - DNA, 21 bp, Artificial Sequence
  source: /note=“Description of Artificial Sequence Synthetic primer”
ggttcacagagcccaagttac

SEQ ID NO: 30 - DNA, 24 bp, Artificial Sequence
  source: /note=“Description of Artificial Sequence Synthetic primer”
tgagtctcttactgatcctgtgac

SEQ ID NO: 31 - DNA, 20 bp, Artificial Sequence
  source: /note=“Description of Artificial Sequence Synthetic primer”
gagtcactcttggggtatca

SEQ ID NO: 32 - DNA, 20 bp, Artificial Sequence
  source: /note=“Description of Artificial Sequence Synthetic primer”
gatgcccagcctcttctctc

SEQ ID NO: 33 - DNA, 20 bp, Artificial Sequence
  source: /note=“Description of Artificial Sequence Synthetic primer”
agagatctccgcatcctgtg

SEQ ID NO: 34 - DNA, 20 bp, Artificial Sequence
  source: /note=“Description of Artificial Sequence Synthetic primer”
gggggccaataactatgctc

SEQ ID NO: 35 - DNA, 20 bp, Artificial Sequence
  source: /note=“Description of Artificial Sequence Synthetic primer”
agtgtgatgtttgagtgagg

SEQ ID NO: 36 - DNA, 23 bp, Artificial Sequence
  source: /note=“Description of Artificial Sequence Synthetic primer”
gtcctatcatcttttatttccaa

SEQ ID NO: 37 - DNA, 21 bp, Artificial Sequence
  source: /note=“Description of Artificial Sequence Synthetic primer”
ttccttggcattttagtttcc

SEQ ID NO: 38 - DNA, 21 bp, Artificial Sequence
  source: /note=“Description of Artificial Sequence Synthetic primer”
tcccatgtaacacctttcaga

SEQ ID NO: 39 - DNA, 23 bp, Artificial Sequence
  source: /note=“Description of Artificial Sequence Synthetic primer”
tcacccattcttcatactctttg

SEQ ID NO: 40 - DNA, 20 bp, Artificial Sequence
  source: /note=“Description of Artificial Sequence Synthetic primer”
aacttttcaggtcggcagtg

SEQ ID NO: 41 - DNA, 21 bp, Artificial Sequence
  source: /note=“Description of Artificial Sequence Synthetic primer”
ggagagaatcccttacccttg

SEQ ID NO: 42 - DNA, 22 bp, Artificial Sequence
  source: /note=“Description of Artificial Sequence Synthetic primer”
ggaattttattagatgttgagg

SEQ ID NO: 43 - DNA, 21 bp, Artificial Sequence
  source: /note=“Description of Artificial Sequence Synthetic primer”
cagcccagattttctctttca

SEQ ID NO: 44 - DNA, 20 bp, Artificial Sequence
  source: /note=“Description of Artificial Sequence Synthetic primer”
tcgaggtaaataggcccaca

SEQ ID NO: 45 - DNA, 23 bp, Artificial Sequence
  source: /note=“Description of Artificial Sequence Synthetic primer”
ttcagctcttctactctggactg

SEQ ID NO: 46 - DNA, 24 bp, Artificial Sequence
  source: /note=“Description of Artificial Sequence Synthetic primer”
tgaaacaagagaagactggatttg

SEQ ID NO: 47 - DNA, 24 bp, Artificial Sequence
  source: /note=“Description of Artificial Sequence Synthetic primer”
gttatatctcttttgtttctctcc

SEQ ID NO: 48 - DNA, 21 bp, Artificial Sequence
  source: /note=“Description of Artificial Sequence Synthetic primer”
ttggattgttagagaataacg

SEQ ID NO: 49 - DNA, 22 bp, Artificial Sequence
  source: /note=“Description of Artificial Sequence Synthetic primer”
tggacaagagagacttcaggag

SEQ ID NO: 50 - DNA, 23 bp, Artificial Sequence
  source: /note=“Description of Artificial Sequence Synthetic primer”
gctgagccttttagatagtgctg

SEQ ID NO: 51 - DNA, 20 bp, Artificial Sequence
  source: /note=“Description of Artificial Sequence Synthetic primer”
tccaactggaaaacacctca

SEQ ID NO: 52 - DNA, 22 bp, Artificial Sequence
  source: /note=“Description of Artificial Sequence Synthetic primer”
gagccaccttcaagactctttc

SEQ ID NO: 53 - DNA, 22 bp, Artificial Sequence
  source: /note=“Description of Artificial Sequence Synthetic primer”
tttaaatctttccagggggttt

SEQ ID NO: 54 - DNA, 20 bp, Artificial Sequence
  source: /note=“Description of Artificial Sequence Synthetic primer”
tgattctcagcctggagttt

SEQ ID NO: 55 - DNA, 20 bp, Artificial Sequence
  source: /note=“Description of Artificial Sequence Synthetic primer”
aggattcagccatccatctg

SEQ ID NO: 56 - DNA, 20 bp, Artificial Sequence
  source: /note=“Description of Artificial Sequence Synthetic primer”
tctgccatgggaggtataga

SEQ ID NO: 57 - DNA, 23 bp, Artificial Sequence
  source: /note=“Description of Artificial Sequence Synthetic primer”
aaaacataattgaacacctagca

SEQ ID NO: 58 - DNA, 20 bp, Artificial Sequence
  source: /note=“Description of Artificial Sequence Synthetic primer”
aataggaggctgctctatgc

SEQ ID NO: 59 - DNA, 23 bp, Artificial Sequence
  source: /note=“Description of Artificial Sequence Synthetic primer”
tgattcacttccagttcttgaca

SEQ ID NO: 60 - DNA, 20 bp, Artificial Sequence
  source: /note=“Description of Artificial Sequence Synthetic primer”
agtgaccttgctggtttgtg

SEQ ID NO: 61 - DNA, 21 bp, Artificial Sequence
  source: /note=“Description of Artificial Sequence Synthetic primer”
gggtaccatatgaggccagtt

SEQ ID NO: 62 - DNA, 21 bp, Artificial Sequence
  source: /note=“Description of Artificial Sequence Synthetic primer”
tcttcttcccaatgtcatgga

SEQ ID NO: 63 - DNA, 21 bp, Artificial Sequence
  source: /note=“Description of Artificial Sequence Synthetic primer”
ccaggcttccaagattattgt

SEQ ID NO: 64 - DNA, 22 bp, Artificial Sequence
  source: /note=“Description of Artificial Sequence Synthetic primer”
aaggcatctcaggtgttatttt

SEQ ID NO: 65 - DNA, 19 bp, Artificial Sequence
  source: /note=“Description of Artificial Sequence Synthetic primer”
cctcgctgtccctgcatac

SEQ ID NO: 66 - DNA, 20 bp, Artificial Sequence
  source: /note=“Description of Artificial Sequence Synthetic primer”
aagtgctgactctgttctgg

SEQ ID NO: 67 - DNA, 22 bp, Artificial Sequence
  source: /note=“Description of Artificial Sequence Synthetic primer”
gaatatctgtctcggaatacca

SEQ ID NO: 68 - DNA, 21 bp, Artificial Sequence
  source: /note=“Description of Artificial Sequence Synthetic primer”
gggatgtgtgatttctgaagg

SEQ ID NO: 69 - DNA, 24 bp, Artificial Sequence
  source: /note=“Description of Artificial Sequence Synthetic primer”
gaacaacatctatcattcatctct

SEQ ID NO: 70 - DNA, 23 bp, Artificial Sequence
  source: /note=“Description of Artificial Sequence Synthetic primer”
caccactctaaagtagaccattg

SEQ ID NO: 71 - DNA, 20 bp, Artificial Sequence
  source: /note=“Description of Artificial Sequence Synthetic primer”
gctttggggttatagctgga

SEQ ID NO: 72 - DNA, 20 bp, Artificial Sequence
  source: /note=“Description of Artificial Sequence Synthetic primer”
agatggccattagctaggaa

SEQ ID NO: 73 - DNA, 23 bp, Artificial Sequence
  source: /note=“Description of Artificial Sequence Synthetic primer”
gcacatagaggtctctctcttct

SEQ ID NO: 74 - DNA, 24 bp, Artificial Sequence
  source: /note=“Description of Artificial Sequence Synthetic primer”
ctatattagaacactcagcagcta

SEQ ID NO: 75 - DNA, 20 bp, Artificial Sequence
  source: /note=“Description of Artificial Sequence Synthetic primer”
agggctgaacaaggaactga

SEQ ID NO: 76 - DNA, 21 bp, Artificial Sequence
  source: /note=“Description of Artificial Sequence Synthetic primer”
ctcatcctgagctctcgtgta

SEQ ID NO: 77 - DNA, 23 bp, Artificial Sequence
  source: /note=“Description of Artificial Sequence Synthetic primer”
tcactcatgttttaccttttagc

SEQ ID NO: 78 - DNA, 23 bp, Artificial Sequence
  source: /note=“Description of Artificial Sequence Synthetic primer”
tgagtcagattcttcataacttt

SEQ ID NO: 79 - DNA, 20 bp, Artificial Sequence
  source: /note=“Description of Artificial Sequence Synthetic primer”
tactgccaacagacaactcg

SEQ ID NO: 80 - DNA, 21 bp, Artificial Sequence
  source: /note=“Description of Artificial Sequence Synthetic primer”
ttagaccgcagacctttagaa

SEQ ID NO: 81 - DNA, 20 bp, Artificial Sequence
  source: /note=“Description of Artificial Sequence Synthetic primer”
ggggcagatcagaaatgttg

SEQ ID NO: 82 - DNA, 20 bp, Artificial Sequence
  source: /note=“Description of Artificial Sequence Synthetic primer”
ggctgttctcaatggtgtca

SEQ ID NO: 83 - DNA, 21 bp, Artificial Sequence
  source: /note=“Description of Artificial Sequence Synthetic primer”
ccccatatgtaacccatcaca

SEQ ID NO: 84 - DNA, 24 bp, Artificial Sequence
  source: /note=“Description of Artificial Sequence Synthetic primer”
tctttggaagagaaatgtgattct

SEQ ID NO: 85 - DNA, 22 bp, Artificial Sequence
  source: /note=“Description of Artificial Sequence Synthetic primer”
ggaatgtatttctgctgtgctg

SEQ ID NO: 86 - DNA, 23 bp, Artificial Sequence
  source: /note=“Description of Artificial Sequence Synthetic primer”
tcactattccttactccaggtga

SEQ ID NO: 87 - DNA, 20 bp, Artificial Sequence
  source: /note=“Description of Artificial Sequence Synthetic primer”
ccattcacgtggcacttttt

SEQ ID NO: 88 - DNA, 22 bp, Artificial Sequence
  source: /note=“Description of Artificial Sequence Synthetic primer”
caccttactgcttcctgctacc

SEQ ID NO: 89 - DNA, 22 bp, Artificial Sequence
  source: /note=“Description of Artificial Sequence Synthetic primer”
ccaaaggctgtattatttatgc

SEQ ID NO: 90 - DNA, 22 bp, Artificial Sequence
  source: /note=“Description of Artificial Sequence Synthetic primer”
gtgttgaagtgatgtaattcag

SEQ ID NO: 91 - DNA, 21 bp, Artificial Sequence
  source: /note=“Description of Artificial Sequence Synthetic primer”
tgaacatatcagctggccatt

SEQ ID NO: 92 - DNA, 20 bp, Artificial Sequence
  source: /note=“Description of Artificial Sequence Synthetic primer”
aaagcccagaattgacttgg

SEQ ID NO: 93 - DNA, 22 bp, Artificial Sequence
  source: /note=“Description of Artificial Sequence Synthetic primer”
caaacctccagggtagtagaca

SEQ ID NO: 94 - DNA, 20 bp, Artificial Sequence
  source: /note=“Description of Artificial Sequence Synthetic primer”
ggggttcataagggaaacca

SEQ ID NO: 95 - DNA, 23 bp, Artificial Sequence
  source: /note=“Description of Artificial Sequence Synthetic primer”
tctcagagcaacatgtaccaaaa

SEQ ID NO: 96 - DNA, 20 bp, Artificial Sequence
  source: /note=“Description of Artificial Sequence Synthetic primer”
gcccaatcagacatcaatcc

SEQ ID NO: 97 - DNA, 20 bp, Artificial Sequence
  source: /note=“Description of Artificial Sequence Synthetic primer”
gtttcccagcaaattcccta

SEQ ID NO: 98 - DNA, 22 bp, Artificial Sequence
  source: /note=“Description of Artificial Sequence Synthetic primer”
tcatcaaaatggatcataacag

SEQ ID NO: 99 - DNA, 21 bp, Artificial Sequence
  source: /note=“Description of Artificial Sequence Synthetic primer”
tttggagtgggtctcttcact

SEQ ID NO: 100 - DNA, 22 bp, Artificial Sequence
  source: /note=“Description of Artificial Sequence Synthetic primer”
aaagagtacattctgccttgct

SEQ ID NO: 101 - DNA, 24 bp, Artificial Sequence
  source: /note=“Description of Artificial Sequence Synthetic primer”
gctcactgttaccctactactctc

SEQ ID NO: 102 - DNA, 21 bp, Artificial Sequence
  source: /note=“Description of Artificial Sequence Synthetic primer”
accacacaaatgattatggta

SEQ ID NO: 103 - DNA, 23 bp, Artificial Sequence
  source: /note=“Description of Artificial Sequence Synthetic primer”
ccacacactgaaaagaatttgtg

SEQ ID NO: 104 - DNA, 21 bp, Artificial Sequence
  source: /note=“Description of Artificial Sequence Synthetic primer”
agtgggctggatatatgaaaa

SEQ ID NO: 105 - DNA, 23 bp, Artificial Sequence
  source: /note=“Description of Artificial Sequence Synthetic primer”
aggcatgtgttaaactagaaaaa

SEQ ID NO: 106 - DNA, 22 bp, Artificial Sequence
  source: /note=“Description of Artificial Sequence Synthetic primer”
ggaggaagctgtgttcttttca

SEQ ID NO: 107 - DNA, 20 bp, Artificial Sequence
  source: /note=“Description of Artificial Sequence Synthetic primer”
ggggatcttaaaagcaccaa

SEQ ID NO: 108 - DNA, 20 bp, Artificial Sequence
  source: /note=“Description of Artificial Sequence Synthetic primer”
gacactcccacttctgccta

SEQ ID NO: 109 - DNA, 20 bp, Artificial Sequence
  source: /note=“Description of Artificial Sequence Synthetic primer”
cagcctaaatttccagtctt

SEQ ID NO: 110 - DNA, 22 bp, Artificial Sequence
  source: /note=“Description of Artificial Sequence Synthetic primer”
agttatgagtaatgaaggaagg

SEQ ID NO: 111 - DNA, 23 bp, Artificial Sequence
  source: /note=“Description of Artificial Sequence Synthetic primer”
atttcttcaagtgtatacagagc

SEQ ID NO: 112 - DNA, 20 bp, Artificial Sequence
  source: /note=“Description of Artificial Sequence Synthetic primer”
caggcaaacattcccttgta

SEQ ID NO: 113 - DNA, 24 bp, Artificial Sequence
  source: /note=“Description of Artificial Sequence Synthetic primer”
tgtctttgctcagttatgaagaga

SEQ ID NO: 114 - DNA, 23 bp, Artificial Sequence
  source: /note=“Description of Artificial Sequence Synthetic primer”
ttgtaaatttttctctaggtgtg

SEQ ID NO: 115 - DNA, 21 bp, Artificial Sequence
  source: /note=“Description of Artificial Sequence Synthetic primer”
ggcatggcaatactcttctga

SEQ ID NO: 116 - DNA, 23 bp, Artificial Sequence
  source: /note=“Description of Artificial Sequence Synthetic primer”
gattttcacatctaattttcacc

SEQ ID NO: 117 - DNA, 22 bp, Artificial Sequence
  source: /note=“Description of Artificial Sequence Synthetic primer”
acaatgagctattttaactcca

SEQ ID NO: 118 - DNA, 23 bp, Artificial Sequence
  source: /note=“Description of Artificial Sequence Synthetic primer”
actaactttgcaagatacagatt

SEQ ID NO: 119 - DNA, 20 bp, Artificial Sequence
  source: /note=“Description of Artificial Sequence Synthetic primer”
tggccacttgcttatttgaa

SEQ ID NO: 120 - DNA, 20 bp, Artificial Sequence
  source: /note=“Description of Artificial Sequence Synthetic primer”
tgttcttaagttgcccataa

SEQ ID NO: 121 - DNA, 22 bp, Artificial Sequence
  source: /note=“Description of Artificial Sequence Synthetic primer”
cccactttcacaatttgaatcc

SEQ ID NO: 122 - DNA, 24 bp, Artificial Sequence
  source: /note=“Description of Artificial Sequence Synthetic primer”
gaagaaatacaaagcagttgctaa

SEQ ID NO: 123 - DNA, 21 bp, Artificial Sequence
  source: /note=“Description of Artificial Sequence Synthetic primer”
gcttaggaaggtgtggagagc

SEQ ID NO: 124 - DNA, 24 bp, Artificial Sequence
  source: /note=“Description of Artificial Sequence Synthetic primer”
ccactatttatgtttattgagtgc

SEQ ID NO: 125 - DNA, 21 bp, Artificial Sequence
  source: /note=“Description of Artificial Sequence Synthetic primer”
gagtcattttgtccaccaacc

SEQ ID NO: 126 - DNA, 23 bp, Artificial Sequence
  source: /note=“Description of Artificial Sequence Synthetic primer”
gctcatagttagaagtggcagca

SEQ ID NO: 127 - DNA, 23 bp, Artificial Sequence
  source: /note=“Description of Artificial Sequence Synthetic primer”
gcaatgataacaagaacacagca

SEQ ID NO: 128 - DNA, 20 bp, Artificial Sequence
  source: /note=“Description of Artificial Sequence Synthetic primer”
tggagccaaagggagtaata

SEQ ID NO: 129 - DNA, 21 bp, Artificial Sequence
  source: /note=“Description of Artificial Sequence Synthetic primer”
ttgctggcttacattcattcc

SEQ ID NO: 130 - DNA, 20 bp, Artificial Sequence
  source: /note=“Description of Artificial Sequence Synthetic primer”
tacagctcagccagttctgc

SEQ ID NO: 131 - DNA, 20 bp, Artificial Sequence
  source: /note=“Description of Artificial Sequence Synthetic primer”
gaaagggatgatggttccaa

SEQ ID NO: 132 - DNA, 20 bp, Artificial Sequence
  source: /note=“Description of Artificial Sequence Synthetic primer”
cccatgaacacattcacagc

SEQ ID NO: 133 - DNA, 20 bp, Artificial Sequence
  source: /note=“Description of Artificial Sequence Synthetic primer”
gtctgtccctgggccattat

SEQ ID NO: 134 - DNA, 21 bp, Artificial Sequence
  source: /note=“Description of Artificial Sequence Synthetic primer”
cacgattcagtaaatggcttg

SEQ ID NO: 135 - DNA, 23 bp, Artificial Sequence
  source: /note=“Description of Artificial Sequence Synthetic primer”
tggagacatgacactatgaattt

SEQ ID NO: 136 - DNA, 21 bp, Artificial Sequence
  source: /note=“Description of Artificial Sequence Synthetic primer”
ccatcctgggattaccaatct

SEQ ID NO: 137 - DNA, 24 bp, Artificial Sequence
  source: /note=“Description of Artificial Sequence Synthetic primer”
ttctgtgttctacaatgtctaggg

SEQ ID NO: 138 - DNA, 21 bp, Artificial Sequence
  source: /note=“Description of Artificial Sequence Synthetic primer”
tcatccatttgagttttccaa

SEQ ID NO: 139 - DNA, 20 bp, Artificial Sequence
  source: /note=“Description of Artificial Sequence Synthetic primer”
tatgagctgtggccaatgaa

SEQ ID NO: 140 - DNA, 21 bp, Artificial Sequence
  source: /note=“Description of Artificial Sequence Synthetic primer”
cctgaagtgtcccctagaagg

SEQ ID NO: 141 - DNA, 21 bp, Artificial Sequence
  source: /note=“Description of Artificial Sequence Synthetic primer”
tttgcagacaggttaagatgc

SEQ ID NO: 142 - DNA, 21 bp, Artificial Sequence
  source: /note=“Description of Artificial Sequence Synthetic primer”
tgcaccaagatgtgttctgtc

SEQ ID NO: 143 - DNA, 20 bp, Artificial Sequence
  source: /note=“Description of Artificial Sequence Synthetic primer”
cctacagtccagggggtctt

SEQ ID NO: 144 - DNA, 22 bp, Artificial Sequence
  source: /note=“Description of Artificial Sequence Synthetic primer”
tctagataaggagaatctggtg

SEQ ID NO: 145 - DNA, 20 bp, Artificial Sequence
  source: /note=“Description of Artificial Sequence Synthetic primer”
cggaattgagctaaccgtct

SEQ ID NO: 146 - DNA, 20 bp, Artificial Sequence
  source: /note=“Description of Artificial Sequence Synthetic primer”
cactggcctgaggctacttc

SEQ ID NO: 147 - DNA, 21 bp, Artificial Sequence
  source: /note=“Description of Artificial Sequence Synthetic primer”
aagtcctggatttcaccagag

SEQ ID NO: 148 - DNA, 21 bp, Artificial Sequence
  source: /note=“Description of Artificial Sequence Synthetic primer”
tcccaagatctgcactaaacg

SEQ ID NO: 149 - DNA, 20 bp, Artificial Sequence
  source: /note=“Description of Artificial Sequence Synthetic primer”
ccctccagagctaactgcat

SEQ ID NO: 150 - DNA, 23 bp, Artificial Sequence
  source: /note=“Description of Artificial Sequence Synthetic primer”
tggatttattcttcatgttgctt

SEQ ID NO: 151 - DNA, 24 bp, Artificial Sequence
  source: /note=“Description of Artificial Sequence Synthetic primer”
tttccaggagtataaaggagtgaa

SEQ ID NO: 152 - DNA, 24 bp, Artificial Sequence
  source: /note=“Description of Artificial Sequence Synthetic primer”
aaccaacacttaggaaaacaaatg

SEQ ID NO: 153 - DNA, 20 bp, Artificial Sequence
  source: /note=“Description of Artificial Sequence Synthetic primer”
gaagcttctgtcccttctgt

SEQ ID NO: 154 - DNA, 20 bp, Artificial Sequence
  source: /note=“Description of Artificial Sequence Synthetic primer”
cctgctgatttcatccttcc

SEQ ID NO: 155 - DNA, 23 bp, Artificial Sequence
  source: /note=“Description of Artificial Sequence Synthetic primer”
tcacatcagtaacctccttcttg

SEQ ID NO: 156 - DNA, 20 bp, Artificial Sequence
  source: /note=“Description of Artificial Sequence Synthetic primer”
tccagaagcctttcttcctg

SEQ ID NO: 157 - DNA, 24 bp, Artificial Sequence
  source: /note=“Description of Artificial Sequence Synthetic primer”
ggcataggaaccatattattgtca

SEQ ID NO: 158 - DNA, 24 bp, Artificial Sequence
  source: /note=“Description of Artificial Sequence Synthetic primer”
ccttctcaacatagttctaattcc

SEQ ID NO: 159 - DNA, 22 bp, Artificial Sequence
  source: /note=“Description of Artificial Sequence Synthetic primer”
ccacaagctcatcatctattcg

SEQ ID NO: 160 - DNA, 21 bp, Artificial Sequence
  source: /note=“Description of Artificial Sequence Synthetic primer”
tttctgaggctgataactgaa

SEQ ID NO: 161 - DNA, 22 bp, Artificial Sequence
  source: /note=“Description of Artificial Sequence Synthetic primer”
gaaggaacatcaaacaaggaaa

SEQ ID NO: 162 - DNA, 21 bp, Artificial Sequence
  source: /note=“Description of Artificial Sequence Synthetic primer”
tgcatatcacagtctccaagg

SEQ ID NO: 163 - DNA, 21 bp, Artificial Sequence
  source: /note=“Description of Artificial Sequence Synthetic primer”
gagcaggtagctacaatgaca

SEQ ID NO: 164 - DNA, 20 bp, Artificial Sequence
  source: /note=“Description of Artificial Sequence Synthetic primer”
tgccacccagatctcttttc

SEQ ID NO: 165 - DNA, 22 bp, Artificial Sequence
  source: /note=“Description of Artificial Sequence Synthetic primer”
cctgatctggaaactcatgaaa

SEQ ID NO: 166 - DNA, 21 bp, Artificial Sequence
  source: /note=“Description of Artificial Sequence Synthetic primer”
tggggatgtgggtaagttaat

SEQ ID NO: 167 - DNA, 20 bp, Artificial Sequence
  source: /note=“Description of Artificial Sequence Synthetic primer”
gcaactggtcttgttccaca

SEQ ID NO: 168 - DNA, 23 bp, Artificial Sequence
  source: /note=“Description of Artificial Sequence Synthetic primer”
gctaagccaatgtctacatcttc

SEQ ID NO: 169 - DNA, 21 bp, Artificial Sequence
  source: /note=“Description of Artificial Sequence Synthetic primer”
tggtgtgttagggatctggag

SEQ ID NO: 170 - DNA, 20 bp, Artificial Sequence
  source: /note=“Description of Artificial Sequence Synthetic primer”
tgacattggttattggcaga

SEQ ID NO: 171 - DNA, 23 bp, Artificial Sequence
  source: /note=“Description of Artificial Sequence Synthetic primer”
cgtattcattatccacagggact

SEQ ID NO: 172 - DNA, 20 bp, Artificial Sequence
  source: /note=“Description of Artificial Sequence Synthetic primer”
tgcagtgaaggattgcaaag

SEQ ID NO: 173 - DNA, 21 bp, Artificial Sequence
  source: /note=“Description of Artificial Sequence Synthetic primer”
cccttcctggacttcacatag

SEQ ID NO: 174 - DNA, 22 bp, Artificial Sequence
  source: /note=“Description of Artificial Sequence Synthetic primer”
gcatctagatctttaccattgc

SEQ ID NO: 175 - DNA, 22 bp, Artificial Sequence
  source: /note=“Description of Artificial Sequence Synthetic primer”
ggagaacatttagtgcctctgc

SEQ ID NO: 176 - DNA, 20 bp, Artificial Sequence
  source: /note=“Description of Artificial Sequence Synthetic primer”
acactcggaacgatctctgc

SEQ ID NO: 177 - DNA, 20 bp, Artificial Sequence
  source: /note=“Description of Artificial Sequence Synthetic primer”
aaacccacggaggtcatttt

SEQ ID NO: 178 - DNA, 22 bp, Artificial Sequence
  source: /note=“Description of Artificial Sequence Synthetic primer”
tgggtctcctatttctgtgtcc

SEQ ID NO: 179 - DNA, 23 bp, Artificial Sequence
  source: /note=“Description of Artificial Sequence Synthetic primer”
tgttaggactaccttatgcagtt

SEQ ID NO: 180 - DNA, 22 bp, Artificial Sequence
  source: /note=“Description of Artificial Sequence Synthetic primer”
tggtatgtctcctttgatcttt

SEQ ID NO: 181 - DNA, 20 bp, Artificial Sequence
  source: /note=“Description of Artificial Sequence Synthetic primer”
ctgagcgggagcttgtagat

SEQ ID NO: 182 - DNA, 20 bp, Artificial Sequence
  source: /note=“Description of Artificial Sequence Synthetic primer”
gctcctgacgaccaataacc

SEQ ID NO: 183 - DNA, 21 bp, Artificial Sequence
  source: /note=“Description of Artificial Sequence Synthetic primer”
ggaccactgtctagaccaagc

SEQ ID NO: 184 - DNA, 21 bp, Artificial Sequence
  source: /note=“Description of Artificial Sequence Synthetic primer”
tgtgtctggtgaggaagatga

SEQ ID NO: 185 - DNA, 20 bp, Artificial Sequence
  source: /note=“Description of Artificial Sequence Synthetic primer”
gggatgaaaccaaacctcct

SEQ ID NO: 186 - DNA, 22 bp, Artificial Sequence
  source: /note=“Description of Artificial Sequence Synthetic primer”
ttttaggaaacctcaccaggac

SEQ ID NO: 187 - DNA, 23 bp, Artificial Sequence
  source: /note=“Description of Artificial Sequence Synthetic primer”
tctctgttcgtgtctctgtcttg

SEQ ID NO: 188 - DNA, 21 bp, Artificial Sequence
  source: /note=“Description of Artificial Sequence Synthetic primer”
ttgagttggcctaaaaccaga

SEQ ID NO: 189 - DNA, 20 bp, Artificial Sequence
  source: /note=“Description of Artificial Sequence Synthetic primer”
cccgaccactaaaaggcata

SEQ ID NO: 190 - DNA, 24 bp, Artificial Sequence
  source: /note=“Description of Artificial Sequence Synthetic primer”
ttgcctctaaaatctagaatagcc

SEQ ID NO: 191 - DNA, 24 bp, Artificial Sequence
  source: /note=“Description of Artificial Sequence Synthetic primer”
tcttaggaatgactcacactggtc

SEQ ID NO: 192 - DNA, 23 bp, Artificial Sequence
  source: /note=“Description of Artificial Sequence Synthetic primer”
cactgaatattgaaaactaatgg

SEQ ID NO: 193 - DNA, 23 bp, Artificial Sequence
  source: /note=“Description of Artificial Sequence Synthetic primer”
gcatgttataattttacaagctc

SEQ ID NO: 194 - DNA, 23 bp, Artificial Sequence
  source: /note=“Description of Artificial Sequence Synthetic primer”
tcacacaggttaggatgtttgtg

SEQ ID NO: 195 - DNA, 20 bp, Artificial Sequence
  source: /note=“Description of Artificial Sequence Synthetic primer”
gcaccctaggagcaaactga

SEQ ID NO: 196 - DNA, 20 bp, Artificial Sequence
  source: /note=“Description of Artificial Sequence Synthetic primer”
gcagttgccttgaaaggagt

SEQ ID NO: 197 - DNA, 23 bp, Artificial Sequence
  source: /note=“Description of Artificial Sequence Synthetic primer”
gcaaataaaatgactctgggaac

SEQ ID NO: 198 - DNA, 23 bp, Artificial Sequence
  source: /note=“Description of Artificial Sequence Synthetic primer”
ggggttgagatacaacatcttca

SEQ ID NO: 199 - DNA, 20 bp, Artificial Sequence
  source: /note=“Description of Artificial Sequence Synthetic primer”
gattcttggggcatcaagtg

SEQ ID NO: 200 - DNA, 20 bp, Artificial Sequence
  source: /note=“Description of Artificial Sequence Synthetic primer”
ggacgtgggtgactatcagg

SEQ ID NO: 201 - DNA, 24 bp, Artificial Sequence
  source: /note=“Description of Artificial Sequence Synthetic primer”
tctagctcctaagttgattgattc

SEQ ID NO: 202 - DNA, 23 bp, Artificial Sequence
  source: /note=“Description of Artificial Sequence Synthetic primer”
tccattatagttcagtcttcaat

SEQ ID NO: 203 - DNA, 21 bp, Artificial Sequence
  source: /note=“Description of Artificial Sequence Synthetic primer”
caggagaaaagcagagaccaa

SEQ ID NO: 204 - DNA, 20 bp, Artificial Sequence
  source: /note=“Description of Artificial Sequence Synthetic primer”
agcgagagcaggctcataat

SEQ ID NO: 205 - DNA, 20 bp, Artificial Sequence
  source: /note=“Description of Artificial Sequence Synthetic primer”
tgacaagggattagggcaaa

SEQ ID NO: 206 - DNA, 24 bp, Artificial Sequence
  source: /note=“Description of Artificial Sequence Synthetic primer”
gaaactacctctgagtgttacaga

SEQ ID NO: 207 - DNA, 20 bp, Artificial Sequence
  source: /note=“Description of Artificial Sequence Synthetic primer”
gaatcctggacggtcagaaa

SEQ ID NO: 208 - DNA, 23 bp, Artificial Sequence
  source: /note=“Description of Artificial Sequence Synthetic primer”
tgaaaatgagtagtggacatctg

SEQ ID NO: 209 - DNA, 24 bp, Artificial Sequence
  source: /note=“Description of Artificial Sequence Synthetic primer”
aaaatgtgaagataagtgaacagc

SEQ ID NO: 210 - DNA, 24 bp, Artificial Sequence
  source: /note=“Description of Artificial Sequence Synthetic primer”
ccctaacttattcaacatcactgc

SEQ ID NO: 211 - DNA, 20 bp, Artificial Sequence
  source: /note=“Description of Artificial Sequence Synthetic primer”
acatattccaggagcatgac

SEQ ID NO: 212 - DNA, 20 bp, Artificial Sequence
  source: /note=“Description of Artificial Sequence Synthetic primer”
cattgagttcattggcctgt

SEQ ID NO: 213 - DNA, 20 bp, Artificial Sequence
  source: /note=“Description of Artificial Sequence Synthetic primer”
ctctcgtggtggattgaaca

SEQ ID NO: 214 - DNA, 24 bp, Artificial Sequence
  source: /note=“Description of Artificial Sequence Synthetic primer”
ccaacaagtactctgaaccaattt

SEQ ID NO: 215 - DNA, 20 bp, Artificial Sequence
  source: /note=“Description of Artificial Sequence Synthetic primer”
aaggagggcttagctagttg

SEQ ID NO: 216 - DNA, 23 bp, Artificial Sequence
  source: /note=“Description of Artificial Sequence Synthetic primer”
gctctttctcatcttaaggcttc

SEQ ID NO: 217 - DNA, 23 bp, Artificial Sequence
  source: /note=“Description of Artificial Sequence Synthetic primer”
gttaaaattactgttccagttgt

SEQ ID NO: 218 - DNA, 23 bp, Artificial Sequence
  source: /note=“Description of Artificial Sequence Synthetic primer”
caggcaaccaaataataacaaaa

SEQ ID NO: 219 - DNA, 21 bp, Artificial Sequence
  source: /note=“Description of Artificial Sequence Synthetic primer”
cccatttccatttaccgtttt

SEQ ID NO: 220 - DNA, 22 bp, Artificial Sequence
  source: /note=“Description of Artificial Sequence Synthetic primer”
ttgtatttacaatagccatcca

SEQ ID NO: 221 - DNA, 24 bp, Artificial Sequence
  source: /note=“Description of Artificial Sequence Synthetic primer”
tgaaagtatcaggaaaaatggatg

SEQ ID NO: 222 - DNA, 23 bp, Artificial Sequence
  source: /note=“Description of Artificial Sequence Synthetic primer”
agcagtcaaagtgaggatatgtt

SEQ ID NO: 223 - DNA, 23 bp, Artificial Sequence
  source: /note=“Description of Artificial Sequence Synthetic primer”
gcagtaacaaataaccccaacag

SEQ ID NO: 224 - DNA, 20 bp, Artificial Sequence
  source: /note=“Description of Artificial Sequence Synthetic primer”
accagcctttgttgttgagc

SEQ ID NO: 225 - DNA, 23 bp, Artificial Sequence
  source: /note=“Description of Artificial Sequence Synthetic primer”
gggttccagcaatattctacctt

SEQ ID NO: 226 - DNA, 22 bp, Artificial Sequence
  source: /note=“Description of Artificial Sequence Synthetic primer”
ggtaatgaagaaagacaaaaca

SEQ ID NO: 227 - DNA, 20 bp, Artificial Sequence
  source: /note=“Description of Artificial Sequence Synthetic primer”
tctaatgcctcaccaagcaa

SEQ ID NO: 228 - DNA, 20 bp, Artificial Sequence
  source: /note=“Description of Artificial Sequence Synthetic primer”
gcacagcagaaacccagatt

SEQ ID NO: 229 - DNA, 22 bp, Artificial Sequence
  source: /note=“Description of Artificial Sequence Synthetic primer”
cactagtccggcttgtgtaaaa

SEQ ID NO: 230 - DNA, 23 bp, Artificial Sequence
  source: /note=“Description of Artificial Sequence Synthetic primer”
tggtgattacagaataccaccag

SEQ ID NO: 231 - DNA, 20 bp, Artificial Sequence
  source: /note=“Description of Artificial Sequence Synthetic primer”
acaggagcggacaatgagag

SEQ ID NO: 232 - DNA, 20 bp, Artificial Sequence
  source: /note=“Description of Artificial Sequence Synthetic primer”
tgatgtgcatgtgtctcagc

SEQ ID NO: 233 - DNA, 20 bp, Artificial Sequence
  source: /note=“Description of Artificial Sequence Synthetic primer”
tggtcctctgcttccctaag

SEQ ID NO: 234 - DNA, 23 bp, Artificial Sequence
  source: /note=“Description of Artificial Sequence Synthetic primer”
catacatgaggtgactaccacca

SEQ ID NO: 235 - DNA, 21 bp, Artificial Sequence
  source: /note=“Description of Artificial Sequence Synthetic primer”
catcagattcccaacattgct

SEQ ID NO: 236 - DNA, 20 bp, Artificial Sequence
  source: /note=“Description of Artificial Sequence Synthetic primer”
agctcatcccaatcatcaca

SEQ ID NO: 237 - DNA, 20 bp, Artificial Sequence
  source: /note=“Description of Artificial Sequence Synthetic primer”
aagggccatgagggtacttt

SEQ ID NO: 238 - DNA, 24 bp, Artificial Sequence
  source: /note=“Description of Artificial Sequence Synthetic primer”
aacccaaacgtctaacaagataca

SEQ ID NO: 239 - DNA, 22 bp, Artificial Sequence
  source: /note=“Description of Artificial Sequence Synthetic primer”
catcgatagtattaggcccaca

SEQ ID NO: 240 - DNA, 24 bp, Artificial Sequence
  source: /note=“Description of Artificial Sequence Synthetic primer”
tgtgatttctttctataggaggtt

SEQ ID NO: 241 - DNA, 21 bp, Artificial Sequence
  source: /note=“Description of Artificial Sequence Synthetic primer”
ggaaggaaagctcttttggaa

SEQ ID NO: 242 - DNA, 22 bp, Artificial Sequence
  source: /note=“Description of Artificial Sequence Synthetic primer”
ttccagccctgaataacaactt

SEQ ID NO: 243 - DNA, 21 bp, Artificial Sequence
  source: /note=“Description of Artificial Sequence Synthetic primer”
tgatcattgctgtgatgtatt

SEQ ID NO: 244 - DNA, 23 bp, Artificial Sequence
  source: /note=“Description of Artificial Sequence Synthetic primer”
aggataccatgattttgtagtgc

SEQ ID NO: 245 - DNA, 20 bp, Artificial Sequence
  source: /note=“Description of Artificial Sequence Synthetic primer”
cttccctgcacatccttttg

SEQ ID NO: 246 - DNA, 24 bp, Artificial Sequence
  source: /note=“Description of Artificial Sequence Synthetic primer”
ctgtttaggaagagtcatgtaacc

SEQ ID NO: 247 - DNA, 22 bp, Artificial Sequence
  source: /note=“Description of Artificial Sequence Synthetic primer”
aactgttttgtcagctgctcat

SEQ ID NO: 248 - DNA, 22 bp, Artificial Sequence
  source: /note=“Description of Artificial Sequence Synthetic primer”
aaaagaccacttgattcagctt

SEQ ID NO: 249 - DNA, 21 bp, Artificial Sequence
  source: /note=“Description of Artificial Sequence Synthetic primer”
tgagcacacacatatggaagc

SEQ ID NO: 250 - DNA, 22 bp, Artificial Sequence
  source: /note=“Description of Artificial Sequence Synthetic primer”
tgcaatgtacatgtggagaatc

SEQ ID NO: 251 - DNA, 20 bp, Artificial Sequence
  source: /note=“Description of Artificial Sequence Synthetic primer”
cccgttctccattctggtta

SEQ ID NO: 252 - DNA, 20 bp, Artificial Sequence
  source: /note=“Description of Artificial Sequence Synthetic primer”
cccagggaagaaaattggta

SEQ ID NO: 253 - DNA, 22 bp, Artificial Sequence
  source: /note=“Description of Artificial Sequence Synthetic primer”
tgaaatagtgcttattgcatcg

SEQ ID NO: 254 - DNA, 20 bp, Artificial Sequence
  source: /note=“Description of Artificial Sequence Synthetic primer”
agccactccagcattcactt

SEQ ID NO: 255 - DNA, 22 bp, Artificial Sequence
  source: /note=“Description of Artificial Sequence Synthetic primer”
ccacatgtttctgagtgaagga

SEQ ID NO: 256 - DNA, 24 bp, Artificial Sequence
  source: /note=“Description of Artificial Sequence Synthetic primer”
ggagttacagttatcaaatgcaga

SEQ ID NO: 257 - DNA, 21 bp, Artificial Sequence
  source: /note=“Description of Artificial Sequence Synthetic primer”
ggaaagaagggagaatggtca

SEQ ID NO: 258 - DNA, 22 bp, Artificial Sequence
  source: /note=“Description of Artificial Sequence Synthetic primer”
ttgcatattctggacctcatct

SEQ ID NO: 259 - DNA, 22 bp, Artificial Sequence
  source: /note=“Description of Artificial Sequence Synthetic primer”
ggaggcaaagaagttagggagt

SEQ ID NO: 260 - DNA, 20 bp, Artificial Sequence
  source: /note=“Description of Artificial Sequence Synthetic primer”
ttttacctccctgccctagt

SEQ ID NO: 261 - DNA, 23 bp, Artificial Sequence
  source: /note=“Description of Artificial Sequence Synthetic primer”
aggaaatgtagtcaggtctagga

SEQ ID NO: 262 - DNA, 20 bp, Artificial Sequence
  source: /note=“Description of Artificial Sequence Synthetic primer”
gcagcttgaaaacagccagt

SEQ ID NO: 263 - DNA, 23 bp, Artificial Sequence
  source: /note=“Description of Artificial Sequence Synthetic primer”
catggtaagtatgctgttaaatc

SEQ ID NO: 264 - DNA, 21 bp, Artificial Sequence
  source: /note=“Description of Artificial Sequence Synthetic primer”
gctgagcagaaaacataagca

SEQ ID NO: 265 - DNA, 22 bp, Artificial Sequence
  source: /note=“Description of Artificial Sequence Synthetic primer”
caaacccacactgtgttagctg

SEQ ID NO: 266 - DNA, 24 bp, Artificial Sequence
  source: /note=“Description of Artificial Sequence Synthetic primer”
agctaatctttggtacttcaatct

SEQ ID NO: 267 - DNA, 21 bp, Artificial Sequence
  source: /note=“Description of Artificial Sequence Synthetic primer”
caagcatcttgctgaatttcc

SEQ ID NO: 268 - DNA, 23 bp, Artificial Sequence
  source: /note=“Description of Artificial Sequence Synthetic primer”
agtgcaaagtgaagataatgaca

SEQ ID NO: 269 - DNA, 20 bp, Artificial Sequence
  source: /note=“Description of Artificial Sequence Synthetic primer”
agtgtctgtcttccagttcc

SEQ ID NO: 270 - DNA, 24 bp, Artificial Sequence
  source: /note=“Description of Artificial Sequence Synthetic primer”
cattcatcccatcttctaacttca

SEQ ID NO: 271 - DNA, 22 bp, Artificial Sequence
  source: /note=“Description of Artificial Sequence Synthetic primer”
gcaaacatgtaaagtgtgagag

SEQ ID NO: 272 - DNA, 23 bp, Artificial Sequence
  source: /note=“Description of Artificial Sequence Synthetic primer”
gcagtcttctgtgattttatatt

SEQ ID NO: 273 - DNA, 22 bp, Artificial Sequence
  source: /note=“Description of Artificial Sequence Synthetic primer”
cagaaggaaggggtaagacaca

SEQ ID NO: 274 - DNA, 21 bp, Artificial Sequence
  source: /note=“Description of Artificial Sequence Synthetic primer”
tcccctcaggtaacttccatc

SEQ ID NO: 275 - DNA, 22 bp, Artificial Sequence
  source: /note=“Description of Artificial Sequence Synthetic primer”
gatttctgtgttgtgccacagt

SEQ ID NO: 276 - DNA, 23 bp, Artificial Sequence
  source: /note=“Description of Artificial Sequence Synthetic primer”
ttggtgtcttacatgtattgtga

SEQ ID NO: 277 - DNA, 21 bp, Artificial Sequence
  source: /note=“Description of Artificial Sequence Synthetic primer”
gctgtagcacatccaaaaacc

SEQ ID NO: 278 - DNA, 24 bp, Artificial Sequence
  source: /note=“Description of Artificial Sequence Synthetic primer”
gaactgaaaaaggaataaagtagg

SEQ ID NO: 279 - DNA, 22 bp, Artificial Sequence
  source: /note=“Description of Artificial Sequence Synthetic primer”
ggcataagcagatacagacagc

SEQ ID NO: 280 - DNA, 22 bp, Artificial Sequence
  source: /note=“Description of Artificial Sequence Synthetic primer”
tgaaacctataagccactgagc

SEQ ID NO: 281 - DNA, 22 bp, Artificial Sequence
  source: /note=“Description of Artificial Sequence Synthetic primer”
tccaaaaagacagctgaaagaa

SEQ ID NO: 282 - DNA, 20 bp, Artificial Sequence
  source: /note=“Description of Artificial Sequence Synthetic primer”
aagccatgcagtgggtatct

SEQ ID NO: 283 - DNA, 24 bp, Artificial Sequence
  source: /note=“Description of Artificial Sequence Synthetic primer”
tccatacaggaagatccattaaga

SEQ ID NO: 284 - DNA, 20 bp, Artificial Sequence
  source: /note=“Description of Artificial Sequence Synthetic primer”
gtgcagtttgggctacaaga

SEQ ID NO: 285 - DNA, 20 bp, Artificial Sequence
  source: /note=“Description of Artificial Sequence Synthetic primer”
tcacacatcattggtgaagg

SEQ ID NO: 286 - DNA, 24 bp, Artificial Sequence
  source: /note=“Description of Artificial Sequence Synthetic primer”
aagtgtcagagggttagtgattcc

SEQ ID NO: 287 - DNA, 21 bp, Artificial Sequence
  source: /note=“Description of Artificial Sequence Synthetic primer”
cacctaaagatttccccacaa

SEQ ID NO: 288 - DNA, 20 bp, Artificial Sequence
  source: /note=“Description of Artificial Sequence Synthetic primer”
gacttacggcctaacccttt

SEQ ID NO: 289 - DNA, 24 bp, Artificial Sequence
  source: /note=“Description of Artificial Sequence Synthetic primer”
gaacaagtatactagcaaaacgaa

SEQ ID NO: 290 - DNA, 24 bp, Artificial Sequence
  source: /note=“Description of Artificial Sequence Synthetic primer”
tttgtctaaagaatttgacagtgg

SEQ ID NO: 291 - DNA, 23 bp, Artificial Sequence
  source: /note=“Description of Artificial Sequence Synthetic primer”
tcttgagaagccttttcttacca

SEQ ID NO: 292 - DNA, 22 bp, Artificial Sequence
  source: /note=“Description of Artificial Sequence Synthetic primer”
gcatgagtgtgtgtctatgcag

SEQ ID NO: 293 - DNA, 24 bp, Artificial Sequence
  source: /note=“Description of Artificial Sequence Synthetic primer”
ttctggactctccactctatttca

SEQ ID NO: 294 - DNA, 24 bp, Artificial Sequence
  source: /note=“Description of Artificial Sequence Synthetic primer”
tggcataagatagacatattcacc

SEQ ID NO: 295 - DNA, 22 bp, Artificial Sequence
  source: /note=“Description of Artificial Sequence Synthetic primer”
gcatctatgtcaccaagcattt

SEQ ID NO: 296 - DNA, 20 bp, Artificial Sequence
  source: /note=“Description of Artificial Sequence Synthetic primer”
gccgttaagcactgagctgt

SEQ ID NO: 297 - DNA, 21 bp, Artificial Sequence
  source: /note=“Description of Artificial Sequence Synthetic primer”
tccactacttcttggagttca

SEQ ID NO: 298 - DNA, 22 bp, Artificial Sequence
  source: /note=“Description of Artificial Sequence Synthetic primer”
tcttgaatagcacccacaagag

SEQ ID NO: 299 - DNA, 20 bp, Artificial Sequence
  source: /note=“Description of Artificial Sequence Synthetic primer”
gacactactgtcctcaaacg

SEQ ID NO: 300 - DNA, 21 bp, Artificial Sequence
  source: /note=“Description of Artificial Sequence Synthetic primer”
gcccaaagaccaagttttaga

SEQ ID NO: 301 - DNA, 21 bp, Artificial Sequence
  source: /note=“Description of Artificial Sequence Synthetic primer”
cgtgtctgtgagctcctttct

SEQ ID NO: 302 - DNA, 22 bp, Artificial Sequence
  source: /note=“Description of Artificial Sequence Synthetic primer”
aggttgtgaaagacactgatgg

SEQ ID NO: 303 - DNA, 22 bp, Artificial Sequence
  source: /note=“Description of Artificial Sequence Synthetic primer”
tccaagctgtttctcatgtttg

SEQ ID NO: 304 - DNA, 20 bp, Artificial Sequence
  source: /note=“Description of Artificial Sequence Synthetic primer”
cagtgggctcacagtaatgg

SEQ ID NO: 305 - DNA, 22 bp, Artificial Sequence
  source: /note=“Description of Artificial Sequence Synthetic primer”
gcaattccagatatctctttat

SEQ ID NO: 306 - DNA, 21 bp, Artificial Sequence
  source: /note=“Description of Artificial Sequence Synthetic primer”
ttatctacccatgcttctctc

SEQ ID NO: 307 - DNA, 21 bp, Artificial Sequence
  source: /note=“Description of Artificial Sequence Synthetic primer”
aacagatcacttaccgctttg

SEQ ID NO: 308 - DNA, 22 bp, Artificial Sequence
  source: /note=“Description of Artificial Sequence Synthetic primer”
ccctacatgcattatctccttt

SEQ ID NO: 309 - DNA, 21 bp, Artificial Sequence
  source: /note=“Description of Artificial Sequence Synthetic primer”
tggtgccatcctagagttctg

SEQ ID NO: 310 - DNA, 21 bp, Artificial Sequence
  source: /note=“Description of Artificial Sequence Synthetic primer”
agtgtgcacttgctcatgact

SEQ ID NO: 311 - DNA, 20 bp, Artificial Sequence
  source: /note=“Description of Artificial Sequence Synthetic primer”
ccagggatttcatcttcacc

SEQ ID NO: 312 - DNA, 20 bp, Artificial Sequence
  source: /note=“Description of Artificial Sequence Synthetic primer”
atgtctatgccctgcctcat

SEQ ID NO: 313 - DNA, 23 bp, Artificial Sequence
  source: /note=“Description of Artificial Sequence Synthetic primer”
tgtagtcgaagcaatgagatgtg

SEQ ID NO: 314 - DNA, 24 bp, Artificial Sequence
  source: /note=“Description of Artificial Sequence Synthetic primer”
tttcactcccttctgtatttagcc

SEQ ID NO: 315 - DNA, 20 bp, Artificial Sequence
  source: /note=“Description of Artificial Sequence Synthetic primer”
aaatgctttgctgcatgtct

SEQ ID NO: 316 - DNA, 20 bp, Artificial Sequence
  source: /note=“Description of Artificial Sequence Synthetic primer”
tcaatggcaatttgaggaga

SEQ ID NO: 317 - DNA, 21 bp, Artificial Sequence
  source: /note=“Description of Artificial Sequence Synthetic primer”
tgaggaagtgacaagttcaga

SEQ ID NO: 318 - DNA, 21 bp, Artificial Sequence
  source: /note=“Description of Artificial Sequence Synthetic primer”
ttttctccccatctgttacta

SEQ ID NO: 319 - DNA, 22 bp, Artificial Sequence
  source: /note=“Description of Artificial Sequence Synthetic primer”
caattttacatccaacagaaga

SEQ ID NO: 320 - DNA, 23 bp, Artificial Sequence
  source: /note=“Description of Artificial Sequence Synthetic primer”
tgggattataaggaggtcaagaa

SEQ ID NO: 321 - DNA, 22 bp, Artificial Sequence
  source: /note=“Description of Artificial Sequence Synthetic primer”
tggtgagtttcttccctaggtt

SEQ ID NO: 322 - DNA, 22 bp, Artificial Sequence
  source: /note=“Description of Artificial Sequence Synthetic primer”
cttgacaccatagtggtcacct

SEQ ID NO: 323 - DNA, 24 bp, Artificial Sequence
  source: /note=“Description of Artificial Sequence Synthetic primer”
tttacttctgagctgaaggtactc

SEQ ID NO: 324 - DNA, 20 bp, Artificial Sequence
  source: /note=“Description of Artificial Sequence Synthetic primer”
cacgcaggcaatagtaggaa

SEQ ID NO: 325 - DNA, 20 bp, Artificial Sequence
  source: /note=“Description of Artificial Sequence Synthetic primer”
agcaccaaaggcaagttcaa

SEQ ID NO: 326 - DNA, 20 bp, Artificial Sequence
  source: /note=“Description of Artificial Sequence Synthetic primer”
ggatgccaagattgcaaata

SEQ ID NO: 327 - DNA, 24 bp, Artificial Sequence
  source: /note=“Description of Artificial Sequence Synthetic primer”
ttctttctacccaggtacttatca

SEQ ID NO: 328 - DNA, 20 bp, Artificial Sequence
  source: /note=“Description of Artificial Sequence Synthetic primer”
tttcaagatgcaaaggcttg

SEQ ID NO: 329 - DNA, 20 bp, Artificial Sequence
  source: /note=“Description of Artificial Sequence Synthetic primer”
cgaaatccataggacctaca

SEQ ID NO: 330 - DNA, 22 bp, Artificial Sequence
  source: /note=“Description of Artificial Sequence Synthetic primer”
agctacactatttccatgtgac

SEQ ID NO: 331 - DNA, 21 bp, Artificial Sequence
  source: /note=“Description of Artificial Sequence Synthetic primer”
cgtcggttgttttatcattgc

SEQ ID NO: 332 - DNA, 23 bp, Artificial Sequence
  source: /note=“Description of Artificial Sequence Synthetic primer”
ggacaggttgtgcataactaaga

SEQ ID NO: 333 - DNA, 24 bp, Artificial Sequence
  source: /note=“Description of Artificial Sequence Synthetic primer”
cctcacttaaggagaacagttaga

SEQ ID NO: 334 - DNA, 23 bp, Artificial Sequence
  source: /note=“Description of Artificial Sequence Synthetic primer”
tgctaatcatcccttattattgc

SEQ ID NO: 335 - DNA, 24 bp, Artificial Sequence
  source: /note=“Description of Artificial Sequence Synthetic primer”
tgacctactagacatcaagcctta

SEQ ID NO: 336 - DNA, 22 bp, Artificial Sequence
  source: /note=“Description of Artificial Sequence Synthetic primer”
tgccagtaacttaatccatagc

SEQ ID NO: 337 - DNA, 21 bp, Artificial Sequence
  source: /note=“Description of Artificial Sequence Synthetic primer”
ccagacaggcacatacagtca

SEQ ID NO: 338 - DNA, 23 bp, Artificial Sequence
  source: /note=“Description of Artificial Sequence Synthetic primer”
gggaactgagtatctctgtgtga

SEQ ID NO: 339 - DNA, 21 bp, Artificial Sequence
  source: /note=“Description of Artificial Sequence Synthetic primer”
gaggtcgaagttgtaggcttg

SEQ ID NO: 340 - DNA, 23 bp, Artificial Sequence
  source: /note=“Description of Artificial Sequence Synthetic primer”
tcaacttagttacaggtcacaca

SEQ ID NO: 341 - DNA, 23 bp, Artificial Sequence
  source: /note=“Description of Artificial Sequence Synthetic primer”
tcaatttttgttgtggtttacct

SEQ ID NO: 342 - DNA, 22 bp, Artificial Sequence
  source: /note=“Description of Artificial Sequence Synthetic primer”
aggttttcctaataagactgct

SEQ ID NO: 343 - DNA, 22 bp, Artificial Sequence
  source: /note=“Description of Artificial Sequence Synthetic primer”
tcagagtaggaatgaacaattt

SEQ ID NO: 344 - DNA, 20 bp, Artificial Sequence
  source: /note=“Description of Artificial Sequence Synthetic primer”
ctcagggcctaaacttgcac

SEQ ID NO: 345 - DNA, 20 bp, Artificial Sequence
  source: /note=“Description of Artificial Sequence Synthetic primer”
gcactcatgtgagtttgcac

SEQ ID NO: 346 - DNA, 24 bp, Artificial Sequence
  source: /note=“Description of Artificial Sequence Synthetic primer”
cacagtgaagtatgtataaattgc

SEQ ID NO: 347 - DNA, 20 bp, Artificial Sequence
  source: /note=“Description of Artificial Sequence Synthetic primer”
gcctagctgtgcgattcttc

SEQ ID NO: 348 - DNA, 20 bp, Artificial Sequence
  source: /note=“Description of Artificial Sequence Synthetic primer”
tgataccagttgatgccaca

SEQ ID NO: 349 - DNA, 23 bp, Artificial Sequence
  source: /note=“Description of Artificial Sequence Synthetic primer”
tgactgaactcaattcaaacagc

SEQ ID NO: 350 - DNA, 22 bp, Artificial Sequence
  source: /note=“Description of Artificial Sequence Synthetic primer”
tggcatctagggtataggaaga

SEQ ID NO: 351 - DNA, 20 bp, Artificial Sequence
  source: /note=“Description of Artificial Sequence Synthetic primer”
ggccaccatctcctgttcta

SEQ ID NO: 352 - DNA, 22 bp, Artificial Sequence
  source: /note=“Description of Artificial Sequence Synthetic primer”
ccttgtttgtctgtatctgagc

SEQ ID NO: 353 - DNA, 20 bp, Artificial Sequence
  source: /note=“Description of Artificial Sequence Synthetic primer”
ccaactctgattgtgcgact

SEQ ID NO: 354 - DNA, 20 bp, Artificial Sequence
  source: /note=“Description of Artificial Sequence Synthetic primer”
gctccaagccatagatccag

SEQ ID NO: 355 - DNA, 20 bp, Artificial Sequence
  source: /note=“Description of Artificial Sequence Synthetic primer”
ggtgtgtgtatgaggcttgg

SEQ ID NO: 356 - DNA, 19 bp, Artificial Sequence
  source: /note=“Description of Artificial Sequence Synthetic primer”
aaccgccagcatagcttct

SEQ ID NO: 357 - DNA, 20 bp, Artificial Sequence
  source: /note=“Description of Artificial Sequence Synthetic primer”
ggtaggaaggggttgtcgtt

SEQ ID NO: 358 - DNA, 24 bp, Artificial Sequence
  source: /note=“Description of Artificial Sequence Synthetic primer”
tttctttctacttctcatcactct

SEQ ID NO: 359 - DNA, 23 bp, Artificial Sequence
  source: /note=“Description of Artificial Sequence Synthetic primer”
ggacatcagcactaactgaagtg

SEQ ID NO: 360 - DNA, 22 bp, Artificial Sequence
  source: /note=“Description of Artificial Sequence Synthetic primer”
ttctcttgtgtgaaccatcctc

SEQ ID NO: 361 - DNA, 20 bp, Artificial Sequence
  source: /note=“Description of Artificial Sequence Synthetic primer”
gccagcgtgtaagacacaag

SEQ ID NO: 362 - DNA, 23 bp, Artificial Sequence
  source: /note=“Description of Artificial Sequence Synthetic primer”
tggcatttgtttacagacttatc

SEQ ID NO: 363 - DNA, 22 bp, Artificial Sequence
  source: /note=“Description of Artificial Sequence Synthetic primer”
tcctccacattggtaattaggg

SEQ ID NO: 364 - DNA, 20 bp, Artificial Sequence
  source: /note=“Description of Artificial Sequence Synthetic primer”
ggtgtccccctcaaattgta

SEQ ID NO: 365 - DNA, 22 bp, Artificial Sequence
  source: /note=“Description of Artificial Sequence Synthetic primer”
caagtttgtacctcagctttca

SEQ ID NO: 366 - DNA, 21 bp, Artificial Sequence
  source: /note=“Description of Artificial Sequence Synthetic primer”
tgagatactgttgtcctctgc

SEQ ID NO: 367 - DNA, 21 bp, Artificial Sequence
  source: /note=“Description of Artificial Sequence Synthetic primer”
ttcccttcttatgtaatctcc

SEQ ID NO: 368 - DNA, 24 bp, Artificial Sequence
  source: /note=“Description of Artificial Sequence Synthetic primer”
gagggttactgaactaggataatg

SEQ ID NO: 369 - DNA, 22 bp, Artificial Sequence
  source: /note=“Description of Artificial Sequence Synthetic primer”
tcctgagagcatggtaagatgt

SEQ ID NO: 370 - DNA, 20 bp, Artificial Sequence
  source: /note=“Description of Artificial Sequence Synthetic primer”
tgcagggcattctatgtgaa

SEQ ID NO: 371 - DNA, 20 bp, Artificial Sequence
  source: /note=“Description of Artificial Sequence Synthetic primer”
tacagctgagcaataacgtg

SEQ ID NO: 372 - DNA, 20 bp, Artificial Sequence
  source: /note=“Description of Artificial Sequence Synthetic primer”
tggctggccaaatctttcta

SEQ ID NO: 373 - DNA, 23 bp, Artificial Sequence
  source: /note=“Description of Artificial Sequence Synthetic primer”
aaactataaaaggacctaggaaa

SEQ ID NO: 374 - DNA, 24 bp, Artificial Sequence
  source: /note=“Description of Artificial Sequence Synthetic primer”
aagtctagtgaatttcttgttagg

SEQ ID NO: 375 - DNA, 23 bp, Artificial Sequence
  source: /note=“Description of Artificial Sequence Synthetic primer”
cttaatgattttgtaatgtcagg

SEQ ID NO: 376 - DNA, 20 bp, Artificial Sequence
  source: /note=“Description of Artificial Sequence Synthetic primer”
atttgagaggttgccagagc

SEQ ID NO: 377 - DNA, 20 bp, Artificial Sequence
  source: /note=“Description of Artificial Sequence Synthetic primer”
gaggttctcattccctcacc

SEQ ID NO: 378 - DNA, 20 bp, Artificial Sequence
  source: /note=“Description of Artificial Sequence Synthetic primer”
agaggggctcacctgagagt

SEQ ID NO: 379 - DNA, 23 bp, Artificial Sequence
  source: /note=“Description of Artificial Sequence Synthetic primer”
cacactagtgggtcctgattaga

SEQ ID NO: 380 - DNA, 20 bp, Artificial Sequence
  source: /note=“Description of Artificial Sequence Synthetic primer”
ttgcggtttcctcattcttc

SEQ ID NO: 381 - DNA, 20 bp, Artificial Sequence
  source: /note=“Description of Artificial Sequence Synthetic primer”
cgtgatgggtaggtcagtcc

SEQ ID NO: 382 - DNA, 20 bp, Artificial Sequence
  source: /note=“Description of Artificial Sequence Synthetic primer”
cgcctctggggataactaaa

SEQ ID NO: 383 - DNA, 20 bp, Artificial Sequence
  source: /note=“Description of Artificial Sequence Synthetic primer”
ggaattgccactctggagaa

SEQ ID NO: 384 - DNA, 20 bp, Artificial Sequence
  source: /note=“Description of Artificial Sequence Synthetic primer”
agtggtccccaacaacttga

SEQ ID NO: 385 - DNA, 21 bp, Artificial Sequence
  source: /note=“Description of Artificial Sequence Synthetic primer”
ataacaatgtctagcaacagg

SEQ ID NO: 386 - DNA, 23 bp, Artificial Sequence
  source: /note=“Description of Artificial Sequence Synthetic primer”
gatcaacacttcaaaattatggt

SEQ ID NO: 387 - DNA, 23 bp, Artificial Sequence
  source: /note=“Description of Artificial Sequence Synthetic primer”
tcagataaaacaattccagttac

SEQ ID NO: 388 - DNA, 20 bp, Artificial Sequence
  source: /note=“Description of Artificial Sequence Synthetic primer”
acccacagaggaaagccttg

SEQ ID NO: 389 - DNA, 23 bp, Artificial Sequence
  source: /note=“Description of Artificial Sequence Synthetic primer”
cagcatatattaccttttctttg

SEQ ID NO: 390 - DNA, 21 bp, Artificial Sequence
  source: /note=“Description of Artificial Sequence Synthetic primer”
tgtgcccagaaagttttagca

SEQ ID NO: 391 - DNA, 22 bp, Artificial Sequence
  source: /note=“Description of Artificial Sequence Synthetic primer”
tcaactgacactggtgtttctc

SEQ ID NO: 392 - DNA, 20 bp, Artificial Sequence
  source: /note=“Description of Artificial Sequence Synthetic primer”
gtgaagggaggacaaaatcg

SEQ ID NO: 393 - DNA, 20 bp, Artificial Sequence
  source: /note=“Description of Artificial Sequence Synthetic primer”
caagtgatctgatggggtga

SEQ ID NO: 394 - DNA, 22 bp, Artificial Sequence
  source: /note=“Description of Artificial Sequence Synthetic primer”
tgctgagtttgagaaacttggt

SEQ ID NO: 395 - DNA, 20 bp, Artificial Sequence
  source: /note=“Description of Artificial Sequence Synthetic primer”
gtaggacttagggcgctcat

SEQ ID NO: 396 - DNA, 20 bp, Artificial Sequence
  source: /note=“Description of Artificial Sequence Synthetic primer”
gcattactgccgagggatct

SEQ ID NO: 397 - DNA, 23 bp, Artificial Sequence
  source: /note=“Description of Artificial Sequence Synthetic primer”
tgacaaagcctagagtgaactga

SEQ ID NO: 398 - DNA, 24 bp, Artificial Sequence
  source: /note=“Description of Artificial Sequence Synthetic primer”
tcctagagtactcctctttgtcca

SEQ ID NO: 399 - DNA, 21 bp, Artificial Sequence
  source: /note=“Description of Artificial Sequence Synthetic primer”
gtacagagtccctgtctcaca

SEQ ID NO: 400 - DNA, 23 bp, Artificial Sequence
  source: /note=“Description of Artificial Sequence Synthetic primer”
catgatctgtctctctcactgaa

SEQ ID NO: 401 - DNA, 20 bp, Artificial Sequence
  source: /note=“Description of Artificial Sequence Synthetic primer”
gcattttctcacagccacag

SEQ ID NO: 402 - DNA, 21 bp, Artificial Sequence
  source: /note=“Description of Artificial Sequence Synthetic primer”
tggcctaaaaattcaccactg

SEQ ID NO: 403 - DNA, 20 bp, Artificial Sequence
  source: /note=“Description of Artificial Sequence Synthetic primer”
aacatttgcacattatcagc

SEQ ID NO: 404 - DNA, 22 bp, Artificial Sequence
  source: /note=“Description of Artificial Sequence Synthetic primer”
gcaaggatcagtcagactacga

SEQ ID NO: 405 - DNA, 22 bp, Artificial Sequence
  source: /note=“Description of Artificial Sequence Synthetic primer”
tgtccatcaatctcaaaagtcg

SEQ ID NO: 406 - DNA, 24 bp, Artificial Sequence
  source: /note=“Description of Artificial Sequence Synthetic primer”
ctgatttctaccagttacttacca

SEQ ID NO: 407 - DNA, 20 bp, Artificial Sequence
  source: /note=“Description of Artificial Sequence Synthetic primer”
gcatgagccaccctaaatct

SEQ ID NO: 408 - DNA, 20 bp, Artificial Sequence
  source: /note=“Description of Artificial Sequence Synthetic primer”
tgcagaccatgaggaatgtt

SEQ ID NO: 409 - DNA, 23 bp, Artificial Sequence
  source: /note=“Description of Artificial Sequence Synthetic primer”
aggattccttatacactgacctc

SEQ ID NO: 410 - DNA, 21 bp, Artificial Sequence
  source: /note=“Description of Artificial Sequence Synthetic primer”
accaaataatggtctactcct

SEQ ID NO: 411 - DNA, 22 bp, Artificial Sequence
  source: /note=“Description of Artificial Sequence Synthetic primer”
aagacattctctgcctttctca

SEQ ID NO: 412 - DNA, 23 bp, Artificial Sequence
  source: /note=“Description of Artificial Sequence Synthetic primer”
ggctctactatggggaaaattca

SEQ ID NO: 413 - DNA, 21 bp, Artificial Sequence
  source: /note=“Description of Artificial Sequence Synthetic primer”
gcaaatcactaggaaagctca

SEQ ID NO: 414 - DNA, 23 bp, Artificial Sequence
  source: /note=“Description of Artificial Sequence Synthetic primer”
gaggttcactctatttctgttcc

SEQ ID NO: 415 - DNA, 20 bp, Artificial Sequence
  source: /note=“Description of Artificial Sequence Synthetic primer”
ctagaaacggctgccaggta

SEQ ID NO: 416 - DNA, 20 bp, Artificial Sequence
  source: /note=“Description of Artificial Sequence Synthetic primer”
ccctgcacttgtaccagctt

SEQ ID NO: 417 - DNA, 24 bp, Artificial Sequence
  source: /note=“Description of Artificial Sequence Synthetic primer”
aggacattcttttgtgtattcaag

SEQ ID NO: 418 - DNA, 20 bp, Artificial Sequence
  source: /note=“Description of Artificial Sequence Synthetic primer”
atcccatataggcacttgct

SEQ ID NO: 419 - DNA, 24 bp, Artificial Sequence
  source: /note=“Description of Artificial Sequence Synthetic primer”
caaataatcaccccaatacaatca

SEQ ID NO: 420 - DNA, 20 bp, Artificial Sequence
  source: /note=“Description of Artificial Sequence Synthetic primer”
gctttcagtgccctcatctc

SEQ ID NO: 421 - DNA, 24 bp, Artificial Sequence
  source: /note=“Description of Artificial Sequence Synthetic primer”
aagatgatcaaagttttgagagca

SEQ ID NO: 422 - DNA, 24 bp, Artificial Sequence
  source: /note=“Description of Artificial Sequence Synthetic primer”
cactcctaaagaacaagatgtcaa

SEQ ID NO: 423 - DNA, 21 bp, Artificial Sequence
  source: /note=“Description of Artificial Sequence Synthetic primer”
gacaagaactgaaggcaaagg

SEQ ID NO: 424 - DNA, 21 bp, Artificial Sequence
  source: /note=“Description of Artificial Sequence Synthetic primer”
gggaggaacagaacaaccttc

SEQ ID NO: 425 - DNA, 20 bp, Artificial Sequence
  source: /note=“Description of Artificial Sequence Synthetic primer”
cccttgcaatacccagcata

SEQ ID NO: 426 - DNA, 21 bp, Artificial Sequence
  source: /note=“Description of Artificial Sequence Synthetic primer”
agttatctgagttggcttacc

SEQ ID NO: 427 - DNA, 20 bp, Artificial Sequence
  source: /note=“Description of Artificial Sequence Synthetic primer”
tcgcagtcttttgcatcatt

SEQ ID NO: 428 - DNA, 23 bp, Artificial Sequence
  source: /note=“Description of Artificial Sequence Synthetic primer”
tccaatagctaccttcaccagaa

SEQ ID NO: 429 - DNA, 23 bp, Artificial Sequence
  source: /note=“Description of Artificial Sequence Synthetic primer”
tggaaaaacacaggcatattctc

SEQ ID NO: 430 - DNA, 22 bp, Artificial Sequence
  source: /note=“Description of Artificial Sequence Synthetic primer”
ccaaaagcatctaaaaacagga

SEQ ID NO: 431 - DNA, 24 bp, Artificial Sequence
  source: /note=“Description of Artificial Sequence Synthetic primer”
caaatatactgattctgtggcaaa

SEQ ID NO: 432 - DNA, 22 bp, Artificial Sequence
  source: /note=“Description of Artificial Sequence Synthetic primer”
tgatgcattgagattttgatga

SEQ ID NO: 433 - DNA, 20 bp, Artificial Sequence
  source: /note=“Description of Artificial Sequence Synthetic primer”
tagcctttggataacagtcc

SEQ ID NO: 434 - DNA, 21 bp, Artificial Sequence
  source: /note=“Description of Artificial Sequence Synthetic primer”
gagggaggaaatggtcaactt

SEQ ID NO: 435 - DNA, 22 bp, Artificial Sequence
  source: /note=“Description of Artificial Sequence Synthetic primer”
aggcaaagaactagacaactct

SEQ ID NO: 436 - DNA, 20 bp, Artificial Sequence
  source: /note=“Description of Artificial Sequence Synthetic primer”
agacgtgctgggttcctaga

SEQ ID NO: 437 - DNA, 23 bp, Artificial Sequence
  source: /note=“Description of Artificial Sequence Synthetic primer”
ggcatgaagatgttaacctacca

SEQ ID NO: 438 - DNA, 24 bp, Artificial Sequence
  source: /note=“Description of Artificial Sequence Synthetic primer”
ttgtctggtcttcatcaagtctct

SEQ ID NO: 439 - DNA, 21 bp, Artificial Sequence
  source: /note=“Description of Artificial Sequence Synthetic primer”
ttgccatgcagcagtacttag

SEQ ID NO: 440 - DNA, 23 bp, Artificial Sequence
  source: /note=“Description of Artificial Sequence Synthetic primer”
tgacttttcattgctagtatcca

SEQ ID NO: 441 - DNA, 21 bp, Artificial Sequence
  source: /note=“Description of Artificial Sequence Synthetic primer”
gcaacaagaacaggaaccaag

SEQ ID NO: 442 - DNA, 22 bp, Artificial Sequence
  source: /note=“Description of Artificial Sequence Synthetic primer”
tgttttgacattgtcctgtgtg

SEQ ID NO: 443 - DNA, 24 bp, Artificial Sequence
  source: /note=“Description of Artificial Sequence Synthetic primer”
cagtgaggtgtgatgtataaagag

SEQ ID NO: 444 - DNA, 24 bp, Artificial Sequence
  source: /note=“Description of Artificial Sequence Synthetic primer”
gagaacacatattcattcctctcc

SEQ ID NO: 445 - DNA, 20 bp, Artificial Sequence
  source: /note=“Description of Artificial Sequence Synthetic primer”
gtggggtccagcagtaaatc

SEQ ID NO: 446 - DNA, 23 bp, Artificial Sequence
  source: /note=“Description of Artificial Sequence Synthetic primer”
gaacttctcacatcacctcaagc

SEQ ID NO: 447 - DNA, 24 bp, Artificial Sequence
  source: /note=“Description of Artificial Sequence Synthetic primer”
tctattaaccctaatcaatctcct

SEQ ID NO: 448 - DNA, 21 bp, Artificial Sequence
  source: /note=“Description of Artificial Sequence Synthetic primer”
ttgctaaatttcaggcacctc

SEQ ID NO: 449 - DNA, 20 bp, Artificial Sequence
  source: /note=“Description of Artificial Sequence Synthetic primer”
cctttgactctggcctcatc

SEQ ID NO: 450 - DNA, 22 bp, Artificial Sequence
  source: /note=“Description of Artificial Sequence Synthetic primer”
agtgaataaccagccttagttg

SEQ ID NO: 451 - DNA, 23 bp, Artificial Sequence
  source: /note=“Description of Artificial Sequence Synthetic primer”
aaataaggacatctggaaaacaa

SEQ ID NO: 452 - DNA, 20 bp, Artificial Sequence
  source: /note=“Description of Artificial Sequence Synthetic primer”
gtgccagctacaaacaatgg

SEQ ID NO: 453 - DNA, 20 bp, Artificial Sequence
  source: /note=“Description of Artificial Sequence Synthetic primer”
gtgcctcatcaaaatgcaac

SEQ ID NO: 454 - DNA, 20 bp, Artificial Sequence
  source: /note=“Description of Artificial Sequence Synthetic primer”
acacagatgacttcagctgg

SEQ ID NO: 455 - DNA, 20 bp, Artificial Sequence
  source: /note=“Description of Artificial Sequence Synthetic primer”
aactcaaacctaagtgcccc

SEQ ID NO: 456 - DNA, 20 bp, Artificial Sequence
  source: /note=“Description of Artificial Sequence Synthetic primer”
ggaatggaatagtgtgtggg

SEQ ID NO: 457 - DNA, 19 bp, Artificial Sequence
  source: /note=“Description of Artificial Sequence Synthetic primer”
acactggtctcaagctccc

SEQ ID NO: 458 - DNA, 20 bp, Artificial Sequence
  source: /note=“Description of Artificial Sequence Synthetic primer”
cacacctgtaattctagccc

SEQ ID NO: 459 - DNA, 20 bp, Artificial Sequence
  source: /note=“Description of Artificial Sequence Synthetic primer”
agaaggaaggatcagagaag

SEQ ID NO: 460 - DNA, 19 bp, Artificial Sequence
  source: /note=“Description of Artificial Sequence Synthetic primer”
agctttcctccccacactg

SEQ ID NO: 461 - DNA, 20 bp, Artificial Sequence
  source: /note=“Description of Artificial Sequence Synthetic primer”
gctgtgtggagccctataaa

SEQ ID NO: 462 - DNA, 20 bp, Artificial Sequence
  source: /note=“Description of Artificial Sequence Synthetic primer”
gaatgaaatggagtttgcag

SEQ ID NO: 463 - DNA, 20 bp, Artificial Sequence
  source: /note=“Description of Artificial Sequence Synthetic primer”
cctcagccaccacttgttag

SEQ ID NO: 464 - DNA, 20 bp, Artificial Sequence
  source: /note=“Description of Artificial Sequence Synthetic primer”
gtgttggtcagacagaaagg

SEQ ID NO: 465 - DNA, 20 bp, Artificial Sequence
  source: /note=“Description of Artificial Sequence Synthetic primer”
gccaattaccccataattag

SEQ ID NO: 466 - DNA, 20 bp, Artificial Sequence
  source: /note=“Description of Artificial Sequence Synthetic primer”
atgcacacttacacacgcac

SEQ ID NO: 467 - DNA, 20 bp, Artificial Sequence
  source: /note=“Description of Artificial Sequence Synthetic primer”
aaggaagtaaaggtatgtgc

SEQ ID NO: 468 - DNA, 20 bp, Artificial Sequence
  source: /note=“Description of Artificial Sequence Synthetic primer”
aggctaactctaacatcctg

SEQ ID NO: 469 - DNA, 20 bp, Artificial Sequence
  source: /note=“Description of Artificial Sequence Synthetic primer”
aagagtgtctcctccctctg

SEQ ID NO: 470 - DNA, 20 bp, Artificial Sequence
  source: /note=“Description of Artificial Sequence Synthetic primer”
aactggaggctgtgttagac

SEQ ID NO: 471 - DNA, 20 bp, Artificial Sequence
  source: /note=“Description of Artificial Sequence Synthetic primer”
cgctcttttctgactagtcc

SEQ ID NO: 472 - DNA, 20 bp, Artificial Sequence
  source: /note=“Description of Artificial Sequence Synthetic primer”
ttgcagcagtcacaggaaac

SEQ ID NO: 473 - DNA, 20 bp, Artificial Sequence
  source: /note=“Description of Artificial Sequence Synthetic primer”
ctctctgtgcacaaaaaacc

SEQ ID NO: 474 - DNA, 20 bp, Artificial Sequence
  source: /note=“Description of Artificial Sequence Synthetic primer”
ggaagacactgccttcaaac

SEQ ID NO: 475 - DNA, 20 bp, Artificial Sequence
  source: /note=“Description of Artificial Sequence Synthetic primer”
aaaaaccccaggctccattg

SEQ ID NO: 476 - DNA, 20 bp, Artificial Sequence
  source: /note=“Description of Artificial Sequence Synthetic primer”
atgtccagctgcttcttttc

SEQ ID NO: 477 - DNA, 20 bp, Artificial Sequence
  source: /note=“Description of Artificial Sequence Synthetic primer”
tccaagtcagaagctatggg

SEQ ID NO: 478 - DNA, 20 bp, Artificial Sequence
  source: /note=“Description of Artificial Sequence Synthetic primer”
agtctgcagacctaacatgg

SEQ ID NO: 479 - DNA, 20 bp, Artificial Sequence
  source: /note=“Description of Artificial Sequence Synthetic primer”
atggcttgtacttcctcctc

SEQ ID NO: 480 - DNA, 20 bp, Artificial Sequence
  source: /note=“Description of Artificial Sequence Synthetic primer”
ttcggtggaatagcagcaag

SEQ ID NO: 481 - DNA, 19 bp, Artificial Sequence
  source: /note=“Description of Artificial Sequence Synthetic primer”
cataatctcagggctacat

SEQ ID NO: 482 - DNA, 20 bp, Artificial Sequence
  source: /note=“Description of Artificial Sequence Synthetic primer”
ttcacctggccttgagggtc

SEQ ID NO: 483 - DNA, 20 bp, Artificial Sequence
  source: /note=“Description of Artificial Sequence Synthetic primer”
gtttattgatgaactggtgc

SEQ ID NO: 484 - DNA, 19 bp, Artificial Sequence
  source: /note=“Description of Artificial Sequence Synthetic primer”
gggcagagtgatatcacag

SEQ ID NO: 485 - DNA, 20 bp, Artificial Sequence
  source: /note=“Description of Artificial Sequence Synthetic primer”
actggcaagtccaggtcttc

SEQ ID NO: 486 - DNA, 20 bp, Artificial Sequence
  source: /note=“Description of Artificial Sequence Synthetic primer”
aaggctcagggcagaagcac

SEQ ID NO: 487 - DNA, 19 bp, Artificial Sequence
  source: /note=“Description of Artificial Sequence Synthetic primer”
tcctcatccggtgtggcaa

SEQ ID NO: 488 - DNA, 21 bp, Artificial Sequence
  source: /note=“Description of Artificial Sequence Synthetic primer”
cagcaaagagagagaggttcc

SEQ ID NO: 489 - DNA, 20 bp, Artificial Sequence
  source: /note=“Description of Artificial Sequence Synthetic primer”
agtatgccatcatgaaagcc

SEQ ID NO: 490 - DNA, 20 bp, Artificial Sequence
  source: /note=“Description of Artificial Sequence Synthetic primer”
cttctttgactaaggctgac

SEQ ID NO: 491 - DNA, 20 bp, Artificial Sequence
  source: /note=“Description of Artificial Sequence Synthetic primer”
ctctgcctattctcctcttc

SEQ ID NO: 492 - DNA, 20 bp, Artificial Sequence
  source: /note=“Description of Artificial Sequence Synthetic primer”
tagacctcaaggcctagagc

SEQ ID NO: 493 - DNA, 20 bp, Artificial Sequence
  source: /note=“Description of Artificial Sequence Synthetic primer”
agtaagagctcccttggttg

SEQ ID NO: 494 - DNA, 20 bp, Artificial Sequence
  source: /note=“Description of Artificial Sequence Synthetic primer”
gctcataacaatctctcccc

SEQ ID NO: 495 - DNA, 20 bp, Artificial Sequence
  source: /note=“Description of Artificial Sequence Synthetic primer”
tcccctctaccccttgaagc

SEQ ID NO: 496 - DNA, 20 bp, Artificial Sequence
  source: /note=“Description of Artificial Sequence Synthetic primer”
cagcactgatgacatctggg

SEQ ID NO: 497 - DNA, 20 bp, Artificial Sequence
  source: /note=“Description of Artificial Sequence Synthetic primer”
aagaacacaggcctggttgg

SEQ ID NO: 498 - DNA, 20 bp, Artificial Sequence
  source: /note=“Description of Artificial Sequence Synthetic primer”
tatggctctggggctctata

SEQ ID NO: 499 - DNA, 20 bp, Artificial Sequence
  source: /note=“Description of Artificial Sequence Synthetic primer”
aacagagagaatgaggaggg

SEQ ID NO: 500 - DNA, 20 bp, Artificial Sequence
  source: /note=“Description of Artificial Sequence Synthetic primer”
tcattctaaaagggctgccg

SEQ ID NO: 501 - DNA, 20 bp, Artificial Sequence
  source: /note=“Description of Artificial Sequence Synthetic primer”
gaaaggtattcagggtggtg

SEQ ID NO: 502 - DNA, 20 bp, Artificial Sequence
  source: /note=“Description of Artificial Sequence Synthetic primer”
gatgctctgagacaatcctg

SEQ ID NO: 503 - DNA, 20 bp, Artificial Sequence
  source: /note=“Description of Artificial Sequence Synthetic primer”
ttaactgtgaggcgttcacc

SEQ ID NO: 504 - DNA, 20 bp, Artificial Sequence
  source: /note=“Description of Artificial Sequence Synthetic primer”
gatcatgggactatccacac

SEQ ID NO: 505 - DNA, 20 bp, Artificial Sequence
  source: /note=“Description of Artificial Sequence Synthetic primer”
ccagccctgctcctttaatc

SEQ ID NO: 506 - DNA, 20 bp, Artificial Sequence
  source: /note=“Description of Artificial Sequence Synthetic primer”
ggagaagatcctacactcag

SEQ ID NO: 507 - DNA, 20 bp, Artificial Sequence
  source: /note=“Description of Artificial Sequence Synthetic primer”
ccaacttcttcccagtctgt

SEQ ID NO: 508 - DNA, 19 bp, Artificial Sequence
  source: /note=“Description of Artificial Sequence Synthetic primer”
ctggagctgaaggacccca

SEQ ID NO: 509 - DNA, 20 bp, Artificial Sequence
  source: /note=“Description of Artificial Sequence Synthetic primer”
ggagaaatccttccctagag

SEQ ID NO: 510 - DNA, 20 bp, Artificial Sequence
  source: /note=“Description of Artificial Sequence Synthetic primer”
ttcaaggtgctgcaggtttg

SEQ ID NO: 511 - DNA, 20 bp, Artificial Sequence
  source: /note=“Description of Artificial Sequence Synthetic primer”
ccccctctacaggaaaattc

SEQ ID NO: 512 - DNA, 20 bp, Artificial Sequence
  source: /note=“Description of Artificial Sequence Synthetic primer”
ttctgaattcttcagccagc

SEQ ID NO: 513 - DNA, 20 bp, Artificial Sequence
  source: /note=“Description of Artificial Sequence Synthetic primer”
catcctacctctaggtacac

SEQ ID NO: 514 - DNA, 20 bp, Artificial Sequence
  source: /note=“Description of Artificial Sequence Synthetic primer”
ggtgtcttagttacatgtgc

SEQ ID NO: 515 - DNA, 20 bp, Artificial Sequence
  source: /note=“Description of Artificial Sequence Synthetic primer”
tggtgacgcaaggactggac

SEQ ID NO: 516 - DNA, 20 bp, Artificial Sequence
  source: /note=“Description of Artificial Sequence Synthetic primer”
atactgtgctgctcttcagg

SEQ ID NO: 517 - DNA, 19 bp, Artificial Sequence
  source: /note=“Description of Artificial Sequence Synthetic primer”
cagctgctgttccctcaga

SEQ ID NO: 518 - DNA, 20 bp, Artificial Sequence
  source: /note=“Description of Artificial Sequence Synthetic primer”
ccaaaaaaccatgccctctg

SEQ ID NO: 519 - DNA, 20 bp, Artificial Sequence
  source: /note=“Description of Artificial Sequence Synthetic primer”
taattggtacaggaggtggg

SEQ ID NO: 520 - DNA, 19 bp, Artificial Sequence
  source: /note=“Description of Artificial Sequence Synthetic primer”
aggcatgggactcagcttg

SEQ ID NO: 521 - DNA, 20 bp, Artificial Sequence
  source: /note=“Description of Artificial Sequence Synthetic primer”
gtgcaggtcattgtgctgag

SEQ ID NO: 522 - DNA, 20 bp, Artificial Sequence
  source: /note=“Description of Artificial Sequence Synthetic primer”
aaacactccacgttaaaggg

SEQ ID NO: 523 - DNA, 20 bp, Artificial Sequence
  source: /note=“Description of Artificial Sequence Synthetic primer”
cagctgagaaaactgagacc

SEQ ID NO: 524 - DNA, 20 bp, Artificial Sequence
  source: /note=“Description of Artificial Sequence Synthetic primer”
tttacagactagcgtgacgg

SEQ ID NO: 525 - DNA, 20 bp, Artificial Sequence
  source: /note=“Description of Artificial Sequence Synthetic primer”
tgctgctccgccatgaaagt

SEQ ID NO: 526 - DNA, 20 bp, Artificial Sequence
  source: /note=“Description of Artificial Sequence Synthetic primer”
atgcagggagagcagcagcc

SEQ ID NO: 527 - DNA, 20 bp, Artificial Sequence
  source: /note=“Description of Artificial Sequence Synthetic primer”
gctgagagttaagtggccaa

SEQ ID NO: 528 - DNA, 20 bp, Artificial Sequence
  source: /note=“Description of Artificial Sequence Synthetic primer”
ctgtggccatatttctgctg

SEQ ID NO: 529 - DNA, 20 bp, Artificial Sequence
  source: /note=“Description of Artificial Sequence Synthetic primer”
gcaatcaggcccagcttatg

SEQ ID NO: 530 - DNA, 20 bp, Artificial Sequence
  source: /note=“Description of Artificial Sequence Synthetic primer”
ttgtctggactctcttcatc

SEQ ID NO: 531 - DNA, 20 bp, Artificial Sequence
  source: /note=“Description of Artificial Sequence Synthetic primer”
cgcctaatttccagcaagaa

SEQ ID NO: 532 - DNA, 20 bp, Artificial Sequence
  source: /note=“Description of Artificial Sequence Synthetic primer”
gacttgcaaaagctctctgg

SEQ ID NO: 533 - DNA, 20 bp, Artificial Sequence
  source: /note=“Description of Artificial Sequence Synthetic primer”
gtctggctgaggaatgctac

SEQ ID NO: 534 - DNA, 20 bp, Artificial Sequence
  source: /note=“Description of Artificial Sequence Synthetic primer”
aagggcagcatgagcttggg

SEQ ID NO: 535 - DNA, 21 bp, Artificial Sequence
  source: /note=“Description of Artificial Sequence Synthetic primer”
gtctacttcaaatcatgcctc

SEQ ID NO: 536 - DNA, 20 bp, Artificial Sequence
  source: /note=“Description of Artificial Sequence Synthetic primer”
ctacatgcatatctggagac

SEQ ID NO: 537 - DNA, 20 bp, Artificial Sequence
  source: /note=“Description of Artificial Sequence Synthetic primer”
cagagatgcaagcagccaag

SEQ ID NO: 538 - DNA, 19 bp, Artificial Sequence
  source: /note=“Description of Artificial Sequence Synthetic primer”
aggaatggggctgccatct

SEQ ID NO: 539 - DNA, 20 bp, Artificial Sequence
  source: /note=“Description of Artificial Sequence Synthetic primer”
gagacaggcaaagatgcaac

SEQ ID NO: 540 - DNA, 19 bp, Artificial Sequence
  source: /note=“Description of Artificial Sequence Synthetic primer”
accacgcctggccagaact

SEQ ID NO: 541 - DNA, 20 bp, Artificial Sequence
  source: /note=“Description of Artificial Sequence Synthetic primer”
gggtttagtctccttacccc

SEQ ID NO: 542 - DNA, 20 bp, Artificial Sequence
  source: /note=“Description of Artificial Sequence Synthetic primer”
aatgtccctggcacagctca

SEQ ID NO: 543 - DNA, 20 bp, Artificial Sequence
  source: /note=“Description of Artificial Sequence Synthetic primer”
gcttcagttgtcactgtgag

SEQ ID NO: 544 - DNA, 20 bp, Artificial Sequence
  source: /note=“Description of Artificial Sequence Synthetic primer”
ctcagatgatgtcccttctt

SEQ ID NO: 545 - DNA, 20 bp, Artificial Sequence
  source: /note=“Description of Artificial Sequence Synthetic primer”
cgatgcaagcttccattcta

SEQ ID NO: 546 - DNA, 20 bp, Artificial Sequence
  source: /note=“Description of Artificial Sequence Synthetic primer”
ggacagagaatggcctgcta

SEQ ID NO: 547 - DNA, 19 bp, Artificial Sequence
  source: /note=“Description of Artificial Sequence Synthetic primer”
ttaaaacagccctgcaacc

SEQ ID NO: 548 - DNA, 19 bp, Artificial Sequence
  source: /note=“Description of Artificial Sequence Synthetic primer”
tgatgagaacagagctgag

SEQ ID NO: 549 - DNA, 20 bp, Artificial Sequence
  source: /note=“Description of Artificial Sequence Synthetic primer”
ctgaagctatgtcctgttag

SEQ ID NO: 550 - DNA, 20 bp, Artificial Sequence
  source: /note=“Description of Artificial Sequence Synthetic primer”
aggtggcacggcacgttcat

SEQ ID NO: 551 - DNA, 20 bp, Artificial Sequence
  source: /note=“Description of Artificial Sequence Synthetic primer”
ctgaagtgcaggaagcttgg

SEQ ID NO: 552 - DNA, 20 bp, Artificial Sequence
  source: /note=“Description of Artificial Sequence Synthetic primer”
accctagaacttgacactgc

SEQ ID NO: 553 - DNA, 20 bp, Artificial Sequence
  source: /note=“Description of Artificial Sequence Synthetic primer”
aaggagctggcaaggcccta

SEQ ID NO: 554 - DNA, 20 bp, Artificial Sequence
  source: /note=“Description of Artificial Sequence Synthetic primer”
acataggcacaatgagatgg

SEQ ID NO: 555 - DNA, 20 bp, Artificial Sequence
  source: /note=“Description of Artificial Sequence Synthetic primer”
tacctttcaagctcaagtgc

SEQ ID NO: 556 - DNA, 20 bp, Artificial Sequence
  source: /note=“Description of Artificial Sequence Synthetic primer”
tttggatggaacgtttgcag

SEQ ID NO: 557 - DNA, 20 bp, Artificial Sequence
  source: /note=“Description of Artificial Sequence Synthetic primer”
gctaccctttaatgtgtctc

SEQ ID NO: 558 - DNA, 20 bp, Artificial Sequence
  source: /note=“Description of Artificial Sequence Synthetic primer”
atgaagagcagctggtcaac

SEQ ID NO: 559 - DNA, 20 bp, Artificial Sequence
  source: /note=“Description of Artificial Sequence Synthetic primer”
cagcccttgtgtgcataaag

SEQ ID NO: 560 - DNA, 20 bp, Artificial Sequence
  source: /note=“Description of Artificial Sequence Synthetic primer”
tacagtggtggacaaggtgg

SEQ ID NO: 561 - DNA, 20 bp, Artificial Sequence
  source: /note=“Description of Artificial Sequence Synthetic primer”
cttgttttgcaggctgattg

SEQ ID NO: 562 - DNA, 20 bp, Artificial Sequence
  source: /note=“Description of Artificial Sequence Synthetic primer”
tcaatcatccccatccccac

SEQ ID NO: 563 - DNA, 20 bp, Artificial Sequence
  source: /note=“Description of Artificial Sequence Synthetic primer”
gcacatcacaagttaagagg

SEQ ID NO: 564 - DNA, 20 bp, Artificial Sequence
  source: /note=“Description of Artificial Sequence Synthetic primer”
ccccagtagggaacacactt

SEQ ID NO: 565 - DNA, 20 bp, Artificial Sequence
  source: /note=“Description of Artificial Sequence Synthetic primer”
caggatgcactttttggatg

SEQ ID NO: 566 - DNA, 20 bp, Artificial Sequence
  source: /note=“Description of Artificial Sequence Synthetic primer”
ggcttctcccagaaaatctc

SEQ ID NO: 567 - DNA, 20 bp, Artificial Sequence
  source: /note=“Description of Artificial Sequence Synthetic primer”
actgcagtgccgggaaaagt

SEQ ID NO: 568 - DNA, 19 bp, Artificial Sequence
  source: /note=“Description of Artificial Sequence Synthetic primer”
tttgctcaccctaccccac

SEQ ID NO: 569 - DNA, 20 bp, Artificial Sequence
  source: /note=“Description of Artificial Sequence Synthetic primer”
tgataacagcctccatttcc

SEQ ID NO: 570 - DNA, 20 bp, Artificial Sequence
  source: /note=“Description of Artificial Sequence Synthetic primer”
tagggatgcaagatgaaagg

SEQ ID NO: 571 - DNA, 19 bp, Artificial Sequence
  source: /note=“Description of Artificial Sequence Synthetic primer”
gatgcaggagggcgtccca

SEQ ID NO: 572 - DNA, 19 bp, Artificial Sequence
  source: /note=“Description of Artificial Sequence Synthetic primer”
tccagccactctgagctgc

SEQ ID NO: 573 - DNA, 20 bp, Artificial Sequence
  source: /note=“Description of Artificial Sequence Synthetic primer”
tctgctgtttgatggatgtg

SEQ ID NO: 574 - DNA, 20 bp, Artificial Sequence
  source: /note=“Description of Artificial Sequence Synthetic primer”
tgggagatcagctaggaatg

SEQ ID NO: 575 - DNA, 20 bp, Artificial Sequence
  source: /note=“Description of Artificial Sequence Synthetic primer”
gctgggatcccatctcaaag

SEQ ID NO: 576 - DNA, 20 bp, Artificial Sequence
  source: /note=“Description of Artificial Sequence Synthetic primer”
tgaatgtcttgcttgagacc

SEQ ID NO: 577 - DNA, 20 bp, Artificial Sequence
  source: /note=“Description of Artificial Sequence Synthetic primer”
ttcccttgtttggaaccctg

SEQ ID NO: 578 - DNA, 19 bp, Artificial Sequence
  source: /note=“Description of Artificial Sequence Synthetic primer”
cagcttccaccctctccac

SEQ ID NO: 579 - DNA, 20 bp, Artificial Sequence
  source: /note=“Description of Artificial Sequence Synthetic primer”
tggccttaaacatgcatgct

SEQ ID NO: 580 - DNA, 20 bp, Artificial Sequence
  source: /note=“Description of Artificial Sequence Synthetic primer”
ggtgacaatctagagaggtg

SEQ ID NO: 581 - DNA, 20 bp, Artificial Sequence
  source: /note=“Description of Artificial Sequence Synthetic primer”
aggtcagctcagggtgaagt

SEQ ID NO: 582 - DNA, 20 bp, Artificial Sequence
  source: /note=“Description of Artificial Sequence Synthetic primer”
gctccattgaagggtaaagg

SEQ ID NO: 583 - DNA, 20 bp, Artificial Sequence
  source: /note=“Description of Artificial Sequence Synthetic primer”
gagggtacctttctttctcc

SEQ ID NO: 584 - DNA, 20 bp, Artificial Sequence
  source: /note=“Description of Artificial Sequence Synthetic primer”
gctcagtgtctgacaaaagc

SEQ ID NO: 585 - DNA, 20 bp, Artificial Sequence
  source: /note=“Description of Artificial Sequence Synthetic primer”
agccatgtttcagggttcag

SEQ ID NO: 586 - DNA, 20 bp, Artificial Sequence
  source: /note=“Description of Artificial Sequence Synthetic primer”
cagtgcctgacagggaaagt

SEQ ID NO: 587 - DNA, 22 bp, Artificial Sequence
  source: /note=“Description of Artificial Sequence Synthetic primer”
ctgttttctcagaagggacttt

SEQ ID NO: 588 - DNA, 24 bp, Artificial Sequence
  source: /note=“Description of Artificial Sequence Synthetic primer”
tcgaaagaaaacactgagaatcaa

SEQ ID NO: 589 - DNA, 21 bp, Artificial Sequence
  source: /note=“Description of Artificial Sequence Synthetic primer”
tggacaaaaataccatcatca

SEQ ID NO: 590 - DNA, 21 bp, Artificial Sequence
  source: /note=“Description of Artificial Sequence Synthetic primer”
agatcatcctgaacataaggt

SEQ ID NO: 591 - DNA, 23 bp, Artificial Sequence
  source: /note=“Description of Artificial Sequence Synthetic primer”
ttccctcttcagtttacctgttt

SEQ ID NO: 592 - DNA, 21 bp, Artificial Sequence
  source: /note=“Description of Artificial Sequence Synthetic primer”
caccaagaagggaatgaaaat

SEQ ID NO: 593 - DNA, 21 bp, Artificial Sequence
  source: /note=“Description of Artificial Sequence Synthetic primer”
tgaagaaagcaagggacagaa

SEQ ID NO: 594 - DNA, 20 bp, Artificial Sequence
  source: /note=“Description of Artificial Sequence Synthetic primer”
aagccgcgtgttattgaaac

SEQ ID NO: 595 - DNA, 19 bp, Artificial Sequence
  source: /note=“Description of Artificial Sequence Synthetic primer”
ttcagtgctttccgttgga

SEQ ID NO: 596 - DNA, 20 bp, Artificial Sequence
  source: /note=“Description of Artificial Sequence Synthetic primer”
cacacacacgcactaagcaa

SEQ ID NO: 597 - DNA, 23 bp, Artificial Sequence
  source: /note=“Description of Artificial Sequence Synthetic primer”
tcacctcatacatgttttctttt

SEQ ID NO: 598 - DNA, 22 bp, Artificial Sequence
  source: /note=“Description of Artificial Sequence Synthetic primer”
aatacctcaaaggactgtaatg

SEQ ID NO: 599 - DNA, 20 bp, Artificial Sequence
  source: /note=“Description of Artificial Sequence Synthetic primer”
tgctgcattcatttgtcaac

SEQ ID NO: 600 - DNA, 20 bp, Artificial Sequence
  source: /note=“Description of Artificial Sequence Synthetic primer”
gaactctggtgttcctagtg

SEQ ID NO: 601 - DNA, 23 bp, Artificial Sequence
  source: /note=“Description of Artificial Sequence Synthetic primer”
tgtatttgcctaaaagtaagagg

SEQ ID NO: 602 - DNA, 20 bp, Artificial Sequence
  source: /note=“Description of Artificial Sequence Synthetic primer”
ggcagagttctcttgacgtg

SEQ ID NO: 603 - DNA, 20 bp, Artificial Sequence
  source: /note=“Description of Artificial Sequence Synthetic primer”
aaggaagtaaaggtatgtgc

SEQ ID NO: 604 - DNA, 20 bp, Artificial Sequence
  source: /note=“Description of Artificial Sequence Synthetic primer”
aggctaactctaacatcctg

SEQ ID NO: 605 - DNA, 19 bp, Artificial Sequence
  source: /note=“Description of Artificial Sequence Synthetic primer”
acttaaaaccaaaccctca

SEQ ID NO: 606 - DNA, 20 bp, Artificial Sequence
  source: /note=“Description of Artificial Sequence Synthetic primer”
ttattgggtgttgcaagtgt

SEQ ID NO: 607 - DNA, 20 bp, Artificial Sequence
  source: /note=“Description of Artificial Sequence Synthetic primer”
gtttattgatgaactggtgc

SEQ ID NO: 608 - DNA, 19 bp, Artificial Sequence
  source: /note=“Description of Artificial Sequence Synthetic primer”
gggcagagtgatatcacag

SEQ ID NO: 609 - DNA, 20 bp, Artificial Sequence
  source: /note=“Description of Artificial Sequence Synthetic primer”
tcccctctaccccttgaagc

SEQ ID NO: 610 - DNA, 20 bp, Artificial Sequence
  source: /note=“Description of Artificial Sequence Synthetic primer”
cagcactgatgacatctggg

SEQ ID NO: 611 - DNA, 20 bp, Artificial Sequence
  source: /note=“Description of Artificial Sequence Synthetic primer”
acggggtcttatggttcctc

SEQ ID NO: 612 - DNA, 21 bp, Artificial Sequence
  source: /note=“Description of Artificial Sequence Synthetic primer”
gcctgagaagcaattaacctg

SEQ ID NO: 613 - DNA, 22 bp, Artificial Sequence
  source: /note=“Description of Artificial Sequence Synthetic primer”
tgctaacctgtgaagtccattc

SEQ ID NO: 614 - DNA, 21 bp, Artificial Sequence
  source: /note=“Description of Artificial Sequence Synthetic primer”
tttatttgcattggtctttgc

SEQ ID NO: 615 - DNA, 23 bp, Artificial Sequence
  source: /note=“Description of Artificial Sequence Synthetic primer”
gcataatttgaaagctctgtttg

SEQ ID NO: 616 - DNA, 23 bp, Artificial Sequence
  source: /note=“Description of Artificial Sequence Synthetic primer”
cgattatgcccattgatattttt

SEQ ID NO: 617 - DNA, 20 bp, Artificial Sequence
  source: /note=“Description of Artificial Sequence Synthetic primer”
tcaagggattgctccaatgt

SEQ ID NO: 618 - DNA, 21 bp, Artificial Sequence
  source: /note=“Description of Artificial Sequence Synthetic primer”
ctccaaaccaatacctaaaaa

SEQ ID NO: 619 - DNA, 20 bp, Artificial Sequence
  source: /note=“Description of Artificial Sequence Synthetic primer”
ccagccctgctcctttaatc

SEQ ID NO: 620 - DNA, 20 bp, Artificial Sequence
  source: /note=“Description of Artificial Sequence Synthetic primer”
ggagaagatcctacactcag

SEQ ID NO: 621 - DNA, 21 bp, Artificial Sequence
  source: /note=“Description of Artificial Sequence Synthetic primer”
gcttctcatctgtgtgcattt

SEQ ID NO: 622 - DNA, 22 bp, Artificial Sequence
  source: /note=“Description of Artificial Sequence Synthetic primer”
cctagaatatgatgcccaaaca

SEQ ID NO: 623 - DNA, 23 bp, Artificial Sequence
  source: /note=“Description of Artificial Sequence Synthetic primer”
cctcctctactagacctctgacg

SEQ ID NO: 624 - DNA, 23 bp, Artificial Sequence
  source: /note=“Description of Artificial Sequence Synthetic primer”
tgtagaataagaaggcagtccaa

SEQ ID NO: 625 - DNA, 18 bp, Artificial Sequence
  source: /note=“Description of Artificial Sequence Synthetic primer”
acctagggaaggggtcac

SEQ ID NO: 626 - DNA, 18 bp, Artificial Sequence
  source: /note=“Description of Artificial Sequence Synthetic primer”
aagccagggttcatctgc

SEQ ID NO: 627 - DNA, 20 bp, Artificial Sequence
  source: /note=“Description of Artificial Sequence Synthetic primer”
catcctacctctaggtacac

SEQ ID NO: 628 - DNA, 20 bp, Artificial Sequence
  source: /note=“Description of Artificial Sequence Synthetic primer”
ggtgtcttagttacatgtgc

SEQ ID NO: 629 - DNA, 22 bp, Artificial Sequence
  source: /note=“Description of Artificial Sequence Synthetic primer”
tccaaaattccttgatgtgtca

SEQ ID NO: 630 - DNA, 22 bp, Artificial Sequence
  source: /note=“Description of Artificial Sequence Synthetic primer”
tcaacctcctacagcaacaaaa

SEQ ID NO: 631 - DNA, 22 bp, Artificial Sequence
  source: /note=“Description of Artificial Sequence Synthetic primer”
ggttcactacagagcgtctcaa

SEQ ID NO: 632 - DNA, 20 bp, Artificial Sequence
  source: /note=“Description of Artificial Sequence Synthetic primer”
atgtaccttttgggccttgc

SEQ ID NO: 633 - DNA, 18 bp, Artificial Sequence
  source: /note=“Description of Artificial Sequence Synthetic primer”
ccaccaacctgcctctgg

SEQ ID NO: 634 - DNA, 24 bp, Artificial Sequence
  source: /note=“Description of Artificial Sequence Synthetic primer”
tgatttgtgatcagtcttcctctt

SEQ ID NO: 635 - DNA, 20 bp, Artificial Sequence
  source: /note=“Description of Artificial Sequence Synthetic primer”
attcttcctcctgcaatcca

SEQ ID NO: 636 - DNA, 20 bp, Artificial Sequence
  source: /note=“Description of Artificial Sequence Synthetic primer”
tttggaagtcggtgctaacc

SEQ ID NO: 637 - DNA, 20 bp, Artificial Sequence
  source: /note=“Description of Artificial Sequence Synthetic primer”
ggcttgcttaacatgtgctg

SEQ ID NO: 638 - DNA, 22 bp, Artificial Sequence
  source: /note=“Description of Artificial Sequence Synthetic primer”
caaagattgcagataaagtgct

SEQ ID NO: 639 - DNA, 21 bp, Artificial Sequence
  source: /note=“Description of Artificial Sequence Synthetic primer”
ttccttggcattttagtttcc

SEQ ID NO: 640 - DNA, 21 bp, Artificial Sequence
  source: /note=“Description of Artificial Sequence Synthetic primer”
tcccatgtaacacctttcaga

SEQ ID NO: 641 - DNA, 23 bp, Artificial Sequence
  source: /note=“Description of Artificial Sequence Synthetic primer”
tttgcattctcctgtctcttttt

SEQ ID NO: 642 - DNA, 21 bp, Artificial Sequence
  source: /note=“Description of Artificial Sequence Synthetic primer”
ggaaccactacaggaaacgaa

SEQ ID NO: 643 - DNA, 20 bp, Artificial Sequence
  source: /note=“Description of Artificial Sequence Synthetic primer”
ttttgccaagctattcacag

SEQ ID NO: 644 - DNA, 21 bp, Artificial Sequence
  source: /note=“Description of Artificial Sequence Synthetic primer”
ctccatcgagggattatcaga

SEQ ID NO: 645 - DNA, 23 bp, Artificial Sequence
  source: /note=“Description of Artificial Sequence Synthetic primer”
gcacctattcacagacagtttga

SEQ ID NO: 646 - DNA, 20 bp, Artificial Sequence
  source: /note=“Description of Artificial Sequence Synthetic primer”
agaatcttccgattctgcat

SEQ ID NO: 647 - DNA, 21 bp, Artificial Sequence
  source: /note=“Description of Artificial Sequence Synthetic primer”
gcccctataatccttggagtc

SEQ ID NO: 648 - DNA, 20 bp, Artificial Sequence
  source: /note=“Description of Artificial Sequence Synthetic primer”
gaggagccaaagagctgaaa

SEQ ID NO: 649 - DNA, 24 bp, Artificial Sequence
  source: /note=“Description of Artificial Sequence Synthetic primer”
ggtttcattactctatgcttcttc

SEQ ID NO: 650 - DNA, 20 bp, Artificial Sequence
  source: /note=“Description of Artificial Sequence Synthetic primer”
aggagatgtgcatttcagca

SEQ ID NO: 651 - DNA, 21 bp, Artificial Sequence
  source: /note=“Description of Artificial Sequence Synthetic primer”
caggacaggaattagtgttgc

SEQ ID NO: 652 - DNA, 23 bp, Artificial Sequence
  source: /note=“Description of Artificial Sequence Synthetic primer”
ttttaaatactacggagtcaaac

SEQ ID NO: 653 - DNA, 20 bp, Artificial Sequence
  source: /note=“Description of Artificial Sequence Synthetic primer”
gcccttgaaaagagggctta

SEQ ID NO: 654 - DNA, 22 bp, Artificial Sequence
  source: /note=“Description of Artificial Sequence Synthetic primer”
gcaggatattctctgactgcaa

SEQ ID NO: 655 - DNA, 24 bp, Artificial Sequence
  source: /note=“Description of Artificial Sequence Synthetic primer”
aaagagtatagggatggacactga

SEQ ID NO: 656 - DNA, 21 bp, Artificial Sequence
  source: /note=“Description of Artificial Sequence Synthetic primer”
cgtgtagtagtcacccggttt

SEQ ID NO: 657 - DNA, 19 bp, Artificial Sequence
  source: /note=“Description of Artificial Sequence Synthetic primer”
gaaagcctacacgcccaag

SEQ ID NO: 658 - DNA, 22 bp, Artificial Sequence
  source: /note=“Description of Artificial Sequence Synthetic primer”
ttttcagtgtcctcacctctga

SEQ ID NO: 659 - DNA, 20 bp, Artificial Sequence
  source: /note=“Description of Artificial Sequence Synthetic primer”
tccaactggaaaacacctca

SEQ ID NO: 660 - DNA, 22 bp, Artificial Sequence
  source: /note=“Description of Artificial Sequence Synthetic primer”
gagccaccttcaagactctttc

SEQ ID NO: 661 - DNA, 20 bp, Artificial Sequence
  source: /note=“Description of Artificial Sequence Synthetic primer”
caaaatttccagcgcattct

SEQ ID NO: 662 - DNA, 20 bp, Artificial Sequence
  source: /note=“Description of Artificial Sequence Synthetic primer”
actgattcctcgcagccttg

SEQ ID NO: 663 - DNA, 20 bp, Artificial Sequence
  source: /note=“Description of Artificial Sequence Synthetic primer”
actgcattgtggcggtatct

SEQ ID NO: 664 - DNA, 23 bp, Artificial Sequence
  source: /note=“Description of Artificial Sequence Synthetic primer”
aaaagtacatgatgcatttaagc

SEQ ID NO: 665 - DNA, 23 bp, Artificial Sequence
  source: /note=“Description of Artificial Sequence Synthetic primer”
aaaacataattgaacacctagca

SEQ ID NO: 666 - DNA, 20 bp, Artificial Sequence
  source: /note=“Description of Artificial Sequence Synthetic primer”
aataggaggctgctctatgc

SEQ ID NO: 667 - DNA, 24 bp, Artificial Sequence
  source: /note=“Description of Artificial Sequence Synthetic primer”
cgctggtaaatacttagagataaa

SEQ ID NO: 668 - DNA, 19 bp, Artificial Sequence
  source: /note=“Description of Artificial Sequence Synthetic primer”
acatgttccccattgctca

SEQ ID NO: 669 - DNA, 24 bp, Artificial Sequence
  source: /note=“Description of Artificial Sequence Synthetic primer”
cagtcttgttgtattccctaaaga

SEQ ID NO: 670 - DNA, 21 bp, Artificial Sequence
  source: /note=“Description of Artificial Sequence Synthetic primer”
gcaactgagaggatgaggttg

SEQ ID NO: 671 - DNA, 22 bp, Artificial Sequence
  source: /note=“Description of Artificial Sequence Synthetic primer”
gacctaagactagtgccgtgaa

SEQ ID NO: 672 - DNA, 20 bp, Artificial Sequence
  source: /note=“Description of Artificial Sequence Synthetic primer”
gtgcaaaggaaaccaggaga

SEQ ID NO: 673 - DNA, 23 bp, Artificial Sequence
  source: /note=“Description of Artificial Sequence Synthetic primer”
ggaataatatatgtggactgctt

SEQ ID NO: 674 - DNA, 21 bp, Artificial Sequence
  source: /note=“Description of Artificial Sequence Synthetic primer”
ttacccttaaaaattccttgg

SEQ ID NO: 675 - DNA, 23 bp, Artificial Sequence
  source: /note=“Description of Artificial Sequence Synthetic primer”
aaagcaaatggttaaatagcaga

SEQ ID NO: 676 - DNA, 23 bp, Artificial Sequence
  source: /note=“Description of Artificial Sequence Synthetic primer”
ttggttctttctctttaattgtg

SEQ ID NO: 677 - DNA, 24 bp, Artificial Sequence
  source: /note=“Description of Artificial Sequence Synthetic primer”
cagagagaaagcagtttgaatttg

SEQ ID NO: 678 - DNA, 21 bp, Artificial Sequence
  source: /note=“Description of Artificial Sequence Synthetic primer”
ccaagataccttgccttctga

SEQ ID NO: 679 - DNA, 24 bp, Artificial Sequence
  source: /note=“Description of Artificial Sequence Synthetic primer”
catcatattcctaactgtgctcat

SEQ ID NO: 680 - DNA, 24 bp, Artificial Sequence
  source: /note=“Description of Artificial Sequence Synthetic primer”
tccttggtaaagagggtaaagaaa

SEQ ID NO: 681 - DNA, 20 bp, Artificial Sequence
  source: /note=“Description of Artificial Sequence Synthetic primer”
tactgccaacagacaactcg

SEQ ID NO: 682 - DNA, 21 bp, Artificial Sequence
  source: /note=“Description of Artificial Sequence Synthetic primer”
ttagaccgcagacctttagaa

SEQ ID NO: 683 - DNA, 21 bp, Artificial Sequence
  source: /note=“Description of Artificial Sequence Synthetic primer”
gtctacttcaaatcatgcctc

SEQ ID NO: 684 - DNA, 20 bp, Artificial Sequence
  source: /note=“Description of Artificial Sequence Synthetic primer”
ctacatgcatatctggagac

SEQ ID NO: 685 - DNA, 21 bp, Artificial Sequence
  source: /note=“Description of Artificial Sequence Synthetic primer”
actgaggagtaattcatgagg

SEQ ID NO: 686 - DNA, 23 bp, Artificial Sequence
  source: /note=“Description of Artificial Sequence Synthetic primer”
tggttttacctttctgaaaaaca

SEQ ID NO: 687 - DNA, 23 bp, Artificial Sequence
  source: /note=“Description of Artificial Sequence Synthetic primer”
cacctcctaagacaaaatggcta

SEQ ID NO: 688 - DNA, 20 bp, Artificial Sequence
  source: /note=“Description of Artificial Sequence Synthetic primer”
cctaatccagcagaccatgt

SEQ ID NO: 689 - DNA, 20 bp, Artificial Sequence
  source: /note=“Description of Artificial Sequence Synthetic primer”
ggaggcagaaattgcatcag

SEQ ID NO: 690 - DNA, 20 bp, Artificial Sequence
  source: /note=“Description of Artificial Sequence Synthetic primer”
gcatagccagccattagcat

SEQ ID NO: 691 - DNA, 23 bp, Artificial Sequence
  source: /note=“Description of Artificial Sequence Synthetic primer”
tctcagagcaacatgtaccaaaa

SEQ ID NO: 692 - DNA, 20 bp, Artificial Sequence
  source: /note=“Description of Artificial Sequence Synthetic primer”
gcccaatcagacatcaatcc

SEQ ID NO: 693 - DNA, 19 bp, Artificial Sequence
  source: /note=“Description of Artificial Sequence Synthetic primer”
cctcaaggcctctccattg

SEQ ID NO: 694 - DNA, 24 bp, Artificial Sequence
  source: /note=“Description of Artificial Sequence Synthetic primer”
gaagagttttgactttttctgagg

SEQ ID NO: 695 - DNA, 18 bp, Artificial Sequence
  source: /note=“Description of Artificial Sequence Synthetic primer”
atccccaagcccaagaag

SEQ ID NO: 696 - DNA, 20 bp, Artificial Sequence
  source: /note=“Description of Artificial Sequence Synthetic primer”
acagccatgaacgaagcatt

SEQ ID NO: 697 - DNA, 24 bp, Artificial Sequence
  source: /note=“Description of Artificial Sequence Synthetic primer”
ggctcatgaactaagatagtttgg

SEQ ID NO: 698 - DNA, 23 bp, Artificial Sequence
  source: /note=“Description of Artificial Sequence Synthetic primer”
aagaaagattgtgggattagaca

SEQ ID NO: 699 - DNA, 21 bp, Artificial Sequence
  source: /note=“Description of Artificial Sequence Synthetic primer”
ccatcagagcttagggttgaa

SEQ ID NO: 700 - DNA, 20 bp, Artificial Sequence
  source: /note=“Description of Artificial Sequence Synthetic primer”
ttggaggagaaaggcatcag

SEQ ID NO: 701 - DNA, 24 bp, Artificial Sequence
  source: /note=“Description of Artificial Sequence Synthetic primer”
ccatcttagttggaaatagcaacc

SEQ ID NO: 702 - DNA, 20 bp, Artificial Sequence
  source: /note=“Description of Artificial Sequence Synthetic primer”
ccatcttcttttcccaagca

SEQ ID NO: 703 - DNA, 20 bp, Artificial Sequence
  source: /note=“Description of Artificial Sequence Synthetic primer”
acatgctcctagggtgcttc

SEQ ID NO: 704 - DNA, 22 bp, Artificial Sequence
  source: /note=“Description of Artificial Sequence Synthetic primer”
ttcttgacggtgttctgttttt

SEQ ID NO: 705 - DNA, 20 bp, Artificial Sequence
  source: /note=“Description of Artificial Sequence Synthetic primer”
cccttgttggggaaataaca

SEQ ID NO: 706 - DNA, 23 bp, Artificial Sequence
  source: /note=“Description of Artificial Sequence Synthetic primer”
ccctatttcctactgaacgctta

SEQ ID NO: 707 - DNA, 21 bp, Artificial Sequence
  source: /note=“Description of Artificial Sequence Synthetic primer”
ttggaatgtccatcctttgag

SEQ ID NO: 708 - DNA, 18 bp, Artificial Sequence
  source: /note=“Description of Artificial Sequence Synthetic primer”
caaacccatggccttgaa

SEQ ID NO: 709 - DNA, 22 bp, Artificial Sequence
  source: /note=“Description of Artificial Sequence Synthetic primer”
ggtatgtatgtgggaagggaat

SEQ ID NO: 710 - DNA, 23 bp, Artificial Sequence
  source: /note=“Description of Artificial Sequence Synthetic primer”
aaggttatgtaagaaagatgtca

SEQ ID NO: 711 - DNA, 22 bp, Artificial Sequence
  source: /note=“Description of Artificial Sequence Synthetic primer”
aaggaagaattctcaatgacct

SEQ ID NO: 712 - DNA, 20 bp, Artificial Sequence
  source: /note=“Description of Artificial Sequence Synthetic primer”
tggggctaaaagtcagacca

SEQ ID NO: 713 - DNA, 24 bp, Artificial Sequence
  source: /note=“Description of Artificial Sequence Synthetic primer”
tttaagatatgctctctcctgact

SEQ ID NO: 714 - DNA, 23 bp, Artificial Sequence
  source: /note=“Description of Artificial Sequence Synthetic primer”
ctattagttaggtttccagttga

SEQ ID NO: 715 - DNA, 24 bp, Artificial Sequence
  source: /note=“Description of Artificial Sequence Synthetic primer”
aggaaatctgtgagtaactatcat

SEQ ID NO: 716 - DNA, 23 bp, Artificial Sequence
  source: /note=“Description of Artificial Sequence Synthetic primer”
cctaatagacctaacaaggatgc

SEQ ID NO: 717 - DNA, 23 bp, Artificial Sequence
  source: /note=“Description of Artificial Sequence Synthetic primer”
gcaatgataacaagaacacagca

SEQ ID NO: 718 - DNA, 20 bp, Artificial Sequence
  source: /note=“Description of Artificial Sequence Synthetic primer”
tggagccaaagggagtaata

SEQ ID NO: 719 - DNA, 21 bp, Artificial Sequence
  source: /note=“Description of Artificial Sequence Synthetic primer”
tctctccacctttccacactg

SEQ ID NO: 720 - DNA, 21 bp, Artificial Sequence
  source: /note=“Description of Artificial Sequence Synthetic primer”
tgtgtgaaacctgtgacttgc

SEQ ID NO: 721 - DNA, 23 bp, Artificial Sequence
  source: /note=“Description of Artificial Sequence Synthetic primer”
catattcataccttcaagccaac

SEQ ID NO: 722 - DNA, 19 bp, Artificial Sequence
  source: /note=“Description of Artificial Sequence Synthetic primer”
tgtggaaacacagcccatt

SEQ ID NO: 723 - DNA, 20 bp, Artificial Sequence
  source: /note=“Description of Artificial Sequence Synthetic primer”
gaaagggatgatggttccaa

SEQ ID NO: 724 - DNA, 20 bp, Artificial Sequence
  source: /note=“Description of Artificial Sequence Synthetic primer”
cccatgaacacattcacagc

SEQ ID NO: 725 - DNA, 20 bp, Artificial Sequence
  source: /note=“Description of Artificial Sequence Synthetic primer”
caggaactcgctgaataccc

SEQ ID NO: 726 - DNA, 20 bp, Artificial Sequence
  source: /note=“Description of Artificial Sequence Synthetic primer”
cagaggagcaccagcctatg

SEQ ID NO: 727 - DNA, 20 bp, Artificial Sequence
  source: /note=“Description of Artificial Sequence Synthetic primer”
gccatgaattaggagccttg

SEQ ID NO: 728 - DNA, 20 bp, Artificial Sequence
  source: /note=“Description of Artificial Sequence Synthetic primer”
caatccaacgaagatgacca

SEQ ID NO: 729 - DNA, 20 bp, Artificial Sequence
  source: /note=“Description of Artificial Sequence Synthetic primer”
accatgacctggcttgaagt

SEQ ID NO: 730 - DNA, 23 bp, Artificial Sequence
  source: /note=“Description of Artificial Sequence Synthetic primer”
ggacgatcaggttacacctaaaa

SEQ ID NO: 731 - DNA, 20 bp, Artificial Sequence
  source: /note=“Description of Artificial Sequence Synthetic primer”
tccacctcctaaccaaggac

SEQ ID NO: 732 - DNA, 21 bp, Artificial Sequence
  source: /note=“Description of Artificial Sequence Synthetic primer”
cagctgaacactgagattttt

SEQ ID NO: 733 - DNA, 20 bp, Artificial Sequence
  source: /note=“Description of Artificial Sequence Synthetic primer”
aagccctgtttccctgtttt

SEQ ID NO: 734 - DNA, 20 bp, Artificial Sequence
  source: /note=“Description of Artificial Sequence Synthetic primer”
catctgcagaagacagactc

SEQ ID NO: 735 - DNA, 19 bp, Artificial Sequence
  source: /note=“Description of Artificial Sequence Synthetic primer”
atcattcccggagctcaca

SEQ ID NO: 736 - DNA, 19 bp, Artificial Sequence
  source: /note=“Description of Artificial Sequence Synthetic primer”
gacacaatgtgccttgaaa

SEQ ID NO: 737 - DNA, 20 bp, Artificial Sequence
  source: /note=“Description of Artificial Sequence Synthetic primer”
gtacagctggaatggccaag

SEQ ID NO: 738 - DNA, 20 bp, Artificial Sequence
  source: /note=“Description of Artificial Sequence Synthetic primer”
cccagtttccatcctcagtc

SEQ ID NO: 739 - DNA, 22 bp, Artificial Sequence
  source: /note=“Description of Artificial Sequence Synthetic primer”
aacaatttgctctgagaacctc

SEQ ID NO: 740 - DNA, 19 bp, Artificial Sequence
  source: /note=“Description of Artificial Sequence Synthetic primer”
tcttggccttcagggtttc

SEQ ID NO: 741 - DNA, 24 bp, Artificial Sequence
  source: /note=“Description of Artificial Sequence Synthetic primer”
cctttgttactaagaattgaagtg

SEQ ID NO: 742 - DNA, 22 bp, Artificial Sequence
  source: /note=“Description of Artificial Sequence Synthetic primer”
tcgtttcttattgtcttctgtt

SEQ ID NO: 743 - DNA, 20 bp, Artificial Sequence
  source: /note=“Description of Artificial Sequence Synthetic primer”
ctcccaatatccgacagctc

SEQ ID NO: 744 - DNA, 20 bp, Artificial Sequence
  source: /note=“Description of Artificial Sequence Synthetic primer”
ccacctcaggacaggagagt

SEQ ID NO: 745 - DNA, 20 bp, Artificial Sequence
  source: /note=“Description of Artificial Sequence Synthetic primer”
tggattgatggcagaacatt

SEQ ID NO: 746 - DNA, 20 bp, Artificial Sequence
  source: /note=“Description of Artificial Sequence Synthetic primer”
ctgagggctttttggctaac

SEQ ID NO: 747 - DNA, 23 bp, Artificial Sequence
  source: /note=“Description of Artificial Sequence Synthetic primer”
ttttatttttctcacaagcctga

SEQ ID NO: 748 - DNA, 24 bp, Artificial Sequence
  source: /note=“Description of Artificial Sequence Synthetic primer”
tcagagagataaagaaggaaagga

SEQ ID NO: 749 - DNA, 20 bp, Artificial Sequence
  source: /note=“Description of Artificial Sequence Synthetic primer”
tctaattaagccatgactcc

SEQ ID NO: 750 - DNA, 20 bp, Artificial Sequence
  source: /note=“Description of Artificial Sequence Synthetic primer”
ggctgtggtatggctagcag

SEQ ID NO: 751 - DNA, 24 bp, Artificial Sequence
  source: /note=“Description of Artificial Sequence Synthetic primer”
cacagagaaagaacagaatctgaa

SEQ ID NO: 752 - DNA, 20 bp, Artificial Sequence
  source: /note=“Description of Artificial Sequence Synthetic primer”
aggcagacagatggacacat

SEQ ID NO: 753 - DNA, 20 bp, Artificial Sequence
  source: /note=“Description of Artificial Sequence Synthetic primer”
gagctttgcatgcagtaggg

SEQ ID NO: 754 - DNA, 20 bp, Artificial Sequence
  source: /note=“Description of Artificial Sequence Synthetic primer”
cccagcctctctgtctatgg

SEQ ID NO: 755 - DNA, 20 bp, Artificial Sequence
  source: /note=“Description of Artificial Sequence Synthetic primer”
tgacttctatcacccctacc

SEQ ID NO: 756 - DNA, 20 bp, Artificial Sequence
  source: /note=“Description of Artificial Sequence Synthetic primer”
gtgcaggagaggaaagcaga

SEQ ID NO: 757 - DNA, 20 bp, Artificial Sequence
  source: /note=“Description of Artificial Sequence Synthetic primer”
gttgatgacacccacattca

SEQ ID NO: 758 - DNA, 20 bp, Artificial Sequence
  source: /note=“Description of Artificial Sequence Synthetic primer”
gcacgacgtacgaatgagtc

SEQ ID NO: 759 - DNA, 19 bp, Artificial Sequence
  source: /note=“Description of Artificial Sequence Synthetic primer”
agcaccaggctttggctat

SEQ ID NO: 760 - DNA, 22 bp, Artificial Sequence
  source: /note=“Description of Artificial Sequence Synthetic primer”
gaaggatgtgagaaaagacctg

SEQ ID NO: 761 - DNA, 20 bp, Artificial Sequence
  source: /note=“Description of Artificial Sequence Synthetic primer”
gcatggtcactgagttttgc

SEQ ID NO: 762 - DNA, 20 bp, Artificial Sequence
  source: /note=“Description of Artificial Sequence Synthetic primer”
caagccacaagaggtgatga

SEQ ID NO: 763 - DNA, 23 bp, Artificial Sequence
  source: /note=“Description of Artificial Sequence Synthetic primer”
cacagaacagcttgtgaaaatca

SEQ ID NO: 764 - DNA, 21 bp, Artificial Sequence
  source: /note=“Description of Artificial Sequence Synthetic primer”
tggtactaagacccaccaaaa

SEQ ID NO: 765 - DNA, 24 bp, Artificial Sequence
  source: /note=“Description of Artificial Sequence Synthetic primer”
aaaaccctctaactaggcattgaa

SEQ ID NO: 766 - DNA, 24 bp, Artificial Sequence
  source: /note=“Description of Artificial Sequence Synthetic primer”
gcttgctcttattattttgacgtt

SEQ ID NO: 767 - DNA, 21 bp, Artificial Sequence
  source: /note=“Description of Artificial Sequence Synthetic primer”
agaatttggaactcactttgg

SEQ ID NO: 768 - DNA, 24 bp, Artificial Sequence
  source: /note=“Description of Artificial Sequence Synthetic primer”
aaacagatcctattgtgtctggaa

SEQ ID NO: 769 - DNA, 20 bp, Artificial Sequence
  source: /note=“Description of Artificial Sequence Synthetic primer”
acctcatgcaccagccctta

SEQ ID NO: 770 - DNA, 20 bp, Artificial Sequence
  source: /note=“Description of Artificial Sequence Synthetic primer”
aagtgttgctccctgctgtc

SEQ ID NO: 771 - DNA, 22 bp, Artificial Sequence
  source: /note=“Description of Artificial Sequence Synthetic primer”
aaaggtctttcaggagaatttg

SEQ ID NO: 772 - DNA, 20 bp, Artificial Sequence
  source: /note=“Description of Artificial Sequence Synthetic primer”
aggtggccatacacatgctt

SEQ ID NO: 773 - DNA, 19 bp, Artificial Sequence
  source: /note=“Description of Artificial Sequence Synthetic primer”
ggttgtccatgtccccaag

SEQ ID NO: 774 - DNA, 20 bp, Artificial Sequence
  source: /note=“Description of Artificial Sequence Synthetic primer”
tttgcagtgtttatgccaca

SEQ ID NO: 775 - DNA, 22 bp, Artificial Sequence
  source: /note=“Description of Artificial Sequence Synthetic primer”
tcatggcaatttaaatgatgag

SEQ ID NO: 776 - DNA, 22 bp, Artificial Sequence
  source: /note=“Description of Artificial Sequence Synthetic primer”
tttaaatggtgccttgtttctt

SEQ ID NO: 777 - DNA, 20 bp, Artificial Sequence
  source: /note=“Description of Artificial Sequence Synthetic primer”
cagggcactaactgaaaaat

SEQ ID NO: 778 - DNA, 23 bp, Artificial Sequence
  source: /note=“Description of Artificial Sequence Synthetic primer”
gggatatggattatctttctcat

SEQ ID NO: 779 - DNA, 20 bp, Artificial Sequence
  source: /note=“Description of Artificial Sequence Synthetic primer”
agccccaaactaagtgctga

SEQ ID NO: 780 - DNA, 19 bp, Artificial Sequence
  source: /note=“Description of Artificial Sequence Synthetic primer”
cccagagccagtgcattta

SEQ ID NO: 781 - DNA, 22 bp, Artificial Sequence
  source: /note=“Description of Artificial Sequence Synthetic primer”
tgatgagaaaacacagaaatgc

SEQ ID NO: 782 - DNA, 20 bp, Artificial Sequence
  source: /note=“Description of Artificial Sequence Synthetic primer”
cctggctgaatcaaggaaga

SEQ ID NO: 783 - DNA, 23 bp, Artificial Sequence
  source: /note=“Description of Artificial Sequence Synthetic primer”
cagtgacagttttctcattaagc

SEQ ID NO: 784 - DNA, 20 bp, Artificial Sequence
  source: /note=“Description of Artificial Sequence Synthetic primer”
taggaacaatccccaatcca

SEQ ID NO: 785 - DNA, 21 bp, Artificial Sequence
  source: /note=“Description of Artificial Sequence Synthetic primer”
tgagaaactcacttggggtca

SEQ ID NO: 786 - DNA, 20 bp, Artificial Sequence
  source: /note=“Description of Artificial Sequence Synthetic primer”
tgacagcaattctggtctgc

SEQ ID NO: 787 - DNA, 21 bp, Artificial Sequence
  source: /note=“Description of Artificial Sequence Synthetic primer”
aggcttgaagaaaagcttcat

SEQ ID NO: 788 - DNA, 23 bp, Artificial Sequence
  source: /note=“Description of Artificial Sequence Synthetic primer”
ctttttcatatccagtatttcag

SEQ ID NO: 789 - DNA, 24 bp, Artificial Sequence
  source: /note=“Description of Artificial Sequence Synthetic primer”
cagctagaatctatacaaggaagg

SEQ ID NO: 790 - DNA, 24 bp, Artificial Sequence
  source: /note=“Description of Artificial Sequence Synthetic primer”
ggatacaacaggaactaggatcaa

SEQ ID NO: 791 - DNA, 23 bp, Artificial Sequence
  source: /note=“Description of Artificial Sequence Synthetic primer”
cccattattatgctgttatgctg

SEQ ID NO: 792 - DNA, 23 bp, Artificial Sequence
  source: /note=“Description of Artificial Sequence Synthetic primer”
tctgagagttaaatccttggtga

SEQ ID NO: 793 - DNA, 22 bp, Artificial Sequence
  source: /note=“Description of Artificial Sequence Synthetic primer”
cacctcttaacagtttcatttt

SEQ ID NO: 794 - DNA, 20 bp, Artificial Sequence
  source: /note=“Description of Artificial Sequence Synthetic primer”
ggccgacagcttctacttta

SEQ ID NO: 795 - DNA, 20 bp, Artificial Sequence
  source: /note=“Description of Artificial Sequence Synthetic primer”
aaggagggcttagctagttg

SEQ ID NO: 796 - DNA, 23 bp, Artificial Sequence
  source: /note=“Description of Artificial Sequence Synthetic primer”
gctctttctcatcttaaggcttc

SEQ ID NO: 797 - DNA, 23 bp, Artificial Sequence
  source: /note=“Description of Artificial Sequence Synthetic primer”
gttaaaattactgttccagttgt

SEQ ID NO: 798 - DNA, 23 bp, Artificial Sequence
  source: /note=“Description of Artificial Sequence Synthetic primer”
caggcaaccaaataataacaaaa

SEQ ID NO: 799 - DNA, 20 bp, Artificial Sequence
  source: /note=“Description of Artificial Sequence Synthetic primer”
ggattctgcagaccctcagt

SEQ ID NO: 800 - DNA, 20 bp, Artificial Sequence
  source: /note=“Description of Artificial Sequence Synthetic primer”
caccttgccactcactgttg

SEQ ID NO: 801 - DNA, 23 bp, Artificial Sequence
  source: /note=“Description of Artificial Sequence Synthetic primer”
gggttccagcaatattctacctt

SEQ ID NO: 802 - DNA, 22 bp, Artificial Sequence
  source: /note=“Description of Artificial Sequence Synthetic primer”
ggtaatgaagaaagacaaaaca

SEQ ID NO: 803 - DNA, 18 bp, Artificial Sequence
  source: /note=“Description of Artificial Sequence Synthetic primer”
ctgtgtggctggggaagc

SEQ ID NO: 804 - DNA, 20 bp, Artificial Sequence
  source: /note=“Description of Artificial Sequence Synthetic primer”
gcacataacctcagaaccag

SEQ ID NO: 805 - DNA, 19 bp, Artificial Sequence
  source: /note=“Description of Artificial Sequence Synthetic primer”
ggagccccaaccctaattt

SEQ ID NO: 806 - DNA, 18 bp, Artificial Sequence
  source: /note=“Description of Artificial Sequence Synthetic primer”
atcctcatcctccgcaca

SEQ ID NO: 807 - DNA, 23 bp, Artificial Sequence
  source: /note=“Description of Artificial Sequence Synthetic primer”
cggtagctaagtatctgcttttt

SEQ ID NO: 808 - DNA, 22 bp, Artificial Sequence
  source: /note=“Description of Artificial Sequence Synthetic primer”
gggcaggaattattatgttcca

SEQ ID NO: 809 - DNA, 20 bp, Artificial Sequence
  source: /note=“Description of Artificial Sequence Synthetic primer”
ggatgtttttgcagtttatt

SEQ ID NO: 810 - DNA, 22 bp, Artificial Sequence
  source: /note=“Description of Artificial Sequence Synthetic primer”
acttgctctgatacctaaatga

SEQ ID NO: 811 - DNA, 20 bp, Artificial Sequence
  source: /note=“Description of Artificial Sequence Synthetic primer”
cggctctctcctcattctgt

SEQ ID NO: 812 - DNA, 20 bp, Artificial Sequence
  source: /note=“Description of Artificial Sequence Synthetic primer”
gcattgccactgagacatga

SEQ ID NO: 813 - DNA, 20 bp, Artificial Sequence
  source: /note=“Description of Artificial Sequence Synthetic primer”
aagaggagggctttgagtcc

SEQ ID NO: 814 - DNA, 24 bp, Artificial Sequence
  source: /note=“Description of Artificial Sequence Synthetic primer”
tttagtagagctactgatcattcc

SEQ ID NO: 815 - DNA, 24 bp, Artificial Sequence
  source: /note=“Description of Artificial Sequence Synthetic primer”
caattaagtcaggtaataatgctg

SEQ ID NO: 816 - DNA, 20 bp, Artificial Sequence
  source: /note=“Description of Artificial Sequence Synthetic primer”
aagccattcatttgggtttg

SEQ ID NO: 817 - DNA, 21 bp, Artificial Sequence
  source: /note=“Description of Artificial Sequence Synthetic primer”
ttgattcctattgagctttca

SEQ ID NO: 818 - DNA, 20 bp, Artificial Sequence
  source: /note=“Description of Artificial Sequence Synthetic primer”
ggcctctgacatcactctca

SEQ ID NO: 819 - DNA, 20 bp, Artificial Sequence
  source: /note=“Description of Artificial Sequence Synthetic primer”
ggcaagggtttaggacttgg

SEQ ID NO: 820 - DNA, 20 bp, Artificial Sequence
  source: /note=“Description of Artificial Sequence Synthetic primer”
ggattgcgcctcaaaataaa

SEQ ID NO: 821 - DNA, 20 bp, Artificial Sequence
  source: /note=“Description of Artificial Sequence Synthetic primer”
cttccctgcacatccttttg

SEQ ID NO: 822 - DNA, 24 bp, Artificial Sequence
  source: /note=“Description of Artificial Sequence Synthetic primer”
ctgtttaggaagagtcatgtaacc

SEQ ID NO: 823 - DNA, 21 bp, Artificial Sequence
  source: /note=“Description of Artificial Sequence Synthetic primer”
tggcctatttctcaaatgcag

SEQ ID NO: 824 - DNA, 20 bp, Artificial Sequence
  source: /note=“Description of Artificial Sequence Synthetic primer”
ctgcaaggcacgatctatga

SEQ ID NO: 825 - DNA, 24 bp, Artificial Sequence
  source: /note=“Description of Artificial Sequence Synthetic primer”
gtgattctaacaggtatgtaatga

SEQ ID NO: 826 - DNA, 22 bp, Artificial Sequence
  source: /note=“Description of Artificial Sequence Synthetic primer”
tgcatgttaacaccacattgag

SEQ ID NO: 827 - DNA, 23 bp, Artificial Sequence
  source: /note=“Description of Artificial Sequence Synthetic primer”
ggagaccatactgaagttatttt

SEQ ID NO: 828 - DNA, 20 bp, Artificial Sequence
  source: /note=“Description of Artificial Sequence Synthetic primer”
tttcgagttggtggtaattt

SEQ ID NO: 829 - DNA, 23 bp, Artificial Sequence
  source: /note=“Description of Artificial Sequence Synthetic primer”
tcgaaggtagaattaaatgtttc

SEQ ID NO: 830 - DNA, 24 bp, Artificial Sequence
  source: /note=“Description of Artificial Sequence Synthetic primer”
gatagtgacttataacaactccaa

SEQ ID NO: 831 - DNA, 20 bp, Artificial Sequence
  source: /note=“Description of Artificial Sequence Synthetic primer”
tgaattgaagggttttggac

SEQ ID NO: 832 - DNA, 22 bp, Artificial Sequence
  source: /note=“Description of Artificial Sequence Synthetic primer”
gcacacgttaagatggtttgaa

SEQ ID NO: 833 - DNA, 20 bp, Artificial Sequence
  source: /note=“Description of Artificial Sequence Synthetic primer”
ggggagggagacgtaaaaac

SEQ ID NO: 834 - DNA, 23 bp, Artificial Sequence
  source: /note=“Description of Artificial Sequence Synthetic primer”
tccagattttcctgttcatgatt

SEQ ID NO: 835 - DNA, 20 bp, Artificial Sequence
  source: /note=“Description of Artificial Sequence Synthetic primer”
gcacatcacaagttaagagg

SEQ ID NO: 836 - DNA, 20 bp, Artificial Sequence
  source: /note=“Description of Artificial Sequence Synthetic primer”
ccccagtagggaacacactt

SEQ ID NO: 837 - DNA, 20 bp, Artificial Sequence
  source: /note=“Description of Artificial Sequence Synthetic primer”
caggatgcactttttggatg

SEQ ID NO: 838 - DNA, 20 bp, Artificial Sequence
  source: /note=“Description of Artificial Sequence Synthetic primer”
ggcttctcccagaaaatctc

SEQ ID NO: 839 - DNA, 20 bp, Artificial Sequence
  source: /note=“Description of Artificial Sequence Synthetic primer”
ggggaggccctacaagttat

SEQ ID NO: 840 - DNA, 19 bp, Artificial Sequence
  source: /note=“Description of Artificial Sequence Synthetic primer”
gaagggaggggcatcttta

SEQ ID NO: 841 - DNA, 23 bp, Artificial Sequence
  source: /note=“Description of Artificial Sequence Synthetic primer”
aaaatcacatctgctaaatatcc

SEQ ID NO: 842 - DNA, 23 bp, Artificial Sequence
  source: /note=“Description of Artificial Sequence Synthetic primer”
tggacgatagaacttgttagtgc

SEQ ID NO: 843 - DNA, 22 bp, Artificial Sequence
  source: /note=“Description of Artificial Sequence Synthetic primer”
ccattaagcagacacacctacg

SEQ ID NO: 844 - DNA, 21 bp, Artificial Sequence
  source: /note=“Description of Artificial Sequence Synthetic primer”
ctcctttgaaagtggatcaaa

SEQ ID NO: 845 - DNA, 22 bp, Artificial Sequence
  source: /note=“Description of Artificial Sequence Synthetic primer”
tctgaaaatggggctaaaactt

SEQ ID NO: 846 - DNA, 20 bp, Artificial Sequence
  source: /note=“Description of Artificial Sequence Synthetic primer”
tccttaaagcagccctaaaa

SEQ ID NO: 847 - DNA, 23 bp, Artificial Sequence
  source: /note=“Description of Artificial Sequence Synthetic primer”
agtttagatttcagtctatgcaa

SEQ ID NO: 848 - DNA, 21 bp, Artificial Sequence
  source: /note=“Description of Artificial Sequence Synthetic primer”
tggagaatagctcctgcagtt

SEQ ID NO: 849 - DNA, 20 bp, Artificial Sequence
  source: /note=“Description of Artificial Sequence Synthetic primer”
tctttctggagacactcagg

SEQ ID NO: 850 - DNA, 23 bp, Artificial Sequence
  source: /note=“Description of Artificial Sequence Synthetic primer”
ctggaatctagaaagaaaaagaa

SEQ ID NO: 851 - DNA, 22 bp, Artificial Sequence
  source: /note=“Description of Artificial Sequence Synthetic primer”
caaagatagatgagatgctttt

SEQ ID NO: 852 - DNA, 23 bp, Artificial Sequence
  source: /note=“Description of Artificial Sequence Synthetic primer”
ctgacattgaaaacttgaaagaa

SEQ ID NO: 853 - DNA, 19 bp, Artificial Sequence
  source: /note=“Description of Artificial Sequence Synthetic primer”
agccctcctccaccgttag

SEQ ID NO: 854 - DNA, 20 bp, Artificial Sequence
  source: /note=“Description of Artificial Sequence Synthetic primer”
gcccagctacgatttctcct

SEQ ID NO: 855 - DNA, 20 bp, Artificial Sequence
  source: /note=“Description of Artificial Sequence Synthetic primer”
ttttatgcagcctgtgatgg

SEQ ID NO: 856 - DNA, 22 bp, Artificial Sequence
  source: /note=“Description of Artificial Sequence Synthetic primer”
cccttagttcaatcaagccaac

SEQ ID NO: 857 - DNA, 21 bp, Artificial Sequence
  source: /note=“Description of Artificial Sequence Synthetic primer”
cactcttgcaatctccctcag

SEQ ID NO: 858 - DNA, 20 bp, Artificial Sequence
  source: /note=“Description of Artificial Sequence Synthetic primer”
ctgacccttgtgggattcat

SEQ ID NO: 859 - DNA, 23 bp, Artificial Sequence
  source: /note=“Description of Artificial Sequence Synthetic primer”
cttttatgatatccaccaagact

SEQ ID NO: 860 - DNA, 21 bp, Artificial Sequence
  source: /note=“Description of Artificial Sequence Synthetic primer”
tggatcatctgtccaaagtca

SEQ ID NO: 861 - DNA, 20 bp, Artificial Sequence
  source: /note=“Description of Artificial Sequence Synthetic primer”
ccaaaacctgctctccaaga

SEQ ID NO: 862 - DNA, 24 bp, Artificial Sequence
  source: /note=“Description of Artificial Sequence Synthetic primer”
aagactactgaggttgtgcaaaga

SEQ ID NO: 863 - DNA, 22 bp, Artificial Sequence
  source: /note=“Description of Artificial Sequence Synthetic primer”
ttcaacttggtaccctgaaaaa

SEQ ID NO: 864 - DNA, 24 bp, Artificial Sequence
  source: /note=“Description of Artificial Sequence Synthetic primer”
agtcagttagtatgcagtacttgg

SEQ ID NO: 865 - DNA, 23 bp, Artificial Sequence
  source: /note=“Description of Artificial Sequence Synthetic primer”
tcttaaaagtgtcttgactgaaa

SEQ ID NO: 866 - DNA, 21 bp, Artificial Sequence
  source: /note=“Description of Artificial Sequence Synthetic primer”
ggtcaatggctaaatcattcg

SEQ ID NO: 867 - DNA, 22 bp, Artificial Sequence
  source: /note=“Description of Artificial Sequence Synthetic primer”
gcaattccagatatctctttat

SEQ ID NO: 868 - DNA, 21 bp, Artificial Sequence
  source: /note=“Description of Artificial Sequence Synthetic primer”
ttatctacccatgcttctctc

SEQ ID NO: 869 - DNA, 24 bp, Artificial Sequence
  source: /note=“Description of Artificial Sequence Synthetic primer”
gcataaacaaatgtgtaacgtggt

SEQ ID NO: 870 - DNA, 22 bp, Artificial Sequence
  source: /note=“Description of Artificial Sequence Synthetic primer”
tgttttcgtagtctttattgct

SEQ ID NO: 871 - DNA, 23 bp, Artificial Sequence
  source: /note=“Description of Artificial Sequence Synthetic primer”
tgctagctatatgtaggtcagtt

SEQ ID NO: 872 - DNA, 22 bp, Artificial Sequence
  source: /note=“Description of Artificial Sequence Synthetic primer”
cgttagttccctggaaagatca

SEQ ID NO: 873 - DNA, 23 bp, Artificial Sequence
  source: /note=“Description of Artificial Sequence Synthetic primer”
ttgcatagatgtagcagtatttc

SEQ ID NO: 874 - DNA, 24 bp, Artificial Sequence
  source: /note=“Description of Artificial Sequence Synthetic primer”
gactttcttaaagctgcacaatca

SEQ ID NO: 875 - DNA, 20 bp, Artificial Sequence
  source: /note=“Description of Artificial Sequence Synthetic primer”
gtttgcttgcttttactttg

SEQ ID NO: 876 - DNA, 21 bp, Artificial Sequence
  source: /note=“Description of Artificial Sequence Synthetic primer”
tgtgaagcaccatttctgttt

SEQ ID NO: 877 - DNA, 22 bp, Artificial Sequence
  source: /note=“Description of Artificial Sequence Synthetic primer”
aacagtgaggctctcctgtagc

SEQ ID NO: 878 - DNA, 19 bp, Artificial Sequence
  source: /note=“Description of Artificial Sequence Synthetic primer”
cccattgtcaccgaggata

SEQ ID NO: 879 - DNA, 24 bp, Artificial Sequence
  source: /note=“Description of Artificial Sequence Synthetic primer”
cagagagctcacttctagttctgc

SEQ ID NO: 880 - DNA, 22 bp, Artificial Sequence
  source: /note=“Description of Artificial Sequence Synthetic primer”
gctatcttgggtcatgaatttg

SEQ ID NO: 881 - DNA, 20 bp, Artificial Sequence
  source: /note=“Description of Artificial Sequence Synthetic primer”
cctcatgcaattcaaaggaa

SEQ ID NO: 882 - DNA, 20 bp, Artificial Sequence
  source: /note=“Description of Artificial Sequence Synthetic primer”
catttcccctaggtttgtgc

SEQ ID NO: 883 - DNA, 19 bp, Artificial Sequence
  source: /note=“Description of Artificial Sequence Synthetic primer”
gtggggcacacagtgtctt

SEQ ID NO: 884 - DNA, 23 bp, Artificial Sequence
  source: /note=“Description of Artificial Sequence Synthetic primer”
cttagatttgttcatctgatggt

SEQ ID NO: 885 - DNA, 20 bp, Artificial Sequence
  source: /note=“Description of Artificial Sequence Synthetic primer”
ttgggtagatgcaatgcaag

SEQ ID NO: 886 - DNA, 21 bp, Artificial Sequence
  source: /note=“Description of Artificial Sequence Synthetic primer”
aacccatatgactaaggtgaa

SEQ ID NO: 887 - DNA, 22 bp, Artificial Sequence
  source: /note=“Description of Artificial Sequence Synthetic primer”
gctgaaaattcacactgtggtc

SEQ ID NO: 888 - DNA, 23 bp, Artificial Sequence
  source: /note=“Description of Artificial Sequence Synthetic primer”
tgtcataatgaagagctagttgc

SEQ ID NO: 889 - DNA, 24 bp, Artificial Sequence
  source: /note=“Description of Artificial Sequence Synthetic primer”
gagaggtaagagagagtatctttg

SEQ ID NO: 890 - DNA, 23 bp, Artificial Sequence
  source: /note=“Description of Artificial Sequence Synthetic primer”
gagttatttcccttaaaaaccag

SEQ ID NO: 891 - DNA, 21 bp, Artificial Sequence
  source: /note=“Description of Artificial Sequence Synthetic primer”
gctacgcttgacacccttaca

SEQ ID NO: 892 - DNA, 22 bp, Artificial Sequence
  source: /note=“Description of Artificial Sequence Synthetic primer”
ggatgctgtgagtgctaaatga

SEQ ID NO: 893 - DNA, 20 bp, Artificial Sequence
  source: /note=“Description of Artificial Sequence Synthetic primer”
ggcactgcgtcagcatacta

SEQ ID NO: 894 - DNA, 18 bp, Artificial Sequence
  source: /note=“Description of Artificial Sequence Synthetic primer”
ctggctccttgccatcat

SEQ ID NO: 895 - DNA, 20 bp, Artificial Sequence
  source: /note=“Description of Artificial Sequence Synthetic primer”
taggcctcagaaagaacgag

SEQ ID NO: 896 - DNA, 21 bp, Artificial Sequence
  source: /note=“Description of Artificial Sequence Synthetic primer”
tgctaggcttacttcgttttc

SEQ ID NO: 897 - DNA, 22 bp, Artificial Sequence
  source: /note=“Description of Artificial Sequence Synthetic primer”
aaaataattccctttggtatgc

SEQ ID NO: 898 - DNA, 21 bp, Artificial Sequence
  source: /note=“Description of Artificial Sequence Synthetic primer”
catcatgaattctcccaatgc

SEQ ID NO: 899 - DNA, 22 bp, Artificial Sequence
  source: /note=“Description of Artificial Sequence Synthetic primer”
ttgggtaaatgtgtgactacgc

SEQ ID NO: 900 - DNA, 20 bp, Artificial Sequence
  source: /note=“Description of Artificial Sequence Synthetic primer”
tacctggggccctgatttat

SEQ ID NO: 901 - DNA, 21 bp, Artificial Sequence
  source: /note=“Description of Artificial Sequence Synthetic primer”
gcactgaaaatgttagtgatt

SEQ ID NO: 902 - DNA, 24 bp, Artificial Sequence
  source: /note=“Description of Artificial Sequence Synthetic primer”
ccttagtgaggtatttaggttaca

SEQ ID NO: 903 - DNA, 20 bp, Artificial Sequence
  source: /note=“Description of Artificial Sequence Synthetic primer”
agggagttatgatgccaagg

SEQ ID NO: 904 - DNA, 22 bp, Artificial Sequence
  source: /note=“Description of Artificial Sequence Synthetic primer”
tgatcaggggtagaagagattt

SEQ ID NO: 905 - DNA, 20 bp, Artificial Sequence
  source: /note=“Description of Artificial Sequence Synthetic primer”
cggcttccaatcgtatcttg

SEQ ID NO: 906 - DNA, 21 bp, Artificial Sequence
  source: /note=“Description of Artificial Sequence Synthetic primer”
gacaagtcagagaacaagctg

SEQ ID NO: 907 - DNA, 23 bp, Artificial Sequence
  source: /note=“Description of Artificial Sequence Synthetic primer”
tcatctgtaactaatgaaccttg

SEQ ID NO: 908 - DNA, 20 bp, Artificial Sequence
  source: /note=“Description of Artificial Sequence Synthetic primer”
tcaggaaagaatgctactca

SEQ ID NO: 909 - DNA, 20 bp, Artificial Sequence
  source: /note=“Description of Artificial Sequence Synthetic primer”
aattggatgctgttttaacc

SEQ ID NO: 910 - DNA, 22 bp, Artificial Sequence
  source: /note=“Description of Artificial Sequence Synthetic primer”
tgccacatgacaaattatcaca

SEQ ID NO: 911 - DNA, 23 bp, Artificial Sequence
  source: /note=“Description of Artificial Sequence Synthetic primer”
ccaaggtttagctacatgtataa

SEQ ID NO: 912 - DNA, 23 bp, Artificial Sequence
  source: /note=“Description of Artificial Sequence Synthetic primer”
ctgatagaaaaatttctgttgtg

SEQ ID NO: 913 - DNA, 18 bp, Artificial Sequence
  source: /note=“Description of Artificial Sequence Synthetic primer”
attccttcccgccttgct

SEQ ID NO: 914 - DNA, 20 bp, Artificial Sequence
  source: /note=“Description of Artificial Sequence Synthetic primer”
attcctgcacaggctcagac

SEQ ID NO: 915 - DNA, 24 bp, Artificial Sequence
  source: /note=“Description of Artificial Sequence Synthetic primer”
aaatgttcagtgtaaaaggctaca

SEQ ID NO: 916 - DNA, 23 bp, Artificial Sequence
  source: /note=“Description of Artificial Sequence Synthetic primer”
aaaggactagcagcatgtaactc

SEQ ID NO: 917 - DNA, 20 bp, Artificial Sequence
  source: /note=“Description of Artificial Sequence Synthetic primer”
cactacttccccttcccaaa

SEQ ID NO: 918 - DNA, 23 bp, Artificial Sequence
  source: /note=“Description of Artificial Sequence Synthetic primer”
aagatctggtagaaataaatgga

SEQ ID NO: 919 - DNA, 20 bp, Artificial Sequence
  source: /note=“Description of Artificial Sequence Synthetic primer”
gcttccaggctaaaagaagg

SEQ ID NO: 920 - DNA, 20 bp, Artificial Sequence
  source: /note=“Description of Artificial Sequence Synthetic primer”
aaaaagaaaagctggttagg

SEQ ID NO: 921 - DNA, 24 bp, Artificial Sequence
  source: /note=“Description of Artificial Sequence Synthetic primer”
cacctctatggtttagtccactcc

SEQ ID NO: 922 - DNA, 20 bp, Artificial Sequence
  source: /note=“Description of Artificial Sequence Synthetic primer”
cctgggattgaaagcaccta

SEQ ID NO: 923 - DNA, 20 bp, Artificial Sequence
  source: /note=“Description of Artificial Sequence Synthetic primer”
ggaattgccactctggagaa

SEQ ID NO: 924 - DNA, 20 bp, Artificial Sequence
  source: /note=“Description of Artificial Sequence Synthetic primer”
agtggtccccaacaacttga

SEQ ID NO: 925 - DNA, 23 bp, Artificial Sequence
  source: /note=“Description of Artificial Sequence Synthetic primer”
tcagataaaacaattccagttac

SEQ ID NO: 926 - DNA, 20 bp, Artificial Sequence
  source: /note=“Description of Artificial Sequence Synthetic primer”
acccacagaggaaagccttg

SEQ ID NO: 927 - DNA, 20 bp, Artificial Sequence
  source: /note=“Description of Artificial Sequence Synthetic primer”
cctgctggcacacgtaagtt

SEQ ID NO: 928 - DNA, 20 bp, Artificial Sequence
  source: /note=“Description of Artificial Sequence Synthetic primer”
ccatgggaatttgaaccact

SEQ ID NO: 929 - DNA, 20 bp, Artificial Sequence
  source: /note=“Description of Artificial Sequence Synthetic primer”
aaccacaatccacctcttgc

SEQ ID NO: 930 - DNA, 23 bp, Artificial Sequence
  source: /note=“Description of Artificial Sequence Synthetic primer”
gccaagtcattaacacaaagtga

SEQ ID NO: 931 - DNA, 22 bp, Artificial Sequence
  source: /note=“Description of Artificial Sequence Synthetic primer”
cccactcttctgctttactcca

SEQ ID NO: 932 - DNA, 21 bp, Artificial Sequence
  source: /note=“Description of Artificial Sequence Synthetic primer”
gagaaggggaaagagaacaaa

SEQ ID NO: 933 - DNA, 20 bp, Artificial Sequence
  source: /note=“Description of Artificial Sequence Synthetic primer”
ggctttttccacccagctta

SEQ ID NO: 934 - DNA, 20 bp, Artificial Sequence
  source: /note=“Description of Artificial Sequence Synthetic primer”
agtgggcaataataaacctt

SEQ ID NO: 935 - DNA, 20 bp, Artificial Sequence
  source: /note=“Description of Artificial Sequence Synthetic primer”
ggtggctggagaaattgaga

SEQ ID NO: 936 - DNA, 22 bp, Artificial Sequence
  source: /note=“Description of Artificial Sequence Synthetic primer”
aaagacaatttggctggtgttt

SEQ ID NO: 937 - DNA, 20 bp, Artificial Sequence
  source: /note=“Description of Artificial Sequence Synthetic primer”
gctaagttgcctccaagctg

SEQ ID NO: 938 - DNA, 20 bp, Artificial Sequence
  source: /note=“Description of Artificial Sequence Synthetic primer”
ttccctatttctgccaaagc

SEQ ID NO: 939 - DNA, 21 bp, Artificial Sequence
  source: /note=“Description of Artificial Sequence Synthetic primer”
ttcatggagatttgaccagtg

SEQ ID NO: 940 - DNA, 24 bp, Artificial Sequence
  source: /note=“Description of Artificial Sequence Synthetic primer”
cagatactcctttttggagagtca

SEQ ID NO: 941 - DNA, 22 bp, Artificial Sequence
  source: /note=“Description of Artificial Sequence Synthetic primer”
cagctaatgcataagggagatg

SEQ ID NO: 942 - DNA, 22 bp, Artificial Sequence
  source: /note=“Description of Artificial Sequence Synthetic primer”
ccagaacatttcatcactccaa

SEQ ID NO: 943 - DNA, 21 bp, Artificial Sequence
  source: /note=“Description of Artificial Sequence Synthetic primer”
gtacagagtccctgtctcaca

SEQ ID NO: 944 - DNA, 23 bp, Artificial Sequence
  source: /note=“Description of Artificial Sequence Synthetic primer”
catgatctgtctctctcactgaa

SEQ ID NO: 945 - DNA, 20 bp, Artificial Sequence
  source: /note=“Description of Artificial Sequence Synthetic primer”
gtggcagaactgacatgcaa

SEQ ID NO: 946 - DNA, 18 bp, Artificial Sequence
  source: /note=“Description of Artificial Sequence Synthetic primer”
tgtgggggcagacagact

SEQ ID NO: 947 - DNA, 19 bp, Artificial Sequence
  source: /note=“Description of Artificial Sequence Synthetic primer”
tccaccagaaaccctttgg

SEQ ID NO: 948 - DNA, 20 bp, Artificial Sequence
  source: /note=“Description of Artificial Sequence Synthetic primer”
cctctgtggaaaggaaggaa

SEQ ID NO: 949 - DNA, 19 bp, Artificial Sequence
  source: /note=“Description of Artificial Sequence Synthetic primer”
cccgctccaggttattctc

SEQ ID NO: 950 - DNA, 21 bp, Artificial Sequence
  source: /note=“Description of Artificial Sequence Synthetic primer”
aagaaatctgaaaagcagagg

SEQ ID NO: 951 - DNA, 21 bp, Artificial Sequence
  source: /note=“Description of Artificial Sequence Synthetic primer”
aactgattcacatgaggttgc

SEQ ID NO: 952 - DNA, 21 bp, Artificial Sequence
  source: /note=“Description of Artificial Sequence Synthetic primer”
tttgagaggcaacattaacaa

SEQ ID NO: 953 - DNA, 20 bp, Artificial Sequence
  source: /note=“Description of Artificial Sequence Synthetic primer”
agtctgtacaaggggccaca

SEQ ID NO: 954 - DNA, 20 bp, Artificial Sequence
  source: /note=“Description of Artificial Sequence Synthetic primer”
taaggctcctgtggtagacg

SEQ ID NO: 955 - DNA, 20 bp, Artificial Sequence
  source: /note=“Description of Artificial Sequence Synthetic primer”
catcatggaaggtccctcac

SEQ ID NO: 956 - DNA, 20 bp, Artificial Sequence
  source: /note=“Description of Artificial Sequence Synthetic primer”
caagatcaaggcattggtag

SEQ ID NO: 957 - DNA, 23 bp, Artificial Sequence
  source: /note=“Description of Artificial Sequence Synthetic primer”
aggttcagattctatttctgtca

SEQ ID NO: 958 - DNA, 23 bp, Artificial Sequence
  source: /note=“Description of Artificial Sequence Synthetic primer”
ccttgcctaagataacacaacca

SEQ ID NO: 959 - DNA, 23 bp, Artificial Sequence
  source: /note=“Description of Artificial Sequence Synthetic primer”
tgttttgtaattcctttcagtca

SEQ ID NO: 960 - DNA, 24 bp, Artificial Sequence
  source: /note=“Description of Artificial Sequence Synthetic primer”
cctcaaatactgaagatagcaagc

SEQ ID NO: 961 - DNA, 21 bp, Artificial Sequence
  source: /note=“Description of Artificial Sequence Synthetic primer”
gacaagaactgaaggcaaagg

SEQ ID NO: 962 - DNA, 21 bp, Artificial Sequence
  source: /note=“Description of Artificial Sequence Synthetic primer”
gggaggaacagaacaaccttc

SEQ ID NO: 963 - DNA, 20 bp, Artificial Sequence
  source: /note=“Description of Artificial Sequence Synthetic primer”
tcgcagtcttttgcatcatt

SEQ ID NO: 964 - DNA, 23 bp, Artificial Sequence
  source: /note=“Description of Artificial Sequence Synthetic primer”
tccaatagctaccttcaccagaa

SEQ ID NO: 965 - DNA, 23 bp, Artificial Sequence
  source: /note=“Description of Artificial Sequence Synthetic primer”
ggttaaattctacttcgcaacca

SEQ ID NO: 966 - DNA, 24 bp, Artificial Sequence
  source: /note=“Description of Artificial Sequence Synthetic primer”
gcagtgtagtctaactagctgtgt

SEQ ID NO: 967 - DNA, 20 bp, Artificial Sequence
  source: /note=“Description of Artificial Sequence Synthetic primer”
cagcttcccagtttctcaca

SEQ ID NO: 968 - DNA, 23 bp, Artificial Sequence
  source: /note=“Description of Artificial Sequence Synthetic primer”
aattgctacattcctgtctattg

SEQ ID NO: 969 - DNA, 20 bp, Artificial Sequence
  source: /note=“Description of Artificial Sequence Synthetic primer”
gcggaaagacattccatgtt

SEQ ID NO: 970 - DNA, 23 bp, Artificial Sequence
  source: /note=“Description of Artificial Sequence Synthetic primer”
tgcatctcaatgatattgctttt

SEQ ID NO: 971 - DNA, 20 bp, Artificial Sequence
  source: /note=“Description of Artificial Sequence Synthetic primer”
tctctgagagcaaagacact

SEQ ID NO: 972 - DNA, 23 bp, Artificial Sequence
  source: /note=“Description of Artificial Sequence Synthetic primer”
tgtgcaatagtaataatgggtct

SEQ ID NO: 973 - DNA, 20 bp, Artificial Sequence
  source: /note=“Description of Artificial Sequence Synthetic primer”
gagggtacctttctttctcc

SEQ ID NO: 974 - DNA, 20 bp, Artificial Sequence
  source: /note=“Description of Artificial Sequence Synthetic primer”
gctcagtgtctgacaaaagc

SEQ ID NO: 975 - DNA, 23 bp, Artificial Sequence
  source: /note=“Description of Artificial Sequence Synthetic primer”
tggctgcctaaaattatttacga

SEQ ID NO: 976 - DNA, 23 bp, Artificial Sequence
  source: /note=“Description of Artificial Sequence Synthetic primer”
aagcaaataaggccatctaagaa

SEQ ID NO: 977 - DNA, 24 bp, Artificial Sequence
  source: /note=“Description of Artificial Sequence Synthetic primer”
tcaaacaaaaacagtgtaggcatt

SEQ ID NO: 978 - DNA, 24 bp, Artificial Sequence
  source: /note=“Description of Artificial Sequence Synthetic primer”
gaaaagttaagtcagaggctatcg

SEQ ID NO: 979 - DNA, 20 bp, Artificial Sequence
  source: /note=“Description of Artificial Sequence Synthetic primer”
aagtcacctaaatggcatga

SEQ ID NO: 980 - DNA, 20 bp, Artificial Sequence
  source: /note=“Description of Artificial Sequence Synthetic primer”
agacacagcaagatgcaaaa

SEQ ID NO: 981 - DNA, 20 bp, Artificial Sequence
  source: /note=“Description of Artificial Sequence Synthetic primer”
cagcaaccctttgaagcaat

SEQ ID NO: 982 - DNA, 20 bp, Artificial Sequence
  source: /note=“Description of Artificial Sequence Synthetic primer”
tgttttctcttcaaatgcaa

SEQ ID NO: 983 - DNA, 21 bp, Artificial Sequence
  source: /note=“Description of Artificial Sequence Synthetic primer”
tgactcagtggtgaactgtct

SEQ ID NO: 984 - DNA, 22 bp, Artificial Sequence
  source: /note=“Description of Artificial Sequence Synthetic primer”
gcagcccattaatactagcaca

SEQ ID NO: 985 - DNA, 21 bp, Artificial Sequence
  source: /note=“Description of Artificial Sequence Synthetic primer”
tgcattcaagaggaagaaagg

SEQ ID NO: 986 - DNA, 20 bp, Artificial Sequence
  source: /note=“Description of Artificial Sequence Synthetic primer”
tcaggacgaattcacaggat

SEQ ID NO: 987 - DNA, 19 bp, Artificial Sequence
  source: /note=“Description of Artificial Sequence Synthetic primer”
atgaaggccaggctgtagg

SEQ ID NO: 988 - DNA, 23 bp, Artificial Sequence
  source: /note=“Description of Artificial Sequence Synthetic primer”
gaacattcactgccttactctca

SEQ ID NO: 989 - DNA, 20 bp, Artificial Sequence
  source: /note=“Description of Artificial Sequence Synthetic primer”
ttcagtgaagggatggacct

SEQ ID NO: 990 - DNA, 20 bp, Artificial Sequence
  source: /note=“Description of Artificial Sequence Synthetic primer”
ggccacaggatctcctatct

SEQ ID NO: 991 - DNA, 23 bp, Artificial Sequence
  source: /note=“Description of Artificial Sequence Synthetic primer”
ccaagtaatcacttcaaccctct

SEQ ID NO: 992 - DNA, 20 bp, Artificial Sequence
  source: /note=“Description of Artificial Sequence Synthetic primer”
gctagctacgcccacgagat

SEQ ID NO: 993 - DNA, 20 bp, Artificial Sequence
  source: /note=“Description of Artificial Sequence Synthetic primer”
aactcaaacctaagtgcccc

SEQ ID NO: 994 - DNA, 20 bp, Artificial Sequence
  source: /note=“Description of Artificial Sequence Synthetic primer”
ggaatggaatagtgtgtggg

SEQ ID NO: 995 - DNA, 19 bp, Artificial Sequence
  source: /note=“Description of Artificial Sequence Synthetic primer”
acactggtctcaagctccc

SEQ ID NO: 996 - DNA, 20 bp, Artificial Sequence
  source: /note=“Description of Artificial Sequence Synthetic primer”
cacacctgtaattctagccc

SEQ ID NO: 997 - DNA, 20 bp, Artificial Sequence
  source: /note=“Description of Artificial Sequence Synthetic primer”
agaaggaaggatcagagaag

SEQ ID NO: 998 - DNA, 19 bp, Artificial Sequence
  source: /note=“Description of Artificial Sequence Synthetic primer”
agctttcctccccacactg

SEQ ID NO: 999 - DNA, 20 bp, Artificial Sequence
  source: /note=“Description of Artificial Sequence Synthetic primer”
taacaaatttgcatgtcatc

SEQ ID NO: 1000 - DNA, 20 bp, Artificial Sequence
  source: /note=“Description of Artificial Sequence Synthetic primer”
agaagccaggtgctgaagtg

SEQ ID NO: 1001 - DNA, 20 bp, Artificial Sequence
  source: /note=“Description of Artificial Sequence Synthetic primer”
gctgtgtggagccctataaa

SEQ ID NO: 1002 - DNA, 20 bp, Artificial Sequence
  source: /note=“Description of Artificial Sequence Synthetic primer”
gaatgaaatggagtttgcag

SEQ ID NO: 1003 - DNA, 20 bp, Artificial Sequence
  source: /note=“Description of Artificial Sequence Synthetic primer”
ggcagtgtttaaggtgttgg

SEQ ID NO: 1004 - DNA, 24 bp, Artificial Sequence
  source: /note=“Description of Artificial Sequence Synthetic primer”
aggtagtgatttctaggcttatca

SEQ ID NO: 1005 - DNA, 23 bp, Artificial Sequence
  source: /note=“Description of Artificial Sequence Synthetic primer”
cctggaagtattcattcatgtgg

SEQ ID NO: 1006 - DNA, 20 bp, Artificial Sequence
  source: /note=“Description of Artificial Sequence Synthetic primer”
gggacatctgggtagcactg

SEQ ID NO: 1007 - DNA, 20 bp, Artificial Sequence
  source: /note=“Description of Artificial Sequence Synthetic primer”
aagagtgtctcctccctctg

SEQ ID NO: 1008 - DNA, 20 bp, Artificial Sequence
  source: /note=“Description of Artificial Sequence Synthetic primer”
aactggaggctgtgttagac

SEQ ID NO: 1009 - DNA, 20 bp, Artificial Sequence
  source: /note=“Description of Artificial Sequence Synthetic primer”
aaaaaccccaggctccattg

SEQ ID NO: 1010 - DNA, 20 bp, Artificial Sequence
  source: /note=“Description of Artificial Sequence Synthetic primer”
atgtccagctgcttcttttc

SEQ ID NO: 1011 - DNA, 20 bp, Artificial Sequence
  source: /note=“Description of Artificial Sequence Synthetic primer”
atggcttgtacttcctcctc

SEQ ID NO: 1012 - DNA, 20 bp, Artificial Sequence
  source: /note=“Description of Artificial Sequence Synthetic primer”
ttcggtggaatagcagcaag

SEQ ID NO: 1013 - DNA, 20 bp, Artificial Sequence
  source: /note=“Description of Artificial Sequence Synthetic primer”
agtatgccatcatgaaagcc

SEQ ID NO: 1014 - DNA, 20 bp, Artificial Sequence
  source: /note=“Description of Artificial Sequence Synthetic primer”
cttctttgactaaggctgac

SEQ ID NO: 1015 - DNA, 18 bp, Artificial Sequence
  source: /note=“Description of Artificial Sequence Synthetic primer”
gatcttccagggggcact

SEQ ID NO: 1016 - DNA, 20 bp, Artificial Sequence
  source: /note=“Description of Artificial Sequence Synthetic primer”
tcattttggtttcgttcatt

SEQ ID NO: 1017 - DNA, 20 bp, Artificial Sequence
  source: /note=“Description of Artificial Sequence Synthetic primer”
ccttttgtggcttttcctca

SEQ ID NO: 1018 - DNA, 20 bp, Artificial Sequence
  source: /note=“Description of Artificial Sequence Synthetic primer”
ggcattccaacatgaaaagg

SEQ ID NO: 1019 - DNA, 19 bp, Artificial Sequence
  source: /note=“Description of Artificial Sequence Synthetic primer”
cagctgctgttccctcaga

SEQ ID NO: 1020 - DNA, 20 bp, Artificial Sequence
  source: /note=“Description of Artificial Sequence Synthetic primer”
ccaaaaaaccatgccctctg

SEQ ID NO: 1021 - DNA, 21 bp, Artificial Sequence
  source: /note=“Description of Artificial Sequence Synthetic primer”
ggttcacagagcccaagttac

SEQ ID NO: 1022 - DNA, 24 bp, Artificial Sequence
  source: /note=“Description of Artificial Sequence Synthetic primer”
tgagtctcttactgatcctgtgac

SEQ ID NO: 1023 - DNA, 21 bp, Artificial Sequence
  source: /note=“Description of Artificial Sequence Synthetic primer”
cttccctctgcctcttttaga

SEQ ID NO: 1024 - DNA, 20 bp, Artificial Sequence
  source: /note=“Description of Artificial Sequence Synthetic primer”
ccaaagagctcaggtctcca

SEQ ID NO: 1025 - DNA, 20 bp, Artificial Sequence
  source: /note=“Description of Artificial Sequence Synthetic primer”
aggtgagcatggggttgata

SEQ ID NO: 1026 - DNA, 20 bp, Artificial Sequence
  source: /note=“Description of Artificial Sequence Synthetic primer”
acctcttccttcctcaccaa

SEQ ID NO: 1027 - DNA, 20 bp, Artificial Sequence
  source: /note=“Description of Artificial Sequence Synthetic primer”
ggcagctccacacaccttag

SEQ ID NO: 1028 - DNA, 23 bp, Artificial Sequence
  source: /note=“Description of Artificial Sequence Synthetic primer”
tcatcttttggttttagattgtg

SEQ ID NO: 1029 - DNA, 19 bp, Artificial Sequence
  source: /note=“Description of Artificial Sequence Synthetic primer”
caactgcccgcttatcctt

SEQ ID NO: 1030 - DNA, 21 bp, Artificial Sequence
  source: /note=“Description of Artificial Sequence Synthetic primer”
aagacagcttgaagattctgg

SEQ ID NO: 1031 - DNA, 20 bp, Artificial Sequence
  source: /note=“Description of Artificial Sequence Synthetic primer”
aaggtctaagggggcacaag

SEQ ID NO: 1032 - DNA, 18 bp, Artificial Sequence
  source: /note=“Description of Artificial Sequence Synthetic primer”
atggccacgctctttgtc

SEQ ID NO: 1033 - DNA, 22 bp, Artificial Sequence
  source: /note=“Description of Artificial Sequence Synthetic primer”
ccagattatcttcttcgcccta

SEQ ID NO: 1034 - DNA, 20 bp, Artificial Sequence
  source: /note=“Description of Artificial Sequence Synthetic primer”
tgattagggttgggaagtgg

SEQ ID NO: 1035 - DNA, 23 bp, Artificial Sequence
  source: /note=“Description of Artificial Sequence Synthetic primer”
ttcagctcttctactctggactg

SEQ ID NO: 1036 - DNA, 24 bp, Artificial Sequence
  source: /note=“Description of Artificial Sequence Synthetic primer”
tgaaacaagagaagactggatttg

SEQ ID NO: 1037 - DNA, 23 bp, Artificial Sequence
  source: /note=“Description of Artificial Sequence Synthetic primer”
caagttagtgagaaacagagtcg

SEQ ID NO: 1038 - DNA, 20 bp, Artificial Sequence
  source: /note=“Description of Artificial Sequence Synthetic primer”
ggcctctactccaagaaagc

SEQ ID NO: 1039 - DNA, 24 bp, Artificial Sequence
  source: /note=“Description of Artificial Sequence Synthetic primer”
gttatatctcttttgtttctctcc

SEQ ID NO: 1040 - DNA, 21 bp, Artificial Sequence
  source: /note=“Description of Artificial Sequence Synthetic primer”
ttggattgttagagaataacg

SEQ ID NO: 1041 - DNA, 20 bp, Artificial Sequence
  source: /note=“Description of Artificial Sequence Synthetic primer”
gtccagctgtgtgattatct

SEQ ID NO: 1042 - DNA, 20 bp, Artificial Sequence
  source: /note=“Description of Artificial Sequence Synthetic primer”
agagggagatggaataaaaa

SEQ ID NO: 1043 - DNA, 20 bp, Artificial Sequence
  source: /note=“Description of Artificial Sequence Synthetic primer”
aaaaataaacatccctgtgg

SEQ ID NO: 1044 - DNA, 20 bp, Artificial Sequence
  source: /note=“Description of Artificial Sequence Synthetic primer”
acatagccaccagccacact

SEQ ID NO: 1045 - DNA, 21 bp, Artificial Sequence
  source: /note=“Description of Artificial Sequence Synthetic primer”
tgctctttttctcacaaatga

SEQ ID NO: 1046 - DNA, 20 bp, Artificial Sequence
  source: /note=“Description of Artificial Sequence Synthetic primer”
atattggtcagtggggcaaa

SEQ ID NO: 1047 - DNA, 20 bp, Artificial Sequence
  source: /note=“Description of Artificial Sequence Synthetic primer”
gcacatgagctgagactgga

SEQ ID NO: 1048 - DNA, 20 bp, Artificial Sequence
  source: /note=“Description of Artificial Sequence Synthetic primer”
tggcagtattacctgagcaa

SEQ ID NO: 1049 - DNA, 18 bp, Artificial Sequence
  source: /note=“Description of Artificial Sequence Synthetic primer”
gcagcgtcttgcctcctt

SEQ ID NO: 1050 - DNA, 20 bp, Artificial Sequence
  source: /note=“Description of Artificial Sequence Synthetic primer”
gcccagctcttaacacaaca

SEQ ID NO: 1051 - DNA, 20 bp, Artificial Sequence
  source: /note=“Description of Artificial Sequence Synthetic primer”
aaaaggctggaggatgaagg

SEQ ID NO: 1052 - DNA, 20 bp, Artificial Sequence
  source: /note=“Description of Artificial Sequence Synthetic primer”
tcagaaggcacctctgtcac

SEQ ID NO: 1053 - DNA, 23 bp, Artificial Sequence
  source: /note=“Description of Artificial Sequence Synthetic primer”
tgcaaccaaaactcagttatcta

SEQ ID NO: 1054 - DNA, 20 bp, Artificial Sequence
  source: /note=“Description of Artificial Sequence Synthetic primer”
tcccttgcctatcattgctt

SEQ ID NO: 1055 - DNA, 18 bp, Artificial Sequence
  source: /note=“Description of Artificial Sequence Synthetic primer”
ttcccagccttccaggag

SEQ ID NO: 1056 - DNA, 20 bp, Artificial Sequence
  source: /note=“Description of Artificial Sequence Synthetic primer”
tacaatggctgactgagcac

SEQ ID NO: 1057 - DNA, 23 bp, Artificial Sequence
  source: /note=“Description of Artificial Sequence Synthetic primer”
tgatttaaacctgatcttggtga

SEQ ID NO: 1058 - DNA, 19 bp, Artificial Sequence
  source: /note=“Description of Artificial Sequence Synthetic primer”
attcctgtccaccctggtc

SEQ ID NO: 1059 - DNA, 20 bp, Artificial Sequence
  source: /note=“Description of Artificial Sequence Synthetic primer”
cctttgatcacaagcaacca

SEQ ID NO: 1060 - DNA, 21 bp, Artificial Sequence
  source: /note=“Description of Artificial Sequence Synthetic primer”
ttactcttgggtcaggtgcat

SEQ ID NO: 1061 - DNA, 19 bp, Artificial Sequence
  source: /note=“Description of Artificial Sequence Synthetic primer”
atggcagaagagcccagag

SEQ ID NO: 1062 - DNA, 20 bp, Artificial Sequence
  source: /note=“Description of Artificial Sequence Synthetic primer”
cgatgctgaccttctggagt

SEQ ID NO: 1063 - DNA, 20 bp, Artificial Sequence
  source: /note=“Description of Artificial Sequence Synthetic primer”
gctgaaaaacccaggaatca

SEQ ID NO: 1064 - DNA, 20 bp, Artificial Sequence
  source: /note=“Description of Artificial Sequence Synthetic primer”
ggagttgagggagagggtct

SEQ ID NO: 1065 - DNA, 20 bp, Artificial Sequence
  source: /note=“Description of Artificial Sequence Synthetic primer”
gacagaatgaaatgctgtgt

SEQ ID NO: 1066 - DNA, 21 bp, Artificial Sequence
  source: /note=“Description of Artificial Sequence Synthetic primer”
ctttctaatccagcagcctct

SEQ ID NO: 1067 - DNA, 20 bp, Artificial Sequence
  source: /note=“Description of Artificial Sequence Synthetic primer”
ctgatccccgtaagatcagc

SEQ ID NO: 1068 - DNA, 18 bp, Artificial Sequence
  source: /note=“Description of Artificial Sequence Synthetic primer”
caggatgaaacggtgcag

SEQ ID NO: 1069 - DNA, 20 bp, Artificial Sequence
  source: /note=“Description of Artificial Sequence Synthetic primer”
tctctgacctgcttcctcgt

SEQ ID NO: 1070 - DNA, 20 bp, Artificial Sequence
  source: /note=“Description of Artificial Sequence Synthetic primer”
taaggcaataggcaccaagc

SEQ ID NO: 1071 - DNA, 19 bp, Artificial Sequence
  source: /note=“Description of Artificial Sequence Synthetic primer”
agcaatggggtcagagtcc

SEQ ID NO: 1072 - DNA, 20 bp, Artificial Sequence
  source: /note=“Description of Artificial Sequence Synthetic primer”
agctgattccttccctggat

SEQ ID NO: 1073 - DNA, 20 bp, Artificial Sequence
  source: /note=“Description of Artificial Sequence Synthetic primer”
cctgatggaggatccacttg

SEQ ID NO: 1074 - DNA, 19 bp, Artificial Sequence
  source: /note=“Description of Artificial Sequence Synthetic primer”
ctgcaaagcttcccatcct

SEQ ID NO: 1075 - DNA, 20 bp, Artificial Sequence
  source: /note=“Description of Artificial Sequence Synthetic primer”
ggggatcttaaaagcaccaa

SEQ ID NO: 1076 - DNA, 20 bp, Artificial Sequence
  source: /note=“Description of Artificial Sequence Synthetic primer”
gacactcccacttctgccta

SEQ ID NO: 1077 - DNA, 23 bp, Artificial Sequence
  source: /note=“Description of Artificial Sequence Synthetic primer”
atttcttcaagtgtatacagagc

SEQ ID NO: 1078 - DNA, 20 bp, Artificial Sequence
  source: /note=“Description of Artificial Sequence Synthetic primer”
caggcaaacattcccttgta

SEQ ID NO: 1079 - DNA, 23 bp, Artificial Sequence
  source: /note=“Description of Artificial Sequence Synthetic primer”
cactgttgactccaaaacaaaaa

SEQ ID NO: 1080 - DNA, 20 bp, Artificial Sequence
  source: /note=“Description of Artificial Sequence Synthetic primer”
cttcccacaacaatgagctg

SEQ ID NO: 1081 - DNA, 22 bp, Artificial Sequence
  source: /note=“Description of Artificial Sequence Synthetic primer”
gcagctaagaaagactctccaa

SEQ ID NO: 1082 - DNA, 19 bp, Artificial Sequence
  source: /note=“Description of Artificial Sequence Synthetic primer”
tctttgctccccacctatt

SEQ ID NO: 1083 - DNA, 20 bp, Artificial Sequence
  source: /note=“Description of Artificial Sequence Synthetic primer”
tgaattcaactgatggcaca

SEQ ID NO: 1084 - DNA, 22 bp, Artificial Sequence
  source: /note=“Description of Artificial Sequence Synthetic primer”
aagatttaatcctttgagatgc

SEQ ID NO: 1085 - DNA, 20 bp, Artificial Sequence
  source: /note=“Description of Artificial Sequence Synthetic primer”
tgaaaggacccaccaaatgt

SEQ ID NO: 1086 - DNA, 20 bp, Artificial Sequence
  source: /note=“Description of Artificial Sequence Synthetic primer”
ttttgttgtgtgtttgcttt

SEQ ID NO: 1087 - DNA, 21 bp, Artificial Sequence
  source: /note=“Description of Artificial Sequence Synthetic primer”
ttgctggcttacattcattcc

SEQ ID NO: 1088 - DNA, 20 bp, Artificial Sequence
  source: /note=“Description of Artificial Sequence Synthetic primer”
tacagctcagccagttctgc

SEQ ID NO: 1089 - DNA, 22 bp, Artificial Sequence
  source: /note=“Description of Artificial Sequence Synthetic primer”
tggttggtatggttattattgg

SEQ ID NO: 1090 - DNA, 24 bp, Artificial Sequence
  source: /note=“Description of Artificial Sequence Synthetic primer”
gccttagtttctctttctgtaaaa

SEQ ID NO: 1091 - DNA, 18 bp, Artificial Sequence
  source: /note=“Description of Artificial Sequence Synthetic primer”
ggccagcacaaacacacc

SEQ ID NO: 1092 - DNA, 21 bp, Artificial Sequence
  source: /note=“Description of Artificial Sequence Synthetic primer”
tcctaggactctccctttaga

SEQ ID NO: 1093 - DNA, 20 bp, Artificial Sequence
  source: /note=“Description of Artificial Sequence Synthetic primer”
aatgggcagatgagagcaag

SEQ ID NO: 1094 - DNA, 20 bp, Artificial Sequence
  source: /note=“Description of Artificial Sequence Synthetic primer”
ccagtacctaccccatgtcc

SEQ ID NO: 1095 - DNA, 21 bp, Artificial Sequence
  source: /note=“Description of Artificial Sequence Synthetic primer”
tccttttgacaggtccacatc

SEQ ID NO: 1096 - DNA, 22 bp, Artificial Sequence
  source: /note=“Description of Artificial Sequence Synthetic primer”
tggcccaattttcagtaacttc

SEQ ID NO: 1097 - DNA, 22 bp, Artificial Sequence
  source: /note=“Description of Artificial Sequence Synthetic primer”
caccaggggtagaagtaagacg

SEQ ID NO: 1098 - DNA, 20 bp, Artificial Sequence
  source: /note=“Description of Artificial Sequence Synthetic primer”
gagtatccatgcccagaacc

SEQ ID NO: 1099 - DNA, 22 bp, Artificial Sequence
  source: /note=“Description of Artificial Sequence Synthetic primer”
tgcatgtctgtatgtgtgttgg

SEQ ID NO: 1100 - DNA, 20 bp, Artificial Sequence
  source: /note=“Description of Artificial Sequence Synthetic primer”
atgctcccactgcatcctta

SEQ ID NO: 1101 - DNA, 20 bp, Artificial Sequence
  source: /note=“Description of Artificial Sequence Synthetic primer”
aaatgaagagccagcagcat

SEQ ID NO: 1102 - DNA, 21 bp, Artificial Sequence
  source: /note=“Description of Artificial Sequence Synthetic primer”
cccaccaacactaacctagca

SEQ ID NO: 1103 - DNA, 20 bp, Artificial Sequence
  source: /note=“Description of Artificial Sequence Synthetic primer”
acatctagctgaggtcagaa

SEQ ID NO: 1104 - DNA, 22 bp, Artificial Sequence
  source: /note=“Description of Artificial Sequence Synthetic primer”
tgtgcagatttatgcaaatcaa

SEQ ID NO: 1105 - DNA, 20 bp, Artificial Sequence
  source: /note=“Description of Artificial Sequence Synthetic primer”
gggaatttctctggttggag

SEQ ID NO: 1106 - DNA, 20 bp, Artificial Sequence
  source: /note=“Description of Artificial Sequence Synthetic primer”
aaacacagcttcatgacaag

SEQ ID NO: 1107 - DNA, 20 bp, Artificial Sequence
  source: /note=“Description of Artificial Sequence Synthetic primer”
ggactgagcatatgtggaaa

SEQ ID NO: 1108 - DNA, 23 bp, Artificial Sequence
  source: /note=“Description of Artificial Sequence Synthetic primer”
cctgaatttttacttctttgctt

SEQ ID NO: 1109 - DNA, 22 bp, Artificial Sequence
  source: /note=“Description of Artificial Sequence Synthetic primer”
ttgctgagtaacaggaaaacaa

SEQ ID NO: 1110 - DNA, 23 bp, Artificial Sequence
  source: /note=“Description of Artificial Sequence Synthetic primer”
tgctaaaccattaaataatctgg

SEQ ID NO: 1111 - DNA, 20 bp, Artificial Sequence
  source: /note=“Description of Artificial Sequence Synthetic primer”
gatgctaagcccatctcctg

SEQ ID NO: 1112 - DNA, 20 bp, Artificial Sequence
  source: /note=“Description of Artificial Sequence Synthetic primer”
agggtaggaaggatgcaatg

SEQ ID NO: 1113 - DNA, 20 bp, Artificial Sequence
  source: /note=“Description of Artificial Sequence Synthetic primer”
ggagcgaccactcttcattt

SEQ ID NO: 1114 - DNA, 19 bp, Artificial Sequence
  source: /note=“Description of Artificial Sequence Synthetic primer”
ctgaagggctcccaggcta

SEQ ID NO: 1115 - DNA, 23 bp, Artificial Sequence
  source: /note=“Description of Artificial Sequence Synthetic primer”
gaagattttgtagctggtcttgg

SEQ ID NO: 1116 - DNA, 21 bp, Artificial Sequence
  source: /note=“Description of Artificial Sequence Synthetic primer”
ccacaatggtttgtaagattt

SEQ ID NO: 1117 - DNA, 22 bp, Artificial Sequence
  source: /note=“Description of Artificial Sequence Synthetic primer”
tgcgttctttggagataagacc

SEQ ID NO: 1118 - DNA, 21 bp, Artificial Sequence
  source: /note=“Description of Artificial Sequence Synthetic primer”
cacatttctcacccatgtcaa

SEQ ID NO: 1119 - DNA, 19 bp, Artificial Sequence
  source: /note=“Description of Artificial Sequence Synthetic primer”
gttccctcatctgcccttc

SEQ ID NO: 1120 - DNA, 21 bp, Artificial Sequence
  source: /note=“Description of Artificial Sequence Synthetic primer”
tgtgagatgagtggagagcaa

SEQ ID NO: 1121 - DNA, 20 bp, Artificial Sequence
  source: /note=“Description of Artificial Sequence Synthetic primer”
taaatgtgcctggcttgatg

SEQ ID NO: 1122 - DNA, 20 bp, Artificial Sequence
  source: /note=“Description of Artificial Sequence Synthetic primer”
ccctttccttccttggatgt

SEQ ID NO: 1123 - DNA, 20 bp, Artificial Sequence
  source: /note=“Description of Artificial Sequence Synthetic primer”
tgcaaggacaccagaacaga

SEQ ID NO: 1124 - DNA, 20 bp, Artificial Sequence
  source: /note=“Description of Artificial Sequence Synthetic primer”
catttgcacagcatctgacc

SEQ ID NO: 1125 - DNA, 22 bp, Artificial Sequence
  source: /note=“Description of Artificial Sequence Synthetic primer”
gggtgagatcaaattcttaggc

SEQ ID NO: 1126 - DNA, 24 bp, Artificial Sequence
  source: /note=“Description of Artificial Sequence Synthetic primer”
ttctaatatgtatttgggagagag

SEQ ID NO: 1127 - DNA, 20 bp, Artificial Sequence
  source: /note=“Description of Artificial Sequence Synthetic primer”
gccatgttttcatcttgtgg

SEQ ID NO: 1128 - DNA, 24 bp, Artificial Sequence
  source: /note=“Description of Artificial Sequence Synthetic primer”
tctgtaaaggacttcatgtttcat

SEQ ID NO: 1129 - DNA, 24 bp, Artificial Sequence
  source: /note=“Description of Artificial Sequence Synthetic primer”
tcctgccatcttaatagtctcaca

SEQ ID NO: 1130 - DNA, 20 bp, Artificial Sequence
  source: /note=“Description of Artificial Sequence Synthetic primer”
cttgtggcctctcattctcc

SEQ ID NO: 1131 - DNA, 23 bp, Artificial Sequence
  source: /note=“Description of Artificial Sequence Synthetic primer”
tgttaatgtaaaattgcctcgat

SEQ ID NO: 1132 - DNA, 20 bp, Artificial Sequence
  source: /note=“Description of Artificial Sequence Synthetic primer”
gagctctggcatttctctgc

SEQ ID NO: 1133 - DNA, 19 bp, Artificial Sequence
  source: /note=“Description of Artificial Sequence Synthetic primer”
tgctggaaagtcattttga

SEQ ID NO: 1134 - DNA, 19 bp, Artificial Sequence
  source: /note=“Description of Artificial Sequence Synthetic primer”
ttggcattatttgtgatcc

SEQ ID NO: 1135 - DNA, 18 bp, Artificial Sequence
  source: /note=“Description of Artificial Sequence Synthetic primer”
ccacactccccagaccag

SEQ ID NO: 1136 - DNA, 22 bp, Artificial Sequence
  source: /note=“Description of Artificial Sequence Synthetic primer”
gggaagaccagaacttcagaaa

SEQ ID NO: 1137 - DNA, 21 bp, Artificial Sequence
  source: /note=“Description of Artificial Sequence Synthetic primer”
ctcttgccttctcattcacaa

SEQ ID NO: 1138 - DNA, 20 bp, Artificial Sequence
  source: /note=“Description of Artificial Sequence Synthetic primer”
ctttcctccctttgggactc

SEQ ID NO: 1139 - DNA, 18 bp, Artificial Sequence
  source: /note=“Description of Artificial Sequence Synthetic primer”
cccacgcactgtaccaca

SEQ ID NO: 1140 - DNA, 20 bp, Artificial Sequence
  source: /note=“Description of Artificial Sequence Synthetic primer”
tcagggcgagatacaccttt

SEQ ID NO: 1141 - DNA, 19 bp, Artificial Sequence
  source: /note=“Description of Artificial Sequence Synthetic primer”
gccagctcagctcctctct

SEQ ID NO: 1142 - DNA, 20 bp, Artificial Sequence
  source: /note=“Description of Artificial Sequence Synthetic primer”
gagggaaattcgagcatcag

SEQ ID NO: 1143 - DNA, 23 bp, Artificial Sequence
  source: /note=“Description of Artificial Sequence Synthetic primer”
ggcactcaataaacattgacaca

SEQ ID NO: 1144 - DNA, 20 bp, Artificial Sequence
  source: /note=“Description of Artificial Sequence Synthetic primer”
gggagagaggtgttctcagc

SEQ ID NO: 1145 - DNA, 20 bp, Artificial Sequence
  source: /note=“Description of Artificial Sequence Synthetic primer”
cgcaataccttcaacagcag

SEQ ID NO: 1146 - DNA, 20 bp, Artificial Sequence
  source: /note=“Description of Artificial Sequence Synthetic primer”
ggtgggctgcattcataaag

SEQ ID NO: 1147 - DNA, 20 bp, Artificial Sequence
  source: /note=“Description of Artificial Sequence Synthetic primer”
tgccaagaatccactccaag

SEQ ID NO: 1148 - DNA, 20 bp, Artificial Sequence
  source: /note=“Description of Artificial Sequence Synthetic primer”
ggggagggagaattggacta

SEQ ID NO: 1149 - DNA, 24 bp, Artificial Sequence
  source: /note=“Description of Artificial Sequence Synthetic primer”
caaagaaacagaatgaaaaagtgg

SEQ ID NO: 1150 - DNA, 21 bp, Artificial Sequence
  source: /note=“Description of Artificial Sequence Synthetic primer”
caccaacctggaatgcttact

SEQ ID NO: 1151 - DNA, 23 bp, Artificial Sequence
  source: /note=“Description of Artificial Sequence Synthetic primer”
tgactgctctaaaatctttgtca

SEQ ID NO: 1152 - DNA, 20 bp, Artificial Sequence
  source: /note=“Description of Artificial Sequence Synthetic primer”
atacgccaaacagtgagatg

SEQ ID NO: 1153 - DNA, 23 bp, Artificial Sequence
  source: /note=“Description of Artificial Sequence Synthetic primer”
tgacctatctataacctgtccac

SEQ ID NO: 1154 - DNA, 23 bp, Artificial Sequence
  source: /note=“Description of Artificial Sequence Synthetic primer”
tgggaattttagtttctctgtct

SEQ ID NO: 1155 - DNA, 23 bp, Artificial Sequence
  source: /note=“Description of Artificial Sequence Synthetic primer”
attgatctatgtgtctgtagctt

SEQ ID NO: 1156 - DNA, 22 bp, Artificial Sequence
  source: /note=“Description of Artificial Sequence Synthetic primer”
aattaagacagtgtggtattgg

SEQ ID NO: 1157 - DNA, 20 bp, Artificial Sequence
  source: /note=“Description of Artificial Sequence Synthetic primer”
ttcagagagggacacccttg

SEQ ID NO: 1158 - DNA, 20 bp, Artificial Sequence
  source: /note=“Description of Artificial Sequence Synthetic primer”
ttcttcgcaaccacactttg

SEQ ID NO: 1159 - DNA, 18 bp, Artificial Sequence
  source: /note=“Description of Artificial Sequence Synthetic primer”
gaggctctctggggcttg

SEQ ID NO: 1160 - DNA, 20 bp, Artificial Sequence
  source: /note=“Description of Artificial Sequence Synthetic primer”
agccttccacctgattgaaa

SEQ ID NO: 1161 - DNA, 20 bp, Artificial Sequence
  source: /note=“Description of Artificial Sequence Synthetic primer”
agagtcatgcatccttcatt

SEQ ID NO: 1162 - DNA, 20 bp, Artificial Sequence
  source: /note=“Description of Artificial Sequence Synthetic primer”
tggtggagacacagatccaa

SEQ ID NO: 1163 - DNA, 19 bp, Artificial Sequence
  source: /note=“Description of Artificial Sequence Synthetic primer”
gcagcaggaaccattcaca

SEQ ID NO: 1164 - DNA, 20 bp, Artificial Sequence
  source: /note=“Description of Artificial Sequence Synthetic primer”
cacttgtgtcctccaacatt

SEQ ID NO: 1165 - DNA, 20 bp, Artificial Sequence
  source: /note=“Description of Artificial Sequence Synthetic primer”
cccctcagagtgatgactgg

SEQ ID NO: 1166 - DNA, 19 bp, Artificial Sequence
  source: /note=“Description of Artificial Sequence Synthetic primer”
ctcctgacccagccacttt

SEQ ID NO: 1167 - DNA, 21 bp, Artificial Sequence
  source: /note=“Description of Artificial Sequence Synthetic primer”
gaaaatcttgtggagcctgaa

SEQ ID NO: 1168 - DNA, 20 bp, Artificial Sequence
  source: /note=“Description of Artificial Sequence Synthetic primer”
agagaggagatgggggaaag

SEQ ID NO: 1169 - DNA, 21 bp, Artificial Sequence
  source: /note=“Description of Artificial Sequence Synthetic primer”
tgagcctacactaacacatca

SEQ ID NO: 1170 - DNA, 24 bp, Artificial Sequence
  source: /note=“Description of Artificial Sequence Synthetic primer”
gccctaatgtaaactaaagacgtt

SEQ ID NO: 1171 - DNA, 20 bp, Artificial Sequence
  source: /note=“Description of Artificial Sequence Synthetic primer”
ggaaatgtgaccctcacagg

SEQ ID NO: 1172 - DNA, 20 bp, Artificial Sequence
  source: /note=“Description of Artificial Sequence Synthetic primer”
ttttccatacctaaagaacg

SEQ ID NO: 1173 - DNA, 24 bp, Artificial Sequence
  source: /note=“Description of Artificial Sequence Synthetic primer”
catcatctcttccttatgttctcc

SEQ ID NO: 1174 - DNA, 18 bp, Artificial Sequence
  source: /note=“Description of Artificial Sequence Synthetic primer”
ggcctgggggtgctaatg

SEQ ID NO: 1175 - DNA, 20 bp, Artificial Sequence
  source: /note=“Description of Artificial Sequence Synthetic primer”
gggtggtctggtgatgtgtt

SEQ ID NO: 1176 - DNA, 21 bp, Artificial Sequence
  source: /note=“Description of Artificial Sequence Synthetic primer”
gctatgccaagggaacctaga

SEQ ID NO: 1177 - DNA, 18 bp, Artificial Sequence
  source: /note=“Description of Artificial Sequence Synthetic primer”
gggagtactctccaaagc

SEQ ID NO: 1178 - DNA, 20 bp, Artificial Sequence
  source: /note=“Description of Artificial Sequence Synthetic primer”
cctcctgtcactttccctca

SEQ ID NO: 1179 - DNA, 20 bp, Artificial Sequence
  source: /note=“Description of Artificial Sequence Synthetic primer”
tgctccacagatgacacagt

SEQ ID NO: 1180 - DNA, 19 bp, Artificial Sequence
  source: /note=“Description of Artificial Sequence Synthetic primer”
tggaatgtgatggatgaga

SEQ ID NO: 1181 - DNA, 19 bp, Artificial Sequence
  source: /note=“Description of Artificial Sequence Synthetic primer”
agccctgcttcagcttctg

SEQ ID NO: 1182 - DNA, 23 bp, Artificial Sequence
  source: /note=“Description of Artificial Sequence Synthetic primer”
ttgactactggaacttggagagg

SEQ ID NO: 1183 - DNA, 20 bp, Artificial Sequence
  source: /note=“Description of Artificial Sequence Synthetic primer”
tggaaacttcttgtggacct

SEQ ID NO: 1184 - DNA, 20 bp, Artificial Sequence
  source: /note=“Description of Artificial Sequence Synthetic primer”
gtgggtggaagacttgctct

SEQ ID NO: 1185 - DNA, 20 bp, Artificial Sequence
  source: /note=“Description of Artificial Sequence Synthetic primer”
tttctgggccacctacaagt

SEQ ID NO: 1186 - DNA, 19 bp, Artificial Sequence
  source: /note=“Description of Artificial Sequence Synthetic primer”
cccaaggttctgggctaag

SEQ ID NO: 1187 - DNA, 20 bp, Artificial Sequence
  source: /note=“Description of Artificial Sequence Synthetic primer”
cctcctcctcacactgcttc

SEQ ID NO: 1188 - DNA, 21 bp, Artificial Sequence
  source: /note=“Description of Artificial Sequence Synthetic primer”
ccctttcttagctcctgacca

SEQ ID NO: 1189 - DNA, 24 bp, Artificial Sequence
  source: /note=“Description of Artificial Sequence Synthetic primer”
ggtctaaagggagagtaggaggtc

SEQ ID NO: 1190 - DNA, 21 bp, Artificial Sequence
  source: /note=“Description of Artificial Sequence Synthetic primer”
gaatggtcttttcgtcattcc

SEQ ID NO: 1191 - DNA, 20 bp, Artificial Sequence
  source: /note=“Description of Artificial Sequence Synthetic primer”
ctttcccaaaaccccacact

SEQ ID NO: 1192 - DNA, 21 bp, Artificial Sequence
  source: /note=“Description of Artificial Sequence Synthetic primer”
cacacacaaggaaaaacagga

SEQ ID NO: 1193 - DNA, 18 bp, Artificial Sequence
  source: /note=“Description of Artificial Sequence Synthetic primer”
gctggatggagggtgagg

SEQ ID NO: 1194 - DNA, 20 bp, Artificial Sequence
  source: /note=“Description of Artificial Sequence Synthetic primer”
tgcctgcctgttagaacatc

SEQ ID NO: 1195 - DNA, 21 bp, Artificial Sequence
  source: /note=“Description of Artificial Sequence Synthetic primer”
ggcaatccgaagtctaagaga

SEQ ID NO: 1196 - DNA, 22 bp, Artificial Sequence
  source: /note=“Description of Artificial Sequence Synthetic primer”
tggaaccaacaacctatcatca

SEQ ID NO: 1197 - DNA, 20 bp, Artificial Sequence
  source: /note=“Description of Artificial Sequence Synthetic primer”
gactggtacttccccaagga

SEQ ID NO: 1198 - DNA, 23 bp, Artificial Sequence
  source: /note=“Description of Artificial Sequence Synthetic primer”
tgaaaatccatttggtagttgct

SEQ ID NO: 1199 - DNA, 20 bp, Artificial Sequence
  source: /note=“Description of Artificial Sequence Synthetic primer”
aaaatgactgtcccctatct

SEQ ID NO: 1200 - DNA, 23 bp, Artificial Sequence
  source: /note=“Description of Artificial Sequence Synthetic primer”
tggtaagtgggatgatactgagc

SEQ ID NO: 1201 - DNA, 24 bp, Artificial Sequence
  source: /note=“Description of Artificial Sequence Synthetic primer”
aagcatagaaggaaaaacagattg

SEQ ID NO: 1202 - DNA, 22 bp, Artificial Sequence
  source: /note=“Description of Artificial Sequence Synthetic primer”
cccctgaatgaaactattgagc

SEQ ID NO: 1203 - DNA, 20 bp, Artificial Sequence
  source: /note=“Description of Artificial Sequence Synthetic primer”
agcaagggagggaagacacc

SEQ ID NO: 1204 - DNA, 21 bp, Artificial Sequence
  source: /note=“Description of Artificial Sequence Synthetic primer”
ttgtcaatccttgctctaccc

SEQ ID NO: 1205 - DNA, 24 bp, Artificial Sequence
  source: /note=“Description of Artificial Sequence Synthetic primer”
tgaagggtagatatgaagtttttc

SEQ ID NO: 1206 - DNA, 20 bp, Artificial Sequence
  source: /note=“Description of Artificial Sequence Synthetic primer”
taatctttggactccttgaa

SEQ ID NO: 1207 - DNA, 21 bp, Artificial Sequence
  source: /note=“Description of Artificial Sequence Synthetic primer”
tgatcccatgtatttaaacct

SEQ ID NO: 1208 - DNA, 20 bp, Artificial Sequence
  source: /note=“Description of Artificial Sequence Synthetic primer”
cccctgaaatgagagtcacc

SEQ ID NO: 1209 - DNA, 21 bp, Artificial Sequence
  source: /note=“Description of Artificial Sequence Synthetic primer”
caaaataaacccaggcaaaaa

SEQ ID NO: 1210 - DNA, 23 bp, Artificial Sequence
  source: /note=“Description of Artificial Sequence Synthetic primer”
ctttaacaaatatagggcgattt

SEQ ID NO: 1211 - DNA, 21 bp, Artificial Sequence
  source: /note=“Description of Artificial Sequence Synthetic primer”
aagtaccaaaaaggcacatcg

SEQ ID NO: 1212 - DNA, 20 bp, Artificial Sequence
  source: /note=“Description of Artificial Sequence Synthetic primer”
tccccctaagatcaggaaca

SEQ ID NO: 1213 - DNA, 20 bp, Artificial Sequence
  source: /note=“Description of Artificial Sequence Synthetic primer”
tggaacagcaacttgcaaac

SEQ ID NO: 1214 - DNA, 21 bp, Artificial Sequence
  source: /note=“Description of Artificial Sequence Synthetic primer”
aagagtgtaaatgggtcctga

SEQ ID NO: 1215 - DNA, 20 bp, Artificial Sequence
  source: /note=“Description of Artificial Sequence Synthetic primer”
ctcccctgaacctgagtgac

SEQ ID NO: 1216 - DNA, 21 bp, Artificial Sequence
  source: /note=“Description of Artificial Sequence Synthetic primer”
tgctcacatttcattgaccag

SEQ ID NO: 1217 - DNA, 20 bp, Artificial Sequence
  source: /note=“Description of Artificial Sequence Synthetic primer”
tgaggtgggaagaaacacaa

SEQ ID NO: 1218 - DNA, 22 bp, Artificial Sequence
  source: /note=“Description of Artificial Sequence Synthetic primer”
tgcgactggatactatttttgg

SEQ ID NO: 1219 - DNA, 19 bp, Artificial Sequence
  source: /note=“Description of Artificial Sequence Synthetic primer”
agttgcatggagtggctga

SEQ ID NO: 1220 - DNA, 20 bp, Artificial Sequence
  source: /note=“Description of Artificial Sequence Synthetic primer”
tgttggtgcattcagagagc

SEQ ID NO: 1221 - DNA, 22 bp, Artificial Sequence
  source: /note=“Description of Artificial Sequence Synthetic primer”
caagtaattcttaccagccttt

SEQ ID NO: 1222 - DNA, 20 bp, Artificial Sequence
  source: /note=“Description of Artificial Sequence Synthetic primer”
aggctacaaaaaggcagcag

SEQ ID NO: 1223 - DNA, 18 bp, Artificial Sequence
  source: /note=“Description of Artificial Sequence Synthetic primer”
aaggaaacggccccagag

SEQ ID NO: 1224 - DNA, 20 bp, Artificial Sequence
  source: /note=“Description of Artificial Sequence Synthetic primer”
gaccctgtggactgagaacc

SEQ ID NO: 1225 - DNA, 20 bp, Artificial Sequence
  source: /note=“Description of Artificial Sequence Synthetic primer”
tcagagcactctgcattcca

SEQ ID NO: 1226 - DNA, 22 bp, Artificial Sequence
  source: /note=“Description of Artificial Sequence Synthetic primer”
ctttttaaagccagaaaaatgg

SEQ ID NO: 1227 - DNA, 21 bp, Artificial Sequence
  source: /note=“Description of Artificial Sequence Synthetic primer”
agaatcatatgacacatggaa

SEQ ID NO: 1228 - DNA, 24 bp, Artificial Sequence
  source: /note=“Description of Artificial Sequence Synthetic primer”
cagcttatctttatctgtttgctt

SEQ ID NO: 1229 - DNA, 20 bp, Artificial Sequence
  source: /note=“Description of Artificial Sequence Synthetic primer”
cactttgcagccaatccata

SEQ ID NO: 1230 - DNA, 19 bp, Artificial Sequence
  source: /note=“Description of Artificial Sequence Synthetic primer”
cagatctgatttcctggag

SEQ ID NO: 1231 - DNA, 24 bp, Artificial Sequence
  source: /note=“Description of Artificial Sequence Synthetic primer”
tccatacaggaagatccattaaga

SEQ ID NO: 1232 - DNA, 20 bp, Artificial Sequence
  source: /note=“Description of Artificial Sequence Synthetic primer”
gtgcagtttgggctacaaga

SEQ ID NO: 1233 - DNA, 20 bp, Artificial Sequence
  source: /note=“Description of Artificial Sequence Synthetic primer”
tgctgccagaagcaacctac

SEQ ID NO: 1234 - DNA, 20 bp, Artificial Sequence
  source: /note=“Description of Artificial Sequence Synthetic primer”
agaaagttgtgccaagtgct

SEQ ID NO: 1235 - DNA, 21 bp, Artificial Sequence
  source: /note=“Description of Artificial Sequence Synthetic primer”
tgtctggaaatcattgcttca

SEQ ID NO: 1236 - DNA, 22 bp, Artificial Sequence
  source: /note=“Description of Artificial Sequence Synthetic primer”
cataaagctaaaagattggaca

SEQ ID NO: 1237 - DNA, 19 bp, Artificial Sequence
  source: /note=“Description of Artificial Sequence Synthetic primer”
caaatcagtgtgccccaac

SEQ ID NO: 1238 - DNA, 20 bp, Artificial Sequence
  source: /note=“Description of Artificial Sequence Synthetic primer”
gttttgcccagaggtcatgt

SEQ ID NO: 1239 - DNA, 20 bp, Artificial Sequence
  source: /note=“Description of Artificial Sequence Synthetic primer”
gctcttccctcagtggctta

SEQ ID NO: 1240 - DNA, 21 bp, Artificial Sequence
  source: /note=“Description of Artificial Sequence Synthetic primer”
ctatcatttctccccaacaca

SEQ ID NO: 1241 - DNA, 20 bp, Artificial Sequence
  source: /note=“Description of Artificial Sequence Synthetic primer”
ctggatttcaaattgtttca

SEQ ID NO: 1242 - DNA, 23 bp, Artificial Sequence
  source: /note=“Description of Artificial Sequence Synthetic primer”
tcaagtatctagttgtgatagcc

SEQ ID NO: 1243 - DNA, 20 bp, Artificial Sequence
  source: /note=“Description of Artificial Sequence Synthetic primer”
tagagcagctaggggactgc

SEQ ID NO: 1244 - DNA, 20 bp, Artificial Sequence
  source: /note=“Description of Artificial Sequence Synthetic primer”
cgagactgttcaccctttgg

SEQ ID NO: 1245 - DNA, 20 bp, Artificial Sequence
  source: /note=“Description of Artificial Sequence Synthetic primer”
atgccagacttcaccactgc

SEQ ID NO: 1246 - DNA, 22 bp, Artificial Sequence
  source: /note=“Description of Artificial Sequence Synthetic primer”
tttcagttttgttatgtggcta

SEQ ID NO: 1247 - DNA, 24 bp, Artificial Sequence
  source: /note=“Description of Artificial Sequence Synthetic primer”
ttgaagttagttctttgtggatgg

SEQ ID NO: 1248 - DNA, 20 bp, Artificial Sequence
  source: /note=“Description of Artificial Sequence Synthetic primer”
atcaactccccacctggaag

SEQ ID NO: 1249 - DNA, 21 bp, Artificial Sequence
  source: /note=“Description of Artificial Sequence Synthetic primer”
ttttccctcattagctgcatt

SEQ ID NO: 1250 - DNA, 23 bp, Artificial Sequence
  source: /note=“Description of Artificial Sequence Synthetic primer”
tgattccagttcacagtagtcca

SEQ ID NO: 1251 - DNA, 20 bp, Artificial Sequence
  source: /note=“Description of Artificial Sequence Synthetic primer”
catttccagctgactggtta

SEQ ID NO: 1252 - DNA, 19 bp, Artificial Sequence
  source: /note=“Description of Artificial Sequence Synthetic primer”
accctgaggaggggctagt

SEQ ID NO: 1253 - DNA, 20 bp, Artificial Sequence
  source: /note=“Description of Artificial Sequence Synthetic primer”
gcccagtagcactgctcttc

SEQ ID NO: 1254 - DNA, 20 bp, Artificial Sequence
  source: /note=“Description of Artificial Sequence Synthetic primer”
agatcaccaaggcagaaacc

SEQ ID NO: 1255 - DNA, 19 bp, Artificial Sequence
  source: /note=“Description of Artificial Sequence Synthetic primer”
ccgagaacgctctgagttg

SEQ ID NO: 1256 - DNA, 20 bp, Artificial Sequence
  source: /note=“Description of Artificial Sequence Synthetic primer”
ggcagcaacaggaaatagca

SEQ ID NO: 1257 - DNA, 18 bp, Artificial Sequence
  source: /note=“Description of Artificial Sequence Synthetic primer”
acaggagtggctcggtca

SEQ ID NO: 1258 - DNA, 20 bp, Artificial Sequence
  source: /note=“Description of Artificial Sequence Synthetic primer”
cactgcaggaaatgcagctt

SEQ ID NO: 1259 - DNA, 20 bp, Artificial Sequence
  source: /note=“Description of Artificial Sequence Synthetic primer”
cgaaatccataggacctaca

SEQ ID NO: 1260 - DNA, 22 bp, Artificial Sequence
  source: /note=“Description of Artificial Sequence Synthetic primer”
agctacactatttccatgtgac

SEQ ID NO: 1261 - DNA, 20 bp, Artificial Sequence
  source: /note=“Description of Artificial Sequence Synthetic primer”
aacaagaaaggcagggaagg

SEQ ID NO: 1262 - DNA, 18 bp, Artificial Sequence
  source: /note=“Description of Artificial Sequence Synthetic primer”
ctgggtcacgcctcttga

SEQ ID NO: 1263 - DNA, 20 bp, Artificial Sequence
  source: /note=“Description of Artificial Sequence Synthetic primer”
tacaaacagtggggcaacaa

SEQ ID NO: 1264 - DNA, 18 bp, Artificial Sequence
  source: /note=“Description of Artificial Sequence Synthetic primer”
gccaggcatgggcttaat

SEQ ID NO: 1265 - DNA, 20 bp, Artificial Sequence
  source: /note=“Description of Artificial Sequence Synthetic primer”
ttcgtctttcagcaatttga

SEQ ID NO: 1266 - DNA, 24 bp, Artificial Sequence
  source: /note=“Description of Artificial Sequence Synthetic primer”
aacagaaagagagttacatctaca

SEQ ID NO: 1267 - DNA, 20 bp, Artificial Sequence
  source: /note=“Description of Artificial Sequence Synthetic primer”
cctcatgacctaaccacctc

SEQ ID NO: 1268 - DNA, 19 bp, Artificial Sequence
  source: /note=“Description of Artificial Sequence Synthetic primer”
cccccaatgcaagagtgtt

SEQ ID NO: 1269 - DNA, 20 bp, Artificial Sequence
  source: /note=“Description of Artificial Sequence Synthetic primer”
tttcacagtggaatgaatcg

SEQ ID NO: 1270 - DNA, 18 bp, Artificial Sequence
  source: /note=“Description of Artificial Sequence Synthetic primer”
gcccaggacacacaaaaa

SEQ ID NO: 1271 - DNA, 20 bp, Artificial Sequence
  source: /note=“Description of Artificial Sequence Synthetic primer”
ctggtcctctgtgaattgaa

SEQ ID NO: 1272 - DNA, 22 bp, Artificial Sequence
  source: /note=“Description of Artificial Sequence Synthetic primer”
caccgaatctatatctgtgagg

SEQ ID NO: 1273 - DNA, 21 bp, Artificial Sequence
  source: /note=“Description of Artificial Sequence Synthetic primer”
tctttatgtggccttcacttg

SEQ ID NO: 1274 - DNA, 20 bp, Artificial Sequence
  source: /note=“Description of Artificial Sequence Synthetic primer”
tatgctgaagctgccatcct

SEQ ID NO: 1275 - DNA, 19 bp, Artificial Sequence
  source: /note=“Description of Artificial Sequence Synthetic primer”
gggcaggaaacagggacta

SEQ ID NO: 1276 - DNA, 22 bp, Artificial Sequence
  source: /note=“Description of Artificial Sequence Synthetic primer”
gctgtcctatttcaggttgcat

SEQ ID NO: 1277 - DNA, 22 bp, Artificial Sequence
  source: /note=“Description of Artificial Sequence Synthetic primer”
tccactggaattggtagacaga

SEQ ID NO: 1278 - DNA, 21 bp, Artificial Sequence
  source: /note=“Description of Artificial Sequence Synthetic primer”
agcaatcatcctaggaggtca

SEQ ID NO: 1279 - DNA, 20 bp, Artificial Sequence
  source: /note=“Description of Artificial Sequence Synthetic primer”
ttctgacttcacagagggta

SEQ ID NO: 1280 - DNA, 20 bp, Artificial Sequence
  source: /note=“Description of Artificial Sequence Synthetic primer”
gggcaagtcacttagcattt

SEQ ID NO: 1281 - DNA, 22 bp, Artificial Sequence
  source: /note=“Description of Artificial Sequence Synthetic primer”
ttctcagacttcaaagcaaagg

SEQ ID NO: 1282 - DNA, 23 bp, Artificial Sequence
  source: /note=“Description of Artificial Sequence Synthetic primer”
tgaaaagatacctaaaatcaagg

SEQ ID NO: 1283 - DNA, 22 bp, Artificial Sequence
  source: /note=“Description of Artificial Sequence Synthetic primer”
gagaagaaccagacagaacacg

SEQ ID NO: 1284 - DNA, 20 bp, Artificial Sequence
  source: /note=“Description of Artificial Sequence Synthetic primer”
atttctgcagccctgtgact

SEQ ID NO: 1285 - DNA, 23 bp, Artificial Sequence
  source: /note=“Description of Artificial Sequence Synthetic primer”
catgaaaaataaggaaatgctga

SEQ ID NO: 1286 - DNA, 23 bp, Artificial Sequence
  source: /note=“Description of Artificial Sequence Synthetic primer”
tcctaagtttttctgatctgtgg

SEQ ID NO: 1287 - DNA, 20 bp, Artificial Sequence
  source: /note=“Description of Artificial Sequence Synthetic primer”
gcctcagtttcctcctcaga

SEQ ID NO: 1288 - DNA, 21 bp, Artificial Sequence
  source: /note=“Description of Artificial Sequence Synthetic primer”
cctctcaacaacccaggtact

SEQ ID NO: 1289 - DNA, 19 bp, Artificial Sequence
  source: /note=“Description of Artificial Sequence Synthetic primer”
actgtggctccagcatgaa

SEQ ID NO: 1290 - DNA, 20 bp, Artificial Sequence
  source: /note=“Description of Artificial Sequence Synthetic primer”
agtccaggcaccactgctac

SEQ ID NO: 1291 - DNA, 20 bp, Artificial Sequence
  source: /note=“Description of Artificial Sequence Synthetic primer”
gctggaaggagagaaacacg

SEQ ID NO: 1292 - DNA, 20 bp, Artificial Sequence
  source: /note=“Description of Artificial Sequence Synthetic primer”
atggccactagaggggagtc

SEQ ID NO: 1293 - DNA, 18 bp, Artificial Sequence
  source: /note=“Description of Artificial Sequence Synthetic primer”
gcatcctgtggtgggaag

SEQ ID NO: 1294 - DNA, 20 bp, Artificial Sequence
  source: /note=“Description of Artificial Sequence Synthetic primer”
tggtcaataagcctgttcca

SEQ ID NO: 1295 - DNA, 21 bp, Artificial Sequence
  source: /note=“Description of Artificial Sequence Synthetic primer”
ggtcaggacctgttttctcaa

SEQ ID NO: 1296 - DNA, 22 bp, Artificial Sequence
  source: /note=“Description of Artificial Sequence Synthetic primer”
tcaataactgctggagatgtgg

SEQ ID NO: 1297 - DNA, 21 bp, Artificial Sequence
  source: /note=“Description of Artificial Sequence Synthetic primer”
gcccaatctaatcatgtgagg

SEQ ID NO: 1298 - DNA, 18 bp, Artificial Sequence
  source: /note=“Description of Artificial Sequence Synthetic primer”
gcagccaagaaaggctgt

SEQ ID NO: 1299 - DNA, 20 bp, Artificial Sequence
  source: /note=“Description of Artificial Sequence Synthetic primer”
ggaaagcagtgaagacagca

SEQ ID NO: 1300 - DNA, 20 bp, Artificial Sequence
  source: /note=“Description of Artificial Sequence Synthetic primer”
tcctcttccccagaacttga

SEQ ID NO: 1301 - DNA, 20 bp, Artificial Sequence
  source: /note=“Description of Artificial Sequence Synthetic primer”
gttggggcagtactcagcag

SEQ ID NO: 1302 - DNA, 22 bp, Artificial Sequence
  source: /note=“Description of Artificial Sequence Synthetic primer”
tcctttactacatcatgggtca

SEQ ID NO: 1303 - DNA, 18 bp, Artificial Sequence
  source: /note=“Description of Artificial Sequence Synthetic primer”
ggcctccccttcattcaa

SEQ ID NO: 1304 - DNA, 24 bp, Artificial Sequence
  source: /note=“Description of Artificial Sequence Synthetic primer”
ttgaactagtttatacacccagaa

SEQ ID NO: 1305 - DNA, 22 bp, Artificial Sequence
  source: /note=“Description of Artificial Sequence Synthetic primer”
cacacatacacaaaataaaggt

SEQ ID NO: 1306 - DNA, 19 bp, Artificial Sequence
  source: /note=“Description of Artificial Sequence Synthetic primer”
caaagaagaaggagcaagg

SEQ ID NO: 1307 - DNA, 20 bp, Artificial Sequence
  source: /note=“Description of Artificial Sequence Synthetic primer”
ttatccaggacaggaagctg

SEQ ID NO: 1308 - DNA, 20 bp, Artificial Sequence
  source: /note=“Description of Artificial Sequence Synthetic primer”
cccggtgataacagaacgat

SEQ ID NO: 1309 - DNA, 21 bp, Artificial Sequence
  source: /note=“Description of Artificial Sequence Synthetic primer”
catgggactctagaggtagaa

SEQ ID NO: 1310 - DNA, 21 bp, Artificial Sequence
  source: /note=“Description of Artificial Sequence Synthetic primer”
ttttaatctctcttgctctcc

SEQ ID NO: 1311 - DNA, 24 bp, Artificial Sequence
  source: /note=“Description of Artificial Sequence Synthetic primer”
tcatagagtaagccagatataagc

SEQ ID NO: 1312 - DNA, 20 bp, Artificial Sequence
  source: /note=“Description of Artificial Sequence Synthetic primer”
tttaccagccagctcagtcc

SEQ ID NO: 1313 - DNA, 20 bp, Artificial Sequence
  source: /note=“Description of Artificial Sequence Synthetic primer”
tcctgaagggtaagcaggaa

SEQ ID NO: 1314 - DNA, 19 bp, Artificial Sequence
  source: /note=“Description of Artificial Sequence Synthetic primer”
accaaggtcttccctctgc

SEQ ID NO: 1315 - DNA, 20 bp, Artificial Sequence
  source: /note=“Description of Artificial Sequence Synthetic primer”
aggtcagctcagggtgaagt

SEQ ID NO: 1316 - DNA, 20 bp, Artificial Sequence
  source: /note=“Description of Artificial Sequence Synthetic primer”
gctccattgaagggtaaagg

SEQ ID NO: 1317 - DNA, 21 bp, Artificial Sequence
  source: /note=“Description of Artificial Sequence Synthetic primer”
tggaatagaatgcaatcctga

SEQ ID NO: 1318 - DNA, 20 bp, Artificial Sequence
  source: /note=“Description of Artificial Sequence Synthetic primer”
agcccacacaggttggtaag

SEQ ID NO: 1319 - DNA, 20 bp, Artificial Sequence
  source: /note=“Description of Artificial Sequence Synthetic primer”
cagatgctgcaggaaacaga

SEQ ID NO: 1320 - DNA, 20 bp, Artificial Sequence
  source: /note=“Description of Artificial Sequence Synthetic primer”
gtggatcacagggtcacctc

SEQ ID NO: 1321 - DNA, 18 bp, Artificial Sequence
  source: /note=“Description of Artificial Sequence Synthetic primer”
cctcaagctggcctgcaa

SEQ ID NO: 1322 - DNA, 19 bp, Artificial Sequence
  source: /note=“Description of Artificial Sequence Synthetic primer”
aaggcaggcaagacgtagc

SEQ ID NO: 1323 - DNA, 24 bp, Artificial Sequence
  source: /note=“Description of Artificial Sequence Synthetic primer”
caaatatactgattctgtggcaaa

SEQ ID NO: 1324 - DNA, 22 bp, Artificial Sequence
  source: /note=“Description of Artificial Sequence Synthetic primer”
tgatgcattgagattttgatga

SEQ ID NO: 1325 - DNA, 20 bp, Artificial Sequence
  source: /note=“Description of Artificial Sequence Synthetic primer”
cgtctcccacattcttttgg

SEQ ID NO: 1326 - DNA, 23 bp, Artificial Sequence
  source: /note=“Description of Artificial Sequence Synthetic primer”
ggtaggctttgtaacttgcactg

SEQ ID NO: 1327 - DNA, 18 bp, Artificial Sequence
  source: /note=“Description of Artificial Sequence Synthetic primer”
tgaatcctggctgggaaa

SEQ ID NO: 1328 - DNA, 23 bp, Artificial Sequence
  source: /note=“Description of Artificial Sequence Synthetic primer”
gcctcacctacaaagcttattca

SEQ ID NO: 1329 - DNA, 22 bp, Artificial Sequence
  source: /note=“Description of Artificial Sequence Synthetic primer”
tgcagtttgctatgcagtcttt

SEQ ID NO: 1330 - DNA, 22 bp, Artificial Sequence
  source: /note=“Description of Artificial Sequence Synthetic primer”
tgaagctacacagataagaagc

SEQ ID NO: 1331 - DNA, 21 bp, Artificial Sequence
  source: /note=“Description of Artificial Sequence Synthetic primer”
tcattctgggttacccttttg

SEQ ID NO: 1332 - DNA, 20 bp, Artificial Sequence
  source: /note=“Description of Artificial Sequence Synthetic primer”
gccaggaaaagacagtgcat

SEQ ID NO: 1333 - DNA, 21 bp, Artificial Sequence
  source: /note=“Description of Artificial Sequence Synthetic primer”
tctcagcacagagaaggtgct

SEQ ID NO: 1334 - DNA, 22 bp, Artificial Sequence
  source: /note=“Description of Artificial Sequence Synthetic primer”
gcacatttattcactcagcaaa

SEQ ID NO: 1335 - DNA, 21 bp, Artificial Sequence
  source: /note=“Description of Artificial Sequence Synthetic primer”
tgtcctctgtaaaccagacaa

SEQ ID NO: 1336 - DNA, 22 bp, Artificial Sequence
  source: /note=“Description of Artificial Sequence Synthetic primer”
cattttccaaggttgtttctgt

SEQ ID NO: 1337 - DNA, 49 bp, Artificial Sequence
  source: /note=“Description of Artificial Sequence Synthetic primer”
tctttccctacacgacgctcttccgatctctcccacatgtaatgtgttg

SEQ ID NO: 1338 - DNA, 55 bp, Artificial Sequence
  source: /note=“Description of Artificial Sequence Synthetic primer”
gtgactggagttcagacgtgtgctcttccgatctcatacttggagaacaaaggac

SEQ ID NO: 1339 - DNA, 60 bp, Artificial Sequence
  source: /note=“Description of Artificial Sequence Synthetic oligonucleotide”
ctcccacatgtaatgtgttgaaaaagcatggataacggtgtcctttgttctccaagtatg

SEQ ID NO: 1340 - DNA, 49 bp, Artificial Sequence
  source: /note=“Description of Artificial Sequence Synthetic primer”
tctttccctacacgacgctcttccgatctccaggaatgtgaccagcaac

SEQ ID NO: 1341 - DNA, 54 bp, Artificial Sequence
  source: /note=“Description of Artificial Sequence Synthetic primer”
gtgactggagttcagacgtgtgctcttccgatctcaatcacaggcaggaagatg

SEQ ID NO: 1342 - DNA, 57 bp, Artificial Sequence
  source: /note=“Description of Artificial Sequence Synthetic oligonucleotide”
ccaggaatgtgaccagcaacgcagcccacaaaaccttcatcttcctgcctgtgattg

(19) United States
(12) Patent Application Publication
(10) Pub. No.: US 2022/0093208 A1
(43) Pub. Date: Mar. 24, 2022

(54) COMPOSITIONS, METHODS, AND SYSTEMS TO DETECT HEMATOPOIETIC STEM CELL TRANSPLANTATION STATUS

(71) Applicant: Sequenom, Inc., San Diego, CA (US)

(72) Inventors: Roy Brian LEFKOWITZ, San Diego, CA (US); John Allen TYNAN, San Diego, CA (US); Chen XU, San Diego, CA (US)

(21) Appl. No.: 17/427,002

(22) PCT Filed: Feb. 18, 2020
(86) PCT No.: PCT/US2020/018641

§ 371 Date: Jul. 29, 2021

Related U.S. Application Data
(60) Provisional application No. 62/807,616, filed on Feb. 19, 2019.

(51) Int. Cl. G16B 20/20; C12Q 1/6883; G16B 40/30

(52) U.S. Cl. CPC G16B 20/20; C12Q 2600/156; G16B 40/30; C12Q 1/6883

(57) ABSTRACT

This application provides methods and systems for determining transplant status. In some embodiments, the method comprises obtaining a biological sample from hematopoietic stem cell transplant (HSCT) recipient; measuring the amount of one or more identified recipient-specific nucleic acids or donor-specific nucleic acids in the sample; and (c) determining transplant status by monitoring the amount of the one or more identified recipient-specific nucleic acids or donor-specific nucleic acids after transplantation. In some approaches, the one or more recipient-specific or the donor-specific nucleic acids are identified based on the amount of one or more polymorphic nucleic acid targets, which can be used to determine the transplant status. Optionally, the biological sample is blood or bone marrow. Optionally the nucleic acid is genomic DNA.

### CROSS REFERENCE TO THE RELATED APPLICATION

This application claims priority to U.S. Provisional Application No. 62/807,616, filed on Feb. 19, 2019. The entire content of said provisional application is herein incorporated by reference for all purposes.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named SEQ-7011-PCT-Sequence-Listing-1173610.txt, created on Feb. 14, 2020, and having a size of 336,288 bytes and is filed concurrently with the specification. The sequence listing contained in this ASCII formatted document is part of the specification and is herein incorporated by reference in its entirety.

### FIELD

The technology in part relates to methods and systems used for determining hematopoietic stem cell transplantation status.

### BACKGROUND

Hematopoietic stem cells transplantation (“HSTC”) has been used to treat a large number of hematological malignancies, autoimmune diseases, immunodeficiencies. HSTC has also been used to mitigate the effects of exposure to high levels of radiation and thus allows administration of high doses of cytotoxic chemotherapeutic agents to patients who suffer from a number of solid organ tumors. However, there are considerable amount of risks associated with HSCT. Recipients of HSCT are typically immunosuppressed before receiving bone marrow from a donor and it may take a long time, some times several days or even weeks, before the recipients can establish mature hematopoietic cells in his or her circulation. During this time, the patients would often be vulnerable to infection or other pathological conditions. Further, the risk for the relapse after initial engraftment of the donor hematopoetic cells is high. Thus, it is important to monitor the status of HSCT after transplantation in order to determine whether re-transplantation is needed or whether intervention should be prescribed to the recipients to minimize adverse effects.

Current methods of monitoring HSCT status involve detection and quantification of functional lymphocyte populations (e.g., neutrophils) in the HSCT recipients. In general, determination of the status using these methods are made at a time when the patient has already experienced significant injury due to from the graft failure. In addition, these methods are also technically challenging and resource demanding. Thus, a need remains to establish a cost-effective and convenient method for early detection of HSCT status, e.g., graft failure.

Other methods of determining transplantation status using cell-free DNA is also not ideal for determining HSCT status. Cell-free DNA from HSCT patients often contains a mixture of nucleic acids from multiple sources, which renders it challenging to conclusively correlate the amount of or change in the donor fraction and recipient fraction in cell-free samples to the status of HSC engraftment. For example, recipients may have cancer or graph-versus-host disease where one or more organs are afflicted. These conditions may result in recipient DNA being shredded to the cell-free portion of the patient DNA and interfere with the accurate quantification of donor fraction and/or recipient fraction for determination engraftment status.

### SUMMARY OF THE INVENTION

In one aspect, provided herein is a method of determining transplant status comprising: (a) obtaining a sample from a hematopoietic stem cell transplant (HSCT) recipient who has received hematopoietic stem cells from an allogenic source; (b) measuring the amount of one or more identified recipient-specific nucleic acids or donor-specific nucleic acids in the sample; and (c) determining transplant status by monitoring the amount of the one or more identified recipient-specific nucleic acids or donor-specific nucleic acids after transplantation. In some approaches, the one or more recipient-specific or the donor-specific nucleic acids are identified based on one or more polymorphic nucleic acid targets. Optionally, the biological sample is blood or bone marrow. Optionally the nucleic acid is genomic DNA. Optionally the genomic DNA is isolated from peripheral white blood cells in the sample. Optionally the genomic DNA is isolated from a cell population purified from the sample. Optionally the cell population is from a group consisting of B-cells, granulocytes, and T-cells. Optionally the cell population is isolated by positive selection of cells expressing markers of one or more of CD3, CD8, CD19, CD20, CD33, CD34, CD56, CD66, CD5, CD294, CD15, CD14, and CD45. Optionally the purified cell population are peripheral blood mononuclear cells. Optionally the genomic DNA is derived from more than one purified cell populations, wherein the more than one purified cell populations are from B-cells, granulocytes, and T-cells, cells expressing one or more markers from the group consisting of CD3, CD8, CD19, CD20, CD33, CD34, CD56, and CD66.

Optionally the HSCT recipient has at least one hematological disorder from a group consisting of leukemias, lymphomas, immune-deficiency illnesses, hemoglobinopathy, congenital metabolic defects, and non-malignant marrow failures.

Optionally the determining the transplant status step (c) comprises determining the transplant status as a graft failure if the one or more recipient-specific nucleic acids are increased during a time interval post-transplantation, or if the one or more donor-specific nucleic acids are decreased during a time interval post-transplantation. Optionally the determining the transplant status step (c) comprises determining the transplant status as engraftment of the HSCT if i) the one or more recipient-specific nucleic acids in the peripheral blood cells is below a threshold post-transplantation, ii) the one or more recipient-specific nucleic acids are decreased during a time interval post-transplantation, iii) the one or more donor-specific nucleic acids in the peripheral blood cells is above a threshold post-transplantation, or iv) the one or more donor-specific nucleic acids are increased during a time interval post-transplantation.

Optionally the threshold is a percentage of recipient-specific nucleic acid relative to a total of recipient-specific and donor-specific nucleic acids. Optionally the threshold is from the group consisting of less than 20%, 15%, 10%, 5%, 1%, 0.5%, and 0.1%.

Optionally the recipient-specific nucleic acid or the donor-specific nucleic acid is determined by measuring the one or more polymorphic nucleic acid targets in at least one assay, and wherein the at least one assay is high-throughput sequencing, capillary electrophoresis or digital polymerase chain reaction (dPCR). Optionally the recipient-specific nucleic acid or the donor-specific nucleic acid is determined by targeted amplification using a forward and a reverse primer designed specifically for a native genomic nucleic acid, and a variant synthetic oligo that contains a variant as compared to the native sequence, wherein the variant can be a substitution of single nucleotides or multiple nucleotides compared to the native sequence, wherein the variant oligo is added to the amplification reaction in a known amount, wherein the method further comprises: determining the ratio of the amount of the amplified native genomic nucleic acid to the amount of the amplified variant oligo, and determining the total copy number of genomic DNA by multiplying the ratio with the amount of the variant oligo added to the amplification reaction. Optionally the method further comprises determining total copy number of genomic DNA in the biological sample, and determining the copy number of the recipient-specific or donor-specific nucleic acid by multiplying the recipient-specific or donor-specific nucleic acid fraction and the total copy number of genomic DNA.

In some approaches, the polymorphic nucleic acid targets comprises one or more SNPs. Optionally each of the one or more SNPs has a minor allele population frequency of 15%-49%. Optionally the SNPs comprise at least one, two, three, four, or more SNPs in Table 1 or Table 6.

In some approaches, the recipient is genotyped prior to transplantation using one or more SNPs in Table 1 or Table 6. Optionally the donor is genotyped prior to transplantation using one or more SNPs in Table 1. In some approaches, the donor is not genotyped, the recipient is not genotyped, or neither the donor nor the recipient is genotyped for any one of the one or more polymorphic nucleic acid targets prior to transplantation.

In some approaches, the high-throughput sequencing is targeted amplification using a forward and a reverse primer designed specifically for the one or more polymorphic nucleic acid targets or targeted hybridization using a probe sequence that contains the one or more polymorphic nucleic acid targets. Optionally the targeted amplification or targeted hybridization is a multiplex reaction.

In some approaches, the allogenic source is from the group comprising bone marrow transplant, peripheral blood stem cell transplant, and umbilical cord blood. In some approaches, if the HSCT status is determined to be graft failure or at risk for graft failure, the method comprises further advising administration of therapy for the hematological disorder to the HSCT recipient or advising the modification of the HSCT recipient's therapy.

In some approaches the one or more nucleic acids from said HSCT recipient are identified as recipient-specific nucleic acid or donor-specific nucleic acid using a computer algorithm based on measurements of one or more polymorphic nucleic acid target. Optionally the algorithm comprises one or more of the following: (i) a fixed cutoff, (ii) a dynamic clustering, and (iii) an individual polymorphic nucleic acid target threshold. Optionally the fixed cutoff algorithm detects donor-specific nucleic acids if the deviation between the measured frequency of a reference allele of the one or more polymorphic nucleic acid targets in the nucleic acids in the sample and the expected frequency of the reference allele in a reference population is greater than a fixed cutoff, wherein the expected frequency for the reference allele is in the range of 0.00-0.03 if the recipient is homozygous for the alternate allele, 0.40-0.60 if the recipient is heterozygous for the alternate allele, or 0.97-1.00 if the recipient is homozygous for the reference allele.

In some cases, the recipient is homozygous for the reference allele and the fixed cutoff algorithm detects donor-specific nucleic acids if the measured allele frequency of the reference allele of the one or more polymorphic nucleic acid targets is greater than the fixed cutoff. Optionally the fixed cutoff is based on the homozygous allele frequency of the reference or alternate allele of the one or more polymorphic nucleic acid targets in a reference population. Optionally the fixed cutoff is based on a percentile value of distribution of the homozygous allele frequency of the reference or alternate allele of the one or more polymorphic nucleic acid targets in the reference population. Optionally the percentile is at least 90. Optionally identifying one or more nucleic acids as donor-specific nucleic acids using the dynamic clustering algorithm comprises (i) stratifying the one or more polymorphic nucleic acid targets in the nucleic acids into recipient homozygous group and recipient heterozygous group based on the measured allele frequency for a reference allele or an alternate allele of each of the polymorphic nucleic acid targets; (ii) further stratifying recipient homozygous groups into non-informative and informative groups; and (iii) measuring the amounts of one or more polymorphic nucleic acid targets in the informative groups. Optionally the dynamic clustering algorithm is a dynamic K-means algorithm. Optionally the individual polymorphic nucleic acid target threshold algorithm identifies the one or more nucleic acids as donor-specific nucleic acids if the allele frequency of each of the one or more of the polymorphic nucleic acid targets is greater than a threshold. Optionally the threshold is based on the homozygous allele frequency of each of the one or more polymorphic nucleic acid targets in a reference population. Optionally the threshold is a percentile value of a distribution of the homozygous allele frequency of each of the one or more polymorphic nucleic acid targets in the reference population.

In some approaches, a system is provided to perform the method in any one or the preceding embodiments. In some approaches, provided herein is a system for determining transplantation status comprising one or more processors; and memory coupled to one or more processors, the memory encoded with a set of instructions configured to perform a process comprising: (a) obtaining measurements of one or more identified recipient-specific nucleic acids or donor-specific nucleic acids in the sample after transplantation, (b) determining the amount of the one or more identified recipient-specific nucleic acids or donor-specific nucleic acids in the sample after transplantation based on (a); and (c) determining a transplantation status based on the amount of the identified recipient-specific nucleic acids or donor-specific nucleic acids. Optionally said the one or more recipient-specific or the donor-specific nucleic acids are identified based on one or more polymorphic nucleic acid targets. Optionally the sample is blood or bone marrow. Optionally the nucleic acid is genomic DNA. Optionally the determining the transplant status step (c) comprises determining the transplant status as a graft failure if the one or more recipient-specific nucleic acids are increased during a time interval post-transplantation, or if the one or more donor-specific nucleic acids are decreased during a time interval post-transplantation. Optionally the determining the transplant status step (c) comprises determining the transplant status as engraftment of the HSCT if i) the one or more recipient-specific nucleic acids in the peripheral blood cells is below a threshold post-transplantation, ii) the one or more recipient-specific nucleic acids are decreased during a time interval post-transplantation, iii) the one or more donor-specific nucleic acids in the peripheral blood cells is above a threshold post-transplantation, or iv) the one or more donor-specific nucleic acids are increased during a time interval post-transplantation.

Certain embodiments are described further in the following description, examples, claims and drawings.

### BRIEF DESCRIPTION OF THE DRAWINGS

The drawings illustrate embodiments of the technology herein and are not limiting. For clarity and ease of illustration, the drawings are not made to scale and, in some instances, various aspects may be shown exaggerated or enlarged to facilitate an understanding of particular embodiments.

FIG. 1 shows an illustrative example of SNP allele frequencies in a pre-transplant patient and a post-transplant patient. Horizontal dotted black lines represent fixed cutoffs of 0.01 and 0.99, respectively. The boxed regions represent SNPs with allele frequency contribution due to the donor-specific nucleic acid.

FIG. 2 shows an illustrative embodiment of a system in which certain embodiments of the technology may be implemented.

FIG. 3 illustrates types of informative SNPs in a model of transplant patient DNA. Solid arrows point to informative clusters of SNPs that are used for the calculation of donor fraction. The dashed arrow points to excluded informative clusters which are not included in donor fraction calculation.

FIG. 4 shows mirrored allele frequency of informative SNPs. The second cluster from the bottom is SNPs where the recipient is homozygous and the donor is heterozygous. The third cluster from the bottom is SNPs where the recipient is homozygous for one allele and the donor is homozygous for the opposite allele. SNPs in these two clusters are informative SNPs and can be used to calculate the donor fraction.

FIG. 5 shows approaches for calculating donor fraction (DF) based on knowledge of donor genotype or recipient genotype. Donor fraction is calculated using approach 1 (DF1) disclosed herein if neither genotype is known, using approach 2 (DF2) disclosed herein if given the donor genotype, using approach 3 (DF3) disclosed herein if given the recipient genotype, and using approach 4 (DF4) disclosed herein if given both genotypes. Values on the X axis represents the donor fraction determined using DF4. Since DF4 represents the most accurate identification of the informative SNPs, it's placed on the X-axis to serve as a reference to which all other approaches are correlated.

FIGS. 6A and 6B show approaches toward classifying informative SNPs. FIG. 6A shows that Informative SNPs that are included in the calculation of donor fraction are SNPs where the recipient is homozygous and the donor is heterozygous (AA_{recipient}/AB_{donor} or BB_{recipient}/AB_{donor} combinations) or SNPs where the recipient is homozygous and the donor is opposite homozygous (AA_{recipient}/BB_{donor}, BB_{recipient}/BB_{donor} combinations). Informative SNPs that are excluded from the donor fraction calculation are cases where the recipient is heterozygous and the donor is homozygous (AB_{recipient}/AA_{donor} or AB_{recipient}/BB_{donor}). Uninformative SNPs are SNPs where the donor and recipient have a matching genotype (AA_{recipient}/AA_{donor}, BB_{recipient}/BB_{donor}, AB_{recipient}/AB_{donor}). After testing each approach, SNPs are classified as either informative or non-informative. This is designated by “o” and “+” symbols, respectively. FIG. 6B is a figure in which the FIG. 6A is re-plotted to highlight misclassified SNPs visible in panels for Approach 1 and 2 at low and high donor fractions (see data points that have been circled).

FIG. 7 shows estimation of less than 5% donor fraction using DF1, DF2, or DF3. Values on the X axis represents the donor fraction determined using DF4. Donor fraction can be overestimated for low donor fractions, but this can be mitigated through knowledge of the donor's genotype and exclusion of AA_{recipient}/AA_{donor} and BB_{recipient}/BB_{donor} recipient-donor's genotype combinations as is done in the calculation of DF 2.

FIG. 8 shows estimation of greater than 25% donor fraction using DF1, DF2, or DF3. Values on the X axis represents the donor fraction determined using DF4. Donor fraction can be underestimated for high donor fractions, but this can be mitigated through knowledge of the recipient genotype and exclusion of AB_{recipient}/AA_{donor} and AB_{recipient}/BB_{donor} donor-recipient genotype combinations as is done in the calculation of DF 3.

FIG. 9 shows Median and MAD for homozygous allele frequencies of SNPs having different reference allele and alternate allele combination (“Ref_Alt combination”). A higher median and a higher MAD for SNPs having A_G, G_A, C_T, or T_C combinations were observed.

FIG. 10 shows a distribution of Ref_Alt combinations. A_G, G_A, C_T, and T_C are the most frequent combinations of reference and alternate allele in a test panel comprising a subset of SNPs in Panel A and Panel B (Table 1). These combinations occurred in 79.5% of the panel's targets (172 out of the 219 donor fraction assays).

FIGS. 11A and 11B show steps of an exemplary method used for determining the status of engraftment in HSCT patients.

FIG. 12 shows a cumulative binomial probability distribution of informative SNPs. X axis represents the numbers (“N”) of SNPs tested. The Y axis represents the probabilities that N informative SNPs can be identified in patients. The values of N for the six curves, from left to right, are 5, 10, 20, 40, 60, and 80, respectively.

FIG. 13 shows an example of performing multiplexed PCRs to amplify DNAs comprising SNPS and preparing the amplified products for sequencing.

### DEFINITIONS

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or fallel a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. Nucleic acids can be in any form useful for conducting processes herein (e.g., linear, circular, supercoiled, single-stranded, double-stranded and the like) or may include variations (e.g., insertions, deletions or substitutions) that do not alter their utility as part of the present technology. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides.

Deoxyribonucleotides include deoxyadenosine, deoxycytidine, deoxyguanosine and deoxythymidine. For RNA, the base cytosine is replaced with uracil. A template nucleic acid may be prepared using a nucleic acid obtained from a subject as a template. Unless explicitly stated to the contrary, the nucleic acids referred to in the disclosure refer to genomic nucleic acids that are isolated from cells in the sample, and they are not cell-free nucleic acids.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

The term “polymorphism” or “polymorphic nucleic acid target” as used herein refers to a sequence variation within different alleles of the same genomic sequence. A sequence that contains a polymorphism is considered a “polymorphic sequence”. Detection of one or more polymorphisms allows differentiation of different alleles of a single genomic sequence or between two or more individuals. As used herein, the term “polymorphic marker”, “polymorphic sequence”, “polymorphic nucleic acid target” refers to segments of genomic DNA that exhibit heritable variation in a DNA sequence between individuals. Such markers include, but are not limited to, single nucleotide polymorphisms (SNPs), restriction fragment length polymorphisms (RFLPs), short tandem repeats, such as di-, tri- or tetra-nucleotide repeats (STRs), variable number of tandem repeats (VNTRs), copy number variants, insertions, deletions, duplications, and the like. Polymorphic markers according to the present technology can be used to specifically differentiate between a recipient and donor allele in the enriched donor-specific nucleic acid sample and may include one or more of the markers described above.

The terms “single nucleotide polymorphism” or “SNP” as used herein refer to the polynucleotide sequence variation present at a single nucleotide residue within different alleles of the same genomic sequence. This variation may occur within the coding region or non-coding region (i.e., in the promoter or intronic region) of a genomic sequence, if the genomic sequence is transcribed during protein production. Detection of one or more SNP allows differentiation of different alleles of a single genomic sequence or between two or more individuals.

The term “allele” as used herein is one of several alternate forms of a gene or non-coding regions of DNA that occupy the same position on a chromosome. The term allele can be used to describe DNA from any organism including but not limited to bacteria, viruses, fungi, protozoa, molds, yeasts, plants, humans, non-humans, animals, and archeabacteria. A polymorphic nucleic acid target disclosed herein may have two, three, four, or more alternate forms of a gene or non-coding regions of DNA that occupy the same position on a chromosome. A polymorphic nucleic acid target that has two alternate forms is commonly referred to bialleilic polymorphic nucleic acid target. For the purpose of this disclosure, one allele is referred to as the reference allele, and the others are referred to alternate alleles. In some embodiments, the reference allele is an allele present in one or more of the reference genomes, as released by the Genome Reference Consortium (https://www.ncbi.nlm.nih.gov/grc). In some embodiments, the reference allele is an allele represents in reference genome GRCh38. See https://www.ncbi.nlm.nih.gov/grc/human. In some embodiments, the reference allele is not an allele present in the one or more of the reference genomes, for example, the reference allele is an alternate allele of an allele found in the one or more of the reference genomes.

The terms “ratio of the alleles” or “allelic ratio” as used herein refer to the ratio of the amount of one allele and the amount of the other allele in a sample.

The term “amount” as used herein with respect to nucleic acids refers to any suitable measurement, including, but not limited to, absolute amount (e.g. copy number), relative amount (e.g. fraction or ratio), weight (e.g., grams), and concentration (e.g., grams per unit volume (e.g., milliliter); molar units).

The term “Ref_Alt” combination with regard to an SNP refers to a combination of the reference allele and the alternate allele for the SNP in the population. For example, a Ref_Alt of C_G refers to that the reference allele is C, and the alternate allele is G for the SNP.

As used herein, when an action such as a determination of something is “triggered by”, “according to”, or “based on” something, this means the action is triggered, according to, or based at least in part on at least a part of the something.

The term “fraction” refers to the proportion of a substance in a mixture or solution (e.g., the proportion of donor-specific nucleic acid in a recipient sample that comprises a mixture of recipient and donor-specific nucleic acid). The fraction may be expressed as a percentage, which is used to express how large/small one quantity is, relative to another quantity as a fraction of 100.

The term “sample” as used herein refers to a specimen containing nucleic acid. Examples of samples include, but are not limited to, tissue, bodily fluid (for example, blood, serum, plasma, saliva, urine, tears, peritoneal fluid, ascitic fluid, vaginal secretion, breast fluid, breast milk, lymph fluid, sputum, cerebrospinal fluid or mucosa secretion), or other body exudate, fecal matter (e.g., stool), an individual cell or extract of the such sources that contain the nucleic acid of the same, and subcellular structures such as mitochondria, using protocols well established within the art.

The term “blood” as used herein refers to a blood sample or preparation from a subject. The term encompasses whole blood or any fractions of blood, such as serum and plasma as conventionally defined.

The term “target nucleic acid” as used herein refers to a nucleic acid examined using the methods disclosed herein to determine if the nucleic acid is donor or recipient-specific nucleic acid.

The term “sequence-specific” or “locus-specific method” as used herein refers to a method that interrogates (for example, quantifies) nucleic acid at a specific location (or locus) in the genome based on the sequence composition. Sequence-specific or locus-specific methods allow for the quantification of specific regions or chromosomes.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), where the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence. At least one of the PCR primers for amplification of a polynucleotide sequence is sequence-specific for the sequence.

The term “template” refers to any nucleic acid molecule that can be used for amplification in the technology herein. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes but is not limited to polymerase chain reaction, self-sustained sequence reaction, ligase chain reaction, rapid amplification of cDNA ends, polymerase chain reaction and ligase chain reaction, Q-beta phage amplification, strand displacement amplification, or splice overlap extension polymerase chain reaction. In some embodiments, a single molecule of nucleic acid is amplified, for example, by digital PCR.

The term “sensitivity” as used herein refers to the number of true positives divided by the number of true positives plus the number of false negatives, where sensitivity (sens) may be within the range of 0≤sens≤1. Ideally, method embodiments herein have the number of false negatives equaling zero or close to equaling zero, so that no subject is wrongly identified as not having graft failure when the transplanted organ has indeed been rejected. Conversely, an assessment often is made of the ability of a prediction algorithm to classify negatives correctly, a complementary measurement to sensitivity.

The term “specificity” as used herein refers to the number of true negatives divided by the number of true negatives plus the number of false positives, where specificity (spec) may be within the range of 0≤spec≤1. Ideally, methods embodiments herein have the number of false positives equaling zero or close to equaling zero, so that no subject wrongly identified as having graft failure when the transplant has not been rejected. Hence, a method that has sensitivity and specificity equaling one, or 100%, sometimes is selected.

As used herein, “reads” are short nucleotide sequences produced by any sequencing process described herein or known in the art. Reads can be generated from one end of nucleic acid fragments (“single-end reads”), and sometimes are generated from both ends of nucleic acids (“double-end reads”). In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

The term “cutoff value” or “threshold” as used herein means a numerical value whose value is used to arbitrate between two or more states (e.g. diseased and non-diseased) of classification for a biological sample. For example, if a parameter is equal to or lower than the cutoff value, a classification of the quantitative data is made (e.g., an amount of recipient nucleic acid detected in the sample derived from the transplant recipient that is equal to or lower than a predetermined threshold indicates engraftment of the HSCT).

Unless explicitly stated otherwise, the term “transplant” or “transplantation” refers to the transfer of hematopoetic stem cells from a donor to a recipient. In some cases, the transplant is an allotransplantation, i.e., hematopoietic stem cell transplant to a recipient from a genetically non-identical donor of the same species. The donor and/or recipient of the hematopoietic stem cell transplant can be a human or an animal. For example, the animal can be a mammal, a primate (e.g., a monkey), a livestock animal (e.g., a horse, a cow, a sheep, a pig, or a goat), a companion animal (e.g., a dog, or a cat), a laboratory test animal (e.g., a mouse, a rat, a guinea pig, or a bird), an animal of verterinary significance or economic significance. In some embodiments, the organ being transplanted is a solid organ. Non-limiting examples of hematopoetic stem cells used for the transplant may be derived from a donor's bone marrow, peripheral blood, and/or umbilical cord blood.

The term “allogeneic” refers to tissues or cells that are genetically dissimilar and hence immunologically incompatible, although from individuals of the same species. An allogeneic transplant is also referred to as an allograft.

The term “minor allele population frequency” or “MAF” refers to the frequency at which the second most common allele occurs in a given population. Single nucleotide polymorphisms (SNPs) are generally biallelic systems, in which case, MAF refers to the frequency at which the lesser common allele occurs in a given population, e.g., human population.

The term “allele frequency”, as used herein, refers to the relative frequency or an allele at a particular locus in the sample, typically expressed as a fraction or a percentage.

The term “expected allele frequency” refers to allele frequency in the recipient before transplantation. Expected allele frequency can be extrapolated from the allele frequencies found in a group of individuals having a single diploid genome, e.g., non-pregnant female and male who have not received a transplant. In some cases, the expected allele frequency is the median or mean of the allele frequencies in the group of individuals. The expected allele frequency is typically around 0.5 for homozygous, and around 0 for homozygous for the alternate allele, and around 1 if homozygous for the reference allele. When the donor and recipient are of the same genotype, the allele frequency in the post-transplantation sample from the recipient is equal to the expected allele frequency.

The term “transplantation status” or “transplant status” used herein refers to the health status of the hematopoetic cells after they have been removed from the donor and implanted into the recipient. Transplantation status includes, e.g., graft failure (graft failure) and engraftment (engraftment of the HSCT). Graft failure refers to either lack of initial engraftment of donor cells or loss of donor cells after initial engraftment (relapse).

### DETAILED DESCRIPTION

Overview

In individuals with a variety of hematopoietic diseases, hematopoietic stem cell (HSC) transplantation may be used to repopulate the patient's hematopoietic cells after ablation of the patient's endogenous HSCs. Bone marrow transplants may be allogeneic. In cases of allogeneic bone marrow transplant (with exception of identical twins) there will be varying levels of genetic differences between the donor and recipient genomes, depending on the level of consanguinity between the donor and recipient. These genetic differences can be monitored in recipients' peripheral blood or leukocyte subtypes thereof to monitor the extent of donor hematopoietic cell engraftment or relapse of disease.

The disclosure provides methods for determining HSCT status by monitoring the amount or fraction of recipient-specific nucleic acids or the amount or fraction of donor-specific nucleic acids in a biological sample obtained from the HSCT recipient. An increase in the recipient-specific nucleic acids, or a decrease in the donor-specific nucleic acids, during a time interval post-transplantation is an indication of graft failure, whereas a decrease in the recipient-specific nucleic acids or an increase in the donor-specific nucleic acids is an indication of successful engraftment of the HSCT.

Prior to transplanting donor bone marrow to a recipient, the recipient typically undergoes immunosuppression (e.g., chemotherapy, radiation, etc.). This is to prevent the recipient from rejecting the bone marrow transplanted from a donor and destroying the subject's existing bone marrow, which is often diseased, damaged, or otherwise non-functional. As a result, the recipient will have no detectable markers related to hematopoietic precursor cells. If the bone marrow transplantation is successful, the donors hematopoietic cells start to grow in the recipient's bone marrow cavity. when triggered by certain hormonal signals, a portion of the cells, will then begin to differentiate into one of multiple lineages to produce precursor cells for red blood cells (RBC) (erythroblast), white blood cell (WBC) (myeloblast, lymphoblast), and platelet (megakaryocyte), respectively. Again, based on certain hormonal signals, these immature “blast” cells, will eventually terminally differentiate into mature cells that are released into the peripheral blood. The differentiation process may take a few days to a few weeks before a recipient presents mature hematopoietic cells (derived from donor hematopoietic stem cells) in their circulation. As such, If a bone marrow transplant is successful, the recipient-specific nucleic acids in the peripheral blood will decrease or maintain at a very low level, for example, at a level that is below a threshold that can be readily determined by a trained medical professional in the bone marrow transplant field. If the bone marrow transplant is unsuccessful, i.e., the donor hematopoietic stem cells fail to engraft, the donor nucleic acids in the bone marrow or the peripheral blood will decrease during a time interval post-transplantation. There are also intermediate transplantation status in which the recipient has not exhibited a clear engraftment of HSCT, such as mixed chimerism and split chimerism; each can be determined based on the amount of recipient-specific or donor-specific nucleic acids in a sample (e.g., peripheral blood sample) from the transplant recipient, as described below.

### SPECIFIC EMBODIMENTS

Practicing the technology herein utilizes routine techniques in the field of molecular biology. Basic texts disclosing the general methods of use in the technology herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Deventer et. al., Nucleic Acids Res. 12: 6159-6168 (1984). Purification of oligonucleotides is performed using any art-recognized strategy, e.g., native acrylamide gel electrophoresis or anion-exchange high performance liquid chromatography (HPLC) as described in Pearson & Reanier, J. Chrom. 255: 137-149 (1983).

Patients

Nucleic acid or a nucleic acid mixture utilized in methods and apparatuses described herein often is isolated from a sample obtained from a subject. A subject can be any living or non-living organism, including but not limited to a human, a non-human animal. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female.

Subjects who may benefit from the methods disclosed herein include those who have received hematopoietic stem cells from an allogeneic source. In some cases, these allogeneic hematopoietic stem cells are collected by direct aspiration from the bone marrow. In some cases, they are harvested from the peripheral blood. Peripheral blood stem cells may be harvested by first treating the donor with hematopoietic growth factors, which cause the stem cells to proliferate and circulate freely in the peripheral blood. The blood may then be collected by venipuncture and subjected to leukapheresis to obtain the cells for transplantation. In some cases, umbilical cord blood cells harvested at the time of delivery may also be used. Thus, in some embodiments, the hematopoietic stem cells from an allogeneic source may be one or more of bone marrow, peripheral blood stem cells, or umbilical cord blood.

Subjects who have received HSCT can be monitored using the methods disclosed herein. The HSCT recipient may have one or more of a number of hematological disorders, including but are not limited to, leukemias, lymphomas, immune-deficiency illnesses, hemoglobinopathy, congenital metabolic defects, and non-malignant marrow failures hematological malignancy, a myeloma, multiple myeloma, a leukemia, acute lymphoblastic leukemia, chronic lymphocytic leukemia, a lymphoma, indolent lymphoma, non-Hodgkin lymphoma, diffuse B cell lymphoma, follicular lymphoma, mantle cell lymphoma, T cell lymphoma, Hodgkin lymphoma, a neuroblastoma, a retinoblastoma, Shwachman Diamond syndrome, a brain tumor, Ewing's Sarcoma, a Desmoplastic small round cell tumor, a relapsed germ cell tumor, a hematological disorder, a hemoglobinopathy, an autoimmune disorder, juvenile idiopathic arthritis, systemic lupus erythematosus, severe combined immunodeficiency, congenital neutropenia with defective stem cells, severe aplastic anemia, a sickle-cell disease, a myelodysplasia syndrome, chronic granulomatous disease, a metabolic disorder, Hurler syndrome, Gaucher disease, osteopetrosis, malignant infantile osteopetrosis, heart disease, HIV, and AIDS and the status of HSCT can be monitored using the methods discloses.

Samples

In some embodiments, the sample that is used for detecting transplantation status is a blood sample or a bone marrow sample from an organ transplant recipient who has received an organ from an allogeneic source. In several embodiments, the blood is whole blood. In several embodiments, the blood sample is heparinized (either during, or after collection). An appropriate amount of peripheral blood, e.g., typically between 5-50 ml, may be collected and stored according to standard procedure prior to further preparation. Blood samples may be collected, stored or transported in a manner known to the person of ordinary skill in the art to minimize degradation or the quality of nucleic acid present in the sample.

The blood sample can be used with pretreatment or can be used “as is”, e.g., without pretreatment. When pretreatment is used, it can take many forms, including sample fractionation, precipitation of unwanted material, etc. For example, some embodiments allow for samples to be taken from donors and used “as-is” for isolation and testing of biomarkers. However, some embodiments allow a user to pretreat samples for certain reasons. These reasons include, but are not limited to, protocols to facilitate storage, facilitating biomarker detection, etc.

In some embodiments, the sample is processed to isolate peripheral white blood cells and genomic nucleic acids can be prepared from the isolated white blood cells. Isolation of white blood cells from peripheral blood can be performed using methods that are well known and kits that are commercially available kits, for example, the blood fractionation protocol for collection of White Blood Cells from Thermofisher Scientific, Inc. (Waltham, Mass.). In some embodiments, the sample is processed to isolate peripheral mononuclear cells (“PBMCs”) from whole blood samples and genomic nucleic acids can be prepared from the isolated PBMCs. Isolation of PBMCs can be performed using methods well known in the art, for example, density centrifugation (Ficoll-Paque), isolation by cell preparation tubes and SepMate tubes with lymphoprep, as described in Grievink et al., Biopreserv. Biobank, 2016 Oct. 14 (5): 410-415.

In some embodiments, T cells, B cells and granulocytes are isolated from the blood sample and genomic nucleic acids are prepared from these cell populations. Methods for purifying these populations are well known in the art, for example, as described in Kremer et al., Veternary immunology and Immunopathology, Vol 31 issues 1-2, Feb. 15, 1992, 189-193. Commercial kits for isolation of these various populations are also available, for example for STEMCELL, EasyStep™ direct Human B cell isolation kit, EasySTep Human CD4+ T cell isolation kit.

In some embodiments, the sample is processed to isolate one or more cell populations that express specific surface markers and genomic nucleic acids can then be prepared from these cell populations. In some embodiments, the cell surface marker is a marker that expressed on T cells, B cells, basophils, granulocytes, monocytes, or other leucocytes. Non-limiting examples include CD3, CD8, CD19, CD20, CD33, Cd34, CD56, CD66, CD5, CD294, CD15, CD14, and CD45. In some embodiments, genomic nucleic acids are isolated from a cell population expressing any one of the markers. In some embodiments, genomic nucleic acids are isolated from a cell population expressing two or more markers above. In some embodiments, genomic nucleic acids are isolated from two or more cell populations expressing different markers, each cell population expressing one or more markers as described above. For example, a myeloid cell population can be isolated based on the expression of CD33 and CD66b.

In some embodiments, the purified cell population are isolated based on expression of one of these markers. These cell populations can be isolated using a positive selection strategy, through which cells expressing the marker of interest are isolated using a reagent that can bind to the marker. The cell populations can also be isolated using a negative strategy, through which cells not expressing the marker of interest are isolated and removed from the sample.

In some embodiments, the samples are typically taken for monitoring the transplantation status at one or more time points post-transplantation, as described below.

DNA Isolation

Various methods for extracting DNA from a biological sample are known and can be used in the methods of determining transplantation status. The general methods of DNA preparation (e.g., described by Sambrook and Russell, Molecular Cloning: A Laboratory Manual 3d ed., 2001) can be followed; various commercially available reagents or kits, such as QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample from a subject. Combinations of more than one of these methods may also be used.

Samples containing cells are typically lysed in order to isolate genomic nucleic acids. Cell lysis procedures and reagents are known in the art and may generally be performed by chemical, physical, or electrolytic lysis methods. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 ug/ml Rnase A; a second solution can contain 0.2N NaOH and 1% SDS; and a third solution can contain 3M KOAc, pH 5.5. These procedures can be found in Current Protocols in Molecular Biology, John Wiley & Sons, N.Y., 6.3.1-6.3.6 (1989), incorporated herein in its entirety.

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid is provided with fewer non-nucleic acid components (e.g., protein, lipid) than the amount of components present in a source sample. A composition comprising isolated nucleic acid can be about 90%, 91%, 92%, 93%, 94%, 95%, 96%, 97%, 98%, 99% or greater than 99% free of non-nucleic acid components. The term “purified” as used herein refers to nucleic acid provided that contains fewer nucleic acid species than in the sample source from which the nucleic acid is derived. A composition comprising nucleic acid may be about 90%, 91%, 92%, 93%, 94%, 95%, 96%, 97%, 98%, 99% or greater than 99% free of other nucleic acid species. The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

The genomic nucleic acids may be isolated at a different time points as compared to another nucleic acid, where each of the samples is from the same or a different source. In some embodiments, the genomic nucleic acids are isolated from the same recipient at different time points post transplantation. The recipient or donor-specific nucleic acid fractions can be determined for each of the time points as described herein, and a comparison between the time points can often reveal the transplantation status. For example, an increase in recipient-specific nucleic acid fractions indicates graft failure. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples). In some embodiments, the pooled samples may be from the same patient, e.g., transplant recipient, but are taken at different time points, or are of different tissue type. In some embodiments, the pooled samples may be from different patients. As described further below, in some embodiments, identifiers are attached to the nucleic acids derived from the each of the one or more samples to distinguish the sources of the sample.

Nucleic acid may be single or double stranded. Single stranded DNA, for example, can be generated by denaturing double stranded DNA by heating or by treatment with alkali, for example. In some cases, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

Nucleic acids may be fragmented using either physical or enzymatic methods known in the art.

Quantifying Recipient-Specific or Donor-Specific Nucleic Acid Content

The methods described herein are based on monitoring the amount of recipient-specific nucleic acid or donor-specific nucleic acid in the total nucleic acids in the sample from the HSCT patient. In some cases, the amount of recipient-specific nucleic acid or donor-specific nucleic acid is determined based on a quantification of sequence read counts described herein. In some cases, the amount of recipient-specific nucleic acid is a fraction of recipient-specific nucleic acid relative to the total nucleic acid in a sample, referred to as “recipient-specific nucleic acid fraction” or “recipient fraction”. In some cases, the amount of donor-specific nucleic acid is a fraction of donor-specific nucleic acid relative to the total nucleic acid in a sample, referred to as “donor-specific nucleic acid fraction” or “donor fraction”. In some embodiments, the recipient fraction or donor fraction is determined according to allelic ratios of polymorphic nucleic acid target sequences.

### Overview of Polymorphism-Based Nucleic Acid Quantifier Assay

Determination of recipient-specific nucleic acid content (e.g., recipient-specific nucleic acid fraction) sometimes is performed using a polymorphism-based nucleic acid quantifier assay, as described herein. This type of assay allows for the detection and quantification of recipient-specific or donor-specific nucleic acid in a sample from a transplant recipient based on allelic ratios of polymorphic nucleic acid target sequences (e.g., single nucleotide polymorphisms (SNPs)).

In some embodiments, the polymorphic nucleic acid targets are one or more of a: (i) single nucleotide polymorphism (SNP); (ii) insertion/deletion polymorphism, (iii) restriction fragment length polymorphism (RFLPs), (iv) short tandem repeat (STR), (v) variable number of tandem repeats (VNTR), (vi) a copy number variant, (vii) an insertion/deletion variant, or (viii) a combination of any of (i)-(vii) thereof.

A polymorphic marker or site is the locus at which divergence occurs. Polymorphic forms also are manifested as different alleles for a gene. In some embodiments, there are two alleles for a polymorphic nucleic acid target and these polymorphic nucleic acid targets are called biallelic polymorphic nucleic acid targets. In some embodiments, there are three, four, or more alleles for a polymorphic nucleic acid target.

In some embodiments, one of these alleles is referred to as a reference allele and the others are referred to as alternate alleles. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

Numerous genes have polymorphic regions. Since individuals have any one of several allelic variants of a polymorphic region, individuals can be identified based on the type of allelic variants of polymorphic regions of genes. This can be used, for example, for forensic purposes. In other situations, it is crucial to know the identity of allelic variants that an individual has. For example, allelic differences in certain genes, for example, major histocompatibility complex (MHC) genes, are involved in graft rejection or graft versus host disease in bone marrow transplantation. Accordingly, it is highly desirable to develop rapid, sensitive, and accurate methods for determining the identity of allelic variants of polymorphic regions of genes or genetic lesions.

In some embodiments, the polymorphic nucleic acid targets are single nucleotide polymorphisms (SNPs). Determining the recipient-specific nucleic acid amount or fraction based on recipient-specific SNPs allows indirect testing (association of haplotypes) and direct testing (functional variants). SNPs are the most abundant and stable genetic markers. Common diseases are best explained by common genetic alterations, and the natural variation in the human population aids in understanding disease, therapy and environmental interactions.

Single nucleotide polymorphisms (SNPs) are generally biallelic systems, that is, there are two alleles that an individual can have for any particular marker, one of which is referred to as a reference allele and the other referred to as an alternate allele. This means that the information content per SNP marker is relatively low when compared to microsatellite markers, which can have upwards of 10 alleles. SNPs also tend to be very population-specific; a marker that is polymorphic in one population sometimes is not very polymorphic in another. SNPs, found approximately every kilobase (see Wang et al. (1998) Science 280:1077-1082), offer the potential for generating very high density genetic maps, which will be extremely useful for developing haplotyping systems for genes or regions of interest, and because of the nature of SNPS, they can in fact be the polymorphisms associated with the disease phenotypes under study. The low mutation rate of SNPs also makes them excellent markers for studying complex genetic traits.

### Identifying the Informative Polymorphic Nucleic Acid Targets

In some embodiments, at least one polymorphic nucleic acid target of the plurality of polymorphic nucleic acid targets is informative for determining the presence of donor-specific or recipient-specific nucleic acid in a given sample. A polymorphic nucleic acid target that is informative for determining the presence of donor-specific nucleic acids or recipient-specific nuclei acid, sometimes referred to as an informative target, or informative polymorphism (e.g., informative SNP), typically differs in some aspect between the donor and the recipient. For example, an informative target may have one allele for the donor and a different allele for the recipient (e.g., the recipient has allele A at the polymorphic nucleic acid target and the donor has allele B at the polymorphic nucleic acid target site). The donor-specific or recipient-specific nucleic acid in the sample can be quantified based on the allelic frequency of the informative polymorphic nucleic acid target sequences for the donor (allele A) or the recipient (allele B) in the sample.

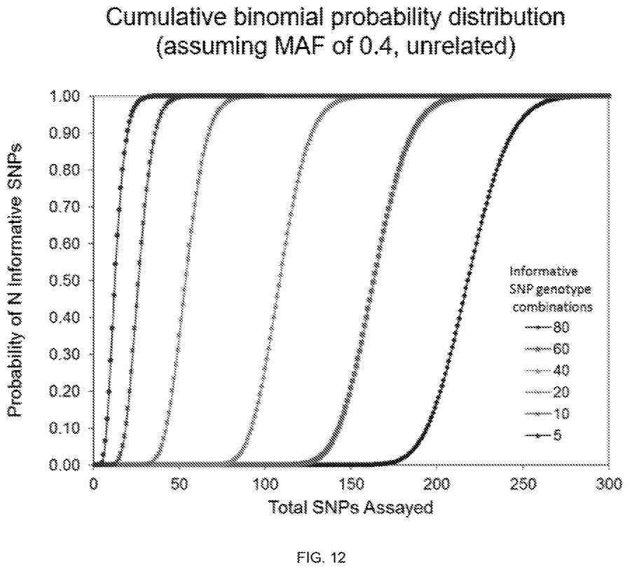

FIG. 12 illustrates the cumulative binomial probability distribution of the informative SNPs when the MAF is 0.4, and the donor is unrelated to the recipient. Other polymorphic nucleic acid targets are expected to have similar distribution. As shown in the FIG. 12, a panel of 250 SNPs should yield 60+ informative genotypes in over >99% of recipients. If the donor and recipient are related, e.g., are in Child:parent/sibling relationship, a panel of 320 SNPs should yield 60+ informative genotypes in over >99% of recipients (not shown in FIG. 12).

In some cases, samples from the recipient and/or the donor prior to transplantation can be obtained and their genotypes at polymorphic nucleic acid targets can be determined (e.g., their genotypes at the SNPs listed in Table 1 or Table 6). Samples that may be collected prior to transplantation include, but are not limited to, peripheral blood, buccal swab, and saliva. Alleles that are specific for the recipient (or the donor) can be identified and quantified as described above. Unless stated explicitly to the contrary, the phrase “genotyping a recipient”, “genotyping a donor”, “a recipient is genotyped”, or “a donor is genotyped” refers to genotyping the recipient or donor based on a sample that contains only recipient or donor nucleic acid. Typically the sample used for genotyping is one that is obtained from the recipient or donor prior to transplantation. In some cases, the sample can also be a sample obtained from a recipient after HSCT, provided that the sample does not contain donor nucleic acid. Samples that may be collected post transplantation for purpose of genotyping the recipient include, but are not limited to, epidermal cells collected from a skin patch or skin swab. Unless stated explicitly to the contrary, the phrase “prior to transplantation”, when used in conjunction with the term “genotype” or “genotyping,” is not to be interpreted as being limited to that the timing of performing the genotyping experiment or obtaining the sample must occur before the transplantation procedure.

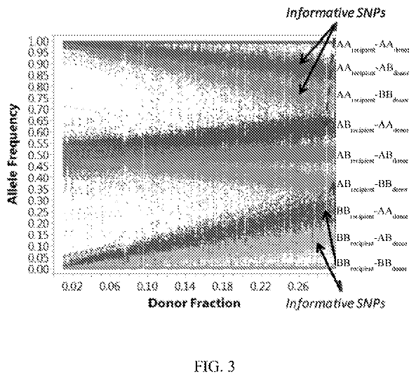

In some cases, informative polymorphic nucleic acid targets (e.g., informative SNPs) are identified based on certain donor/recipient genotype combinations. For a biallelic polymorphic nucleic acid target (i.e., two possible alleles (e.g., A and B, wherein A is a reference allele and B is an alternate allele, or vice versa)), possible recipient/donor genotype combinations include: 1) recipient AA, donor AA; 2) recipient AA, donor AB; 3) recipient AA, donor BB; 4) recipient AB, donor AA; 5) recipient AB, donor AB; 6) recipient AB; donor BB; 7) recipient BB, donor AA; 8) recipient BB, donor AB; and 9) recipient BB, donor BB. Genotypes AA and BB are considered homozygous genotypes and genotype AB is considered a heterozygous genotype. In some cases, informative genotype combinations (i.e., genotype combinations for a polymorphic nucleic acid target that may be informative for determining donor-specific nucleic acid fraction) include combinations where the recipient is homozygous and the donor is heterozygous or homozygous for the alternate allele (e.g., recipient AA, donor AB; or recipient BB, donor AB; or recipient AA, donor BB). Such genotype combinations may be referred to as Type 1 informative genotypes. In some cases, informative genotype combinations (i.e., genotype combinations for a polymorphic nucleic acid target that may be informative for determining donor-specific nucleic acid fraction) include combinations where the recipient is heterozygous and the donor is homozygous (e.g., recipient AB, donor AA; or recipient AB, donor BB). Such genotype combinations may be referred to as Type 2 informative genotypes. In some cases, non-informative genotype combinations (i.e., genotype combinations for a polymorphic nucleic acid target that may not be informative for determining donor-specific nucleic acid fraction) include combinations where the recipient is heterozygous and the donor is heterozygous (e.g., recipient AB, donor AB). Such genotype combinations may be referred to as non-informative genotypes or non-informative heterozygotes. In some cases, non-informative genotype combinations (i.e., genotype combinations for a polymorphic nucleic acid target that may not be informative for determining donor-specific nucleic acid fraction) include combinations where the recipient is homozygous and the donor is homozygous (e.g., recipient AA, donor AA; or recipient BB, donor BB). Such genotype combinations may be referred to as non-informative genotypes or non-informative homozygotes. In some embodiments, both the recipient genotype and the donor genotype for the polymorphic nucleic acid targets are determined prior to transplantation. The presence of donor-specific nucleic acids can be readily determined by selecting the informative polymorphic nucleic acid targets as described above, and detecting and/or quantifying the donor-specific alleles of the polymorphic nucleic acid targets using the assays described herein. FIG. 3 shows the distribution of the various SNP genotype combinations and also indicates informative SNPs that are useful for determining the donor-specific nucleic acids.

In one embodiment, both the donor and the recipient are genotyped prior to transplantation. In one embodiment, the method comprises genotyping the HSCT recipient and the HSCT donor prior to transplantation, obtaining a sample from the HSCT recipient who has received hematopoietic stem cells from an allogenic source; measuring the amount of one or more identified recipient-specific nucleic acids or donor-specific nucleic acids in the sample; and determining transplantation status by monitoring the amount of the one or more identified recipient-specific nucleic acids or donor-specific nucleic acids after transplantation. The transplantation status may be determined as described herein, e.g., in the section entitled “Determining Transplantation Status”.

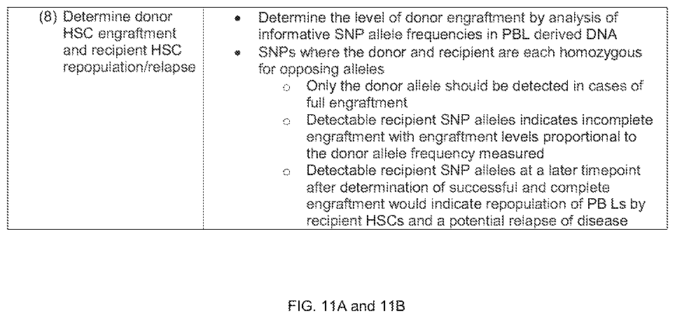

In one embodiment, the method comprises genotying the HSCT recipient and donor prior to transplantation, determining one or more informative SNPs from the recipient and donor genotypes, obtaining a sample from the recipient, isolating genomic nucleic acid from the sample, obtaining sequence reads spanning the one or more information SNPs, determining the allele frequencies of the informative SNPs from the recipient and donor, determining the fraction of door and recipient-specific nucleic acid based on the measured frequencies of informative SNPs. In some embodiments, the informative SNPs are amplified before sequencing. In some embodiments, the amplification is a multiplex PCR. In some embodiments, the one or more information SNPs are in Table 1 or Table 6. In some embodiments, the fraction or load of the recipient-specific nucleic acid or donor-specific nucleic acid in the patient is monitored during a time period interval post-transplantation to determine the transplantation status as described herein. One exemplary method for determining the transplantation status in which the donor and recipient are genotyped prior to transplantation is illustrated in FIG. 11A and FIG. 11B.

In some cases, obtaining samples from the donor and recipient for genotyping at various polymorphic nucleic acids targets prior to transplantation may not be possible or practical. Thus, in some cases, donor and/or recipient genotypes of the one or more polymorphic nucleic acid targets are not determined prior to determination of transplantation status. In some cases, the recipient genotype for one or more polymorphic nucleic acid targets is not determined prior to transplantation status determination. In some cases, the donor genotype for one or more polymorphic nucleic acid targets is not determined prior to transplantation status determination. In some cases, the recipient genotype and the donor genotype for one or more polymorphic nucleic acid targets are not determined prior to transplantation status determination. In some embodiments, donor and recipient genotypes are not determined for any of the polymorphic nucleic acid targets prior to determination of transplantation status. In some cases, the recipient genotype for each of the polymorphic nucleic acid targets is not determined prior to transplantation. In some cases, the donor genotype for each of the polymorphic nucleic acid targets is not determined prior to transplantation status determination. In some cases, the recipient genotype and the donor genotype for each of the polymorphic nucleic acid targets are not determined prior to transplantation status determination. In some embodiments, this disclosure provides methods and systems that can be used to detect and/or quantify donor-specific nucleic acids, even in the absence of information of donor or recipient genotype of one or more polymorphic nucleic acid targets.

As described above, after engraftment, donors hematopoietic stem cells start to grow in the recipient's bone marrow cavity. A portion of the cells, when triggered by certain hormonal signals, will then begin to differentiate into one of multiple lineages to produce precursor cells for red blood cells (RBC) (erythroblast), white blood cell (WBC) (myeloblast, lymphoblast), and platelet (megakaryocyte), respectively. However, it takes days, or even weeks before these immature cells, will eventually terminally differentiate into mature cells that are released into the peripheral blood. Thus, in the period soon after the transplantation, for example, within 0 to 30 days, e.g., 2 to 20 days, 3 to 15 days, or within 4 to 10 days from transplantation, the contribution of donor-specific nucleic acids to the mixture of donor and recipient-specific nucleic acids will be relatively minor, and informative polymorphic nucleic acid targets (indicating the presence of the donor-specific nucleic acids) can be identified accordingly based on the allelic frequencies, as described below.

In some cases, donor-specific alleles are identified by a deviation of the measured allele frequency in the total nucleic acids from an expected allele frequency, as described below. This is based on the fact that each of the SNPs allele frequencies before transplantation will cluster around heterozygous (0.5) or homozygous (0 or 1). When there is a difference in donor & recipient genotype, there'll be a deviation (proportional to donor fraction) from heterozygous or homozygous. When there is a match in donor & recipient genotype, the allele frequency in the mixed DNAs will be the same as the allele frequency in the genotype of the recipient before transplantation. Various recipient-donor genotype combinations are further illustrated below and also illustrated in FIG. 3.

Donor genotype & recipient genotype are different (results in a donor-specific deviation of the allele frequency):

AA_{recipient}/AB_{donor}

AA_{recipient}/BB_{donor}

AB_{recipient}/AA_{donor}

AB_{recipient}/BB_{donor}

BB_{recipient}/AA_{donor}

BB_{recipient}/AB_{donor}

Donor genotype & recipient genotype are the same (so the resulting allele frequency is the “expected” recipient genotype):

AA_{recipient}/AA_{donor}

AB_{recipient}/AB_{donor}

BB_{recipient}/BB_{donor}

(A represents the reference allele and B represents the alternate allele.)

In some embodiments, an allele frequency is determined for one or more alleles of the polymorphic nucleic acid targets in a sample. This sometimes is referred to as measured allele frequency. Allele frequency can be determined, for example, by counting the number of sequence reads for an allele (e.g., allele B) and dividing by the total number of sequence reads for that locus (e.g., allele B+allele A). In some cases, an allele frequency average, mean or median is determined. In some cases, donor-specific nucleic acid fraction can be determined based on the allele frequency mean (e.g., allele frequency mean multiplied by two).

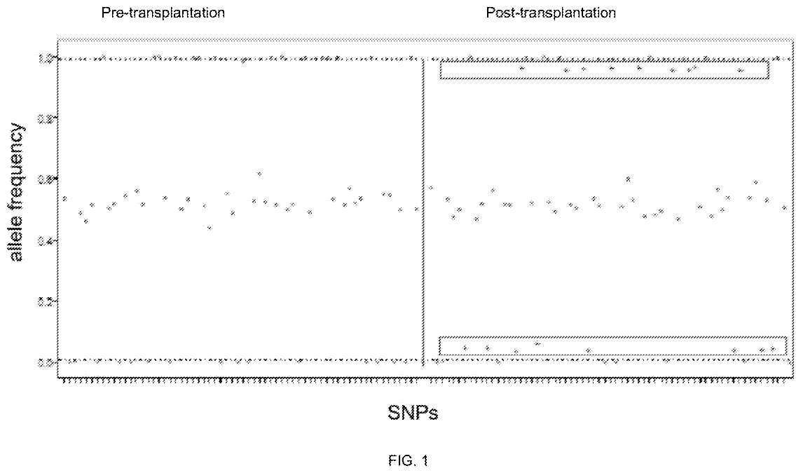

In some embodiments, quantification data (e.g., sequencing data) covering the polymorphic nucleic acid target are used to count the number of times the genomic positions of the polymorphic nucleic acid target (e.g., an SNP) are sequenced. The number of sequencing reads containing the reference allele and the alternate allele of the polymorphic nucleic acid target, respectively, can be determined. For example, in a sample homozygous for the reference allele of a SNP, there would ideally be a reference SNP allele frequency of about 1.0 (e.g. 0.99-1.00) where all sequencing reads covering the SNP contain the reference SNP allele (FIG. 1 left panel, top group of allele frequencies). When the sample is heterozygous for both the reference and alternate allele, the expected allele frequency for the reference SNP allele is about 0.5 (e.g., 0.46-0.53) (FIG. 1 left panel, middle group of allele frequencies). When the sample is homozygous for the alternate allele, the expected reference SNP allele frequency would be 0 (FIG. 1 left panel, bottom group of allele frequencies). These values of 1.0, 0.5, and 0 are idealized though, and while measurements will generally approach these values, real-world SNP allele frequency measurement will be influenced by biochemical, sequencing, and process error. In the case of heterozygous allele frequencies, these will also be influenced by molecular sampling error.

The deviation is the difference between the allele frequency in the DNA sample from the recipient where the donor genotype matches with the recipient genotype (i.e., the expected allele frequency) and the allele frequency in the DNA sample obtained from the transplant patient, where the donor genotype does not match the recipient genotype (i.e., the measured allele frequency). In some cases, an allele frequency average, mean or median is determined for the expected allele frequency and measured allele frequency and used for calculation of the deviation. Thus, for SNPs where the recipient is homozygous for the alternate allele (the reference allele frequency is about 0, or is in the range of 0.00-0.03, 0.00-0.02, e.g., 0.00-0.01), the deviation is the difference in mean or median of allele frequencies where the donor is homozygous for the alternate allele (matching recipient genotype) vs. the mean or median of allele frequencies where the donor is either heterozygous or homozygous for the reference allele (differing form recipient genotype).

For SNPs where the recipient is heterozygous for the alternate allele (the reference allele frequency is about 0.5, or is in the range of 0.40-0.60, 0.42-0.56, or 0.46-0.53), the deviation is the difference in mean or median of allele frequencies where the donor is heterozygous for the alternate allele (matching recipient genotype) vs. the mean or median of allele frequencies where the donor is either homozygous for the alternate allele or homozygous for the reference allele (differing form recipient genotype).

For SNPs where the recipient is homozygous for the reference allele (the reference allele frequency is about 1.00, or in the range of 0.97-1.00, or 0.98-1.00, e.g., 0.99-1.00), the deviation is the difference in mean or median of allele frequencies where the donor is homozygous for the reference allele (matching recipient genotype) vs. the mean or median of allele frequencies where the donor is either heterozygous or homozygous for the alternate allele (differing form recipient genotype).”

Whether a particular transplant donor/recipient belong to one or another category can be determined based on a single assay, without genotyping the donor or genotyping the recipient before receiving the transplant by using the methods as described below.

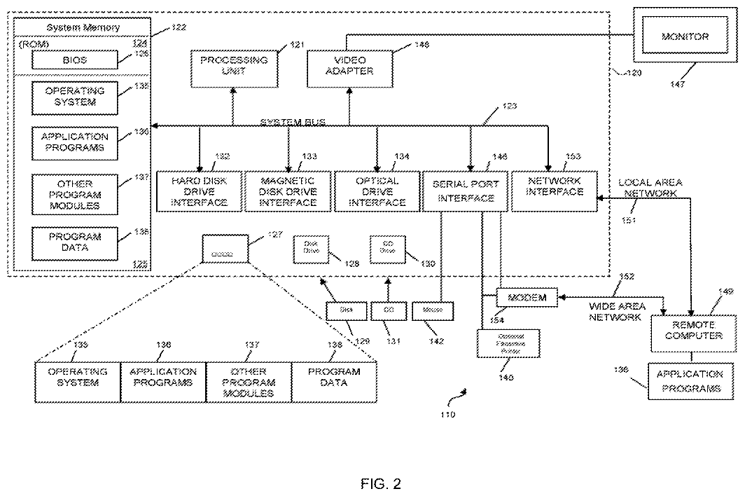

In these cases, these methods assume that normal SNP allele frequencies (allele frequencies associated with homozygous alternate allele genotypes, heterozygous alternate and reference allele genotypes, or homozygous reference allele genotypes) are present from recipient allele background. In these cases, the donor-specific nucleic acids can be identified using, for example, one or more of a fixed cutoff approach, a dynamic clustering approach, and an individual polymorphic nucleic acid target threshold approach, as described below. In some cases, sequence reads generated from sequencing the SNPs in a panel are filtered to first remove SNPs that have low quality sequence reads. This can decrease background noise in SNP allele frequency measurement and enable a more precise genotype frequency calculation. Table 2 shows the features of the various exemplary approaches that can be used for these purposes. In general, such approaches are performed by a processor, a micro-processor, a computer system, in conjunction with memory and/or by a microprocessor controlled apparatus. In various embodiments, the approaches are performed as a sequence of events or steps (e.g., a method or process) in the operating environment 110 described with respect to FIG. 2 herein.

TABLE 2

| Methods | Description |
|---|---|
| Fixed cutoff | Establish a fixed cutoff level for homozygous allele |
| for | frequencies defined as a fixed percentile of homozygous |
| homozygous | SNP allele frequencies |
| variance | Easily established by analysis of a moderate sized cohort |
| | Does not allow for differences in variance across SNPs |
| | within a panel |
| Dynamic | Use clustering algorithm (k-means) on a per sample basis |
| k-means | Two tiered approach to dynamically stratify SNPs based |
| clustering | on recipient homozygous or heterozygous genotype and |
| | then stratify recipient homozygous SNPs into non- |
| | informative and informative groups |
| SNP specific | Establish specific homozygous allele frequencies threshold |
| variance | for each individual SNP in the panel |
| threshold | Established by analysis of a large cohort of genome DNA |
| | to collect data on homozygous SNP genotypes |
| | Allows for differences in variance across SNPs within a |
| | panel |

The Fixed Cutoff Method

In some embodiments, determining whether a polymorphic nucleic acid target is informative and/or detect donor-specific nucleic acids comprises comparing its measured allele frequency in a recipient to a fixed cutoff frequency. In some cases, determining which polymorphic nucleic acid targets are informative comprises identifying informative genotypes by comparing each allele frequency to one or more fixed cutoff frequencies. Fixed cutoff frequencies may be predetermined threshold values based on one or more qualifying data sets from a population of subjects who have not received transplant, for example, and represent the variance of the measured allele frequencies in subjects who have not received transplant.

In some cases, the fixed cutoff for identifying informative genotypes from non-informative genotypes is expressed as a percent (%) shift in allele frequency from an expected allele frequency. Generally, expected allele frequencies for a given allele (e.g., allele A) are 0 (for a BB genotype), 0.5 (for an AB genotype) and 1.0 (for an AA genotype), or equivalent values on any numerical scale. If a polymorphic nucleic acid target allele frequency in the recipient deviate from an expected allele frequency and such deviation is beyond one or more fixed cutoff frequencies, the polymorphic nucleic acid target may be considered informative. The degree of deviation generally is proportional to donor-specific nucleic acid fraction (i.e., large deviations from expected allele frequency may be observed in samples having high donor-specific nucleic acid fraction). The deviation between the expected allele frequency and measured allele frequency can be determined as described above.

In some cases, the polymorphic nucleic acid targets in the recipient before transplantation are homozygous and the expected allele frequency, either the reference allele or the alternate allele, is, e.g., 0. In these circumstances, the deviation between the measured allele frequency in transplant recipient and expected allele frequency is equal to the measured allele frequency. The polymorphic nucleic acid targets are identified as informative if the measured allele frequency is greater than the fixed cutoff.

In some cases, the fixed cutoff is a percentile value of the measure allele frequencies of all the polymorphic nucleic acid targets used in the assay. In some embodiments, the percentile value is a 90, 95 or 98 percentile value.

In some cases, the fixed cutoff for identifying informative genotypes from non-informative homozygotes is about a 0.5% or greater shift in allele frequency from the median of expected allele frequencies. For example, a fixed cutoff may be about a 0.6%, 0.7%, 0.8%, 0.9%, 1%, 1.5%, 2%, 3%, 4%, 5%, 10% or greater shift in allele frequency. In some cases, the fixed cutoff for identifying informative genotypes from non-informative homozygotes is about a 1% or greater shift in allele frequency. In some cases, the fixed cutoff for identifying informative genotypes from non-informative homozygotes is about a 2% or greater shift in allele frequency. In some embodiments, the fixed cutoff for identifying informative genotypes from non-informative heterozygotes is about a 10% or greater shift in allele frequency. For example, a fixed cutoff may be about a 10%, 15%, 20%, 21%, 22%, 23%, 24%, 25%, 26%, 27%, 28%, 29%, 30%, 35%, 40%, 45%, 50%, 55%, 60%, 70%, 80% or greater shift in allele frequency. In some cases, the fixed cutoff for identifying informative genotypes from non-informative heterozygotes is about a 25% or greater shift in allele frequency. In some cases, the fixed cutoff for identifying informative genotypes from non-informative heterozygotes is about a 50% or greater shift in allele frequency.

Target-Specific Threshold Method

In some embodiments, determining whether a polymorphic nucleic acid target is informative and/or detecting the donor-specific allele comprises comparing its measured allele frequency to a target-specific threshold (e.g., a cutoff value). In some embodiments, target-specific threshold frequencies are determined for each polymorphic nucleic acid target. Typically, target-specific threshold frequency is determined based on the allele frequency variance for the corresponding polymorphic nucleic acid target. In some embodiments, variance of individual polymorphic nucleic acid targets can be represented by a median absolute deviation (MAD), for example. In some cases, determining a MAD value for each polymorphic nucleic acid target can generate unique (i.e., target-specific) threshold values. To determine median absolute deviation, measured allele frequency can be determined, for example, for multiple replicates (e.g., 5, 6, 7, 8, 9, 10, 15, 20 or more replicates) of a recipient only nucleic acid sample (e.g., buffy coat sample). Each polymorphic nucleic acid target in each replicate will typically have a slightly different measured allele frequency due to PCR and/or sequencing errors, for example. A median allele frequency value can be identified for each polymorphic nucleic acid target. A deviation from the median for the remaining replicates can be calculated (i.e., the difference between the observed allele frequency and the median allele frequency). The absolute value of the deviations (i.e., negative values become positive) is taken and the median value of the absolute deviations is calculated to provide a median absolute deviation (MAD) for each polymorphic nucleic acid target. A target-specific threshold can be assigned, for example, as a multiple of the MAD (e.g., 1×MAD, 2×MAD, 3×MAD, 4×MAD or 5×MAD). Typically, polymorphic nucleic acid targets having less variance have a lower MAD and therefore a lower threshold value than more variable targets.

In some embodiments, the target-specific threshold is a percentile value of the measured allele frequencies of the polymorphic nucleic acid target used in the assay. In some embodiments, the percentile value is a 90, 95 or 98 percentile value.

Dynamic Clustering Algorithm

In some embodiments, determining whether a polymorphic nucleic acid target is informative and/or detecting the donor-specific allele comprises a dynamic clustering algorithm. Non-limiting examples of dynamic clustering algorithms include K-means, affinity propagation, mean-shift, spectral clustering, ward hierarchical clustering, agglomerative clustering, DBSCAN, Gaussian mixtures, and Birch. See, http://scikit-learn.org/stable/modules/clustering.html#k-means. Such algorithms may be implemented with a processor, a micro-processor, a computer system, in conjunction with memory and/or by a microprocessor controlled apparatus.

In some embodiments, the dynamic clustering algorithm is a k-means clustering. The k-means algorithm divides a set of samples into disjoint clusters, each described by the mean position of the samples in the cluster. The means are commonly referred to as cluster “centroids”. The k-means algorithm aims to choose centroids that minimize the inertia, or within-cluster sum of squares criterion. k-means is often referred to as Lloyd's algorithm. In basic terms, the algorithm has three steps. The first step chooses the initial centroids, with the most basic method being to choose k samples from a datasetX. After initialization; k-means consists of looping between the two other steps. The first step assigns each sample to its nearest centroid. The second step creates new centroids by taking the mean value of all of the samples assigned to each previous centroid. The difference between the old and the new centroids are computed and the algorithm repeats these last two steps unto this value is less than a threshold. In other words, it repeats until the centroids do not move significantly.

In some embodiments, the dynamic clustering comprises stratifying the one or more polymorphic nucleic acid targets in the nucleic acids into recipient homozygous group and recipient heterozygous group, based on the measured allele frequency for a reference allele or an alternate allele for each of the polymorphic nucleic acid targets. Homozygous groups are clustered having a mean position of close to 0 or 1, and heterozygous group are clustered having a mean position of close to 0.5.

The method may further comprise stratifying recipient homozygous groups into non-informative and informative groups; and measuring the amounts of one or more polymorphic nucleic acid targets in the informative groups. In some embodiments, stratifying the recipient homozygous groups into non-informative and informative groups is based on whether the group contains donor-specific alleles—informative groups are the groups that comprise distinct donor alleles derived from the donor that are not present in the recipients genome and non-informative groups comprise alleles from the donor, where the informative SNPs are defined as those within the cluster with higher mean or median allele frequency. These informative SNPs can be used to determine the fractional concentration of donor-specific nucleic acids.

In some embodiments, the k-means clustering process is repeated as described above to identify a cutoff for the informative SNPS. To find a cutoff, clustering is performed on SNPs with allele frequencies in the range of (0, 0.25). This results in 2 clusters where cluster 1 (the lower cluster) are non-informative SNPs (donor & recipient alleles match) and cluster 2 (the higher cluster) are informative SNPs (donor has at least one different allele than the recipient). The cutoff is calculated as the average of the maximum of the first/lower cluster and the minimum of the second/upper cluster.

In some embodiments, the informative SNPs are determined substantially as follows:

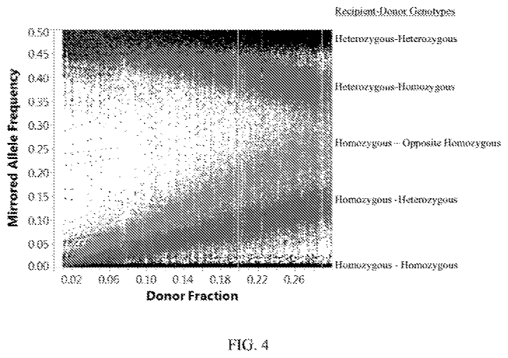

As a first step in calculating donor fraction, allele frequencies are first mirrored to generate mirrored allele frequencies. A mirrored allele frequency is the lesser value of the allele frequency of an allele and (1−the allele frequency). This mirrors allele frequencies larger than 0.5 into a range of [0,0.5] and groups similar donor-recipient genotype combinations together (e.g. AA_{recipient}/AB_{donor} with BB_{recipient}/AB_{donor}). Next, an “informative” SNPs is identified as an SNP where the donor's genotype and the recipient's genotype for the SNP are different. Defining the reference alleles as A and alternate alleles as B, there are 3 categories of informative SNPs (FIG. 3 and FIG. 4):
1) Informative category 1 refers to the “Homo-Het” category, in which the recipient is homozygous and the donor is heterozygous (e.g. AA_{recipient}/AB_{donor} or BB_{recipient}/AB_{donor}).
2) Informative category 2 refers to the “Homo-Opp Homo” category, in which the recipient is homozygous and the donor is homozygous for the opposite allele (e.g. AA_{recipient}/BB_{donor} or BB_{recipient}/AA_{donor}). This occurs when the donor and recipient are unrelated.
3) Informative category 3 refers to the “Het-Homo” category, in which the recipient is heterozygous and the donor is homozygous (e.g. AB_{recipient}/AA_{donor} or AB_{recipient}/BB_{donor}).

In some embodiments, the informative SNPs selected for detecting donor specific nucleic acid and/or determining the donor specific nucleic acid fraction do not include the category 3 SNPs.

The data shown in FIG. 3 and FIG. 4 utilize 91 mixtures of genomic DNA and non-pregnant plasma cfDNA to simulate donor-recipient mixtures. The mirrored allele frequencies increase with higher donor fraction for SNPs in category 1 and 2, but decreases for category 3 SNPs (FIG. 4). To focus on a positive correlation, the category 3 SNPs are excluded and re-classified as non-informative for the sake of calculating donor fraction (FIG. 3 and FIG. 4). The non-informative SNPs can then be identified and removed by different approaches, some of which depend on a two-step clustering analysis. When clustering is employed, the first step is an iteration of fuzzy K-means in the range of mirrored allele frequencies between 0 and 0.3 in order to determine a lower cutoff separating non-informative SNPs (e.g. AA_{recipient}/AA_{donor}) from informative SNPs (e.g. AA_{recipient}/AB_{donor}, AA_{recipient}/BB_{donor}). In a second round of clustering, hard K-means clustering is performed between this lower cutoff and an allele frequency of 0.49 to determine the upper bound of the desired informative SNPs (e.g. separating AA_{recipient}/AB_{donor} and AA_{recipient}/BB_{donor} from AB_{recipient}/AA_{donor} and AB_{recipient}/AB_{donor}).

Four different approaches are detailed as follows, depending on availability of the genotype for the donor or recipient:

1) Approach 1 (“DF1”):

If neither donor nor recipient's genotype is known, use K-means clustering to identify and remove non-informative SNPs (AA_{recipient}/AA_{donor}, BB_{recipient}/BB_{donor}, and AB_{recipient}/AB_{donor}, AB_{recipient}/AA_{donor}, and AB_{recipient}/BB_{donor} combinations). The 2 clusters are expected to contain the following recipient/donor's genotype combinations:
a. Cluster 1=(AA_{recipient}/AB_{donor}, BB_{recipient}/AB_{donor}, AA_{recipient}/BB_{donor}, BB_{recipient}/AA_{donor}).
b. Cluster 2=(AB_{recipient}/AB_{donor}, AB_{recipient}/AA_{donor}, AB_{recipient}/BB_{donor}).
Retain only the SNPs in the cluster 1 as those are relevant to the donor fraction calculation.

Accordingly, using the DF1 approach, under the circumstances where neither the donor nor the recipient's genotype is known, the method of determining transplant status comprises:
I) isolating cell-free nucleic acids from a biological sample;
II) measuring the amount of each allele of the one or more SNPs in the biological sample to generate a data set consisting of measurements of the amounts of the one or more SNPs; an “informative” SNPs is identified as an SNP where the donor's genotype and the recipient's genotype for the SNP are different.
III) performing a computer algorithm on the data set to form a first cluster and a second cluster, wherein the first cluster comprising informative SNPs and the second cluster comprising non-informative SNPs,
wherein the informative SNPs are present in the recipient and the donor in a genotype combination of AA_{recipient}/AB_{donor}, BB_{recipient}/AB_{donor}, AA_{recipient}/BB_{donor}, or BB_{recipient}/AA_{donor}, and
wherein the non-informative SNPs are present in the recipient and the donor in a genotype combination of AB_{recipient}/AB_{donor}, AB_{recipient}/AA_{donor}, or AB_{recipient}/BB_{donor}, and
IV) detecting the donor specific allele based on the presence of the informative SNPs. In some embodiments, the method further comprises determining the donor-specific nucleic acid fraction based on the amount of the donor specific alleles.

2) Approach 2 (“DF2”):

If only the donor's genotype is known, filter out cases where the donor is homozygous for the alternate allele for (non-mirrored) allele frequencies less than 0.5 and homozygous for the reference allele for allele frequencies larger than 0.5. This excludes BB_{recipient}/BB_{donor}, and AB_{recipient}/BB_{donor} in the [0,0.5) allele frequency range and AA_{recipient}/AA_{donor} and AB_{recipient}/AA_{donor} clusters in the (0.5,1] allele frequency range.

Accordingly, using the DF2 approach, under the circumstances where the donor's genotype is known but the recipient's genotype is unknown, the disclosure provides a method of determining transplant status comprises:
I) isolating cell-free nucleic acids from a biological sample;
II) measuring the amount of each allele of the one or more SNPs in the biological sample to generate a data set consisting of measurements of the amounts of the one or more SNPs;
III) filtering out 1) SNPs which are present in the recipient and the donor in a genotype combination of AA_{recipient}/AA_{donor} or AB_{recipient}/AA_{donor} and the donor allele frequency is less than 0.5, and 2) SNPs which are present in the recipient and the donor in a genotype combination of BB_{recipient}/BB_{donor}, and AB_{recipient}/BB_{donor}, and the donor allele frequency is larger than 0.5; and
IV) detecting the donor specific alleles based on the presence of the remaining SNPs in the one or more SNPs in the biological sample. In some embodiments, the method further comprises determining the donor-specific nucleic acid fraction based on the amount of the donor specific alleles.

3) Approach 3 (“DF3”):

If only the recipient's genotype is known, filter out cases where the recipient is heterozygous (so AB_{recipient}/AB_{donor}, AB_{recipient}/AA_{donor}, and AB_{recipient}/BB_{donor} are excluded). Then perform clustering on the remaining SNPs to remove uninformative SNPs. The 2 clusters are expected to contain the following genotype combinations:
a. Cluster 1: AA_{recipient}/AB_{donor}, BB_{recipient}/AB_{donor}.
b. Cluster 2: AA_{recipient}/BB_{donor}, BB_{recipient}/AA_{donor}.
SNPs in both clusters are relevant to the donor fraction calculation and should be combined.

Accordingly, using the DF3 approach, under the circumstances where the recipient's genotype is known but the donor's genotype is unknown, the disclosure provides a method of determining transplant status comprises:
I) isolating cell-free nucleic acids from a biological sample;
   measuring the amount of each allele of the one or more SNPs in the biological sample to generate a data set consisting of measurements of the amounts of the one or more SNPs;
II) filtering out 1) SNPs which are present in the recipient and the donor in a genotype combination of AB_{recipient}/AB_{donor}, AB_{recipient}/AA_{donor}, and AB_{recipient}/BB_{donor},
III) performing a computer algorithm on the data set of the remaining SNPs to form a first cluster and a second cluster, both comprising informative SNPs. The first cluster comprises SNPs that are present in the recipient and the donor in a genotype combination of AA_{recipient}/AB_{donor}, or BB_{recipient}/AB_{donor}. The second cluster comprises SNPs that are present in the recipient and the donor in a genotype combination of AA_{recipient}/BB_{donor} or BB_{recipient}/AA_{donor}, and
IV) detecting the donor specific allele based on the presence of the remaining SNPs in the one or more SNPs in the biological sample.
In some embodiments, the method further comprises determining donor-specific nucleic acid fraction in the biological sample based on the amount of the donor specific alleles.

4) Approach 4 (“DF4”):

If both donor and recipient's genotypes are known, non-informative SNPs are precisely identified and excluded. Informative SNPs (AA_{recipient}/AB_{donor}, AA_{recipient}/BB_{donor}; AB_{recipient}/AA_{donor}, AB_{recipient}/BB_{donor}, BB_{recipient}/AA_{donor}, BB_{recipient}/AB_{donor}) are selected to determine the donor or recipient fraction.

Once non-informative SNPs are removed, the median is calculated on the remaining informative SNPs. Donor fraction is then estimated as a correction factor K times the median of the mirrored allele frequencies (Donor fraction=K*median(mirrored allele frequency)) for informative SNPs. The correction factor K is then used in cases where there is a 1 allele difference between the donor and the recipient (informative categories 1 and 3). K is then set to 2 to correct for there being 2 alleles in a diploid genome while the allele frequency only counts the fraction of alleles that are the reference allele. As an example, a 10% donor fraction would have 10 copies of donor AB for every 90 copies of recipient AA, but the allele frequency is 5% (10 A_{donor}/(10 A_{donor}+10 B_{donor}+90 A_{recipient}+90 A_{recipient})) and needs to be multiplied by 2 in order to obtain the donor fraction.

Ideally, K should be set to 1 for category 2 SNPs, which have a 2 allele difference between the donor and recipient. Given the potential challenge of resolving category 1 and 2 informative SNPs, the correction factor is applied to the grouping of both categories 1 and 2. This should not result in much error in the calculation of donor fraction as there should be a higher proportion of SNPs in category 1. Furthermore, it's not the absolute value of donor fraction that's important for transplant monitoring, but the measure of donor fraction increasing over the time elapsed since a transplant procedure.

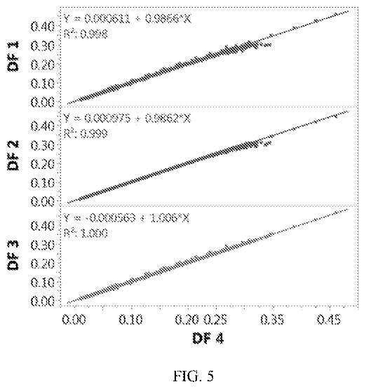

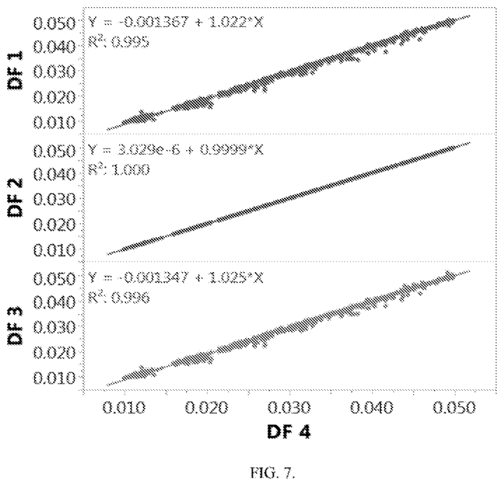

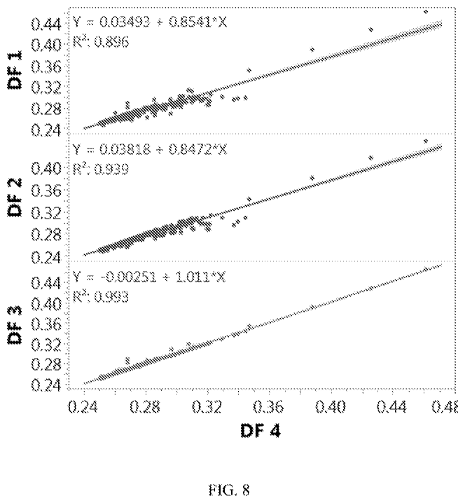

The data shown in FIG. 5 (as well as in FIG. 7 and FIG. 8) utilize 86 mixtures of genomic DNA and non-pregnant plasma cfDNA to simulate donor-recipient mixtures. FIG. 5 compares the donor fraction calculated by Approaches 1-3 with that of the most accurate determination using Approach 4. Approaches 1-3 correlate highly (R²>0.97) and match closely in value (slope=0.971-0.996), indicating overall excellent agreement between all the strategies for measuring moderate levels (e.g. 5%-25%) of donor fraction. It also indicates that K-means clustering of SNP allele frequencies is sufficient to identify informative SNPs in such a range. There's little advantage in knowing either the donor's or recipient's genotype in calculating the donor fraction unless the donor fraction is very low or very high.

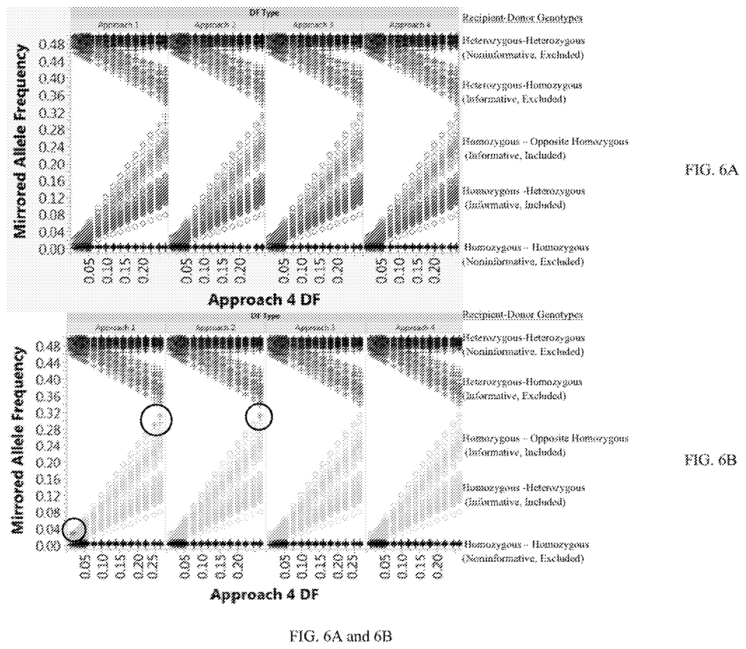

At very low (down to 0.5%) and very high donor fractions (near 30%), where different SNP allele frequency clusters can merge into each other, there can be misclassification of informative SNPs (FIG. 6). For example, at low donor fractions, AA_{recipient}/AB_{donor} SNPs could be regarded as AA_{recipient}/AA_{donor} SNPs, a false negative in detecting informative SNPs. This causes an overestimation of donor fraction by an average of 2%-3% for donor fractions less than 5% (FIG. 7, DF1 and DF3 panels). Approach 2 should be more accurate here as it removes AA_{recipient}/AA_{donor} and BB_{recipient}/BB_{donor} combinations through knowledge of the donor's genotype. This is verified by having the slope closest to 1 in the measurement using Approach 2 (FIG. 7, DF2 panel).

At higher donor fractions, AA_{recipient}/BB_{donor} SNPs could be classified as AB_{recipient}/AA_{donor} SNPs and BB_{recipient}/AA_{donor} SNPs could be classified as AB_{recipient}/BB_{donor}. Those are considered non-informative in this approach for donor fraction calculation, so another cause for false negatives. This causes a 25%-30% underestimation of donor fraction for donor fractions larger than 15% (FIG. 8). Approach 3, with knowledge of the recipient's genotype, could eliminate this issue through exclusion of AB_{recipient}/AA_{donor} and AB_{recipient}/BB_{donor} SNPs.

This is verified by having the slope closest to 1 in the measurement using Approach 3 (FIG. 8, DF3 panel).

Thus, the methods disclosed herein can be used to determine HSCT status in the absence of information of donor genotype and recipient genotypes with regard to the one or more polymorphic nucleic acid targets. The advantage of not having to genotype the recipient before the transplant and not having to genotype the donor is tremendous especially in situations where the patient is not submitted to testing until after transplantation, at which point the donor cannot be located and no pre-transplant samples from recipient was accessible for genotyping. Dispensing the need for genotyping before transplantation also saves costs in tracking the patient information. Without being bound to a particular theory, the present invention can determine the recipient genotype before transplant from a mixture of DNAs that include both donor and recipient DNA from post-transplant samples.

Other Considerations for Selecting Informative Polymorphism-Based Nucleic Acid Targets

Additional considerations may also be accounted for when selecting informative polymorphism-based nucleic acid targets for the purpose of detecting HSCT status. In some embodiments, individual polymorphic nucleic acid targets and/or panels of polymorphic nucleic acid targets are selected based on certain criteria, such as, for example, minor allele frequency, variance, coefficient of variance, MAD value, and the like. In some cases, polymorphic nucleic acid targets are selected so that at least one polymorphic nucleic acid target within a panel of polymorphic nucleic acid targets has a high probability of being informative for a majority of samples tested. Additionally, in some cases, the number of polymorphic nucleic acid targets (i.e., number of targets in a panel) is selected so that at least one polymorphic nucleic acid target has a high probability of being informative for a majority of samples tested. For example, selection of a larger number of polymorphic nucleic acid targets generally increases the probability that least one polymorphic nucleic acid target will be informative for a majority of samples tested. In some cases, the polymorphic nucleic acid targets and number thereof (e.g., number of polymorphic nucleic acid targets selected for enrichment) result in at least about 2 to about 50 or more polymorphic nucleic acid targets being informative for determining the donor-specific nucleic acid fraction for at least about 80% to about 100% of samples. For example, the polymorphic nucleic acid targets and number thereof result in at least about 5, 10, 15, 20, 25, 30, 35, 40, 45, 50 or more polymorphic nucleic acid targets being informative for determining the donor-specific nucleic acid fraction for at least about 81%, 82%, 83%, 84%, 85%, 86%, 87%, 88%, 89%, 90%, 91%, 92%, 93%, 94%, 95%, 96%, 97%, 98%, or 99% of samples. Using higher number informative polymorphic nucleic acids for the assay may boost accuracy and confidence in determine the amount of donor-specific or recipient-specific nucleic acid targets. In some cases, the polymorphic nucleic acid targets and number thereof result in at least five polymorphic nucleic acid targets being informative for determining the donor-specific nucleic acid fraction for at least 90% of samples. In some cases, the polymorphic nucleic acid targets and number thereof result in at least five polymorphic nucleic acid targets being informative for determining the donor-specific nucleic acid fraction for at least 95% of samples. In some cases, the polymorphic nucleic acid targets and number thereof result in at least five polymorphic nucleic acid targets being informative for determining the donor-specific nucleic acid fraction for at least 99% of samples. In some cases, the polymorphic nucleic acid targets and number thereof result in at least ten polymorphic nucleic acid targets being informative for determining the donor-specific nucleic acid fraction for at least 90% of samples. In some cases, the polymorphic nucleic acid targets and number thereof result in at least ten polymorphic nucleic acid targets being informative for determining the donor-specific nucleic acid fraction for at least 95% of samples. In some cases, the polymorphic nucleic acid targets and number thereof result in at least ten polymorphic nucleic acid targets being informative for determining the donor-specific nucleic acid fraction for at least 99% of samples.

In some embodiments, individual polymorphic nucleic acid targets are selected based, in part, on minor allele frequency. In some cases, polymorphic nucleic acid targets having minor allele frequencies of about 10% to about 50% are selected. For example, polymorphic nucleic acid targets having minor allele frequencies that ranges between 15-49%, e.g., 20-49%, 25-45%, 35-49%, or 40-40%. In some embodiments, the polymorphic nucleic acid target has a minor allele allele frequency of about 15%, 20%, 25%, 30%, 35%, 36%, 37%, 38%, 39%, 40%, 41%, 42%, 43%, 44%, 45%, 46%, 47%, 48%, or 49% are selected. In some embodiments, polymorphic nucleic acid targets having a minor allele population frequency of about 40% or more are selected. In some cases, the minor allele frequencies of the polymorphic nucleic acid targets can be identified from published databases or based on study results from a reference population.

By analyzing a panel of multiple polymorphic nucleic acid targets (e.g., SNPs) (for instance on the order of 100, 200, 300, etc.) with high minor allele frequencies (for instance from 0.4-0.5), a significant number of ‘informative’ donor and recipient genotype combinations (with donor genotypes differing from recipient genotype) may be seen (represent in FIG. 1 right panel). In some embodiments, polymorphic nucleic acid targets of the type I Informative genotypes, where the recipient is homozygous for one allele and the donor is heterozygous or homozygous for the other allele (compared to the recipient genotype), are used to determine a change in allele frequency due to the minimal impact of molecular sampling error on the background recipient homozygous allele frequency. In some embodiments, about 25% of the polymorphic nucleic acid targets in a panel are informative where the recipient is homozygous for one reference allele or one alternate allele and the donor is heterozygous. In cases of non-related donor/recipient pairs, the rate of informative polymorphic nucleic acid targets would be expected to be higher. Monozygotic twin donor/recipient pairs would be the exception with no informative genotype combinations present.

In some embodiments, the polymorphic nucleic acid targets are selected based on the GC content of the region surrounding the polymorphic nucleic acid targets and the amplification efficiency of the polymorphic nucleic acid targets. In some embodiments, the GC content is in a range of 10% to 80%, e.g., 20% to 70%, or 25% to 70%, 21% to 61% or 30% to 61%.

In some embodiments, individual polymorphic nucleic acid targets and/or panels of polymorphic nucleic acid targets are selected based, in part, on degree of variance for an individual polymorphic nucleic acid target or a panel of polymorphic nucleic acid targets. Variance, in some cases, can be specific for certain polymorphic nucleic acid targets or panels of polymorphic nucleic acid targets and can be from systematic, experimental, procedural, and or inherent errors or biases (e.g., sampling errors, sequencing errors, PCR bias, and the like). Variance of an individual polymorphic nucleic acid target or a panel of polymorphic nucleic acid targets can be determined by any method known in the art for assessing variance and may be expressed, for example, in terms of a calculated variance, an error, standard deviation, p-value, mean absolute deviation, median absolute deviation, median adjusted deviation (MAD score), coefficient of variance (CV), and the like. In some embodiments, measured allele frequency variance (i.e., background allele frequency) for certain SNPs (when homozygous, for example) can be from about 0.001 to about 0.01 (i.e., 0.1% to about 1.0%). For example, measured allele frequency variance can be about 0.002, 0.003, 0.004, 0.005, 0.006, 0.007, 0.008, or 0.009. In some cases, measured allele frequency variance is about 0.007.

In some cases, noisy polymorphic nucleic acid targets are excluded from a panel of polymorphic nucleic acid targets selected for determining donor-specific nucleic acid fraction. The term “noisy polymorphic nucleic acid targets” or “noisy SNPs” refers to (a) targets or SNPs that have significant variance between data points (e.g., measured donor-specific nucleic acid fraction, measured allele frequency) when analyzed or plotted, (b) targets or SNPs that have significant standard deviation (e.g., greater than 1, 2, or 3 standard deviations), (c) targets or SNPs that have a significant standard error of the mean, the like, and combinations of the foregoing. Noise for certain polymorphic nucleic acid targets or SNPs sometimes occurs due to the quantity and/or quality of starting material (e.g., nucleic acid sample), sometimes occurs as part of processes for preparing or replicating DNA used to generate sequence reads, and sometimes occurs as part of a sequencing process. In certain embodiments, noise for some polymorphic nucleic acid targets or SNPs results from certain sequences being over represented when prepared using PCR-based methods. In some cases, noise for some polymorphic nucleic acid targets or SNPs results from one or more inherent characteristics of the site such as, for example, certain nucleotide sequences and/or base compositions surrounding, or being adjacent to, a polymorphic nucleic acid target or SNP. A SNP having a measured allele frequency variance (when homozygous, for example) of about 0.005 or more may be considered noisy. For example, a SNP having a measured allele frequency variance of about 0.006, 0.007, 0.008, 0.009, 0.01 or more may be considered noisy.

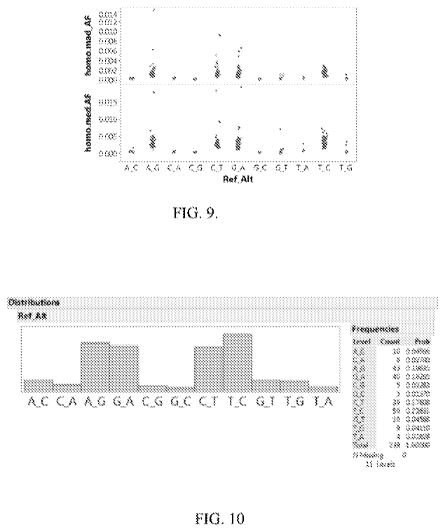

In some embodiments, the reference allele and alternate allele combination of one or more SNPs selected for determining the transplant status is not any one of A_G, G_A, C_T, and T_C (the first letter refers to the reference allele and the second letter refers to the alternate allele). As shown in FIG. 9 and Example 2, SNPs having the above reference allele and alternate allele combination showed higher amount of bias and variability; thus they are not suitable for use in the method disclosed herein for determining the donor fraction and transplant status.

In some embodiments, the one or more SNPs selected for determining the transplant status meet one or more, or all of the following criteria:
1. Biallelic.
2. The SNP is not located within the primer annealing regions.
3. Validated by the 1000 Genomes Project.
4. The ref_alt combination is not any of the A_G, G_A, C_T or T_C.
5. Minor allele frequency is at least 0.3.
6. The sequence for amplified target region is unique and cannot be found elsewhere in the genome.

In some embodiments, variance of an individual polymorphic nucleic acid target or a panel of polymorphic nucleic acid targets can be represented using coefficient of variance (CV).

Coefficient of variance (i.e., standard deviation divided by the mean) can be determined, for example, by determining donor-specific nucleic acid fraction for several aliquots of a single recipient sample comprising recipient and donor-specific nucleic acid, and calculating the mean donor-specific nucleic acid fraction and standard deviation. In some cases, individual polymorphic nucleic acid targets and/or panels of polymorphic nucleic acid targets are selected so that donor-specific nucleic acid fraction is determined with a coefficient of variance (CV) of 0.30 or less. For example, donor-specific nucleic acid fraction may be determined with a coefficient of variance (CV) of 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01 or less, in some embodiments. In some cases, donor-specific nucleic acid fraction is determined with a coefficient of variance (CV) of 0.20 or less. In some cases, donor-specific nucleic acid fraction is determined with a coefficient of variance (CV) of 0.10 or less. In some cases, donor-specific nucleic acid fraction is determined with a coefficient of variance (CV) of 0.05 or less.

The informative SNPs can be selected based on any of the combination of criteria. In some embodiments, the informative SNPs comprise at least one, two, three, four or more SNPs in Table 1. These SNPs have alternative alleles occurring frequently in individuals within a population. As well, these SNPs are diverse and present in multiple populations. Informative analysis indicates that possibility to design specific nucleic acid primers to these SNPs with low potential for off-target non-specific amplification.

TABLE 1
Exemplary SNPs

| | |
|---|---|
| Panel | rs10737900, rs1152991, rs10914803, rs4262533, rs686106, |
| A | rs3118058, rs4147830, rs12036496, rs1281182, rs863368, |
| | rs765772, rs6664967, rs12045804, rs1160530, rs11119883, |
| | rs751128, rs7519121, rs9432040, rs7520974, rs1879744, |
| | rs6739182, rs4074280, rs7608890, rs6758291, rs13026162, |
| | rs2863205, rs11126021, rs9678488, rs10168354, rs13383149, |
| | rs955105, rs2377442, rs13019275, rs967252, rs16843261, |
| | rs2049711, rs2389557, rs6434981, rs1821662, rs1563127, |
| | rs7422573, rs6802060, rs9879945, rs7652856, rs1030842, |
| | rs614004, rs1456078, rs6599229, rs1795321, rs4928005, |
| | rs9870523, rs7612860, rs11925057, rs792835, rs9867153, |
| | rs602763, rs12630707, rs2713575, rs9682157, rs13095064, |
| | rs2622744, rs12635131, rs7650361, rs16864316, rs9810320, |
| | rs9841174, rs7626686, rs9864296, rs2377769, rs4687051, |
| | rs1510900, rs6788448, rs11941814, rs4696758, rs7440228, |
| | rs13145150, rs17520130, rs11733857, rs6828639, rs6834618, |
| | rs16996144, rs376293, rs11098234, rs975405, rs1346065, |
| | rs1992695, rs6849151, rs11099924, rs6857155, rs10033133, |
| | rs7673939, rs7700025, rs6850094, rs11132383, rs7716587, |
| | rs38062, rs582991, rs2388129, rs9293030, rs11738080, |
| | rs13171234, rs309622, rs253229, rs11744596, rs4703730, |
| | rs10040600, rs11953653, rs163446, rs4920944, rs11134897, |
| | rs226447, rs12194118, rs4959364, rs4712253, rs2457322, |
| | rs7767910, rs2814122, rs6930785, rs1145814, rs1341111, |
| | rs2615519, rs1894642, rs6570404, rs9479877, rs9397828, |
| | rs6927758, rs6461264, rs6947796, rs1347879, rs10246622, |
| | rs10232758, rs756668, rs2709480, rs1983496, rs1665105, |
| | rs11785007, rs10089460, rs1390028, rs4738223, rs6981577, |
| | rs10958016, rs9298424, rs517811, rs1442330, rs1002142, |
| | rs2922446, rs1514221, rs387413, rs10758875, rs10759102, |
| | rs2183830, rs1566838, rs12553648, rs10781432, rs11141878, |
| | rs2756921, rs1885968, rs10980011, rs1002607, rs10987505, |
| | rs1334722, rs723211, rs4335444, rs7917095, rs10509211, |
| | rs10881838, rs2286732, rs4980204, rs12286769, rs4282978, |
| | rs7112050, rs7932189, rs7124405, rs7111400, rs1938985, |
| | rs7925970, rs7104748, rs10790402, rs2509616, rs4609618, |
| | rs12321766, rs2920833, rs10133739, rs10134053, rs7159423, |
| | rs2064929, rs1298730, rs2400749, rs12902281, rs11074843, |
| | rs9924912, rs1562109, rs2051985, rs8067791, rs12603144, |
| | rs16950913, rs1486748, rs2570054, rs2215006, rs4076588, |
| | rs7229946, rs9945902, rs1893691, rs930189, rs3745009, |
| | rs1646594, rs7254596, rs511654, rs427982, rs10518271, |
| | rs1452321, rs6080070, rs6075517, rs6075728, rs6023939, |
| | rs3092601, rs6069767, rs2426800, rs2826676, rs2251381, |
| | rs2833579, rs1981392, rs1399591, rs2838046, rs8130292, |
| | rs241713 |
| Panel | rs10413687, rs10949838, rs1115649, rs11207002, rs11632601, |
| B | rs11971741, rs12660563, rs13155942, rs1444647, rs1572801, |
| | rs17773922, rs1797700, rs1921681, rs1958312, rs196008, |
| | rs2001778, rs2323659, rs2427099, rs243992, rs251344, |
| | rs254264, rs2827530, rs290387 , rs321949, rs348971, |
| | rs390316, rs3944117, rs425002, rs432586, rs444016, |
| | rs4453265, rs447247, rs4745577, rs484312, rs499946, |
| | rs500090, rs500399, rs505349, rs505662, rs516084, |
| | rs517316, rs517914, rs522810, rs531423, rs537330, rs539344, |
| | rs551372, rs567681, rs585487, rs600933, rs619208, |
| | rs622994, rs639298, rs642449, rs6700732, rs677866, |
| | rs683922, rs686851, rs6941942, rs7045684, rs7176924, |
| | rs7525374, rs870429, rs949312, rs9563831, rs970022, |
| | rs985462, rs1005241, rs1006101, rs10745725, rs10776856, |
| | rs10790342, rs11076499, rs11103233, rs11133637, |
| | rs11974817, rs12102203, rs12261, rs12460763, rs12543040, |
| | rs12695642, rs13137088, rs13139573, rs1327501, rs13438255, |
| | rs1360258, rs1421062, rs1432515, rs1452396, rs1518040, |
| | rs16853186, rs1712497, rs1792205, rs1863452, rs1991899, |
| | rs2022958, rs2099875, rs2108825, rs2132237, rs2195979, |
| | rs2248173, rs2250246, rs2268697, rs2270893, rs244887, |
| | rs2736966, rs2851428, rs2906237, rs2929724, rs3742257, |
| | rs3764584, rs3814332, rs4131376, rs4363444, rs4461567, |
| | rs4467511, rs4559013, rs4714802, rs4775899, rs4817609, |
| | rs488446, rs4950877, rs530913, rs6020434, rs6442703, |
| | rs6487229, rs6537064, rs654065, rs6576533, rs6661105, |
| | rs669161, rs6703320, rs675828, rs6814242, rs6989344, |
| | rs7120590, rs7131676, rs7214164, rs747583, rs768255, |
| | rs768708, rs7828904, rs7899772, rs7900911, rs7925270, |
| | rs7975781, rs8111589, rs849084, rs873870, rs9386151, |
| | rs9504197, rs9690525, rs9909561, rs10839598, rs10875295, |
| | rs12102760, rs12335000, rs12346725, rs12579042, |
| | rs12582518, rs17167582, rs1857671, rs2027963, |
| | rs2037921, rs2074292, rs2662800, rs2682920, |
| | rs2695572, rs2713594, rs2838361, rs315113, |
| | rs3735114, rs3784607, rs3817, rs3850890, rs3934591, |
| | rs4027384, rs405667, rs4263667, rs4328036, rs4399565, |
| | rs4739272, rs4750494, rs4790519, rs4805406, |
| | rs4815533, rs483081, rs4940791, rs4948196, rs582111, |
| | rs596868, rs6010063, rs6014601, rs6050798, rs6131030, |
| | rs631691, rs6439563, rs6554199, rs6585677, rs6682717, |
| | rs6720135, rs6727055, rs6744219, rs6768281, rs681836, |
| | rs6940141, rs6974834, rs718464, rs7222829, rs7310931, |
| | rs732478, rs7422573, rs7639145, rs7738073, rs7844900, |
| | rs7997656, rs8069699, rs8078223, rs8080167, rs8103778, |
| | rs8128, rs8191288, rs886984, rs896511, rs931885, |
| | rs9426840, rs9920714, rs9976123, rs999557, rs9997674 |

In some embodiments, the polymorphic nucleic acid targets selected for determining transplant rejection are a combination of any of the polymorphic nucleic acid targets in Table 1 (Panel A and/or panel B) or Table 6.

A plurality of polymorphic nucleic acid targets is sometimes referred to as a collection or a panel (e.g., target panel, SNP panel, and SNP collection). A plurality of polymorphic nucleic acid targets can comprise two or more targets. For example, a plurality of polymorphic nucleic acid targets can comprise 2, 3, 4, 5, 6, 7, 8, 9, 10, 20, 30, 40, 50, 60, 70, 80, 90, 100, 200, 300, 400, 500, 600, 700, 800, 900, 1000, or more targets.

In some cases, 10 or more polymorphic nucleic acid targets are enriched using the methods described herein. In some cases, 50 or more polymorphic nucleic acid targets are enriched. In some cases, 100 or more polymorphic nucleic acid targets are enriched. In some cases, 500 or more polymorphic nucleic acid targets are enriched. In some cases, about 10 to about 500 polymorphic nucleic acid targets are enriched. In some cases, about 20 to about 400 polymorphic nucleic acid targets are enriched. In some cases, about 30 to about 200 polymorphic nucleic acid targets are enriched. In some cases, about 40 to about 100 polymorphic nucleic acid targets are enriched. In some cases, about 60 to about 90 polymorphic nucleic acid targets are enriched. For example, in certain embodiments, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 polymorphic nucleic acid targets are enriched.

Determining Transplantation Status

Calculating Recipient-Specific Nucleic Acid Fraction and Donor-Specific Nucleic Acid Fraction

In some cases, the amount of a target nucleic acid in the sample (donor-specific nucleic acid or recipient-specific nucleic acid) can be determined as a parameter of the total number of unique sequence reads mapped to the target nucleic acid sequence on a reference genome for each of the alleles (a reference allele and one or more alternate alleles) of a polymorphic site. In some embodiments, the relative abundance or a fraction of the target nucleic acid is determined based on the frequencies of one or more polymorphic nucleic acid targets (either donor-specific or recipient-specific) in the sample. In some embodiments, the relative abundance of the recipient-specific nucleic acid or donor-specific nucleic acid can be represented by the frequency of the recipient-specific polymorphic nucleic acid target sequence or donor-specific polymorphic nucleic acid target sequence in the sample. For example, if the recipient specific allele is A and the donor-specific allele is T for the same polymorphic site, and the A appears in a frequency of 30% of the reads and T appears in 70% of the reads generated from sequencing the polymorphic site, then the fraction of recipient-specific nucleic acid is 30% and the fraction of donor-specific nucleic acid is 70%.

In some embodiments, the donor-specific nucleic acid fraction (or the recipient-specific nucleic acid fraction) is calculated as the median of the allele frequencies across all informative SNPs specific for the donor (or for the recipient).

In some embodiments, the donor fraction is obtained by multiplying a correction factor to frequencies of informative SNPs. A correction factor of either 1 or 2 applies depending on the types of informative SNPs: if the SNP can be identified as such that the donor has one different allele from the recipient, a correction factor of 2 is applied; if the SNP can be identified as where the donor has two different alleles from the recipient, a correction factor of 1 is applied. The type of SNPs can be typically determined from analyzing the resulting allele frequency from a mixture of donor and recipient DNA, the donor genotype is not needed to obtain such information. In some embodiments, whether the SNP is one that the donor has one or two different alleles from the recipient can be determined based on relatedness between the recipient and donor. For example, if the recipient is the parent of the donor, the donor can only have one allele different from the recipient. If the recipient and donor are unrelated, ⅓ of the SNPs will be cases where the donor has one differing allele and the correction factor will be 2 for those SNPs. The other ⅔rd of the SNPs will be cases where the donor has 2 differing alleles and the correction factor will be 1 for those SNPs. K-means clustering can be used to separate those 2 categories of SNPs, or they can be simply separated into an upper ⅓rd and lower ⅔rd groups for applying the correction factor. After correction factors are applied, the donor fraction is the median across all corrected informative SNPs. Recipient-specific nucleic acid fraction can be calculated in the same manner using SNPs that are identified as specific for the recipient.

In some embodiments, the donor-specific fraction is inferred by the recipient-specific fraction by subtracting the recipient-specific fraction by 100%. Conversely, the recipient-specific fraction can also be inferred by the donor-specific fraction by subtracting the donor-specific fraction by 100%. For example, if the recipient specific allele is A and the donor-specific allele is T for the same polymorphic site, and the A appears in a frequency of 30% of the reads, then the fraction of donor-specific nucleic acid is 70% (100%-30%). For example, if the donor specific allele is A and the A appears in a frequency of 30% of the reads, then the fraction of recipient-specific nucleic acid is 70% (100%-30%).

In some embodiments, a fraction can be determined for the amount of one nucleic acid relative to the total amount of mixed nucleic acids. In some embodiments, the fraction of donor-specific nucleic acid or recipient-specific nucleic acid in a sample relative to the total amount of nucleic acid in the sample is determined. In general, to calculate the fraction of donor-specific nucleic acid or recipient-specific nucleic acid in a sample relative to the total amount of the nucleic acid in the sample, the following equation can be applied:

The fraction of donor-specific nucleic acid=(amount of donor-specific nucleic acid)/(amount of total nucleic acid).

The fraction of recipient-specific nucleic acid=(amount of recipient-specific nucleic acid)/(amount of total nucleic acid).

Calculating the Copy Number of Donor-Specific Nucleic Acids and/or Recipient-Specific Nucleic Acids

In some embodiments, the total copy of genomic DNA is determined using a reference genomic nucleic acid and a variant oligo, which is designed to contain a single nucleotide substitution as compared to the reference genomic nucleic acid and which is co-amplified with one or more polymorphic nucleic acid targets. The variant oligo is added to the amplification mixture at a known quantity. After sequencing, the number of sequences containing the variant are compared to the number of sequences containing the reference genomic nucleic acid and the ratio of the two is determined. Since the variant oligo's quantity is known, the total copies of genomic DNA can be calculated based on the quantity of the variant oligo and the ratio of the number of sequences containing the variant to the number of sequences containing the reference genomic nucleic acid. In one embodiment, the reference genomic nucleic acid is ApoE. In one embodiment, the reference genomic nucleic acid is RNasP.

In some embodiments the total copy number of the genomic DNA in the sample and the donor-specific nucleic acid fraction or the recipient-specific nucleic acid fraction is multiplied to generate the total copy number of donor-specific or recipient specific nucleic acid, which is used to indicate the status of transplant. The total copy number of donor-specific nucleic acids or the total copy number of recipient-specific nucleic acids in some instances can be a better indicator of rejection, since, for example, a high recipient genomic copy number may be masked as a low fractional concentration in a recipient having a high body mass index (BMI), or the increase of copy number of recipient specific DNA may be masked as a decrease or unchanged fractional concentration as the patient gains weight.

Determining Transplantation Status

Transplantation status, i.e. whether the transplant is rejected or accepted, can be determined by monitoring the donor-specific nucleic acid fraction (“donor fraction”) or donor-specific nucleic acid copy number (“donor load”) in the transplant patient. Likewise, the transplantation status can also be determined by monitoring the recipient-specific nucleic acid fraction (“recipient fraction”) or recipient-specific nucleic acid copy number (“recipient load”) in the transplant patient.

Determining Engraftment of HSCT (“Successful Engraftment” or “Full Chimerism”) Versus Graft Failure

In some embodiments, the donor fraction or donor load of the transplant patient is compared with a predetermined threshold: the transplantation status is determined as engraftment of the HSCT if the donor fraction is equal to or greater than a first predetermined threshold. In some cases, the first predetermined threshold is a value selected from the group consisting of 91%, 95%, 99% 99.5%, and 100%. In some cases, the transplantation status is determined as engraftment of the HSCT if the donor fraction is within a range from 91% to 100%, for example, from 95% to 100%, or from 99% to 100%. The transplantation status may be determined as graft failure or at risk for graft failure if the donor fraction is lower than a second predetermined threshold. In some case the second predetermined threshold is a value selected from the group consisting of 5%, 4%, 3%, 2%, and 1%.

Conversely, the recipient fraction or recipient load of the transplant patient can be compared with a predetermined threshold; and transplantation status is determined as engraftment of the HSCT if the recipient fraction or recipient load is equal to or lower than the third predetermined threshold, In some case, the threshold for determining transplantation status using the recipient fraction is a value a value selected from the group consisting of 10%, 9%, 5%, 2.5%, 1%, 0.5%, 0.1%, or 0%. In some cases, the transplantation status is determined to be engraftment of the HSCT if the recipient fraction ranges from 10% to 0%, e.g., from 9% to 0.1%, or from 5% to 0.1%. The transplantation status may be determined as graft failure if the recipient fraction is greater than a fourth predetermined threshold. In some case the second predetermined threshold is a value selected from the group consisting of 95%, 96%, 97%, 98%, and 99%.

Any of the thresholds described above can be predetermined based on the background levels of allele frequencies in a control patient(s), for example, a patient(s) who has (have) not received an organ transplant. In some embodiments, the control patient is one who is within the same gender, age, and ethnic group as the subject for which transplantation status is to be determined and the control patient has similar BMI as the subject.

In some embodiments, the donor fraction or donor load is determined for samples taken at various time points after transplant. An increase in donor fraction or donor load over time is an indication of engraftment of the HSCT and a decrease over time is an indication of graft failure. Conversely, a decrease in recipient fraction or recipient load over time is an indication of engraftment of the HSCT and an increase is an indication of graft failure. In some embodiments, the transplantation status is monitored at two or more time points. The two or more time points may comprise an earlier time point and a later time point after the first time point, both time points being post transplantation. In an embodiment, an increase in donor-specific nucleic acid from the earlier time point to the later time point is indicative of engraftment of the HSCT and a decrease is indicative of graft failure.

In some embodiments, the time interval between the earlier time point and the later time point is at least 7 days. In some embodiments, the earlier time point is within 0 days to one year following transplantation. In some embodiments, the later time point is within 7 days to five years following transplantation, e.g., within 7 days to 1 year after transplantation, within 7 days to 30 days, within between 10 days to 8 months after transplantation, or within 1 month to 6 months after transplantation. In some embodiments, the time points are on or after the one year anniversary of the transplantation. Sampling may vary depending upon the nature of the transplant, patient progress or other factors. In some embodiments, samples may be taken every week, once every two weeks, once every 3 weeks, once a month, once every two months, once every three months, once every four months, once every five months, once every six months, once every year, and the donor-specific nucleic acid fraction for two or more of the time points are determined; an increase in donor-specific nucleic acid fraction over time indicates graft failure. In some embodiments, the transplantation status is monitored more frequently in the first year following transplantation than in the subsequent years. For example, samples may be taken at more than 5, more than 6, more than 7, more than 8, more than 9, or more than 10 time points for analysis of transplantation status during the first year. In some cases, during the initial 3 months after the transplantation, recipients are assessed on the weekly basis and thereafter, the recipients who have not experiencing serous complications are assessed in the clinic every 3 to 6 months.

In some embodiments, the status of the engraftment of HSCT may be determined based on a combination of the indications for engraftment of HSCT as described above. In some embodiments, the status of the status of graft failure may be determined based on a combination of the indications for graft failure as described above.

Patients who have been determined to have a graft failure status is typically prescribed additional treatment or retransplantation. In some cases, retransplantation can be performed using another donor. In some cases, retransplantation can be performed using the same donor. In some cases, the recipient receives more intensified conditioning regimens before receiving the retransplant to reduce the risk of graft failure. Non-limiting examples of conditioning regimen include myeblative treatment, total lymphoid irradiation, thoraco-abdominal irradiation, combination treatment with fludarabine and cyclophosphamide.

Intermediate Graft Status

In cases where the recipient did not show successful engraftment of HSCT, there are a number of scenarios: mixed chimerism and split chimerism. These are referred to herein as intermediate graft status.

Mixed chimerism is a phenomenon that both donor and recipient-specific nucleic acid are detectable in a post-transplant sample, and that the donor fraction is below a threshold that is determined to be qualified as successful engraftment, e.g., 91%. Mixed chimerism can be identified by the determination that the donor faction in the post-transplant sample from a recipient ranges from 5% to 90%, e.g., from 10% to 90%, from 20% to 80%, or from 30% to 70%; and/or that the recipient fraction in the post-transplant sample ranges from 95% to 10%, e.g., from 90% to 10%, from 80% of 20%, or from 30% to 70%.

HSCT recipients who showed mixed chimerism are typically monitored to for any changes of the donor and recipient nucleic acid within his or her circulation system. These patients may be followed up according to the schedule described above, e.g., on a weekly basis. In some cases, the recipient fraction may decrease over time; and the recipient is monitored until a successful engraftment is confirmed. In some cases, the donor fraction and the recipient fraction remain substantially unchanged over time, or the recipient fraction increases over time and the donor fraction decreases over time. This generally indicate that a graft failure may occur at a later stage and a preemptive immunotherapy or cellular therapy is beneficial to overcome the pending graft failure. Non-limiting examples of immunotherapy or cellular therapy include donor lymphocyte infusions (DLI) as described in Mattsson et al., Biol. Blood Marrow Transplant. January 1, (2009). DLI is often used in combination with monoclonal anti CD3 receptor antibody (OKT3).

Split chimerism can be identified as recipient-specific nucleic acid is not detectable in all cell lines. That is to say that in some cell types (e.g., T cells) the donor fraction is at a level that indicates successful engraftment, for example, 91% to 100%, however, in other cell lines the donor fraction is less than 91%. Thus, in one embodiment, cell populations can be isolated from the patient, and DNAs from these cell populations are isolated. The amounts of donor-specific nucleic acids and recipient-specific nucleic acids are measured using methods as described above. A transplant patient is determined to have split chimerism if the donor fraction in one isolated cell population is at a level that is above 91%, while the donor fraction in another isolated cell population is at a level that is below 91%.

In one example, the split chimerism may be observed in a recipient in which donor fraction is 0% (conversely, recipient fraction is 100%) in CD3-expressing T cells. In the same patient, the donor fraction is 100% (conversely, recipient is 0% in myeloid cells that are isolated from the use of antibody-mediated positive selection of cells bearing either the CD33 or CD66 markers). In this case, it may require analysis of donor fraction of additional isolated cell populations from the blood sample from the patient (CD56 NK cells) to determine status of engraftment. Furthermore, the patient may be tested for minimal residue disease marker associated with the hematological disorder the recipient is receiving the hematopoetic stem cell transplant for. For example, if a split chimerism of 100% recipient fraction in T-cells (CD3) and 100% donor fraction in myeloid cells (CD33 CD66), is observed in a chronic myelogenous leukemia recipient, then the recipient should further be tested with the BCR-ABL-based assay (a MRD marker for CML) to determine relapse.

As described further below, in some embodiments, the amount of the reference allele or alternate allele can be determined by various assays described herein. In one embodiment, the amount of the allele (e.g., reference allele or alternate allele) corresponds to the sequence reads for that allele from sequencing reactions.

If the transplant status of the recipient is determined to be rejection (including mixed chimerism, or no chimerism), immunosuppressive therapy will be prescribed or administered to the patient. If the patient was already under immunosuppressive therapy, the regimen and the type of existing immunotherapy may be modified in order to improve engraftment results.

FIGS. 11A and 11B show an exemplary method of determining HSCT status.

Quantification of Polymorphic Nucleic Acid Targets

Quantification of polymorphic nucleic acid targets (e.g., SNPs) may be achieved by direct counting of sequence reads covering particular target sites, or by competitive PCR (i.e., co-amplification of competitor oligonucleotides of known quantity, as described herein), or in some cases, the polymorphic nucleic acid targets are first amplified (“targeted amplification”) by using a forward primer and a reverse primer that bind to the genomic nucleic acid such that the amplification product encompass the one or more polymorphic nucleic acid targets.

Amplification of Polymorphic Nucleic Acid Targets

Polymorphic nucleic acid targets can be amplified using any of several nucleic acid amplification procedures which are well known in the art. Nucleic acid amplification is especially beneficial when the amount of target sequence present in a sample is very low. By amplifying the target sequences and detecting the amplicon synthesized, the sensitivity of an assay can be vastly improved, since fewer target sequences are needed at the beginning of the assay to better ensure detection of nucleic acid in the sample belonging to the organism or virus of interest.

The terms “amplify”, “amplification”, “amplification reaction”, or “amplifying” refer to any in vitro process for multiplying the copies of a nucleic acid. Amplification sometimes refers to an “exponential” increase in nucleic acid. However, “amplifying” as used herein can also refer to linear increases in the numbers of a select nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions, for example, and also may reduce amplification biases due to nucleotide sequence or abundance of the nucleic acid. In some embodiments a one-time primer extension may be performed as a prelude to linear or exponential amplification.

A variety of polynucleotide amplification methods are well established and frequently used in research. For instance, the general methods of polymerase chain reaction (PCR) for polynucleotide sequence amplification are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

Although PCR amplification of a polynucleotide sequence is typically used in practicing the present technology, one of skill in the art will recognize that the amplification of a genomic sequence found in a recipient blood sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to qualitatively demonstrate the presence of a particular genomic sequence of the technology herein, which represents a particular methylation pattern, or to quantitatively determine the amount of this particular genomic sequence in the recipient blood. For a review of branched-DNA signal amplification for direct quantitation of nucleic acid sequences in clinical samples, see Nolte, Adv. Clin. Chem. 33:201-235, 1998.

The compositions and processes of the technology herein are also particularly useful when practiced with digital PCR. Digital PCR was first developed by Kalinina and colleagues (Kalinina et al., “Nanoliter scale PCR with TaqMan detection.” Nucleic Acids Research. 25; 1999-2004, (1997)) and further developed by Vogelstein and Kinzler (Digital PCR. Proc. Natl. Acad. Sci. U.S.A 96; 9236-41, (1999)). The application of digital PCR for use with fetal diagnostics was first described by Cantor et al. (PCT Patent Publication No. WO05023091A2) and subsequently described by Quake et al. (US Patent Publication No. US 20070202525), which are both hereby incorporated by reference. Digital PCR takes advantage of nucleic acid (DNA, cDNA or RNA) amplification on a single molecule level, and offers a highly sensitive method for quantifying low copy number nucleic acid.

Any suitable amplification technique can be utilized. Amplification of polynucleotides include, but are not limited to, polymerase chain reaction (PCR); ligation amplification (or ligase chain reaction (LCR)); amplification methods based on the use of Q-beta replicase or template-dependent polymerase (see US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, digital PCR, combinations thereof, and the like. For example, amplification can be accomplished using digital PCR, in certain embodiments (see e.g. Kalinina et al., “Nanoliter scale PCR with TaqMan detection.” Nucleic Acids Research. 25; 1999-2004, (1997); Vogelstein and Kinzler (Digital PCR. Proc Natl Acad Sci USA. 96; 9236-41, (1999); PCT Patent Publication No. WO05023091A2; US Patent Publication No. US 20070202525). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation). Reagents and hardware for conducting PCR are commercially available.

A generalized description of an amplification process is presented herein. Primers and nucleic acid are contacted, and complementary sequences anneal to one another, for example. Primers can anneal to a nucleic acid, at or near (e.g., adjacent to, abutting, and the like) a sequence of interest. In some embodiments, the primers in a set hybridize within about 10 to 30 nucleotides from a nucleic acid sequence of interest and produce amplified products. In some embodiments, the primers hybridize within the nucleic acid sequence of interest.

A reaction mixture, containing components necessary for enzymatic functionality, is added to the primer-nucleic acid hybrid, and amplification can occur under suitable conditions. Components of an amplification reaction may include, but are not limited to, e.g., primers (e.g., individual primers, primer pairs, primer sets and the like) a polynucleotide template, polymerase, nucleotides, dNTPs and the like. In some embodiments, non-naturally occurring nucleotides or nucleotide analogs, such as analogs containing a detectable label (e.g., fluorescent or colorimetric label), may be used for example. Polymerases can be selected by a person of ordinary skill and include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

PCR conditions can be dependent upon primer sequences, abundance of nucleic acid, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990). Digital PCR is also known in the art; see, e.g., United States Patent Application Publication no. 20070202525, filed Feb. 2, 2007, which is hereby incorporated by reference). PCR is typically carried out as an automated process with a thermostable enzyme. In this process, the temperature of the reaction mixture is cycled through a denaturing step, a primer-annealing step, and an extension reaction step automatically. Some PCR protocols also include an activation step and a final extension step. Machines specifically adapted for this purpose are commercially available. A non-limiting example of a PCR protocol that may be suitable for embodiments described herein is, treating the sample at 95° C. for 5 minutes; repeating thirty-five cycles of 95° C. for 45 seconds and 68° C. for 30 seconds; and then treating the sample at 72° C. for 3 minutes. A completed PCR reaction can optionally be kept at 4° C. until further action is desired. Multiple cycles frequently are performed using a commercially available thermal cycler. Suitable isothermal amplification processes known and selected by the person of ordinary skill in the art also may be applied, in certain embodiments.

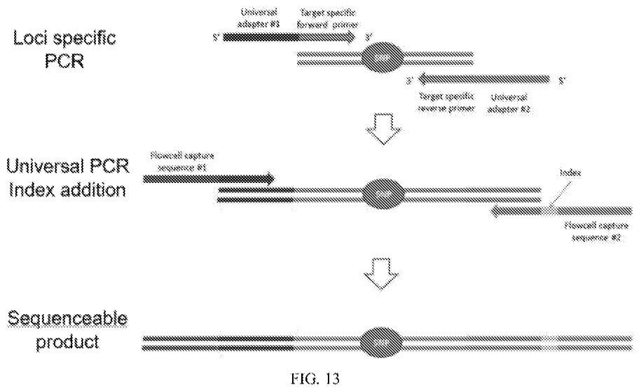

In some embodiments, multiple polymorphic nucleic acid targets are amplified in a single-tube multiplexed PCR. One illustrative example is shown in FIG. 13. Typically the target-specific forward primer contains a common adapter sequence on the 5′ end of the adapter to enable subsequent incorporation of sequencing adapters. Similarly, the target-specific reverse primer also contains a common adapter sequence (distinct from that on the forward primers) on the 5′ end of the adapter to enable subsequent incorporation of sequencing adapters. The PCR reactions can be performed using pairs of target specific forward primer and target specific reverse primer to simultaneously amplify multiple targets. The number of targets that can be amplified in one tube may be at least 10, at least 50, at least 100, at least 200, at least 250, at least 300, at least 500, at least 1000 polymorphic nucleic acid targets, in one single tube. Products from these PCR reactions are also referred to as Loci PCR products in this disclosure. In some cases, these Loci PCR products are be quantified by, e.g., capillary electrophoresis and normalized to a standard concentration. Loci PCR products or normalized loci PCR products can then be amplified using universal primers. The universal primers typically comprise 1) sequences that are compatible with the desired sequencing platform (for example, the flow cell capture sequence #1 and flow cell capture sequence #2 as shown in FIG. 13) and 2) sequences that can hybridize the adaptor sequences on the target-specific forward and reverse primers. The universal primers may further comprise one or more unique barcodes, e.g., dual-index barcodes, that can be used to distinguish individual targets. The barcoded, amplified products (“universal PCR product”) are then quantified and sequenced.

Primers

Primers useful for detection, amplification, quantification, sequencing and analysis of nucleic acid are provided. The term “primer” as used herein refers to a nucleic acid that includes a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence or copy number of a sequence), or feature thereof, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of primers can be effected by native acrylamide gel electrophoresis or by anion-exchange high-performance liquid chromatography (HPLC), for example, as described in Pearson and Regnier, J. Chrom., 255:137-149, 1983.

All or a portion of a primer nucleic acid sequence (naturally occurring or synthetic) may be substantially complementary to a target nucleic acid, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are target and primer sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the complement of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

A primer, in certain embodiments, may contain a modification such as one or more inosines, abasic sites, locked nucleic acids, minor groove binders, duplex stabilizers (e.g., acridine, spermidine), Tm modifiers or any modifier that changes the binding properties of the primers or probes. A primer, in certain embodiments, may contain a detectable molecule or entity (e.g., a fluorophore, radioisotope, colorimetric agent, particle, enzyme and the like, as described above for labeled competitor oligonucleotides).

A primer also may refer to a polynucleotide sequence that hybridizes to a subsequence of a target nucleic acid or another primer and facilitates the detection of a primer, a target nucleic acid or both, as with molecular beacons, for example. The term “molecular beacon” as used herein refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In some embodiments, the primers are complementary to genomic DNA target sequences. In some cases, the forward and reverse primers hybridize to the 5′ and 3′ ends of the genomic DNA target sequences. In some embodiments, primers that hybridize to the genomic DNA target sequences also hybridize to competitor oligonucleotides that were designed to compete with corresponding genomic DNA target sequences for binding of the primers. In some cases, the primers hybridize or anneal to the genomic DNA target sequences and the corresponding competitor oligonucleotides with the same or similar hybridization efficiencies. In some cases the hybridization efficiencies are different. The ratio between genomic DNA target amplicons and competitor amplicons can be measured during the reaction. For example if the ratio is 1:1 at 28 cycles but 2:1 at 35, this could indicate that during the end of the amplification reaction the primers for one target (i.e. genomic DNA target or competitor) are either reannealing faster than the other, or the denaturation is less effective than the other.

In some embodiments primers are used in sets. As used herein, an amplification primer set is one or more pairs of forward and reverse primers for a given region. Thus, for example, primers that amplify nucleic acid targets for region 1 (i.e. targets 1a and 1b) are considered a primer set. Primers that amplify nucleic acid targets for region 2 (i.e. targets 2a and 2b) are considered a different primer set. In some embodiments, the primer sets that amplify targets within a particular region also amplify the corresponding competitor oligonucleotide(s). A plurality of primer pairs may constitute a primer set in certain embodiments (e.g., about 2, 3, 4, 5, 6, 7, 8, 9, 10, 15, 20, 25, 30, 35, 40, 45, 50, 55, 60, 65, 70, 75, 80, 85, 90, 95 or 100 pairs). In some embodiments a plurality of primer sets, each set comprising pair(s) of primers, may be used.

In some cases, loci-specific amplification methods can be used (e.g., using loci-specific amplification primers). In some cases, a multiplex SNP allele PCR approach can be used.

In some cases, a multiplex SNP allele PCR approach can be used in combination with uniplex sequencing. For example, such an approach can involve the use of multiplex PCR (e.g., MASSARRAY system) and incorporation of capture probe sequences into the amplicons followed by sequencing using, for example, the Illumina MPSS system. In some cases, a multiplex SNP allele PCR approach can be used in combination with a three-primer system and indexed sequencing. For example, such an approach can involve the use of multiplex PCR (e.g., MASSARRAY system) with primers having a first capture probe incorporated into certain loci-specific forward PCR primers and adapter sequences incorporated into loci-specific reverse PCR primers, to thereby generate amplicons, followed by a secondary PCR to incorporate reverse capture sequences and molecular index barcodes for sequencing using, for example, the Illumina MPSS system. In some cases, a multiplex SNP allele PCR approach can be used in combination with a four-primer system and indexed sequencing. For example, such an approach can involve the use of multiplex PCR (e.g., MASSARRAY system) with primers having adaptor sequences incorporated into both loci-specific forward and loci-specific reverse PCR primers, followed by a secondary PCR to incorporate both forward and reverse capture sequences and molecular index barcodes for sequencing using, for example, the Illumina MPSS system. In some cases, a microfluidics approach can be used. In some cases, an array-based microfluidics approach can be used. For example, such an approach can involve the use of a microfluidics array (e.g., Fluidigm) for amplification at low plex and incorporation of index and capture probes, followed by sequencing. In some cases, an emulsion microfluidics approach can be used, such as, for example, digital droplet PCR.

In some cases, universal amplification methods can be used (e.g., using universal or non-loci-specific amplification primers). In some cases, universal amplification methods can be used in combination with pull-down approaches. In some cases, the method can include biotinylated ultramer pull-down (e.g., biotinylated pull-down assays from Agilent or IDT) from a universally amplified sequencing library. For example, such an approach can involve preparation of a standard library, enrichment for selected regions by a pull-down assay, and a secondary universal amplification step. In some cases, pull-down approaches can be used in combination with ligation-based methods. In some cases, the method can include biotinylated ultramer pull down with sequence specific adapter ligation (e.g., HALOPLEX PCR, Halo Genomics). For example, such an approach can involve the use of selector probes to capture restriction enzyme-digested fragments, followed by ligation of captured products to an adaptor, and universal amplification followed by sequencing. In some cases, pull-down approaches can be used in combination with extension and ligation-based methods. In some cases, the method can include molecular inversion probe (MIP) extension and ligation. For example, such an approach can involve the use of molecular inversion probes in combination with sequence adapters followed by universal amplification and sequencing. In some cases, complementary DNA can be synthesized and sequenced without amplification.

In some cases, extension and ligation approaches can be performed without a pull-down component. In some cases, the method can include loci-specific forward and reverse primer hybridization, extension and ligation. Such methods can further include universal amplification or complementary DNA synthesis without amplification, followed by sequencing. Such methods can reduce or exclude background sequences during analysis, in some cases.

Table 3 and Table 4 show exemplary primers that can be used to amplify a number of SNPs suitable for use in determination of the HSCT status.

Assays for Detecting Polymorphic Nucleic Acid Targets

In some embodiments, the one or more polymorphic nucleic acid targets can be determined using one or more assays that are known in the art. In some embodiments, the assay is a high throughput sequencing. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin. Chem. 55:641-658 (2009)). Such sequencing methods also can provide digital quantitative information, where each sequence read is a countable “sequence tag” or “count” representing an individual clonal DNA template or a single DNA molecule. High-throughput sequencing technologies include, for example, sequencing-by-synthesis with reversible dye terminators, sequencing by oligonucleotide probe ligation, pyrosequencing and real time sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Iliumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in the methods provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g. about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more.

In some embodiments, nucleic acids may include a fluorescent signal or sequence tag information. Quantification of the signal or tag may be used in a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, the assay is a digital polymerase chain reaction (dPCR). In some embodiments, the assay is a microarray analysis. Other non-limiting examples of methods of detection, quantification, sequencing and the like include mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), direct DNA sequencing, Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization using at least one probe, hybridization using at least one fluorescently labeled probe, cloning and sequencing, electrophoresis, the use of hybridization probes and quantitative real time polymerase chain reaction (QRT-PCR), digital PCR, nanopore sequencing, chips and combinations thereof. In some embodiments the amount of each amplified nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

In some embodiments, the amount of the polymorphic nucleic acid targets are quantified based on sequence reads, e.g., sequence reads generated by high throughout sequencing. In certain embodiments the quantity of sequence reads that are mapped to a polymorphic nucleic acid target on a reference genome for each allele is referred to as a count or read density. In certain embodiments, a count is determined from some or all of the sequence reads mapped to the polymorphic nucleic acid target.

A count can be determined by a suitable method, operation or mathematical process. A count sometimes is the direct sum of all sequence reads mapped to a genomic portion or a group of genomic portions corresponding to a segment, a group of portions corresponding to a sub-region of a genome (e.g., copy number variation region, copy number alteration region, copy number duplication region, copy number deletion region, microduplication region, microdeletion region, chromosome region, autosome region, sex chromosome region or other chromosomal rearrangement) and/or sometimes is a group of portions corresponding to a genome.

In some embodiments, a count is derived from raw sequence reads and/or filtered sequence reads. In certain embodiments a count is determined by a mathematical process. In certain embodiments a count is an average, mean or sum of sequence reads mapped to a target nucleic acid sequence on a reference genome for each of the two alleles (a reference allele and an alternate allele) of a polymorphic site. In some embodiments, a count is associated with an uncertainty value. A count sometimes is adjusted. A count may be adjusted according to sequence reads associated with a target nucleic acid sequence on a reference genome for each of the two alleles (a reference allele and an alternate allele) of a polymorphic site that have been weighted, removed, filtered, normalized, adjusted, averaged, derived as a mean, derived as a median, added, or combination thereof.

In some embodiments, a sequence read quantification is a read density. A read density may be determined and/or generated for one or more segments of a genome. In certain instances, a read density may be determined and/or generated for one or more chromosomes. In some embodiments a read density comprises a quantitative measure of counts of sequence reads mapped to a target nucleic acid sequence on a reference genome for each of the two alleles (a reference allele and an alternate allele) of a polymorphic site. A read density can be determined by a suitable process. In some embodiments a read density is determined by a suitable distribution and/or a suitable distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. A read density may be a density estimation derived from a suitable probability density function. A density estimation is the construction of an estimate, based on observed data, of an underlying probability density function. In some embodiments a read density comprises a density estimation (e.g., a probability density estimation, a kernel density estimation). A read density may be generated according to a process comprising generating a density estimation for each of the one or more portions of a genome where each portion comprises counts of sequence reads. A read density may be generated for normalized and/or weighted counts mapped to a portion or segment. In some instances, each read mapped to a portion or segment may contribute to a read density, a value (e.g., a count) equal to its weight obtained from a normalization process described herein. In some embodiments read densities for one or more portions or segments are adjusted. Read densities can be adjusted by a suitable method. For example, read densities for one or more portions can be weighted and/or normalized.

Sequencing, mapping and related analytical methods are known in the art (e.g., United States Patent Application Publication US2009/0029377, incorporated by reference). Certain aspects of such processes are described hereafter.

In some embodiments, the sequencing process is a sequencing by synthesis method, as described herein. Typically, sequencing by synthesis methods comprise a plurality of synthesis cycles, whereby a complementary nucleotide is added to a single stranded template and identified during each cycle. The number of cycles generally corresponds to read length. In some cases, polymorphic nucleic acid targets are selected such that a minimal read length (i.e., minimal number of cycles) is required to include amplification primer sequence and the polymorphic nucleic acid target site (e.g., SNP) in the read. In some cases, amplification primer sequence includes about 10 to about 30 nucleotides. For example, amplification primer sequence may include about 11, 12, 13, 14, 15, 16, 17, 18, 19, 20, 21, 22, 23, 24, 25, 26, 27, 28, or 29 nucleotides, in some embodiments. In some cases, amplification primer sequence includes about 20 nucleotides. In some embodiments, a SNP site is located within 1 nucleotide base position (i.e., adjacent to) to about 30 base positions from the 3′ terminus of an amplification primer. For example, a SNP site may be within 2, 3, 4, 5, 6, 7, 8, 9, 10, 11, 12, 13, 14, 15, 16, 17, 18, 19, 20, 21, 22, 23, 24, 25, 26, 27, 28, or 29 nucleotides of an amplification primer terminus. Read lengths can be any length that is inclusive of an amplification primer sequence and a polymorphic sequence or position. In some embodiments, read lengths can be about 10 nucleotides in length to about 50 nucleotides in length. For example, read lengths can be about 15, 20, 21, 22, 23, 24, 25, 26, 27, 28, 29, 30, 31, 32, 33, 34, 35, 36, 37, 38, 39, 40, or 45 nucleotides in length. In some cases, read length is about 36 nucleotides. In some cases, read length is about 27 nucleotides. Thus, in some cases, the sequencing by synthesis method comprises about 36 cycles and sometimes comprises about 27 cycles.

In some embodiments, a plurality of samples is sequenced in a single compartment (e.g., flow cell), which sometimes is referred to herein as sample multiplexing. Thus, in some embodiments, donor-specific nucleic acid fraction is determined for a plurality of samples in a multiplexed assay. For example, donor-specific nucleic acid fraction may be determined for about 10, 20, 30, 40, 50, 60, 70, 80, 90, 100, 200, 300, 400, 500, 600, 700, 800, 900, 1000, 2000 or more samples. In some cases, donor-specific nucleic acid fraction is determined for about 10 or more samples. In some cases, donor-specific nucleic acid fraction is determined for about 100 or more samples. In some cases, donor-specific nucleic acid fraction is determined for about 1000 or more samples.

Typically, sequence reads are monitored and filtered to exclude low quality sequence reads. The term “filtering” as used herein refers to removing a portion of data or a set of data from consideration and retaining a subset of data. Sequence reads can be selected for removal based on any suitable criteria, including but not limited to redundant data (e.g., redundant or overlapping mapped reads), non-informative data, over represented or underrepresented sequences, noisy data, the like, or combinations of the foregoing. A filtering process often involves removing one or more reads and/or read pairs (e.g., discordant read pairs) from consideration. Reducing the number of reads, pairs of reads and/or reads comprising candidate SNPs from a data set analyzed for the presence or absence of an informative SNP often reduces the complexity and/or dimensionality of a data set, and sometimes increases the speed of searching for and/or identifying informative SNPs by two or more orders of magnitude.

Nucleic acid detection and/or quantification also may include, for example, solid support array based detection of fluorescently labeled nucleic acid with fluorescent labels incorporated during or after PCR, single molecule detection of fluorescently labeled molecules in solution or captured on a solid phase, or other sequencing technologies such as, for example, sequencing using ION TORRENT or MISEQ platforms or single molecule sequencing technologies using instrumentation such as, for example, PACBIO sequencers, HELICOS sequencer, or nanopore sequencing technologies.

In some embodiments, the polymorphic nucleic acid targets are restriction fragment length polymorphisms (RFLPs). RFLPs detection may be performed by cleaving the nucleic acid with an enzyme and evaluated with a probe that hybridize to the cleaved products and thus defines a uniquely sized restriction fragment corresponding to an allele. RFLPs can be used to detect donor nucleic acids. As an illustrative example, where a homozygous recipient would have only a single fragment generated by a particular restriction enzyme which hybridizes to a restriction fragment length polymorphism probe, after receiving a transplant from a heterozygous donor, the nucleic acids in the recipient would have two distinctly sized fragments which hybridize to the same probe generated by the enzyme. Therefore detecting the RFLPs can be used to identify the presence of the donor-specific nucleic acids.

Use of a primer extension reaction also can be applied in methods of the technology herein. A primer extension reaction operates, for example, by discriminating the SNP alleles by the incorporation of deoxynucleotides and/or dideoxynucleotides to a primer extension primer which hybridizes to a region adjacent to the SNP site. The primer is extended with a polymerase. The primer extended SNP can be detected physically by mass spectrometry or by a tagging moiety such as biotin. As the SNP site is only extended by a complementary deoxynucleotide or dideoxynucleotide that is either tagged by a specific label or generates a primer extension product with a specific mass, the SNP alleles can be discriminated and quantified.

Mass spectrometry may also be used for the detection of a polynucleotide of the technology herein, for example a PCR amplicon, a primer extension product or a detector probe that is cleaved from a target nucleic acid. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. For a review of genotyping methods using Sequenom® standard iPLEX™ assay and MassARRAY® technology, see Jurinke, C., Oeth, P., van den Boom, D., “MALDI-TOF mass spectrometry: a versatile tool for high-performance DNA analysis.” Mol. Biotechnol. 26, 147-164 (2004); and Oeth, P. et al., “iPLEX™ Assay: Increased Plexing Efficiency and Flexibility for MassARRAY® System through single base primer extension with mass-modified Terminators.” SEQUENOM Application Note (2005), both of which are hereby incorporated by reference. For a review of detecting and quantifying target nucleic acids using cleavable detector probes that are cleaved during the amplification process and detected by mass spectrometry, see U.S. patent application Ser. No. 11/950,395, which was filed Dec. 4, 2007, and is hereby incorporated by reference.

Various sequencing techniques that are suitable for use include, but not limited to sequencing-by-synthesis, reversible terminator-based sequencing, 454 sequencing (Roche) (Margulies, M. et al. 2005 Nature 437, 376-380), Applied Biosystems' SOLiD™ technology, Helicos True Single Molecule Sequencing (tSMS), single molecule, real-time (SMRT™) sequencing technology of Pacific Biosciences, ION TORRENT (Life Technologies) single molecule sequencing, chemical-sensitive field effect transistor (CHEMFET) array, electron microscopy sequencing technology, digital PCR, sequencing by hybridization, nanopore sequencing, Illumina Genome Analyzer (or Solexa platform) or SOLID System (Applied Biosystems) or the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001). Many of these methods allow the sequencing of many nucleic acid molecules isolated from a specimen at high orders of multiplexing in a parallel fashion (Dear Brief Funct Genomic Proteomic 2003; 1: 397-416).

Many sequencing platforms that allow sequencing of clonally expanded or non-amplified single molecules of nucleic acid fragments can be used for detecting the donor-specific nucleic acids. Certain platforms involve, for example, (i) sequencing by ligation of dye-modified probes (including cyclic ligation and cleavage), (ii) pyrosequencing, and (iii) single-molecule sequencing. Nucleotide sequence species, amplification nucleic acid species and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label (e.g., 1 fluorescent label, 2, 3, or 4 fluorescent labels).

An example of a system that can be used by a person of ordinary skill based on sequencing by ligation generally involves the following steps. Clonal bead populations can be prepared in emulsion microreactors containing study nucleic acid (“template”), amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination.

An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radioactively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer—released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer—released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons sometimes are performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis is facilitated by sequence analysis apparatus and components known to the person of ordinary skill in the art.

Methods provided herein allow for high-throughput detection of nucleic acid species in a plurality of nucleic acids (e.g., nucleotide sequence species, amplified nucleic acid species and detectable products generated from the foregoing). Multiplexing refers to the simultaneous detection of more than one nucleic acid species. General methods for performing multiplexed reactions in conjunction with mass spectrometry, are known (see, e.g., U.S. Pat. Nos. 6,043,031, 5,547,835 and International PCT application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301-303, 303-305, 305-307, 307-309, 309-311, 311-313, 313-315, 315-317, 317-319, 319-321, 321-323, 323-325, 325-327, 327-329, 329-331, 331-333, 333-335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401-403, 403-405, 405-407, 407-409, 409-411, 411-413, 413-415, 415-417, 417-419, 419-421, 421-423, 423-425, 425-427, 427-429, 429-431, 431-433, 433-435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays can include primer and oligonucleotide design methods and reaction design methods. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

Adaptors

In some embodiments, nucleic acids (e.g., PCR primers, PCR amplicons, and sample nucleic acid) may include an adaptor sequence and/or complement thereof. Adaptor sequences often are useful for certain sequencing methods such as, for example, a sequencing-by-synthesis process described herein. Adaptors sometimes are referred to as sequencing adaptors or adaptor oligonucleotides. Adaptor sequences typically include one or more sites useful for attachment to a solid support (e.g., flow cell). Adaptors also may include sequencing primer hybridization sites (i.e. sequences complementary to primers used in a sequencing reaction) and identifiers (e.g., indices) as described below. Adaptor sequences can be located at the 5′ and/or 3′ end of a nucleic acid and sometimes can be located within a larger nucleic acid sequence. Adaptors can be any length and any sequence, and may be selected based on standard methods in the art for adaptor design.

Identifiers

In some embodiments, nucleic acids (e.g., PCR primers, PCR amplicons, and sample nucleic acid, sequencing adaptors) may include an identifier. In some cases, an identifier is located within or adjacent to an adaptor sequence. An identifier can be any feature that can identify a particular origin or aspect of a nucleic acid target sequence. For example, an identifier (e.g., a sample identifier) can identify the sample from which a particular nucleic acid target sequence originated. In another example, an identifier (e.g., a sample aliquot identifier) can identify the sample aliquot from which a particular nucleic acid target sequence originated. In another example, an identifier (e.g., chromosome identifier) can identify the chromosome from which a particular nucleic acid target sequence originated. An identifier may be referred to herein as a tag, index, barcode, identification tag, index primer, and the like. An identifier may be a unique sequence of nucleotides (e.g., sequence-based identifiers), a detectable label such as the labels described below (e.g., identifier labels), and/or a particular length of polynucleotide (e.g., length-based identifiers; size-based identifiers) such as a stuffer sequence. Identifiers for a collection of samples or plurality of chromosomes, for example, may each comprise a unique sequence of nucleotides. Identifiers (e.g., sequence-based identifiers, length-based identifiers) may be of any length suitable to distinguish certain target genomic sequences from other target genomic sequences. In some embodiments, identifiers may be from about one to about 100 nucleotides in length. For example, identifiers independently may be about 1, 2, 3, 4, 5, 6, 7, 8, 9, 10, 20, 30, 40, 50, 60, 70, 80, 90 or 100 nucleotides in length. In some embodiments, an identifier contains a sequence of six nucleotides. In some cases, an identifier is part of an adaptor sequence for a sequencing process, such as, for example, a sequencing-by-synthesis process described in further detail herein. In some cases, an identifier may be a repeated sequence of a single nucleotide (e.g., poly-A, poly-T, poly-G, and poly-C). Such identifiers may be detected and distinguished from each other, for example, using nanopore technology, as described herein.

In some embodiments, the analysis includes analyzing (e.g., detecting, counting, processing counts for, and the like) the identifier. In some embodiments, the detection process includes detecting the identifier and sometimes not detecting other features (e.g., sequences) of a nucleic acid. In some embodiments, the counting process includes counting each identifier. In some embodiments, the identifier is the only feature of a nucleic acid that is detected, analyzed and/or counted.

Data Processing and Normalization

In some embodiments, sequence read data that are used to represent the amount of a polymorphic nucleic acid target can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative portions or portions of a reference genome (e.g., portions of a reference genome with uninformative data, redundant mapped reads, portions with zero median counts, over represented or underrepresented sequences). Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing.” Processing can render data more amenable to further analysis, and can generate an outcome in some embodiments. In some embodiments one or more or all processing methods (e.g., normalization methods, portion filtering, mapping, validation, the like or combinations thereof) are performed by a processor, a micro-processor, a computer, in conjunction with memory and/or by a microprocessor controlled apparatus.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being overrepresented when prepared using PCR-based methods. Methods described herein can reduce or eliminate the contribution of noisy data, and therefore reduce the effect of noisy data on the provided outcome.

The terms “uninformative data,” “uninformative portions of a reference genome,” and “uninformative portions” as used herein refer to portions, or data derived therefrom, having a numerical value that is significantly different from a predetermined threshold value or falls outside a predetermined cutoff range of values. The terms “threshold” and “threshold value” herein refer to any number that is calculated using a qualifying data set and serves as a limit of diagnosis of a genetic variation or genetic alteration (e.g., a copy number alteration, an aneuploidy, a microduplication, a microdeletion, a chromosomal aberration, and the like). In certain embodiments, a threshold is exceeded by results obtained by methods described herein and a subject is diagnosed with a copy number alteration. A threshold value or range of values often is calculated by mathematically and/or statistically manipulating sequence read data (e.g., from a reference and/or subject), in some embodiments, and in certain embodiments, sequence read data manipulated to generate a threshold value or range of values is sequence read data (e.g., from a reference and/or subject). In some embodiments, an uncertainty value is determined. An uncertainty value generally is a measure of variance or error and can be any suitable measure of variance or error. In some embodiments an uncertainty value is a standard deviation, standard error, calculated variance, p-value, or mean absolute deviation (MAD). In some embodiments an uncertainty value can be calculated according to a formula described herein.

Any suitable procedure can be utilized for processing data sets described herein. Non-limiting examples of procedures suitable for use for processing data sets include filtering, normalizing, weighting, monitoring peak heights, monitoring peak areas, monitoring peak edges, peak level analysis, peak width analysis, peak edge location analysis, peak lateral tolerances, determining area ratios, mathematical processing of data, statistical processing of data, application of statistical algorithms, analysis with fixed variables, analysis with optimized variables, plotting data to identify patterns or trends for additional processing, the like and combinations of the foregoing. In some embodiments, data sets are processed based on various features (e.g., GC content, redundant mapped reads, centromere regions, telomere regions, the like and combinations thereof) and/or variables (e.g., subject gender, subject age, subject ploidy, percent contribution of cancer cell nucleic acid, fetal gender, maternal age, maternal ploidy, percent contribution of fetal nucleic acid, the like or combinations thereof). In certain embodiments, processing data sets as described herein can reduce the complexity and/or dimensionality of large and/or complex data sets. A non-limiting example of a complex data set includes sequence read data generated from one or more test subjects and a plurality of reference subjects of different ages and ethnic backgrounds. In some embodiments, data sets can include from thousands to millions of sequence reads for each test and/or reference subject.

Data processing can be performed in any number of steps, in certain embodiments. For example, data may be processed using only a single processing procedure in some embodiments, and in certain embodiments data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times (e.g., filtering two or more times, normalizing two or more times), and in certain embodiments, processing steps may be two or more different processing steps (e.g., filtering, normalizing; normalizing, monitoring peak heights and edges; filtering, normalizing, normalizing to a reference, statistical manipulation to determine p-values, and the like), carried out simultaneously or sequentially. In some embodiments, any suitable number and/or combination of the same or different processing steps can be utilized to process sequence read data to facilitate providing an outcome. In certain embodiments, processing data sets by the criteria described herein may reduce the complexity and/or dimensionality of a data set.

In some embodiments one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments normalization comprises aligning distributions to a normal distribution. In certain embodiments normalization comprises mathematical adjustments that allow comparison of corresponding normalized values for different datasets in a way that eliminates the effects of certain gross influences (e.g., error and anomalies). In certain embodiments normalization comprises scaling. Normalization sometimes comprises division of one or more data sets by a predetermined variable or formula. Normalization sometimes comprises subtraction of one or more data sets by a predetermined variable or formula. Non-limiting examples of normalization methods include portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn and/or combinations thereof. In some embodiments, the determination of a presence or absence of a copy number alteration (e.g., an aneuploidy, a microduplication, a microdeletion) utilizes a normalization method (e.g., portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn, a normalization method known in the art and/or a combination thereof). Described in greater detail hereafter are certain examples of normalization processes that can be utilized, such as LOESS normalization, principal component normalization, and hybrid normalization methods, for example. Aspects of certain normalization processes also are described, for example, in International Patent Application Publication No. WO2013/052913 and International Patent Application Publication No. WO2015/051163, each of which is incorporated by reference herein.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Non-limiting examples of types of data normalizations that can be used include normalizing raw count data for one or more selected test or reference portions to the total number of counts mapped to the chromosome or the entire genome on which the selected portion or sections are mapped; normalizing raw count data for one or more selected portions to a median reference count for one or more portions or the chromosome on which a selected portion is mapped; normalizing raw count data to previously normalized data or derivatives thereof; and normalizing previously normalized data to one or more other predetermined normalization variables. Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data. Normalizing portions, or portions of a reference genome, with respect to a normalizing value sometimes is referred to as “portion-wise normalization.”

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. A mathematical and/or statistical manipulation can be performed on all or a portion of sequence read data, or processed products thereof. Non-limiting examples of data set variables or features that can be statistically manipulated include raw counts, filtered counts, normalized counts, peak heights, peak widths, peak areas, peak edges, lateral tolerances, P-values, median levels, mean levels, count distribution within a genomic region, relative representation of nucleic acid species, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include principal component analysis, decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include raw counts, filtered counts, normalized counts, peak heights, peak widths, peak edges, lateral tolerances, P-values, median levels, mean levels, count distribution within a genomic region, relative representation of nucleic acid species, the like or combinations thereof.

In certain embodiments, a data set can be analyzed by utilizing multiple (e.g., 2 or more) statistical algorithms (e.g., least squares regression, principal component analysis, linear discriminant analysis, quadratic discriminant analysis, bagging, neural networks, support vector machine models, random forests, classification tree models, K-nearest neighbors, logistic regression and/or smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a reference data set can generate an N-dimensional space (e.g., probability plot) that can be used to represent the presence or absence of a genetic variation/genetic alteration and/or copy number alteration, depending on the status of the reference samples (e.g., positive or negative for a selected copy number alteration). Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the reference data. Test sample data that fall within the N-dimensional space populated by the reference subject data are indicative of a genetic status substantially similar to that of the reference subjects. Test sample data that fall outside of the N-dimensional space populated by the reference subject data are indicative of a genetic status substantially dissimilar to that of the reference subjects. In some embodiments, references are euploid or do not otherwise have a genetic variation/genetic alteration and/or copy number alteration and/or medical condition.

After data sets have been counted, optionally filtered, normalized, and optionally weighted the processed data sets can be further manipulated by one or more filtering and/or normalizing and/or weighting procedures, in some embodiments. A data set that has been further manipulated by one or more filtering and/or normalizing and/or weighting procedures can be used to generate a profile, in certain embodiments. The one or more filtering and/or normalizing and/or weighting procedures sometimes can reduce data set complexity and/or dimensionality, in some embodiments. An outcome can be provided based on a data set of reduced complexity and/or dimensionality. In some embodiments, a profile plot of processed data further manipulated by weighting, for example, is generated to facilitate classification and/or providing an outcome. An outcome can be provided based on a profile plot of weighted data, for example.

Filtering or weighting of portions can be performed at one or more suitable points in an analysis. For example, portions may be filtered or weighted before or after sequence reads are mapped to portions of a reference genome. Portions may be filtered or weighted before or after an experimental bias for individual genome portions is determined in some embodiments. In certain embodiments, portions may be filtered or weighted before or after levels are calculated.

After data sets have been counted, optionally filtered, normalized, and optionally weighted, the processed data sets can be manipulated by one or more mathematical and/or statistical (e.g., statistical functions or statistical algorithm) manipulations, in some embodiments. In certain embodiments, processed data sets can be further manipulated by calculating Z-scores for one or more selected portions, chromosomes, or portions of chromosomes. In some embodiments, processed data sets can be further manipulated by calculating P-values. In certain embodiments, mathematical and/or statistical manipulations include one or more assumptions pertaining to ploidy and/or fraction of a minority species (e.g., fraction of cancer cell nucleic acid; fetal fraction). In some embodiments, a profile plot of processed data further manipulated by one or more statistical and/or mathematical manipulations is generated to facilitate classification and/or providing an outcome. An outcome can be provided based on a profile plot of statistically and/or mathematically manipulated data. An outcome provided based on a profile plot of statistically and/or mathematically manipulated data often includes one or more assumptions pertaining to ploidy and/or fraction of a minority species (e.g., fraction of cancer cell nucleic acid; fetal fraction).

In some embodiments, analysis and processing of data can include the use of one or more assumptions. A suitable number or type of assumptions can be utilized to analyze or process a data set. Non-limiting examples of assumptions that can be used for data processing and/or analysis include subject ploidy, cancer cell contribution, maternal ploidy, fetal contribution, prevalence of certain sequences in a reference population, ethnic background, prevalence of a selected medical condition in related family members, parallelism between raw count profiles from different patients and/or runs after GC-normalization and repeat masking (e.g., GCRM), identical matches represent PCR artifacts (e.g., identical base position), the like and combinations thereof.

In those instances where the quality and/or depth of mapped sequence reads does not permit an outcome prediction of the presence or absence of a genetic variation/genetic alteration and/or copy number alteration at a desired confidence level (e.g., 95% or higher confidence level), based on the normalized count profiles, one or more additional mathematical manipulation algorithms and/or statistical prediction algorithms, can be utilized to generate additional numerical values useful for data analysis and/or providing an outcome. The term “normalized count profile” as used herein refers to a profile generated using normalized counts. Examples of methods that can be used to generate normalized counts and normalized count profiles are described herein. As noted, mapped sequence reads that have been counted can be normalized with respect to test sample counts or reference sample counts. In some embodiments, a normalized count profile can be presented as a plot.

Described in greater detail hereafter are non-limiting examples of processing steps and normalization methods that can be utilized, such as normalizing to a window (static or sliding), weighting, determining bias relationship, LOESS normalization, principal component normalization, hybrid normalization, generating a profile and performing a comparison.

Normalizing to a Window (Static or Sliding)

In certain embodiments, a processing step comprises normalizing to a static window, and in some embodiments, a processing step comprises normalizing to a moving or sliding window. The term “window” as used herein refers to one or more portions chosen for analysis, and sometimes is used as a reference for comparison (e.g., used for normalization and/or other mathematical or statistical manipulation). The term “normalizing to a static window” as used herein refers to a normalization process using one or more portions selected for comparison between a test subject and reference subject data set. In some embodiments the selected portions are utilized to generate a profile. A static window generally includes a predetermined set of portions that do not change during manipulations and/or analysis. The terms “normalizing to a moving window” and “normalizing to a sliding window” as used herein refer to normalizations performed to portions localized to the genomic region (e.g., immediate surrounding portions, adjacent portion or sections, and the like) of a selected test portion, where one or more selected test portions are normalized to portions immediately surrounding the selected test portion. In certain embodiments, the selected portions are utilized to generate a profile. A sliding or moving window normalization often includes repeatedly moving or sliding to an adjacent test portion, and normalizing the newly selected test portion to portions immediately surrounding or adjacent to the newly selected test portion, where adjacent windows have one or more portions in common. In certain embodiments, a plurality of selected test portions and/or chromosomes can be analyzed by a sliding window process.

In some embodiments, normalizing to a sliding or moving window can generate one or more values, where each value represents normalization to a different set of reference portions selected from different regions of a genome (e.g., chromosome). In certain embodiments, the one or more values generated are cumulative sums (e.g., a numerical estimate of the integral of the normalized count profile over the selected portion, domain (e.g., part of chromosome), or chromosome). The values generated by the sliding or moving window process can be used to generate a profile and facilitate arriving at an outcome. In some embodiments, cumulative sums of one or more portions can be displayed as a function of genomic position. Moving or sliding window analysis sometimes is used to analyze a genome for the presence or absence of microdeletions and/or microduplications. In certain embodiments, displaying cumulative sums of one or more portions is used to identify the presence or absence of regions of copy number alteration (e.g., microdeletion, microduplication).

Weighting

In some embodiments, a processing step comprises a weighting. The terms “weighted,” “weighting” or “weight function” or grammatical derivatives or equivalents thereof, as used herein, refer to a mathematical manipulation of a portion or all of a data set sometimes utilized to alter the influence of certain data set features or variables with respect to other data set features or variables (e.g., increase or decrease the significance and/or contribution of data contained in one or more portions or portions of a reference genome, based on the quality or usefulness of the data in the selected portion or portions of a reference genome). A weighting function can be used to increase the influence of data with a relatively small measurement variance, and/or to decrease the influence of data with a relatively large measurement variance, in some embodiments. For example, portions of a reference genome with underrepresented or low quality sequence data can be “down weighted” to minimize the influence on a data set, whereas selected portions of a reference genome can be “up weighted” to increase the influence on a data set. A non-limiting example of a weighting function is [1/(standard deviation)²]. Weighting portions sometimes removes portion dependencies. In some embodiments one or more portions are weighted by an eigen function (e.g., an eigenfunction). In some embodiments an eigen function comprises replacing portions with orthogonal eigen-portions. A weighting step sometimes is performed in a manner substantially similar to a normalizing step. In some embodiments, a data set is adjusted (e.g., divided, multiplied, added, and subtracted) by a predetermined variable (e.g., weighting variable). In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

Bias Relationships

In some embodiments, a processing step comprises determining a bias relationship. For example, one or more relationships may be generated between local genome bias estimates and bias frequencies. The term “relationship” as use herein refers to a mathematical and/or a graphical relationship between two or more variables or values. A relationship can be generated by a suitable mathematical and/or graphical process. Non-limiting examples of a relationship include a mathematical and/or graphical representation of a function, a correlation, a distribution, a linear or non-linear equation, a line, a regression, a fitted regression, the like or a combination thereof. Sometimes a relationship comprises a fitted relationship. In some embodiments a fitted relationship comprises a fitted regression. Sometimes a relationship comprises two or more variables or values that are weighted. In some embodiments a relationship comprise a fitted regression where one or more variables or values of the relationship a weighted. Sometimes a regression is fitted in a weighted fashion. Sometimes a regression is fitted without weighting. In certain embodiments, generating a relationship comprises plotting or graphing.

In certain embodiments, a relationship is generated between GC densities and GC density frequencies. In some embodiments generating a relationship between (i) GC densities and (ii) GC density frequencies for a sample provides a sample GC density relationship. In some embodiments generating a relationship between (i) GC densities and (ii) GC density frequencies for a reference provides a reference GC density relationship. In some embodiments, where local genome bias estimates are GC densities, a sample bias relationship is a sample GC density relationship and a reference bias relationship is a reference GC density relationship. GC densities of a reference GC density relationship and/or a sample GC density relationship are often representations (e.g., mathematical or quantitative representation) of local GC content.

In some embodiments a relationship between local genome bias estimates and bias frequencies comprises a distribution. In some embodiments a relationship between local genome bias estimates and bias frequencies comprises a fitted relationship (e.g., a fitted regression). In some embodiments a relationship between local genome bias estimates and bias frequencies comprises a fitted linear or non-linear regression (e.g., a polynomial regression). In certain embodiments a relationship between local genome bias estimates and bias frequencies comprises a weighted relationship where local genome bias estimates and/or bias frequencies are weighted by a suitable process. In some embodiments a weighted fitted relationship (e.g., a weighted fitting) can be obtained by a process comprising a quantile regression, parameterized distributions or an empirical distribution with interpolation. In certain embodiments a relationship between local genome bias estimates and bias frequencies for a test sample, a reference or part thereof, comprises a polynomial regression where local genome bias estimates are weighted. In some embodiments a weighed fitted model comprises weighting values of a distribution. Values of a distribution can be weighted by a suitable process. In some embodiments, values located near tails of a distribution are provided less weight than values closer to the median of the distribution. For example, for a distribution between local genome bias estimates (e.g., GC densities) and bias frequencies (e.g., GC density frequencies), a weight is determined according to the bias frequency for a given local genome bias estimate, where local genome bias estimates comprising bias frequencies closer to the mean of a distribution are provided greater weight than local genome bias estimates comprising bias frequencies further from the mean.

In some embodiments, a processing step comprises normalizing sequence read counts by comparing local genome bias estimates of sequence reads of a test sample to local genome bias estimates of a reference (e.g., a reference genome, or part thereof). In some embodiments, counts of sequence reads are normalized by comparing bias frequencies of local genome bias estimates of a test sample to bias frequencies of local genome bias estimates of a reference. In some embodiments counts of sequence reads are normalized by comparing a sample bias relationship and a reference bias relationship, thereby generating a comparison.

Counts of sequence reads may be normalized according to a comparison of two or more relationships. In certain embodiments two or more relationships are compared thereby providing a comparison that is used for reducing local bias in sequence reads (e.g., normalizing counts). Two or more relationships can be compared by a suitable method. In some embodiments a comparison comprises adding, subtracting, multiplying and/or dividing a first relationship from a second relationship. In certain embodiments comparing two or more relationships comprises a use of a suitable linear regression and/or a non-linear regression. In certain embodiments comparing two or more relationships comprises a suitable polynomial regression (e.g., a 3^{rd} order polynomial regression). In some embodiments a comparison comprises adding, subtracting, multiplying and/or dividing a first regression from a second regression. In some embodiments two or more relationships are compared by a process comprising an inferential framework of multiple regressions. In some embodiments two or more relationships are compared by a process comprising a suitable multivariate analysis. In some embodiments two or more relationships are compared by a process comprising a basis function (e.g., a blending function, e.g., polynomial bases, Fourier bases, or the like), splines, a radial basis function and/or wavelets.

In certain embodiments a distribution of local genome bias estimates comprising bias frequencies for a test sample and a reference is compared by a process comprising a polynomial regression where local genome bias estimates are weighted. In some embodiments a polynomial regression is generated between (i) ratios, each of which ratios comprises bias frequencies of local genome bias estimates of a reference and bias frequencies of local genome bias estimates of a sample and (ii) local genome bias estimates. In some embodiments a polynomial regression is generated between (i) a ratio of bias frequencies of local genome bias estimates of a reference to bias frequencies of local genome bias estimates of a sample and (ii) local genome bias estimates. In some embodiments a comparison of a distribution of local genome bias estimates for reads of a test sample and a reference comprises determining a log ratio (e.g., a log 2 ratio) of bias frequencies of local genome bias estimates for the reference and the sample. In some embodiments a comparison of a distribution of local genome bias estimates comprises dividing a log ratio (e.g., a log 2 ratio) of bias frequencies of local genome bias estimates for the reference by a log ratio (e.g., a log 2 ratio) of bias frequencies of local genome bias estimates for the sample.

Normalizing counts according to a comparison typically adjusts some counts and not others. Normalizing counts sometimes adjusts all counts and sometimes does not adjust any counts of sequence reads. A count for a sequence read sometimes is normalized by a process that comprises determining a weighting factor and sometimes the process does not include directly generating and utilizing a weighting factor. Normalizing counts according to a comparison sometimes comprises determining a weighting factor for each count of a sequence read. A weighting factor is often specific to a sequence read and is applied to a count of a specific sequence read. A weighting factor is often determined according to a comparison of two or more bias relationships (e.g., a sample bias relationship compared to a reference bias relationship). A normalized count is often determined by adjusting a count value according to a weighting factor. Adjusting a count according to a weighting factor sometimes includes adding, subtracting, multiplying and/or dividing a count for a sequence read by a weighting factor. A weighting factor and/or a normalized count sometimes are determined from a regression (e.g., a regression line). A normalized count is sometimes obtained directly from a regression line (e.g., a fitted regression line) resulting from a comparison between bias frequencies of local genome bias estimates of a reference (e.g., a reference genome) and a test sample. In some embodiments each count of a read of a sample is provided a normalized count value according to a comparison of (i) bias frequencies of a local genome bias estimates of reads compared to (ii) bias frequencies of a local genome bias estimates of a reference. In certain embodiments, counts of sequence reads obtained for a sample are normalized and bias in the sequence reads is reduced.

Machines, Systems, Software and Interfaces

Certain processes and methods described herein (e.g., obtaining and filtering sequencing reads, determining if a polymorphic nucleic acid target is an informative, or determining if one or more nucleic acid is a donor-specific nucleic acid or recipient-specific nucleic acid, e.g., using the fixed cutoff, dynamic k-means clustering, or individual polymorphic nucleic acid target threshold) often cannot be performed without a computer, microprocessor, software, module or other machine. Methods described herein typically are computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a computer system configured to perform the any of the embodiments of the methods for determining the HSCT status disclosed herein. In some embodiments, this disclosure provides a system for determining HSCT status comprising one or more processors and non-transitory machine readable storage medium and/or memory coupled to one or more processors, and the memory or the non-transitory machine readable storage medium encoded with a set of instructions configured to perform a process comprising: (a) obtaining measurements of one or more identified recipient-specific nucleic acids or donor-specific nucleic acids in the sample after transplantation

(b) determining the amount of the one or more identified recipient-specific nucleic acids or donor-specific nucleic acids in the sample after transplantation based on (a); and
(c) determining a transplantation status based on the amount of the identified recipient-specific nucleic acids or donor-specific nucleic acids

In some embodiments, the set of instructions further comprise instructions for determining whether a polymorphic nucleic acid target is informative, and/or detecting donor-specific nucleic acids in a sample from a test subject's sample according to, for example, one of more of the fixed cutoff approach, a dynamic clustering approach, and/or an individual polymorphic nucleic acid target threshold approach as described above. In some cases, the instructions to reduce experimental bias is according to a GC normalized quantification of sequence reads.

Also provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein. In some embodiments, the program module instructs the microprocessor to perform a process comprising: (a) obtaining measurements of one or more identified recipient-specific nucleic acids or donor-specific nucleic acids in the sample after transplantation; (b) determining the amount of the one or more identified recipient-specific nucleic acids or donor-specific nucleic acids in the sample after transplantation based on (a); and

(c) determining a transplantation status based on the amount of the identified recipient-specific nucleic acids or donor-specific nucleic acids. The executable program stored on the computer readable storage media may further instruct the microprocessor to determine whether a polymorphic nucleic acid target is informative, and/or detect donor-specific nucleic acids or recipient-specific nucleic acids in a sample from a test subject's sample according to, for example, one of more of the fixed cutoff approach, a dynamic clustering approach, and/or an individual polymorphic nucleic acid target threshold approach as described above.

In some embodiments, the executable program stored in the computer may further instruct the microprocessor to determine the transplantation status as engraftment of the HSCT if i) the one or more recipient-specific nucleic acids in the peripheral blood cells is below a threshold post-transplantation, ii) the one or more recipient-specific nucleic acids are decreased during a time interval post-transplantation, iii) the one or more donor-specific nucleic acids in the peripheral blood cells is above a threshold post-transplantation, or iv) the one or more donor-specific nucleic acids are increased during a time interval post-transplantation.

In some embodiments, the executable program stored in the computer may further instruct the microprocessor to determine the transplantation status as graft failure if the one or more recipient-specific nucleic acids are increased during a time interval post-transplantation, or if the one or more donor-specific nucleic acids are decreased during a time interval post-transplantation.

In some embodiments, the disclosure provides a non-transitory machine readable storage medium comprising program instructions that when executed by one or more processors cause the one or more processors to perform a method, the method comprising: (a) obtaining measurements of one or more identified recipient-specific nucleic acids or donor-specific nucleic acids in the sample after transplantation

(b) determining the amount of the one or more identified recipient-specific nucleic acids or donor-specific nucleic acids in the sample after transplantation based on (a); and (c) determining a transplantation status based on the amount of the identified recipient-specific nucleic acids or donor-specific nucleic acids The program instructions may further comprise instructions for the one or more processors to determine whether a polymorphic nucleic acid target is informative, and/or detect donor-specific nucleic acids in a sample from a test subject's sample according to, for example, one of more of the fixed cutoff approach, a dynamic clustering approach, and/or an individual polymorphic nucleic acid target threshold approach as described above.

The non-transitory machine readable storage medium may further comprise program instructions that when executed by one or more processors cause the one or more processors to perform a method comprising: adjusting the quantified sequence reads for each of the one or more polymorphic nucleic acid targets by an adjustment process that reduces experimental bias, wherein the adjustment process generates a normalized quantification of sequence reads for each of the polymorphic nucleic acid targets.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., (e.g., obtaining and filtering sequencing reads, determining if a polymorphic nucleic acid target is an informative, or determining if one or more nucleic acid is a donor-specific nucleic acid, using the fixed cutoff, dynamic k-means clustering, or individual polymorphic nucleic acid target threshold) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or measurements sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, and data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein.

A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A module can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, adjusting a profile of a chromosome or a part thereof, and/or padding), providing identification (e.g., identifying a copy number alteration, genetic variation/genetic alteration or aneuploidy), categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., counts) sometimes are accessed by a microprocessor that executes instructions configured to carry out a method described herein. Sequence read quantifications that are accessed by a microprocessor can be within memory of a system, and the counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, FORTRAN, and the like. In some embodiments, an algorithm can be configured or modified to include margin of errors, statistical analysis, statistical significance, and/or comparison to other information or data sets (e.g., applicable when using, for example, algorithms described herein to determine donor-specific nucleic acids such as a fixed cutoff algorithm, a dynamic clustering algorithm, or an individual polymorphic nucleic acid target threshold algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

FIG. 2 illustrates a non-limiting example of a computing environment 110 in which various systems, methods, algorithms, and data structures described herein may be implemented. The computing environment 110 is only one example of a suitable computing environment and is not intended to suggest any limitation as to the scope of use or functionality of the systems, methods, and data structures described herein. Neither should computing environment 110 be interpreted as having any dependency or requirement relating to any one or combination of components illustrated in computing environment 110. A subset of systems, methods, and data structures shown in FIG. 2 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 110 of FIG. 2 includes a general purpose computing device in the form of a computer 120, including a processing unit 121, a system memory 122, and a system bus 123 that operatively couples various system components including the system memory 122 to the processing unit 121. There may be only one or there may be more than one processing unit 121, such that the processor of computer 120 includes a single central-processing unit (CPU), or a plurality of processing units, commonly referred to as a parallel processing environment. The computer 120 may be a conventional computer, a distributed computer, or any other type of computer.

The system bus 123 may be any of several types of bus structures including a memory bus or memory controller, a peripheral bus, and a local bus using any of a variety of bus architectures. The system memory may also be referred to as simply the memory, and includes read only memory (ROM) 124 and random access memory (RAM). A basic input/output system (BIOS) 126, containing the basic routines that help to transfer information between elements within the computer 120, such as during start-up, is stored in ROM 124. The computer 120 may further include a hard disk drive interface 127 for reading from and writing to a hard disk, not shown, a magnetic disk drive 128 for reading from or writing to a removable magnetic disk 129, and an optical disk drive 130 for reading from or writing to a removable optical disk 131 such as a CD ROM or other optical media.

The hard disk drive 127, magnetic disk drive 128, and optical disk drive 130 are connected to the system bus 123 by a hard disk drive interface 132, a magnetic disk drive interface 133, and an optical disk drive interface 134, respectively. The drives and their associated computer-readable media provide nonvolatile storage of computer-readable instructions, data structures, program modules and other data for the computer 120. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 129, optical disk 131, ROM 124, or RAM, including an operating system 135, one or more application programs 136, other program modules 137, and program data 138. A user may enter commands and information into the personal computer 120 through input devices such as a keyboard 140 and pointing device 142. Other input devices (not shown) may include a microphone, joystick, game pad, satellite dish, scanner, or the like. These and other input devices are often connected to the processing unit 121 through a serial port interface 146 that is coupled to the system bus, but may be connected by other interfaces, such as a parallel port, game port, or a universal serial bus (USB). A monitor 147 or other type of display device is also connected to the system bus 123 via an interface, such as a video adapter 148. In addition to the monitor, computers typically include other peripheral output devices (not shown), such as speakers and printers.

The computer 120 may operate in a networked environment using logical connections to one or more remote computers, such as remote computer 149. These logical connections may be achieved by a communication device coupled to or a part of the computer 120, or in other manners. The remote computer 149 may be another computer, a server, a router, a network PC, a client, a peer device or other common network node, and typically includes many or all of the elements described above relative to the computer 120, although only a memory storage device 150 has been illustrated in FIG. 2. The logical connections depicted in FIG. 2 include a local-area network (LAN) 151 and a wide-area network (WAN) 152. Such networking environments are commonplace in office networks, enterprise-wide computer networks, intranets and the Internet, which all are types of networks.

When used in a LAN-networking environment, the computer 120 is connected to the local network 151 through a network interface or adapter 153, which is one type of communications device. When used in a WAN-networking environment, the computer 120 often includes a modem 154, a type of communications device, or any other type of communications device for establishing communications over the wide area network 152. The modem 154, which may be internal or external, is connected to the system bus 123 via the serial port interface 146. In a networked environment, program modules depicted relative to the personal computer 120, or portions thereof, may be stored in the remote memory storage device. It is appreciated that the network connections shown are non-limiting examples and other communications devices for establishing a communications link between computers may be used.

Transformations

As noted above, data sometimes is transformed from one form into another form. The terms “transformed,” “transformation,” and grammatical derivations or equivalents thereof, as used herein refer to an alteration of data from a physical starting material (e.g., test subject and/or reference subject sample nucleic acid) into a digital representation of the physical starting material (e.g., sequence read data), and in some embodiments includes a further transformation into one or more numerical values or graphical representations of the digital representation that can be utilized to provide an outcome. In certain embodiments, the one or more numerical values and/or graphical representations of digitally represented data can be utilized to represent the appearance of a test subject's physical genome (e.g., virtually represent or visually represent the presence or absence of a genomic insertion, duplication or deletion; represent the presence or absence of a variation in the physical amount of a sequence associated with medical conditions). A virtual representation sometimes is further transformed into one or more numerical values or graphical representations of the digital representation of the starting material. These methods can transform physical starting material into a numerical value or graphical representation, or a representation of the physical appearance of a test subject's nucleic acid.

In some embodiments, transformation of a data set facilitates providing an outcome by reducing data complexity and/or data dimensionality. Data set complexity sometimes is reduced during the process of transforming a physical starting material into a virtual representation of the starting material (e.g., sequence reads representative of physical starting material). A suitable feature or variable can be utilized to reduce data set complexity and/or dimensionality. Non-limiting examples of features that can be chosen for use as a target feature for data processing include GC content, fragment size (e.g., length of fragments, reads or a suitable representation thereof (e.g., FRS)), fragment sequence, identification of particular genes or proteins, identification of cancer, diseases, inherited genes/traits, chromosomal abnormalities, a biological category, a chemical category, a biochemical category, a category of genes or proteins, a gene ontology, a protein ontology, co-regulated genes, cell signaling genes, cell cycle genes, proteins pertaining to the foregoing genes, gene variants, protein variants, co-regulated genes, co-regulated proteins, amino acid sequence, nucleotide sequence, protein structure data and the like, and combinations of the foregoing. Non-limiting examples of data set complexity and/or dimensionality reduction include; reduction of a plurality of sequence reads to profile plots, reduction of a plurality of sequence reads to numerical values (e.g., allele frequencies, normalized values, Z-scores, p-values); reduction of multiple analysis methods to probability plots or single points; principal component analysis of derived quantities; and the like or combinations thereof.

### EXEMPLARY EMBODIMENTS OF THE INVENTION

The following are some exemplary embodiments of the invention.

1. A method of determining transplant status comprising:

(a) obtaining a sample from a hematopoietic stem cell transplant (HSCT) recipient who has received hematopoietic stem cells from an allogenic source;

(b) measuring the amount of one or more identified recipient-specific nucleic acids or donor-specific nucleic acids in the sample; and

(c) determining transplant status by monitoring the amount of the one or more identified recipient-specific nucleic acids or donor-specific nucleic acids after transplantation.

2. The method of embodiment 1, wherein said the one or more recipient-specific or the donor-specific nucleic acids are identified based on one or more polymorphic nucleic acid targets.

3 The method of embodiment 1 or 2, the method further comprising determining a donor-specific nucleic acid fraction based on the amount of the polymorphic nucleic acid targets that are specific for donor and the total amount of the polymorphic nucleic acid targets in the biological sample.

4 The method of embodiment 5, wherein the one or more SNPs does not comprise a SNP for which the reference allele and alternate allele combination is selected from the group consisting of A_G, G_A, C_T, and T_C.

5. The method of embodiment 1, wherein the biological sample is blood or bone marrow.

6. The method of embodiment 5, wherein the nucleic acid is genomic DNA.

7 The method of embodiment 6, wherein the genomic DNA is isolated from peripheral white blood cells in the sample.

8. The method of embodiment 35 wherein the genomic DNA is isolated from a cell population purified from the sample.

9. The method of embodiment 8, wherein the cell population is from a group consisting of B-cells, granulocytes, and T-cells.

10. The method of embodiment 8, wherein the cell population is isolated by positive selection of cells expressing markers of one or more of CD3, CD8, CD19, CD20, CD33, CD34, CD56, CD66, CD5, CD294, CD15, CD14, and CD45.

11. The method of embodiment 8, wherein the purified cell population are peripheral blood mononuclear cells.

12. The method of embodiment 1, wherein the HSCT recipient has at least one hematological disorder from a group consisting of leukemias, lymphomas, immune-deficiency illnesses, hemoglobinopathy, congenital metabolic defects, and non-malignant marrow failures.

13 The method of embodiment 1, wherein the determining the transplant status step (c) comprises determining the transplant status as a graft failure if the one or more recipient-specific nucleic acids are increased during a time interval post-transplantation, or if the one or more donor-specific nucleic acids are decreased during a time interval post-transplantation.

14. The method of embodiment 6, wherein the genomic DNA is derived from more than one purified cell populations, wherein the more than one purified cell populations are from B-cells, granulocytes, and T-cells, cells expressing one or more markers from the group consisting of CD3, CD8, CD19, CD20, CD33, CD34, CD56, and CD66.

15. The method of embodiment 7 wherein the determining the transplant status step (c) comprises determining the transplant status as engraftment of the HSCT if

i) the one or more recipient-specific nucleic acids in the peripheral blood cells is below a threshold post-transplantation,

ii) the one or more recipient-specific nucleic acids are decreased during a time interval post-transplantation,

iii) the one or more donor-specific nucleic acids in the peripheral blood cells is above a threshold post-transplantation, or

iv) the one or more donor-specific nucleic acids are increased during a time interval post-transplantation.

16. The method of embodiment 15 wherein the threshold is a percentage of recipient-specific nucleic acid relative to a total of recipient-specific and donor-specific nucleic acids.

17. The method of embodiment 16, wherein the threshold is from the group consisting of less than 20%, 15%, 10%, 5%, 1%, 0.5%, and 0.1%.

18. The method of embodiments 1-17, wherein the recipient-specific nucleic acid or the donor-specific nucleic acid is determined by measuring the one or more polymorphic nucleic acid targets in at least one assay, and

wherein the at least one assay is high-throughput sequencing, capillary electrophoresis or digital polymerase chain reaction (dPCR).

19. The method of embodiments 1-17, wherein the recipient-specific nucleic acid or the donor-specific nucleic acid is determined by targeted amplification using a forward and a reverse primer designed specifically for a native genomic nucleic acid, and a variant synthetic oligo that contains a variant as compared to the native sequence,

wherein the variant can be a substitution of single nucleotides or multiple nucleotides compared to the native sequence

wherein the variant oligo is added to the amplification reaction in a known amount

wherein the method further comprises:
determining the ratio of the amount of the amplified native genomic nucleic acid to the amount of the amplified variant oligo,
determining the total copy number of genomic DNA by multiplying the ratio with the amount of the variant oligo added to the amplification reaction.

20. The method of any of embodiments 19, wherein the method further comprises determining total copy number of genomic DNA in the biological sample, and determining the copy number of the recipient-specific or donor-specific nucleic acid by multiplying the recipient-specific or donor-specific nucleic acid fraction and the total copy number of genomic DNA.

21. The method of any one of embodiments 1-20, wherein said polymorphic nucleic acid targets comprises one or more SNPs.

22. The method of embodiment 21, wherein each of the one or more SNPs has a minor allele frequency of 15%-49%.

23. The method of embodiment 22, wherein the SNPs comprise at least one, two, three, four, or more SNPs in Table 1 or Table 6.

24. The method of embodiment 1, wherein the recipient is genotyped prior to transplantation using one or more SNPs in Table 1 or Table 6.

25. The method of embodiment 1, wherein the donor is genotyped prior to transplantation using one or more SNPs in Table 1 or Table 6.

26. The method of embodiment 2, wherein the donor genotype is not known, the recipient genotype is not known, or neither the donor nor the recipient genotype is known for any one of the one or more polymorphic nucleic acid targets prior to transplantation.

27 The method of embodiment 24, wherein the recipient genotype is known for the one or more polymorphic nucleic acid targets and the donor genotype is not known for the one or more polymorphic nucleic acid targets prior to the transplant status determination, wherein the (d) identifying donor-specific allele and/or determining the donor specific nucleic acid fraction comprises:
I) filtering out 1) polymorphic nucleic acid targets which have a genotype combination of AB_{recipient}/AB_{donor}, AB_{recipient}/AA_{donor}, and AB_{recipient}/BB_{donor},
II) performing a computer algorithm on a data set consisting of measurements of the remaining polymorphic nucleic acid targets to form a first cluster and a second cluster,
wherein the first cluster comprises polymorphic nucleic acid targets that are present in the recipient and the donor in a genotype combination of AA_{recipient}/AB_{donor}, or BB_{recipient}/AB_{donor}, and
wherein the second cluster comprises SNPs that have a genotype combination of AA_{recipient}/BB_{donor} or BB_{recipient}/AA_{donor}, and

detecting the donor specific allele based on the presence of the remaining polymorphic nucleic acid targets in the one or more polymorphic nucleic acid targets in the biological sample.

28. The method of embodiment 1-26, wherein the recipient's genotype is not known for the one or more polymorphic nucleic acid targets and wherein the donor's genotype is known for the one or more polymorphic nucleic acid targets prior to the transplant status determination,

wherein the (d) detecting the donor specific allele comprise:

I) filtering out polymorphic nucleic acid targets which are present in the recipient and the donor in a genotype combination of AA_{recipient}/AA_{donor} or AB_{recipient}/AA_{donor} and the donor allele frequency is less than 0.5, and 2) SNPs which are present in the recipient and the donor in a genotype combination of BB_{recipient}/BB_{donor}, and AB_{recipient}/BB_{donor}, and the donor allele frequency is larger than 0.5; and

II) detecting the donor specific alleles based on the presence of the remaining polymorphic nucleic acid targets in the biological sample.

29. The method of embodiments 1-26, wherein neither the recipient nor the organ donor's genotype is known for the one or more polymorphic nucleic acid targets prior to the transplant status determination,

wherein the (d) detecting donor-specific allele and/or determining donor-specific nucleic acid fraction comprises:

I) performing a computer algorithm on a data set consisting of measurements of the amounts of the one or more polymorphic nucleic acid targets to form a first cluster and a second cluster,
wherein the first cluster comprises polymorphic nucleic acid targets that are present in the recipient and the donor in a genotype combination of AA_{recipient}/AB_{donor}, BB_{recipient}/AB_{donor}, AA_{recipient}/BB_{donor}, or BB_{recipient}/AA_{donor}, and
wherein the second cluster comprises polymorphic nucleic acid targets that are present in the recipient and the donor in a genotype combination of AB_{recipient}/AB_{donor}, AB_{recipient}/AA_{donor}, or AB_{recipient}/BB_{donor} and

II) detecting the donor specific allele based on the presence of the polymorphic nucleic acid targets in the first cluster.

30. The method of embodiment 18, wherein the high-throughput sequencing is targeted amplification using a forward and a reverse primer designed specifically for the one or more polymorphic nucleic acid targets or targeted hybridization using a probe sequence that contains the one or more polymorphic nucleic acid targets.

31. The method of embodiment 30, wherein the targeted amplification or targeted hybridization is a multiplex reaction.

32. The method of embodiment 1, wherein the allogenic source is from the group comprising bone marrow transplant, peripheral blood stem cell transplant, and umbilical cord blood.

33. The method of embodiment 9, further advising administration of therapy for the hematological disorder to the HSCT recipient or advising the modification of the HSCT recipient's therapy.

34. The methods of embodiment 26, wherein the one or more nucleic acids from said HSCT recipient are identified as recipient-specific nucleic acid or donor-specific nucleic acid using a computer algorithm based on measurements of one or more polymorphic nucleic acid target.

35. The method of embodiment 34, wherein the algorithm comprises one or more of the following: (i) a fixed cutoff, (ii) a dynamic clustering, and (iii) an individual polymorphic nucleic acid target threshold.

36. The method of embodiment 35, wherein the fixed cutoff algorithm detects donor-specific nucleic acids if the deviation between the measured frequency of a reference allele of the one or more polymorphic nucleic acid targets in the nucleic acids in the sample and the expected frequency of the reference allele in a reference population is greater than a fixed cutoff,

wherein the expected frequency for the reference allele is in the range of

0.00-0.03 if the recipient is homozygous for the alternate allele,

0.40-0.60 if the recipient is heterozygous for the alternate allele, or

0.97-1.00 if the recipient is homozygous for the reference allele.

37. The method of embodiment 35 or 36, wherein the recipient is homozygous for the reference allele and the fixed cutoff algorithm detects donor-specific nucleic acids if the measured allele frequency of the reference allele of the one or more polymorphic nucleic acid targets is greater than the fixed cutoff.

38. The method of embodiment 35 or 36, wherein the recipient is homozygous for the alternate allele, and the fixed cutoff algorithm detects donor-specific nucleic acids if the measured allele frequency of the reference allele of the one or more polymorphic nucleic acid targets is greater than the fixed cutoff.

39. The method of any of embodiments 35-37, wherein the fixed cutoff is based on the homozygous allele frequency of the reference or alternate allele of the one or more polymorphic nucleic acid targets in a reference population.

40. The method of embodiment 35-38, wherein the fixed cutoff is based on a percentile value of distribution of the homozygous allele frequency of the reference or alternate allele of the one or more polymorphic nucleic acid targets in the reference population.

41. The method of embodiment 40, wherein the percentile is at least 90.

42. The method of embodiment 35, wherein identifying one or more nucleic acids as donor-specific nucleic acids using the dynamic clustering algorithm comprises

(i) stratifying the one or more polymorphic nucleic acid targets in the nucleic acids into recipient homozygous group and recipient heterozygous group based on the measured allele frequency for a reference allele or an alternate allele of each of the polymorphic nucleic acid targets;

(ii) further stratifying recipient homozygous groups into non-informative and informative groups; and

(iii) measuring the amounts of one or more polymorphic nucleic acid targets in the informative groups.

43. The method of embodiment 35, wherein the dynamic clustering algorithm is a dynamic K-means algorithm.

44. The method of embodiment 35, wherein the individual polymorphic nucleic acid target threshold algorithm identifies the one or more nucleic acids as donor-specific nucleic acids if the allele frequency of each of the one or more of the polymorphic nucleic acid targets is greater than a threshold.

45. The method of embodiment 44, wherein the threshold is based on the homozygous allele frequency of each of the one or more polymorphic nucleic acid targets in a reference population.

46. The method of embodiment 44, wherein the threshold is a percentile value of a distribution of the homozygous allele frequency of each of the one or more polymorphic nucleic acid targets in the reference population.

47 The method of any of embodiments above, wherein the donor's genotype is not known for the one of more polymorphic nucleic acid targets prior to the transplant status determination and wherein the recipient's genotype is known for the one or more polymorphic nucleic acid targets prior to the transplant status determination, and identifying donor-specific allele and/or determining the donor-specific nucleic acid fraction is by DF3.

48 The method of any of embodiments above, wherein the recipient's genotype is not known for the one of more polymorphic nucleic acid targets prior to transplant status determination, and wherein the donor's genotype is known for the one or more polymorphic nucleic acid targets prior to the transplant status determination, wherein the method comprises identifying donor-specific allele and/or determining donor-specific nucleic acid fraction by DF2.

49. The method of any of embodiments above, wherein neither the recipient nor the donor's genotype is known for the one of more polymorphic nucleic acid targets prior to the transplant status determination. wherein identifying donor-specific allele and/or determining donor-specific nucleic acid fraction is by DF1.

50. A system to perform the method in any one or the preceding embodiments.

51. A system for determining transplantation status comprising one or more processors; and memory coupled to one or more processors, the memory encoded with a set of instructions configured to perform a process comprising:

(a) obtaining measurements of one or more identified recipient-specific nucleic acids or donor-specific nucleic acids in the sample after transplantation

(b) determining the amount of the one or more identified recipient-specific nucleic acids or donor-specific nucleic acids in the sample after transplantation based on (a); and

(c) determining a transplantation status based on the amount of the identified recipient-specific nucleic acids or donor-specific nucleic acids.

52. The system of embodiment 50, wherein said the one or more recipient-specific or the donor-specific nucleic acids are identified based on one or more polymorphic nucleic acid targets.

53 The system of embodiment 50, wherein the one or more SNPs does not comprise a SNP for which the reference allele and alternate allele combination is selected from the group consisting of A_G, G_A, C_T, and T_C.

54. The system of embodiment 50, wherein the sample is blood or bone marrow.

55. The system of embodiment 50, wherein the nucleic acid is genomic DNA.

56 The system of embodiment 50, wherein the genomic DNA is isolated from peripheral white blood cells in the sample.

57. The system of embodiment 50, wherein the determining the transplant status step (c) comprises determining the transplant status as a graft failure if the one or more recipient-specific nucleic acids are increased during a time interval post-transplantation, or if the one or more donor-specific nucleic acids are decreased during a time interval post-transplantation.

58. The system of embodiment 50 wherein the determining the transplant status step (c) comprises determining the transplant status as engraftment of the HSCT if

i) the one or more recipient-specific nucleic acids in the peripheral blood cells is below a threshold post-transplantation,

ii) the one or more recipient-specific nucleic acids are decreased during a time interval post-transplantation,

iii) the one or more donor-specific nucleic acids in the peripheral blood cells is above a threshold post-transplantation, or

iv) the one or more donor-specific nucleic acids are increased during a time interval post-transplantation.

59. The system of embodiments 50-58, wherein the recipient-specific nucleic acid or the recipient-specific nucleic acid is determined by measuring the one or more polymorphic nucleic acid targets in at least one assay, and wherein the at least one assay is high-throughput sequencing, capillary electrophoresis or digital polymerase chain reaction (dPCR).

60. The system of embodiments 50-59, wherein the recipient-specific nucleic acid or the donor-specific nucleic acid is determined by targeted amplification using a forward and a reverse primer designed specifically for a native genomic nucleic acid, and a variant synthetic oligo that contains a variant as compared to the native sequence,

wherein the variant can be a substitution of single nucleotides or multiple nucleotides compared to the native sequence

wherein the variant oligo is added to the amplification reaction in a known amount

wherein the method further comprises:
determining the ratio of the amount of the amplified native genomic nucleic acid to the amount of the amplified variant oligo,
determining the total copy number of genomic DNA by multiplying the ratio with the amount of the variant oligo added to the amplification reaction.

61. The system of any of embodiments 50-60, wherein the method further comprises determining total copy number of genomic DNA in the biological sample and determining the copy number of the recipient-specific nucleic acid by multiplying the donor-specific nucleic acid fraction and the total copy number of genomic DNA.

62. The system of any one of embodiments 50-61, wherein said polymorphic nucleic acid targets comprises one or more SNPs.

The following examples of specific aspects for carrying out the present invention are offered for illustrative purposes only, and are not intended to limit the scope of the present invention in any way.

### EXAMPLES

### Example 1 Developing SNP Panels for Determining Transplant Rejection

Blood samples are drawn from a HSCT recipient at various time points: prior to the transplantation, two days after transplantation, and nine days after the transplantation. The blood samples are placed in a tube containing EDTA or a specialized commercial product such as Vacutainer SST (Becton Dickinson, Franklin Lakes, and N.J.) to prevent blood clotting. PBMCs are isolated using Ficoll density gradient separation. Nucleic acids were extracted from the isolated PMBCs using QIAamp DNA Blood Mini Kit. (QIAGEN, Inc., Germantown, Md.).

A PCR reaction is set up with primers that are specific to the SNP panels (the sequences of the SNPs and respective primers are provided in Table 3 and Table 4) to amplify the SNPs. In addition, an RNAsP variant oligo that has a single nucleotide substitution relative to the native RNAsP, and ApoE variant oligo that has a single nucleotide substitution relative to the native ApoE, also added in the PCR reaction at known amounts to be amplified simultaneously with the SNP panel. The RNAsP and ApoE variant oligo sequences are provided in Table 5.

The amplification products are sequenced and copy numbers of the amplification products comprising the SNPs are determined to calculate the relative frequencies of the reference allele and alternative allele for each of the SNPs.

A SNP is chosen as informative SNP i) if the frequency distribution of the alleles for the SNP indicates that the recipient is homozygous for the reference allele and that the donor is homozygous or heterozygous for the alternative allele, and ii) if the alternative allele frequency is greater than a fixed cutoff frequency, which is expressed as a percent (%) shift of the alternative allele frequency from an expected frequency. Donor fraction and recipient fractions are then determined based on the frequencies of the alternative alleles of the selected, informative SNPs.

The amplified native RNAsP and the RNAsP variant and the amplified native ApoE and the ApoE variant are quantified by sequencing, and the ratios of the respective native nucleic acids to the variant oligos are calculated. The total copies of genomic DNA is determined based on the following formula:

Total copy number of genomic DNA in the sample=ratio of the amount of amplified native ApoE (or RNAsP) to the amount of amplified ApoE (or RNAsP) variant x the amount of the variant oligos added before amplification.

The copy number of the donor-specific nucleic acid=total copy number of genomic DNA in the sample x donor-specific nucleic acid fraction

The copy number of the recipient-specific nucleic acid=total copy number of genomic DNA in the sample x recipient-specific nucleic acid fraction

The amount of recipient-specific nucleic acids from plasma samples derived from blood samples drawn at various time points are determined as above and compared. If the amount of recipient-specific nucleic acids in samples post-transplant increases over time, i.e., the level in the sample from later time point is higher than the level in the sample from the earlier time point post transplantation, the transplant is being rejected. If the recipient-specific nucleic acids amount is lower than a predetermine threshold at various times post-transplantation, the engraftment is successful.

TABLE 3
Panel A SNPs and amplification primers

| | | First | | Second |
|---|---|---|---|---|
| | SEQ ID | Primer | SEQ ID | Primer |
| SNP | NO | Sequence | NO | Sequence |
| rs38062 | 1 | AAAAA | 2 | TCTAT |
| | | CTGCT | | GGGTT |
| | | TGCCT | | CTCAC |
| | | TCTTC | | AACTC |
| | | TT | | AAC |
| rs163446 | 3 | TGGAC | 4 | AGATC |
| | | AAAAA | | ATCCT |
| | | TACCA | | GAACA |
| | | TCATC | | TAAGG |
| | | A | | T |
| rs226447 | 5 | CATCT | 6 | TCAAG |
| | | AAATA | | TATCC |
| | | CATGA | | AGGAC |
| | | AAAAG | | TTGTT |
| | | GAG | | CG |
| rs241713 | 7 | GGACC | 8 | AGGGT |
| | | CAAGA | | GAGCT |
| | | TCTGA | | GTTCT |
| | | TTCTA | | CAGGA |
| | | GC | | |
| rs253229 | 9 | TCCCC | 10 | TCACT |
| | | AGACT | | TTACT |
| | | AATTA | | GTTCA |
| | | TGGAA | | CCAAA |
| | | AAA | | CG |
| rs309622 | 11 | GGATT | 12 | GAGAG |
| | | TTAGG | | TTTTT |
| | | GCACT | | AAAGA |
| | | AGGAA | | GTGTC |
| | | GG | | GTT |
| rs376293 | 13 | TGTAT | 14 | GGCAG |
| | | TTGCC | | AGTTC |
| | | TAAAA | | TCTTG |
| | | GTAAG | | ACGTG |
| | | AGG | | |
| rs387413 | 15 | CAGCT | 16 | TCTCT |
| | | AAAGG | | TTGTC |
| | | AAAAC | | TGTTA |
| | | TATTA | | GGGTT |
| | | ATGC | | TT |
| rs427982 | 17 | TCATC | 18 | GCTCT |
| | | TGTGA | | TAAAA |
| | | AATAG | | CTCAT |
| | | GGACA | | CCCAA |
| | | CC | | GC |
| rs511654 | 19 | AGAAA | 20 | TCCTG |
| | | TTATT | | ACAAG |
| | | CAGGA | | ACAGT |
| | | CACAG | | TATCA |
| | | AGA | | TCT |
| rs517811 | 21 | GAGAA | 22 | ACAAG |
| | | GAATG | | AGTAC |
| | | ATTAG | | ACGAG |
| | | ACCTT | | AGAAA |
| | | GCT | | AA |
| rs582991 | 23 | TGATG | 24 | TCCAA |
| | | TGGAA | | AAGGT |
| | | TAGTT | | AATTC |
| | | TAGGT | | CAATA |
| | | GA | | TGC |
| rs602763 | 25 | GGATA | 26 | GCTAA |
| | | TGCCG | | GTAAA |
| | | CTTTT | | TAATT |
| | | CCTCT | | TGGCA |
| | | | | GTT |
| rs614004 | 27 | TCACA | 28 | CAGCA |
| | | GTGTT | | GCTAG |
| | | TCTCA | | TGTTG |
| | | TAGTT | | CACTA |
| | | TTA | | AT |
| rs686106 | 29 | GGTTC | 30 | TGAGT |
| | | ACAGA | | CTCTT |
| | | GCCCA | | ACTGA |
| | | AGTTA | | TCCTG |
| | | C | | TGAC |
| rs723211 | 31 | GAGTC | 32 | GATGC |
| | | ACTCT | | CCAGC |
| | | TGGGG | | CTCTT |
| | | TATCA | | CTCTC |
| rs751128 | 33 | AGAGA | 34 | GGGGG |
| | | TCTCC | | CCAAT |
| | | GCATC | | AACTA |
| | | CTGTG | | TGCTC |
| rs756668 | 35 | AGTGT | 36 | GTCCT |
| | | GATGT | | ATCAT |
| | | TTGAG | | CTTTT |
| | | TGAGG | | ATTTC |
| | | | | CAA |
| rs765772 | 37 | TTCCT | 38 | TCCCA |
| | | TGGCA | | TGTAA |
| | | TTTTA | | CACCT |
| | | GTTTC | | TTCAG |
| | | C | | A |
| rs792835 | 39 | TCACC | 40 | AACTT |
| | | CATTC | | TTCAG |
| | | TTCAT | | GTCGG |
| | | ACTCT | | CAGTG |
| | | TTG | | |
| rs863368 | 41 | GGAGA | 42 | GGAAT |
| | | GAATC | | TTTAT |
| | | CCTTA | | TAGAT |
| | | CCCTT | | GTTGA |
| | | G | | GG |
| rs930189 | 43 | CAGCC | 44 | TCGAG |
| | | CAGAT | | GTAAA |
| | | TTTCT | | TAGGC |
| | | CTTTC | | CCACA |
| | | A | | |
| rs955105 | 45 | TTCAG | 46 | TGAAA |
| | | CTCTT | | CAAGA |
| | | CTACT | | GAAGA |
| | | CTGGA | | CTGGA |
| | | CTG | | TTTG |
| rs967252 | 47 | GTTAT | 48 | TTGGA |
| | | ATCTC | | TTGTT |
| | | TTTTG | | AGAGA |
| | | TTTCT | | ATAAC |
| | | CTCC | | G |
| rs975405 | 49 | TGGAC | 50 | GCTGA |
| | | AAGAG | | GCCTT |
| | | AGACT | | TTAGA |
| | | TCAGG | | TAGTG |
| | | AG | | CTG |
| rs1002142 | 51 | TCCAA | 52 | GAGCC |
| | | CTGGA | | ACCTT |
| | | AAACA | | CAAGA |
| | | CCTCA | | CTCTT |
| | | | | TC |
| rs1002607 | 53 | TTTAA | 54 | TGATT |
| | | ATCTT | | CTCAG |
| | | TCCAG | | CCTGG |
| | | GGGGT | | AGTTT |
| | | TT | | |
| rs1030842 | 55 | AGGAT | 56 | TCTGC |
| | | TCAGC | | CATGG |
| | | CATCC | | GAGGT |
| | | ATCTG | | ATAGA |
| rs1145814 | 57 | AAAAC | 58 | AATAG |
| | | ATAAT | | GAGGC |
| | | TGAAC | | TGCTC |
| | | ACCTA | | TATGC |
| | | GCA | | |
| rs1152991 | 59 | TGATT | 60 | AGTGA |
| | | CACTT | | CCTTG |
| | | CCAGT | | CTGGT |
| | | TCTTG | | TTGTG |
| | | ACA | | |
| rs1160530 | 61 | GGGTA | 62 | TCTTC |
| | | CCATA | | TTCCC |
| | | TGAGG | | AATGT |
| | | CCAGT | | CATGG |
| | | T | | A |
| rs1281182 | 63 | CCAGG | 64 | AAGGC |
| | | CTTCC | | ATCTC |
| | | AAGAT | | AGGTG |
| | | TATTG | | TTATT |
| | | T | | TT |
| rs1298730 | 65 | CCTCG | 66 | AAGTG |
| | | CTGTC | | CTGAC |
| | | CCTGC | | TCTGT |
| | | ATAC | | TCTGG |
| rs1334722 | 67 | GAATA | 68 | GGGAT |
| | | TCTGT | | GTGTG |
| | | CTCGG | | ATTTC |
| | | AATAC | | TGAAG |
| | | CA | | G |
| rs1341111 | 69 | GAACA | 70 | CACCA |
| | | ACATC | | CTCTA |
| | | TATCA | | AAGTA |
| | | TTCAT | | GACCA |
| | | CTCT | | TTG |
| rs1346065 | 71 | GCTTT | 72 | AGATG |
| | | GGGGT | | GCCAT |
| | | TATAG | | TAGCT |
| | | CTGGA | | AGGAA |
| rs1347879 | 73 | GCACA | 74 | CTATA |
| | | TAGAG | | TTAGA |
| | | GTCTC | | ACACT |
| | | TCTCT | | CAGCA |
| | | TCT | | GCTA |
| rs1390028 | 75 | AGGGC | 76 | CTCAT |
| | | TGAAC | | CCTGA |
| | | AAGGA | | GCTCT |
| | | ACTGA | | CGTGT |
| | | | | A |
| rs1399591 | 77 | TCACT | 78 | TGAGT |
| | | CATGT | | CAGAT |
| | | TTTAC | | TCTTC |
| | | CTTTT | | ATAAC |
| | | AGC | | TTT |
| rs1442330 | 79 | TACTG | 80 | TTAGA |
| | | CCAAC | | CCGCA |
| | | AGACA | | GACCT |
| | | ACTCG | | TTAGA |
| | | | | A |
| rs1452321 | 81 | GGGGC | 82 | GGCTG |
| | | AGATC | | TTCTC |
| | | AGAAA | | AATGG |
| | | TGTTG | | TGTCA |
| rs1456078 | 83 | CCCCA | 84 | TCTTT |
| | | TATGT | | GGAAG |
| | | AACCC | | AGAAA |
| | | ATCAC | | TGTGA |
| | | A | | TTCT |
| rs1486748 | 85 | GGAAT | 86 | TCACT |
| | | GTATT | | ATTCC |
| | | TCTGC | | TTACT |
| | | TGTGC | | CCAGG |
| | | TG | | TGA |
| rs1510900 | 87 | CCATT | 88 | CACCT |
| | | CACGT | | TACTG |
| | | GGCAC | | CTTCC |
| | | TTTTT | | TGCTA |
| | | | | CC |
| rs1514221 | 89 | CCAAA | 90 | GTGTT |
| | | GGCTG | | GAAGT |
| | | TATTA | | GATGT |
| | | TTTAT | | AATTC |
| | | GC | | AG |
| rs1562109 | 91 | TGAAC | 92 | AAAGC |
| | | ATATC | | CCAGA |
| | | AGCTG | | ATTGA |
| | | GCCAT | | CTTGG |
| | | T | | |
| rs1563127 | 93 | CAAAC | 94 | GGGGT |
| | | CTCCA | | TCATA |
| | | GGGTA | | AGGGA |
| | | GTAGA | | AACCA |
| | | CA | | |
| rs1566838 | 95 | TCTCA | 96 | GCCCA |
| | | GAGCA | | ATCAG |
| | | ACATG | | ACATC |
| | | TACCA | | AATCC |
| | | AAA | | |
| rs1646594 | 97 | GTTTC | 98 | TCATC |
| | | CCAGC | | AAAAT |
| | | AAATT | | GGATC |
| | | CCCTA | | ATAAC |
| | | | | AG |
| rs1665105 | 99 | TTTGG | 100 | AAAGA |
| | | AGTGG | | GTACA |
| | | GTCTC | | TTCTG |
| | | TTCAC | | CCTTG |
| | | T | | CT |
| rs1795321 | 101 | GCTCA | 102 | ACCAC |
| | | CTGTT | | ACAAA |
| | | ACCCT | | TGATT |
| | | ACTAC | | ATGGT |
| | | TCTC | | A |
| rs1821662 | 103 | CCACA | 104 | AGTGG |
| | | CACTG | | GCTGG |
| | | AAAAG | | ATATA |
| | | AATTT | | TGAAA |
| | | GTG | | A |
| rs1879744 | 105 | AGGCA | 106 | GGAGG |
| | | TGTGT | | AAGCT |
| | | TAAAC | | GTGTT |
| | | TAGAA | | CTTTT |
| | | AAA | | CA |
| rs1885968 | 107 | GGGGA | 108 | GACAC |
| | | TCTTA | | TCCCA |
| | | AAAGC | | CTTCT |
| | | ACCAA | | GCCTA |
| rs1893691 | 109 | CAGCC | 110 | AGTTA |
| | | TAAAT | | TGAGT |
| | | TTCCA | | AATGA |
| | | GTCTT | | AGGAA |
| | | | | GG |
| rs1894642 | 111 | ATTTC | 112 | CAGGC |
| | | TTCAA | | AAACA |
| | | GTGTA | | TTCCC |
| | | TACAG | | TTGTA |
| | | AGC | | |
| rs1938985 | 113 | TGTCT | 114 | TTGTA |
| | | TTGCT | | AATTT |
| | | CAGTT | | TTCTC |
| | | ATGAA | | TAGGT |
| | | GAGA | | GTG |
| rs1981392 | 115 | GGCAT | 116 | GATTT |
| | | GGCAA | | TCACA |
| | | TACTC | | TCTAA |
| | | TTCTG | | TTTTC |
| | | A | | ACC |
| rs1983496 | 117 | ACAAT | 118 | ACTAA |
| | | GAGCT | | CTTTG |
| | | ATTTT | | CAAGA |
| | | AACTC | | TACAG |
| | | CA | | ATT |
| rs1992695 | 119 | TGGCC | 120 | TGTTC |
| | | ACTTG | | TTAAG |
| | | CTTAT | | TTGCC |
| | | TTGAA | | CATAA |
| rs2049711 | 121 | CCCAC | 122 | GAAGA |
| | | TTTCA | | AATAC |
| | | CAATT | | AAAGC |
| | | TGAAT | | AGTTG |
| | | CC | | CTAA |
| rs2051985 | 123 | GCTTA | 124 | CCACT |
| | | GGAAG | | ATTTA |
| | | GTGTG | | TGTTT |
| | | GAGAG | | ATTGA |
| | | C | | GTGC |
| rs2064929 | 125 | GAGTC | 126 | GCTCA |
| | | ATTTT | | TAGTT |
| | | GTCCA | | AGAAG |
| | | CCAAC | | TGGCA |
| | | C | | GCA |
| rs2183830 | 127 | GCAAT | 128 | TGGAG |
| | | GATAA | | CCAAA |
| | | CAAGA | | GGGAG |
| | | ACACA | | TAATA |
| | | GCA | | |
| rs2215006 | 129 | TTGCT | 130 | TACAG |
| | | GGCTT | | CTCAG |
| | | ACATT | | CCAGT |
| | | CATTC | | TCTGC |
| | | C | | |
| rs2251381 | 131 | GAAAG | 132 | CCCAT |
| | | GGATG | | GAACA |
| | | ATGGT | | CATTC |
| | | TCCAA | | ACAGC |
| rs2286732 | 133 | GTCTG | 134 | CACGA |
| | | TCCCT | | TTCAG |
| | | GGGCC | | TAAAT |
| | | ATTAT | | GGCTT |
| | | | | G |
| rs2377442 | 135 | TGGAG | 136 | CCATC |
| | | ACATG | | CTGGG |
| | | ACACT | | ATTAC |
| | | ATGAA | | CAATC |
| | | TTT | | T |
| rs2377769 | 137 | TTCTG | 138 | TCATC |
| | | TGTTC | | CATTT |
| | | TACAA | | GAGTT |
| | | TGTCT | | TTCCA |
| | | AGGG | | A |
| rs2388129 | 139 | TATGA | 140 | CCTGA |
| | | GCTGT | | AGTGT |
| | | GGCCA | | CCCCT |
| | | ATGAA | | AGAAG |
| | | | | G |
| rs2389557 | 141 | TTTGC | 142 | TGCAC |
| | | AGACA | | CAAGA |
| | | GGTTA | | TGTGT |
| | | AGATG | | TCTGT |
| | | C | | C |
| rs2400749 | 143 | CCTAC | 144 | TCTAG |
| | | AGTCC | | ATAAG |
| | | AGGGG | | GAGAA |
| | | GTCTT | | TCTGG |
| | | | | TG |
| rs2426800 | 145 | CGGAA | 146 | CACTG |
| | | TTGAG | | GCCTG |
| | | CTAAC | | AGGCT |
| | | CGTCT | | ACTTC |
| rs2457322 | 147 | AAGTC | 148 | TCCCA |
| | | CTGGA | | AGATC |
| | | TTTCA | | TGCAC |
| | | CCAGA | | TAAAC |
| | | G | | G |
| rs2509616 | 149 | CCCTC | 150 | TGGAT |
| | | CAGAG | | TTATT |
| | | CTAAC | | CTTCA |
| | | TGCAT | | TGTTG |
| | | | | CTT |
| rs2570054 | 151 | TTTCC | 152 | AACCA |
| | | AGGAG | | ACACT |
| | | TATAA | | TAGGA |
| | | AGGAG | | AAACA |
| | | TGAA | | AATG |
| rs2615519 | 153 | GAAGC | 154 | CCTGC |
| | | TTCTG | | TGATT |
| | | TCCCT | | TCATC |
| | | TCTGT | | CTTCC |
| rs2622744 | 155 | TCACA | 156 | TCCAG |
| | | TCAGT | | AAGCC |
| | | AACCT | | TTTCT |
| | | CCTTC | | TCCTG |
| | | TTG | | |
| rs2709480 | 157 | GGCAT | 158 | CCTTC |
| | | AGGAA | | TCAAC |
| | | CCATA | | ATAGT |
| | | TTATT | | TCTAA |
| | | GTCA | | TTCC |
| rs2713575 | 159 | CCACA | 160 | TTTCT |
| | | AGCTC | | GAGGC |
| | | ATCAT | | TGATA |
| | | CTATT | | ACTGA |
| | | CG | | A |
| rs2756921 | 161 | GAAGG | 162 | TGCAT |
| | | AACAT | | ATCAC |
| | | CAAAC | | AGTCT |
| | | AAGGA | | CCAAG |
| | | AA | | G |
| rs2814122 | 163 | GAGCA | 164 | TGCCA |
| | | GGTAG | | CCCAG |
| | | CTACA | | ATCTC |
| | | ATGAC | | TTTTC |
| | | A | | |
| rs2826676 | 165 | CCTGA | 166 | TGGGG |
| | | TCTGG | | ATGTG |
| | | AAACT | | GGTAA |
| | | CATGA | | GTTAA |
| | | AA | | T |
| rs2833579 | 167 | GCAAC | 168 | GCTAA |
| | | TGGTC | | GCCAA |
| | | TTGTT | | TGTCT |
| | | CCACA | | ACATC |
| | | | | TTC |
| rs2838046 | 169 | TGGTG | 170 | TGACA |
| | | TGTTA | | TTGGT |
| | | GGGAT | | TATTG |
| | | CTGGA | | GCAGA |
| | | G | | |
| rs2863205 | 171 | CGTAT | 172 | TGCAG |
| | | TCATT | | TGAAG |
| | | ATCCA | | GATTG |
| | | CAGGG | | CAAAG |
| | | ACT | | |
| rs2920833 | 173 | CCCTT | 174 | GCATC |
| | | CCTGG | | TAGAT |
| | | ACTTC | | CTTTA |
| | | ACATA | | CCATT |
| | | G | | GC |
| rs2922446 | 175 | GGAGA | 176 | ACACT |
| | | ACATT | | CGGAA |
| | | TAGTG | | CGATC |
| | | CCTCT | | TCTGC |
| | | GC | | |
| rs3092601 | 177 | AAACC | 178 | TGGGT |
| | | CACGG | | CTCCT |
| | | AGGTC | | ATTTC |
| | | ATTTT | | TGTGT |
| | | | | CC |
| rs3118058 | 179 | TGTTA | 180 | TGGTA |
| | | GGACT | | TGTCT |
| | | ACCTT | | CCTTT |
| | | ATGCA | | GATCT |
| | | GTT | | TT |
| rs3745009 | 181 | CTGAG | 182 | GCTCC |
| | | CGGGA | | TGACG |
| | | GCTTG | | ACCAA |
| | | TAGAT | | TAACC |
| rs4074280 | 183 | GGACC | 184 | TGTGT |
| | | ACTGT | | CTGGT |
| | | CTAGA | | GAGGA |
| | | CCAAG | | AGATG |
| | | C | | A |
| rs4076588 | 185 | GGGAT | 186 | TTTTA |
| | | GAAAC | | GGAAA |
| | | CAAAC | | CCTCA |
| | | CTCCT | | CCAGG |
| | | | | AC |
| rs4147830 | 187 | TCTCT | 188 | TTGAG |
| | | GTTCG | | TTGGC |
| | | TGTCT | | CTAAA |
| | | CTGTC | | ACCAG |
| | | TTG | | A |
| rs4262533 | 189 | CCCGA | 190 | TTGCC |
| | | CCACT | | TCTAA |
| | | AAAAG | | AATCT |
| | | GCATA | | AGAAT |
| | | | | AGCC |
| rs4282978 | 191 | TCTTA | 192 | CACTG |
| | | GGAAT | | AATAT |
| | | GACTC | | TGAAA |
| | | ACACT | | ACTAA |
| | | GGTC | | TGG |
| rs4335444 | 193 | GCATG | 194 | TCACA |
| | | TTATA | | CAGGT |
| | | ATTTT | | TAGGA |
| | | ACAAG | | TGTTT |
| | | CTC | | GTG |
| rs4609618 | 195 | GCACC | 196 | GCAGT |
| | | CTAGG | | TGCCT |
| | | AGCAA | | TGAAA |
| | | ACTGA | | GGAGT |
| rs4687051 | 197 | GCAAA | 198 | GGGGT |
| | | TAAAA | | TGAGA |
| | | TGACT | | TACAA |
| | | CTGGG | | CATCT |
| | | AAC | | TCA |
| rs4696758 | 199 | GATTC | 200 | GGACG |
| | | TTGGG | | TGGGT |
| | | GCATC | | GACTA |
| | | AAGTG | | TCAGG |
| rs4703730 | 201 | TCTAG | 202 | TCCAT |
| | | CTCCT | | TATAG |
| | | AAGTT | | TTCAG |
| | | GATTG | | TCTTC |
| | | ATTC | | AAT |
| rs4712253 | 203 | CAGGA | 204 | AGCGA |
| | | GAAAA | | GAGCA |
| | | GCAGA | | GGCTC |
| | | GACCA | | ATAAT |
| | | A | | |
| rs4738223 | 205 | TGACA | 206 | GAAAC |
| | | AGGGA | | TACCT |
| | | TTAGG | | CTGAG |
| | | GCAAA | | TGTTA |
| | | | | CAGA |
| rs4920944 | 207 | GAATC | 208 | TGAAA |
| | | CTGGA | | ATGAG |
| | | CGGTC | | TAGTG |
| | | AGAAA | | GACAT |
| | | | | CTG |
| rs4928005 | 209 | AAAAT | 210 | CCCTA |
| | | GTGAA | | ACTTA |
| | | GATAA | | TTCAA |
| | | GTGAA | | CATCA |
| | | CAGC | | CTGC |
| rs4959364 | 211 | ACATA | 212 | CATTG |
| | | TTCCA | | AGTTC |
| | | GGAGC | | ATTGG |
| | | ATGAC | | CCTGT |
| rs4980204 | 213 | CTCTC | 214 | CCAAC |
| | | GTGGT | | AAGTA |
| | | GGATT | | CTCTG |
| | | GAACA | | AACCA |
| | | | | ATTT |
| rs6023939 | 215 | AAGGA | 216 | GCTCT |
| | | GGGCT | | TTCTC |
| | | TAGCT | | ATCTT |
| | | AGTTG | | AAGGC |
| | | | | TTC |
| rs6069767 | 217 | GTTAA | 218 | CAGGC |
| | | AATTA | | AACCA |
| | | CTGTT | | AATAA |
| | | CCAGT | | TAACA |
| | | TGT | | AAA |
| rs6075517 | 219 | CCCAT | 220 | TTGTA |
| | | TTCCA | | TTTAC |
| | | TTTAC | | AATAG |
| | | CGTTT | | CCATC |
| | | T | | CA |
| rs6075728 | 221 | TGAAA | 222 | AGCAG |
| | | GTATC | | TCAAA |
| | | AGGAA | | GTGAG |
| | | AAATG | | GATAT |
| | | GATG | | GTT |
| rs6080070 | 223 | GCAGT | 224 | ACCAG |
| | | AACAA | | CCTTT |
| | | ATAAC | | GTTGT |
| | | CCCAA | | TGAGC |
| | | CAG | | |
| rs6434981 | 225 | GGGTT | 226 | GGTAA |
| | | CCAGC | | TGAAG |
| | | AATAT | | AAAGA |
| | | TCTAC | | CAAAA |
| | | CTT | | CA |
| rs6461264 | 227 | TCTAA | 228 | GCACA |
| | | TGCCT | | GCAGA |
| | | CACCA | | AACCC |
| | | AGCAA | | AGATT |
| rs6570404 | 229 | CACTA | 230 | TGGTG |
| | | GTCCG | | ATTAC |
| | | GCTTG | | AGAAT |
| | | TGTAA | | ACCAC |
| | | AA | | CAG |
| rs6599229 | 231 | ACAGG | 232 | TGATG |
| | | AGCGG | | TGCAT |
| | | ACAAT | | GTGTC |
| | | GAGAG | | TCAGC |
| rs6664967 | 233 | TGGTC | 234 | CATAC |
| | | CTCTG | | ATGAG |
| | | CTTCC | | GTGAC |
| | | CTAAG | | TACCA |
| | | | | CCA |
| rs6739182 | 235 | CATCA | 236 | AGCTC |
| | | GATTC | | ATCCC |
| | | CCAAC | | AATCA |
| | | ATTGC | | TCACA |
| | | T | | |
| rs6758291 | 237 | AAGGG | 238 | AACCC |
| | | CCATG | | AAACG |
| | | AGGGT | | TCTAA |
| | | ACTTT | | CAAGA |
| | | | | TACA |
| rs6788448 | 239 | CATCG | 240 | TGTGA |
| | | ATAGT | | TTTCT |
| | | ATTAG | | TTCTA |
| | | GCCCA | | TAGGA |
| | | CA | | GGTT |
| rs6802060 | 241 | GGAAG | 242 | TTCCA |
| | | GAAAG | | GCCCT |
| | | CTCTT | | GAATA |
| | | TTGGA | | ACAAC |
| | | A | | TT |
| rs6828639 | 243 | TGATC | 244 | AGGAT |
| | | ATTGC | | ACCAT |
| | | TGTGA | | GATTT |
| | | TGTAT | | TGTAG |
| | | T | | TGC |
| rs6834618 | 245 | CTTCC | 246 | CTGTT |
| | | CTGCA | | TAGGA |
| | | CATCC | | AGAGT |
| | | TTTTG | | CATGT |
| | | | | AACC |
| rs6849151 | 247 | AACTG | 248 | AAAAG |
| | | TTTTG | | ACCAC |
| | | TCAGC | | TTGAT |
| | | TGCTC | | TCAGC |
| | | AT | | TT |
| rs6850094 | 249 | TGAGC | 250 | TGCAA |
| | | ACACA | | TGTAC |
| | | CATAT | | ATGTG |
| | | GGAAG | | GAGAA |
| | | C | | TC |
| rs6857155 | 251 | CCCGT | 252 | CCCAG |
| | | TCTCC | | GGAAG |
| | | ATTCT | | AAAAT |
| | | GGTTA | | TGGTA |
| rs6927758 | 253 | TGAAA | 254 | AGCCA |
| | | TAGTG | | CTCCA |
| | | CTTAT | | GCATT |
| | | TGCAT | | CACTT |
| | | CG | | |
| rs6930785 | 255 | CCACA | 256 | GGAGT |
| | | TGTTT | | TACAG |
| | | CTGAG | | TTATC |
| | | TGAAG | | AAATG |
| | | GA | | CAGA |
| rs6947796 | 257 | GGAAA | 258 | TTGCA |
| | | GAAGG | | TATTC |
| | | GAGAA | | TGGAC |
| | | TGGTC | | CTCAT |
| | | A | | CT |
| rs6981577 | 259 | GGAGG | 260 | TTTTA |
| | | CAAAG | | CCTCC |
| | | AAGTT | | CTGCC |
| | | AGGGA | | CTAGT |
| | | GT | | |
| rs7104748 | 261 | AGGAA | 262 | GCAGC |
| | | ATGTA | | TTGAA |
| | | GTCAG | | AACAG |
| | | GTCTA | | CCAGT |
| | | GGA | | |
| rs7111400 | 263 | CATGG | 264 | GCTGA |
| | | TAAGT | | GCAGA |
| | | ATGCT | | AAACA |
| | | GTTAA | | TAAGC |
| | | ATC | | A |
| rs7112050 | 265 | CAAAC | 266 | AGCTA |
| | | CCACA | | ATCTT |
| | | CTGTG | | TGGTA |
| | | TTAGC | | CTTCA |
| | | TG | | ATCT |
| rs7124405 | 267 | CAAGC | 268 | AGTGC |
| | | ATCTT | | AAAGT |
| | | GCTGA | | GAAGA |
| | | ATTTC | | TAATG |
| | | C | | ACA |
| rs7159423 | 269 | AGTGT | 270 | CATTC |
| | | CTGTC | | ATCCC |
| | | TTCCA | | ATCTT |
| | | GTTCC | | CTAAC |
| | | | | TTCA |
| rs7229946 | 271 | GCAAA | 272 | GCAGT |
| | | CATGT | | CTTCT |
| | | AAAGT | | GTGAT |
| | | GTGAG | | TTTAT |
| | | AG | | ATT |
| rs7254596 | 273 | CAGAA | 274 | TCCCC |
| | | GGAAG | | TCAGG |
| | | GGGTA | | TAACT |
| | | AGACA | | TCCAT |
| | | CA | | C |
| rs7422573 | 275 | GATTT | 276 | TTGGT |
| | | CTGTG | | GTCTT |
| | | TTGTG | | ACATG |
| | | CCACA | | TATTG |
| | | GT | | TGA |
| rs7440228 | 277 | GCTGT | 278 | GAACT |
| | | AGCAC | | GAAAA |
| | | ATCCA | | AGGAA |
| | | AAAAC | | TAAAG |
| | | C | | TAGG |
| rs7519121 | 279 | GGCAT | 280 | TGAAA |
| | | AAGCA | | CCTAT |
| | | GATAC | | AAGCC |
| | | AGACA | | ACTGA |
| | | GC | | GC |
| rs7520974 | 281 | TCCAA | 282 | AAGCC |
| | | AAAGA | | ATGCA |
| | | CAGCT | | GTGGG |
| | | GAAAG | | TATCT |
| | | AA | | |
| rs7608890 | 283 | TCCAT | 284 | GTGCA |
| | | ACAGG | | GTTTG |
| | | AAGAT | | GGCTA |
| | | CCATT | | CAAGA |
| | | AAGA | | |
| rs7612860 | 285 | TCACA | 286 | AAGTG |
| | | CATCA | | TCAGA |
| | | TTGGT | | GGGTT |
| | | GAAGG | | AGTGA |
| | | | | TTCC |
| rs7626686 | 287 | CACCT | 288 | GACTT |
| | | AAAGA | | ACGGC |
| | | TTTCC | | CTAAC |
| | | CCACA | | CCTTT |
| | | A | | |
| rs7650361 | 289 | GAACA | 290 | TTTGT |
| | | AGTAT | | CTAAA |
| | | ACTAG | | GAATT |
| | | CAAAA | | TGACA |
| | | CGAA | | GTGG |
| rs7652856 | 291 | TCTTG | 292 | GCATG |
| | | AGAAG | | AGTGT |
| | | CCTTT | | GTGTC |
| | | TCTTA | | TATGC |
| | | CCA | | AG |
| rs7673939 | 293 | TTCTG | 294 | TGGCA |
| | | GACTC | | TAAGA |
| | | TCCAC | | TAGAC |
| | | TCTAT | | ATATT |
| | | TTCA | | CACC |
| rs7700025 | 295 | GCATC | 296 | GCCGT |
| | | TATGT | | TAAGC |
| | | CACCA | | ACTGA |
| | | AGCAT | | GCTGT |
| | | TT | | |
| rs7716587 | 297 | TCCAC | 298 | TCTTG |
| | | TACTT | | AATAG |
| | | CTTGG | | CACCC |
| | | AGTTC | | ACAAG |
| | | A | | AG |
| rs7767910 | 299 | GACAC | 300 | GCCCA |
| | | TACTG | | AAGAC |
| | | TCCTC | | CAAGT |
| | | AAACG | | TTTAG |
| | | | | A |
| rs7917095 | 301 | CGTGT | 302 | AGGTT |
| | | CTGTG | | GTGAA |
| | | AGCTC | | AGACA |
| | | CTTTC | | CTGAT |
| | | T | | GG |
| rs7925970 | 303 | TCCAA | 304 | CAGTG |
| | | GCTGT | | GGCTC |
| | | TTCTC | | ACAGT |
| | | ATGTT | | AATGG |
| | | TG | | |
| rs7932189 | 305 | GCAAT | 306 | TTATC |
| | | TCCAG | | TACCC |
| | | ATATC | | ATGCT |
| | | TCTTT | | TCTCT |
| | | AT | | C |
| rs8067791 | 307 | AACAG | 308 | CCCTA |
| | | ATCAC | | CATGC |
| | | TTACC | | ATTAT |
| | | GCTTT | | CTCCT |
| | | G | | TT |
| rs8130292 | 309 | TGGTG | 310 | AGTGT |
| | | CCATC | | GCACT |
| | | CTAGA | | TGCTC |
| | | GTTCT | | ATGAC |
| | | G | | T |
| rs9293030 | 311 | CCAGG | 312 | ATGTC |
| | | GATTT | | TATGC |
| | | CATCT | | CCTGC |
| | | TCACC | | CTCAT |
| rs9298424 | 313 | TGTAG | 314 | TTTCA |
| | | TCGAA | | CTCCC |
| | | GCAAT | | TTCTG |
| | | GAGAT | | TATTT |
| | | GTG | | AGCC |
| rs9397828 | 315 | AAATG | 316 | TCAAT |
| | | CTTTG | | GGCAA |
| | | CTGCA | | TTTGA |
| | | TGTCT | | GGAGA |
| rs9432040 | 317 | TGAGG | 318 | TTTTC |
| | | AAGTG | | TCCCC |
| | | ACAAG | | ATCTG |
| | | TTCAG | | TTACT |
| | | A | | A |
| rs9479877 | 319 | CAATT | 320 | TGGGA |
| | | TTACA | | TTATA |
| | | TCCAA | | AGGAG |
| | | CAGAA | | GTCAA |
| | | GA | | GAA |
| rs9678488 | 321 | TGGTG | 322 | CTTGA |
| | | AGTTT | | CACCA |
| | | CTTCC | | TAGTG |
| | | CTAGG | | GTCAC |
| | | TT | | CT |
| rs9682157 | 323 | TTTAC | 324 | CACGC |
| | | TTCTG | | AGGCA |
| | | AGCTG | | ATAGT |
| | | AAGGT | | AGGAA |
| | | ACTC | | |
| rs9810320 | 325 | AGCAC | 326 | GGATG |
| | | CAAAG | | CCAAG |
| | | GCAAG | | ATTGC |
| | | TTCAA | | AAATA |
| rs9841174 | 327 | TTCTT | 328 | TTTCA |
| | | TCTAC | | AGATG |
| | | CCAGG | | CAAAG |
| | | TACTT | | GCTTG |
| | | ATCA | | |
| rs9864296 | 329 | CGAAA | 330 | AGCTA |
| | | TCCAT | | CACTA |
| | | AGGAC | | TTTCC |
| | | CTACA | | ATGTG |
| | | | | AC |
| rs9867153 | 331 | CGTCG | 332 | GGACA |
| | | GTTGT | | GGTTG |
| | | TTTAT | | TGCAT |
| | | CATTG | | AACTA |
| | | C | | AGA |
| rs9870523 | 333 | CCTCA | 334 | TGCTA |
| | | CTTAA | | ATCAT |
| | | GGAGA | | CCCTT |
| | | ACAGT | | ATTAT |
| | | TAGA | | TGC |
| rs9879945 | 335 | TGACC | 336 | TGCCA |
| | | TACTA | | GTAAC |
| | | GACAT | | TTAAT |
| | | CAAGC | | CCATA |
| | | CTTA | | GC |
| rs9924912 | 337 | CCAGA | 338 | GGGAA |
| | | CAGGC | | CTGAG |
| | | ACATA | | TATCT |
| | | CAGTC | | CTGTG |
| | | A | | TGA |
| rs9945902 | 339 | GAGGT | 340 | TCAAC |
| | | CGAAG | | TTAGT |
| | | TTGTA | | TACAG |
| | | GGCTT | | GTCAC |
| | | G | | ACA |
| rs10033133 | 341 | TCAAT | 342 | AGGTT |
| | | TTTTG | | TTCCT |
| | | TTGTG | | AATAA |
| | | GTTTA | | GACTG |
| | | CCT | | CT |
| rs10040600 | 343 | TCAGA | 344 | CTCAG |
| | | GTAGG | | GGCCT |
| | | AATGA | | AAACT |
| | | ACAAT | | TGCAC |
| | | TT | | |
| rs10089460 | 345 | GCACT | 346 | CACAG |
| | | CATGT | | TGAAG |
| | | GAGTT | | TATGT |
| | | TGCAC | | ATAAA |
| | | | | TTGC |
| rs10133739 | 347 | GCCTA | 348 | TGATA |
| | | GCTGT | | CCAGT |
| | | GCGAT | | TGATG |
| | | TCTTC | | CCACA |
| rs10134053 | 349 | TGACT | 350 | TGGCA |
| | | GAACT | | TCTAG |
| | | CAATT | | GGTAT |
| | | CAAAC | | AGGAA |
| | | AGC | | GA |
| rs10168354 | 351 | GGCCA | 352 | CCTTG |
| | | CCATC | | TTTGT |
| | | TCCTG | | CTGTA |
| | | TTCTA | | TCTGA |
| | | | | GC |
| rs10232758 | 353 | CCAAC | 354 | GCTCC |
| | | TCTGA | | AAGCC |
| | | TTGTG | | ATAGA |
| | | CGACT | | TCCAG |
| rs10246622 | 355 | GGTGT | 356 | AACCG |
| | | GTGTA | | CCAGC |
| | | TGAGG | | ATAGC |
| | | CTTGG | | TTCT |
| rs10509211 | 357 | GGTAG | 358 | TTTCT |
| | | GAAGG | | TTCTA |
| | | GGTTG | | CTTCT |
| | | TCGTT | | CATCA |
| | | | | CTCT |
| rs10518271 | 359 | GGACA | 360 | TTCTC |
| | | TCAGC | | TTGTG |
| | | ACTAA | | TGAAC |
| | | CTGAA | | CATCC |
| | | GTG | | TC |
| rs10737900 | 361 | GCCAG | 362 | TGGCA |
| | | CGTGT | | TTTGT |
| | | AAGAC | | TTACA |
| | | ACAAG | | GACTT |
| | | | | ATC |
| rs10758875 | 363 | TCCTC | 364 | GGTGT |
| | | CACAT | | CCCCC |
| | | TGGTA | | TCAAA |
| | | ATTAG | | TTGTA |
| | | GG | | |
| rs10759102 | 365 | CAAGT | 366 | TGAGA |
| | | TTGTA | | TACTG |
| | | CCTCA | | TTGTC |
| | | GCTTT | | CTCTG |
| | | CA | | C |
| rs10781432 | 367 | TTCCC | 368 | GAGGG |
| | | TTCTT | | TTACT |
| | | ATGTA | | GAACT |
| | | ATCTC | | AGGAT |
| | | C | | AATG |
| rs10790402 | 369 | TCCTG | 370 | TGCAG |
| | | AGAGC | | GGCAT |
| | | ATGGT | | TCTAT |
| | | AAGAT | | GTGAA |
| | | GT | | |
| rs10881838 | 371 | TACAG | 372 | TGGCT |
| | | CTGAG | | GGCCA |
| | | CAATA | | AATCT |
| | | ACGTG | | TTCTA |
| rs10914803 | 373 | AAACT | 374 | AAGTC |
| | | ATAAA | | TAGTG |
| | | AGGAC | | AATTT |
| | | CTAGG | | CTTGT |
| | | AAA | | TAGG |
| rs10958016 | 375 | CTTAA | 376 | ATTTG |
| | | TGATT | | AGAGG |
| | | TTGTA | | TTGCC |
| | | ATGTC | | AGAGC |
| | | AGG | | |
| rs10980011 | 377 | GAGGT | 378 | AGAGG |
| | | TCTCA | | GGCTC |
| | | TTCCC | | ACCTG |
| | | TCACC | | AGAGT |
| rs10987505 | 379 | CACAC | 380 | TTGCG |
| | | TAGTG | | GTTTC |
| | | GGTCC | | CTCAT |
| | | TGATT | | TCTTC |
| | | AGA | | |
| rs11074843 | 381 | CGTGA | 382 | CGCCT |
| | | TGGGT | | CTGGG |
| | | AGGTC | | GATAA |
| | | AGTCC | | CTAAA |
| rs11098234 | 383 | GGAAT | 384 | AGTGG |
| | | TGCCA | | TCCCC |
| | | CTCTG | | AACAA |
| | | GAGAA | | CTTGA |
| rs11099924 | 385 | ATAAC | 386 | GATCA |
| | | AATGT | | ACACT |
| | | CTAGC | | TCAAA |
| | | AACAG | | ATTAT |
| | | G | | GGT |
| rs11119883 | 387 | TCAGA | 388 | ACCCA |
| | | TAAAA | | CAGAG |
| | | CAATT | | GAAAG |
| | | CCAGT | | CCTTG |
| | | TAC | | |
| rs11126021 | 389 | CAGCA | 390 | TGTGC |
| | | TATAT | | CCAGA |
| | | TACCT | | AAGTT |
| | | TTTCT | | TTAGC |
| | | TTG | | A |
| rs11132383 | 391 | TCAAC | 392 | GTGAA |
| | | TGACA | | GGGAG |
| | | CTGGT | | GACAA |
| | | GTTTC | | AATCG |
| | | TC | | |
| rs11134897 | 393 | CAAGT | 394 | TGCTG |
| | | GATCT | | AGTTT |
| | | GATGG | | GAGAA |
| | | GGTGA | | ACTTG |
| | | | | GT |
| rs11141878 | 395 | GTAGG | 396 | GCATT |
| | | ACTTA | | ACTGC |
| | | GGGCG | | CGAGG |
| | | CTCAT | | GATCT |
| rs11733857 | 397 | TGACA | 398 | TCCTA |
| | | AAGCC | | GAGTA |
| | | TAGAG | | CTCCT |
| | | TGAAC | | CTTTG |
| | | TGA | | TCCA |
| rs11738080 | 399 | GTACA | 400 | CATGA |
| | | GAGTC | | TCTGT |
| | | CCTGT | | CTCTC |
| | | CTCAC | | TCACT |
| | | A | | GAA |
| rs11744596 | 401 | GCATT | 402 | TGGCC |
| | | TTCTC | | TAAAA |
| | | ACAGC | | ATTCA |
| | | CACAG | | CCACT |
| | | | | G |
| rs11785007 | 403 | AACAT | 404 | GCAAG |
| | | TTGCA | | GATCA |
| | | CATTA | | GTCAG |
| | | TCAGC | | ACTAC |
| | | | | GA |
| rs11925057 | 405 | TGTCC | 406 | CTGAT |
| | | ATCAA | | TTCTA |
| | | TCTCA | | CCAGT |
| | | AAAGT | | TACTT |
| | | CG | | ACCA |
| rs11941814 | 407 | GCATG | 408 | TGCAG |
| | | AGCCA | | ACCAT |
| | | CCCTA | | GAGGA |
| | | AATCT | | ATGTT |
| rs11953653 | 409 | AGGAT | 410 | ACCAA |
| | | TCCTT | | ATAAT |
| | | ATACA | | GGTCT |
| | | CTGAC | | ACTCC |
| | | CTC | | T |
| rs12036496 | 411 | AAGAC | 412 | GGCTC |
| | | ATTCT | | TACTA |
| | | CTGCC | | TGGGG |
| | | TTTCT | | AAAAT |
| | | CA | | TCA |
| rs12045804 | 413 | GCAAA | 414 | GAGGT |
| | | TCACT | | TCACT |
| | | AGGAA | | CTATT |
| | | AGCTC | | TCTGT |
| | | A | | TCC |
| rs12194118 | 415 | CTAGA | 416 | CCCTG |
| | | AACGG | | CACTT |
| | | CTGCC | | GTACC |
| | | AGGTA | | AGCTT |
| rs12286769 | 417 | AGGAC | 418 | ATCCC |
| | | ATTCT | | ATATA |
| | | TTTGT | | GGCAC |
| | | GTATT | | TTGCT |
| | | CAAG | | |
| rs12321766 | 419 | CAAAT | 420 | GCTTT |
| | | AATCA | | CAGTG |
| | | CCCCA | | CCCTC |
| | | ATACA | | ATCTC |
| | | ATCA | | |
| rs12553648 | 421 | AAGAT | 422 | CACTC |
| | | GATCA | | CTAAA |
| | | AAGTT | | GAACA |
| | | TTGAG | | AGATG |
| | | AGCA | | TCAA |
| rs12603144 | 423 | GACAA | 424 | GGGAG |
| | | GAACT | | GAACA |
| | | GAAGG | | GAACA |
| | | CAAAG | | ACCTT |
| | | G | | C |
| rs12630707 | 425 | CCCTT | 426 | AGTTA |
| | | GCAAT | | TCTGA |
| | | ACCCA | | GTTGG |
| | | GCATA | | CTTAC |
| | | | | C |
| rs12635131 | 427 | TCGCA | 428 | TCCAA |
| | | GTCTT | | TAGCT |
| | | TTGCA | | ACCTT |
| | | TCATT | | CACCA |
| | | | | GAA |
| rs12902281 | 429 | TGGAA | 430 | CCAAA |
| | | AAACA | | AGCAT |
| | | CAGGC | | CTAAA |
| | | ATATT | | AACAG |
| | | CTC | | GA |
| rs13019275 | 431 | CAAAT | 432 | TGATG |
| | | ATACT | | CATTG |
| | | GATTC | | AGATT |
| | | TGTGG | | TTGAT |
| | | CAAA | | GA |
| rs13026162 | 433 | TAGCC | 434 | GAGGG |
| | | TTTGG | | AGGAA |
| | | ATAAC | | ATGGT |
| | | AGTCC | | CAACT |
| | | | | T |
| rs13095064 | 435 | AGGCA | 436 | AGACG |
| | | AAGAA | | TGCTG |
| | | CTAGA | | GGTTC |
| | | CAACT | | CTAGA |
| | | CT | | |
| rs13145150 | 437 | GGCAT | 438 | TTGTC |
| | | GAAGA | | TGGTC |
| | | TGTTA | | TTCAT |
| | | ACCTA | | CAAGT |
| | | CCA | | CTCT |
| rs13171234 | 439 | TTGCC | 440 | TGACT |
| | | ATGCA | | TTTCA |
| | | GCAGT | | TTGCT |
| | | ACTTA | | AGTAT |
| | | G | | CCA |
| rs13383149 | 441 | GCAAC | 442 | TGTTT |
| | | AAGAA | | TGACA |
| | | CAGGA | | TTGTC |
| | | ACCAA | | CTGTG |
| | | G | | TG |
| rs16843261 | 443 | CAGTG | 444 | GAGAA |
| | | AGGTG | | CACAT |
| | | TGATG | | ATTCA |
| | | TATAA | | TTCCT |
| | | AGAG | | CTCC |
| rs16864316 | 445 | GTGGG | 446 | GAACT |
| | | GTCCA | | TCTCA |
| | | GCAGT | | CATCA |
| | | AAATC | | CCTCA |
| | | | | AGC |
| rs16950913 | 447 | TCTAT | 448 | TTGCT |
| | | TAACC | | AAATT |
| | | CTAAT | | TCAGG |
| | | CAATC | | CACCT |
| | | TCCT | | C |
| rs16996144 | 449 | CCTTT | 450 | AGTGA |
| | | GACTC | | ATAAC |
| | | TGGCC | | CAGCC |
| | | TCATC | | TTAGT |
| | | | | TG |
| rs17520130 | 451 | AAATA | 452 | GTGCC |
| | | AGGAC | | AGCTA |
| | | ATCTG | | CAAAC |
| | | GAAAA | | AATGG |
| | | CAA | | |

TABLE 4
Panel B SNPs and amplification primers

| | | First | | Second |
|---|---|---|---|---|
| | SEQ ID | Primer | SEQ ID | Primer |
| SNP | NO | Sequence | NO | Sequence |
| rs196008 | 453 | GTGCC | 454 | ACACA |
| | | TCATC | | GATGA |
| | | AAAAT | | CTTCA |
| | | GCAAC | | GCTGG |
| rs243992 | 455 | AACTC | 456 | GGAAT |
| | | AAACC | | GGAAT |
| | | TAAGT | | AGTGT |
| | | GCCCC | | GTGGG |
| rs251344 | 457 | ACACT | 458 | CACAC |
| | | GGTCT | | CTGTA |
| | | CAAGC | | ATTCT |
| | | TCCC | | AGCCC |
| rs254264 | 459 | AGAAG | 460 | AGCTT |
| | | GAAGG | | TCCTC |
| | | ATCAG | | CCCAC |
| | | AGAAG | | ACTG |
| rs290387 | 461 | GCTGT | 462 | GAATG |
| | | GTGGA | | AAATG |
| | | GCCCT | | GAGTT |
| | | ATAAA | | TGCAG |
| rs321949 | 463 | CCTCA | 464 | GTGTT |
| | | GCCAC | | GGTCA |
| | | CACTT | | GACAG |
| | | GTTAG | | AAAGG |
| rs348971 | 465 | GCCAA | 466 | ATGCA |
| | | TTACC | | CACTT |
| | | CCATA | | ACACA |
| | | ATTAG | | CGCAC |
| rs390316 | 467 | AAGGA | 468 | AGGCT |
| | | AGTAA | | AACTC |
| | | AGGTA | | TAACA |
| | | TGTGC | | TCCTG |
| rs425002 | 469 | AAGAG | 470 | AACTG |
| | | TGTCT | | GAGGC |
| | | CCTCC | | TGTGT |
| | | CTCTG | | TAGAC |
| rs432586 | 471 | CGCTC | 472 | TTGCA |
| | | TTTTC | | GCAGT |
| | | TGACT | | CACAG |
| | | AGTCC | | GAAAC |
| rs444016 | 473 | CTCTC | 474 | GGAAG |
| | | TGTGC | | ACACT |
| | | ACAAA | | GCCTT |
| | | AAACC | | CAAAC |
| rs447247 | 475 | AAAAA | 476 | ATGTC |
| | | CCCCA | | CAGCT |
| | | GGCTC | | GCTTC |
| | | CATTG | | TTTTC |
| rs484312 | 477 | TCCAA | 478 | AGTCT |
| | | GTCAG | | GCAGA |
| | | AAGCT | | CCTAA |
| | | ATGGG | | CATGG |
| rs499946 | 479 | ATGGC | 480 | TTCGG |
| | | TTGTA | | TGGAA |
| | | CTTCC | | TAGCA |
| | | TCCTC | | GCAAG |
| rs500090 | 481 | CATAA | 482 | TTCAC |
| | | TCTCA | | CTGGC |
| | | GGGCT | | CTTGA |
| | | ACAT | | GGGTC |
| rs500399 | 483 | GTTTA | 484 | GGGCA |
| | | TTGAT | | GAGTG |
| | | GAACT | | ATATC |
| | | GGTGC | | ACAG |
| rs505349 | 485 | ACTGG | 486 | AAGGC |
| | | CAAGT | | TCAGG |
| | | CCAGG | | GCAGA |
| | | TCTTC | | AGCAC |
| rs505662 | 487 | TCCTC | 488 | CAGCA |
| | | ATCCG | | AAGAG |
| | | GTGTG | | AGAGA |
| | | GCAA | | GGTTC |
| | | | | C |
| rs516084 | 489 | AGTAT | 490 | CTTCT |
| | | GCCAT | | TTGAC |
| | | CATGA | | TAAGG |
| | | AAGCC | | CTGAC |
| rs517316 | 491 | CTCTG | 492 | TAGAC |
| | | CCTAT | | CTCAA |
| | | TCTCC | | GGCCT |
| | | TCTTC | | AGAGC |
| rs517914 | 493 | AGTAA | 494 | GCTCA |
| | | GAGCT | | TAACA |
| | | CCCTT | | ATCTC |
| | | GGTTG | | TCCCC |
| rs522810 | 495 | TCCCC | 496 | CAGCA |
| | | TCTAC | | CTGAT |
| | | CCCTT | | GACAT |
| | | GAAGC | | CTGGG |
| rs531423 | 497 | AAGAA | 498 | TATGG |
| | | CACAG | | CTCTG |
| | | GCCTG | | GGGCT |
| | | GTTGG | | CTATA |
| rs537330 | 499 | AACAG | 500 | TCATT |
| | | AGAGA | | CTAAA |
| | | ATGAG | | AGGGC |
| | | GAGGG | | TGCCG |
| rs539344 | 501 | GAAAG | 502 | GATGC |
| | | GTATT | | TCTGA |
| | | CAGGG | | GACAA |
| | | TGGTG | | TCCTG |
| rs551372 | 503 | TTAAC | 504 | GATCA |
| | | TGTGA | | TGGGA |
| | | GGCGT | | CTATC |
| | | TCACC | | CACAC |
| rs567681 | 505 | CCAGC | 506 | GGAGA |
| | | CCTGC | | AGATC |
| | | TCCTT | | CTACA |
| | | TAATC | | CTCAG |
| rs585487 | 507 | CCAAC | 508 | CTGGA |
| | | TTCTT | | GCTGA |
| | | CCCAG | | AGGAC |
| | | TCTGT | | CCCA |
| rs600933 | 509 | GGAGA | 510 | TTCAA |
| | | AATCC | | GGTGC |
| | | TTCCC | | TGCAG |
| | | TAGAG | | GTTTG |
| rs619208 | 511 | CCCCC | 512 | TTCTG |
| | | TCTAC | | AATTC |
| | | AGGAA | | TTCAG |
| | | AATTC | | CCAGC |
| rs622994 | 513 | CATCC | 514 | GGTGT |
| | | TACCT | | CTTAG |
| | | CTAGG | | TTACA |
| | | TACAC | | TGTGC |
| rs639298 | 515 | TGGTG | 516 | ATACT |
| | | ACGCA | | GTGCT |
| | | AGGAC | | GCTCT |
| | | TGGAC | | TCAGG |
| rs642449 | 517 | CAGCT | 518 | CCAAA |
| | | GCTGT | | AAACC |
| | | TCCCT | | ATGCC |
| | | CAGA | | CTCTG |
| rs677866 | 519 | TAATT | 520 | AGGCA |
| | | GGTAC | | TGGGA |
| | | AGGAG | | CTCAG |
| | | GTGGG | | CTTG |
| rs683922 | 521 | GTGCA | 522 | AAACA |
| | | GGTCA | | CTCCA |
| | | TTGTG | | CGTTA |
| | | CTGAG | | AAGGG |
| rs686851 | 523 | CAGCT | 524 | TTTAC |
| | | GAGAA | | AGACT |
| | | AACTG | | AGCGT |
| | | AGACC | | GACGG |
| rs870429 | 525 | TGCTG | 526 | ATGCA |
| | | CTCCG | | GGGAG |
| | | CCATG | | AGCAG |
| | | AAAGT | | CAGCC |
| rs949312 | 527 | GCTGA | 528 | CTGTG |
| | | GAGTT | | GCCAT |
| | | AAGTG | | ATTTC |
| | | GCCAA | | TGCTG |
| rs970022 | 529 | GCAAT | 530 | TTGTC |
| | | CAGGC | | TGGAC |
| | | CCAGC | | TCTCT |
| | | TTATG | | TCATC |
| rs985462 | 531 | CGCCT | 532 | GACTT |
| | | AATTT | | GCAAA |
| | | CCAGC | | AGCTC |
| | | AAGAA | | TCTGG |
| rs1115649 | 533 | GTCTG | 534 | AAGGG |
| | | GCTGA | | CAGCA |
| | | GGAAT | | TGAGC |
| | | GCTAC | | TTGGG |
| rs1444647 | 535 | GTCTA | 536 | CTACA |
| | | CTTCA | | TGCAT |
| | | AATCA | | ATCTG |
| | | TGCCT | | GAGAC |
| | | C | | |
| rs1572801 | 537 | CAGAG | 538 | AGGAA |
| | | ATGCA | | TGGGG |
| | | AGCAG | | CTGCC |
| | | CCAAG | | ATCT |
| rs1797700 | 539 | GAGAC | 540 | ACCAC |
| | | AGGCA | | GCCTG |
| | | AAGAT | | GCCAG |
| | | GCAAC | | AACT |
| rs1921681 | 541 | GGGTT | 542 | AATGT |
| | | TAGTC | | CCCTG |
| | | TCCTT | | GCACA |
| | | ACCCC | | GCTCA |
| rs1958312 | 543 | GCTTC | 544 | CTCAG |
| | | AGTTG | | ATGAT |
| | | TCACT | | GTCCC |
| | | GTGAG | | TTCTT |
| rs2001778 | 545 | CGATG | 546 | GGACA |
| | | CAAGC | | GAGAA |
| | | TTCCA | | TGGCC |
| | | TTCTA | | TGCTA |
| rs2323659 | 547 | TTAAA | 548 | TGATG |
| | | ACAGC | | AGAAC |
| | | CCTGC | | AGAGC |
| | | AACC | | TGAG |
| rs2427099 | 549 | CTGAA | 550 | AGGTG |
| | | GCTAT | | GCACG |
| | | GTCCT | | GCACG |
| | | GTTAG | | TTCAT |
| rs2827530 | 551 | CTGAA | 552 | ACCCT |
| | | GTGCA | | AGAAC |
| | | GGAAG | | TTGAC |
| | | CTTGG | | ACTGC |
| rs3944117 | 553 | AAGGA | 554 | ACATA |
| | | GCTGG | | GGCAC |
| | | CAAGG | | AATGA |
| | | CCCTA | | GATGG |
| rs4453265 | 555 | TACCT | 556 | TTTGG |
| | | TTCAA | | ATGGA |
| | | GCTCA | | ACGTT |
| | | AGTGC | | TGCAG |
| rs4745577 | 557 | GCTAC | 558 | ATGAA |
| | | CCTTT | | GAGCA |
| | | AATGT | | GCTGG |
| | | GTCTC | | TCAAC |
| rs6700732 | 559 | CAGCC | 560 | TACAG |
| | | CTTGT | | TGGTG |
| | | GTGCA | | GACAA |
| | | TAAAG | | GGTGG |
| rs6941942 | 561 | CTTGT | 562 | TCAAT |
| | | TTTGC | | CATCC |
| | | AGGCT | | CCATC |
| | | GATTG | | CCCAC |
| rs7045684 | 563 | GCACA | 564 | CCCCA |
| | | TCACA | | GTAGG |
| | | AGTTA | | GAACA |
| | | AGAGG | | CACTT |
| rs7176924 | 565 | CAGGA | 566 | GGCTT |
| | | TGCAC | | CTCCC |
| | | TTTTT | | AGAAA |
| | | GGATG | | ATCTC |
| rs7525374 | 567 | ACTGC | 568 | TTTGC |
| | | AGTGC | | TCACC |
| | | CGGGA | | CTACC |
| | | AAAGT | | CCAC |
| rs9563831 | 569 | TGATA | 570 | TAGGG |
| | | ACAGC | | ATGCA |
| | | CTCCA | | AGATG |
| | | TTTCC | | AAAGG |
| rs10413687 | 571 | GATGC | 572 | TCCAG |
| | | AGGAG | | CCACT |
| | | GGCGT | | CTGAG |
| | | CCCA | | CTGC |
| rs10949838 | 573 | TCTGC | 574 | TGGGA |
| | | TGTTT | | GATCA |
| | | GATGG | | GCTAG |
| | | ATGTG | | GAATG |
| rs11207002 | 575 | GCTGG | 576 | TGAAT |
| | | GATCC | | GTCTT |
| | | CATCT | | GCTTG |
| | | CAAAG | | AGACC |
| rs11632601 | 577 | TTCCC | 578 | CAGCT |
| | | TTGTT | | TCCAC |
| | | TGGAA | | CCTCT |
| | | CCCTG | | CCAC |
| rs11971741 | 579 | TGGCC | 580 | GGTGA |
| | | TTAAA | | CAATC |
| | | CATGC | | TAGAG |
| | | ATGCT | | AGGTG |
| rs12660563 | 581 | AGGTC | 582 | GCTCC |
| | | AGCTC | | ATTGA |
| | | AGGGT | | AGGGT |
| | | GAAGT | | AAAGG |
| rs13155942 | 583 | GAGGG | 584 | GCTCA |
| | | TACCT | | GTGTC |
| | | TTCTT | | TGACA |
| | | TCTCC | | AAAGC |
| rs17773922 | 585 | AGCCA | 586 | CAGTG |
| | | TGTTT | | CCTGA |
| | | CAGGG | | CAGGG |
| | | TTCAG | | AAAGT |

TABLE 5
reference nucleic acids and
oligos and primers

| | |
|---|---|
| RNaseP Loci | TCTTTCCCTACACGACG |
| PCR forward | CTCTTCCGATCTCTCCC |
| primer sequence | ACATGTAATGTGTTG |
| | (SEQ ID NO: 1337) |
| RNaseP Loci | GTGACTGGAGTTCAGAC |
| PCR reverse | GTGTGCTCTTCCGATCT |
| primer sequence | CATACTTGGAGAACAAA |
| | GGAC |
| | (SEQ ID NO: 1338) |
| RNaseP variant | CTCCCACATGTAATGTG |
| (rev_comp)* | TTGAAAAAGCATGGATA |
| | ACGGTGTCCTTTGTTCT |
| | CCAAGTATG |
| | (SEQ ID NO: 1339) |
| ApoE Loci PCR | TCTTTCCCTACACGACGC |
| forward primer | TCTTCCGATCTCCAGGAA |
| sequence | TGTGACCAGCAAC |
| | (SEQ ID NO: 1340) |
| ApoE Loci PCR | GTGACTGGAGTTCAGACG |
| reverse primer | TGTGCTCTTCCGATCTCA |
| sequence | ATCACAGGCAGGAAGATG |
| | (SEQ ID NO: 1341) |
| ApoE variant | CCAGGAATGTGACCAGCA |
| (rev_comp)* | ACGCAGCCCACAAAACCT |
| | TCATCTTCCTGCCTGTGA |
| | TTG |
| | (SEQ ID NO: 1342) |

*The underlined nucleotide is one that is different from the native sequences.

### Example 2. Design SNP Panels with Improved Sensitivity

The Transplant Monitoring v1 228plex panel, which include the 226 SNPs in Panel A described above is a highly multiplexed PCR-based target enrichment designed for non-invasive detection of donor-derived DNA (dd-DNA) in HSCT patients. The panel targets 226 SNPs for measuring donor fraction and 2 synthetic competitors (i.e., ApoE and RNase P variant oligonucleotide sequences, as disclosed in Example 1) for measuring the total amount of copies of DNA input. The donor fraction, the percent of DNA that is donor-derived in recipient plasma, is used as a biomarker for organ injury and acute rejection. During the course of a transplant rejection and subsequent cell damage in a graft, dd-DNA is released and the donor fraction increases. The total copies are used as a quality control metric for the donor fraction measurement as the measurement of donor fraction will lose accuracy if there are insufficient amounts of DNA used in the PCR reaction.

The key variable used for measuring both total copies and donor fraction is the allele frequency of each of 228 targets. This is the ratio of counts of the reference allele to the sum of both reference and alternate allele counts. In a pure sample, with DNA from a single individual, a biallelic SNP can only have an allele frequency of 0 (homozygous for alternate allele), 0.5 (heterozygous for reference and alternate allele), or 1 (homozygous for reference allele). An HSTC transplant patient's DNA is a mixture of donor and recipient DNA. Donor fraction is determined from “informative” SNPs—where the allele frequency is shifted from 0, 0.5, or 1 due to a difference in donor genotype and recipient genotype. This occurs for example when the recipient is homozygous for an allele (e.g. AA) and the recipient is either heterozygous (e.g. AB) or homozygous for a different allele (e.g. BB).

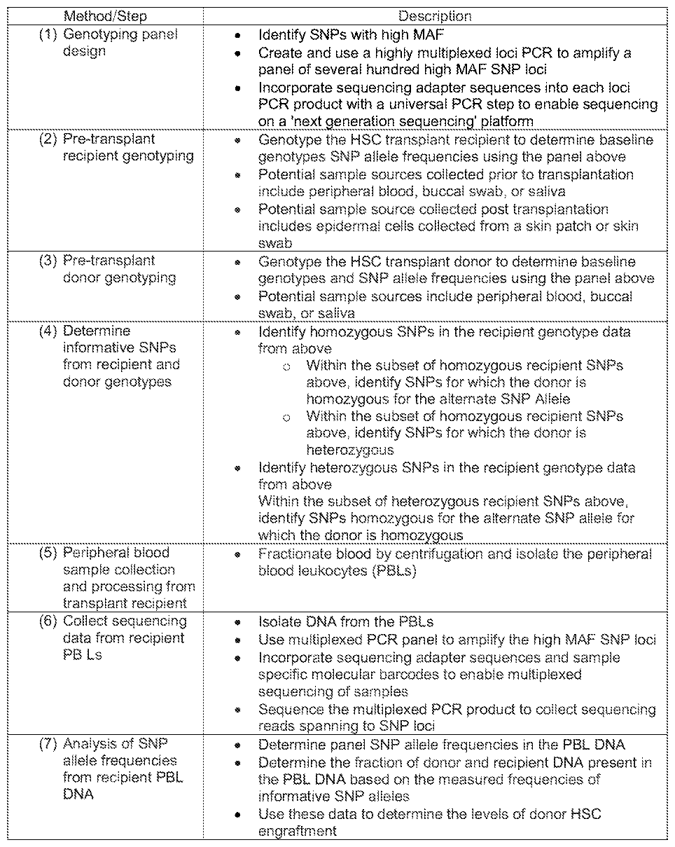

During characterization of the v1 panel (the v1 panel refers to the SNP panel A in Table 1 and two synthetic competitors for measuring the total amount of copies of DNA input, as described in Example 1), it was determined that certain categories of SNPs had higher amount of bias and variability in their allele frequencies. For a homozygous SNP, the allele frequency should be equal to 0 or 1. Background is defined as a median bias away from 0 or 1. This is caused in part by sequencing error or PCR error. The variability is the median absolute deviation (MAD) of the homozygous allele frequencies—in an error free measurement, this would be 0. When these biallelic SNPs are categorized by their combinations of reference and alternate alleles (abbreviated as Ref_Alt), it is observed that A_G, G_A, C_T, and T_C have the highest median and MAD for homozygous SNPs FIG. 9) and represent 78.5% of the panel (FIG. 10). These Ref_Alt combinations serve as a lower limit to the donor fraction that can be detected.

This motivated the development of a v2 panel that has only lower background Ref_Alt combinations in order to improve sensitivity for low levels of donor fraction. The v2 panel retains 47 SNPs from the v1 panel and adds in 328 new assays that all have the desired Ref_Alt combinations (not any of A_G, G_A, C_T, or T_C).

The first step in the design process was to identify SNPs that can serve as a universal individual identification panel. The goal was to be able to distinguish dd-DNA from recipient DNA regardless of the population (e.g. Asian, European, African, etc.). The ALlele FREquency Database (ALFRED, site: http:Hafred.med.yale.edu/afred/sitesWithfst.asp) provides allele frequency data on human populations. The Fixation Index (FST) is the proportion of total genetic variance contained in a subpopulation relative to the total genetic variance. A low value is desirable for obtaining a SNP that will have similar genetic variance in most populations. The first step in panel development was to filter this database to obtain SNPs with a FST lower than 0.06 based on a minimum of 50 populations. The SNPs were further filtered to ensure a minimum average heterozygosity of 0.4 (the maximum possible is 0.5). This increases the proportion of SNPs in the panel that will be “informative,” increasing the confidence in the measurement of donor fraction. This filtering resulted in 3618 SNPs.

FASTA sequences were obtained for these SNPs from dbSNP (site: ncbi.nlm.nih.gov/projects/SNP/dbSNP.cgi?list=rslist). On average, this provided a 1001 bp flanking sequence that included the SNP plus 500 bp both upstream and downstream of the SNP. These sequences were used in the primer design tool BatchPrimer3 (site: probes.pw.usda.gov/batchprimer3/) along with the following parameters to obtain candidate primers for each SNP:

Product size Min: 40; Product size Max: 54;
Number of Return: 1; Max 3′ stability: 9.0;

### Max Mispriming: 12.00; Pair Max Mispriming: 24.00;

### Primer Size Min: 18; Primer Size Opt: 20; Primer Size Max: 24;

### Primer Tm Min: 52.0; Primer Tm Opt.: 60.0; Primer Tm Max: 64.0; Max Tm Difference: 10.0;

### Primer GC % Min: 30.0; Primer GC % Max: 70.0;

Max Self complementarity: 8.00; Max 3′ Self Complementarity: 3.00;

### Max #Ns: 0; Max-Poly-X: 5;

### Outside Target Penalty: 0;

### CG Clamp: 0;

### Salt Concentraion: 50.0;

### Annealing Oligo Concentration: 50.0.

Processing through BatchPrimer3 resulted in 2645 assays that met the design criteria. These SNPs were further filtered based on additional characteristics obtained from the dbSNP database. SNPs were selected if they met all of the following criteria:
1. Biallelic.
2. The SNP is not located within the primer annealing regions.
3. Validated by the 1000 Genomes Project.
4. The ref_alt combination is not any of A_G, G_A, C_T or T_C.
5. minor allele frequency is at least 0.3.
6. The sequence for amplified target region is unique and cannot be found elsewhere in the genome.

The result is a 377plex panel that includes the 2 assays for total copy calculation and 375 assays for donor fraction measurement. The donor fraction assays consist of 47 primers from the v1 panel and 328 newly designed primers. This panel was further filtered to obtain a 198plex (2 for total copies, 196 for donor fraction) (Table 6) after removing assays with low depth, high allele frequency bias (deviation from 0, 0.5, or 1 in a test with pure samples), or having a significant role in lowering the alignment or on-target rate (determined from re-aligning unaligned or off-target reads to first 18 bp of each of the primers). Table 7 lists the excluded SNPs and provides reasons for their exclusion. The first primer and the second primer were used as a primer pair to amplify the region containing the SNP in the same row in Tables 6 and 7.

TABLE 6
Panel v2

| | | First | | Second |
|---|---|---|---|---|
| | SEQ ID | Primer | SEQ ID | Primer |
| SNP | NO | Sequence | NO | Sequence |
| rs150917 | 587 | CTGTT | 588 | TCGAA |
| | | TTCTC | | AGAAA |
| | | AGAAG | | ACACT |
| | | GGACT | | GAGAA |
| | | TT | | TCAA |
| rs163446 | 589 | TGGAC | 590 | AGATC |
| | | AAAAA | | ATCCT |
| | | TACCA | | GAACA |
| | | TCATC | | TAAGG |
| | | A | | T |
| rs191454 | 591 | TTCCC | 592 | CACCA |
| | | TCTTC | | AGAAG |
| | | AGTTT | | GGAAT |
| | | ACCTG | | GAAAA |
| | | TTT | | T |
| rs224870 | 593 | TGAAG | 594 | AAGCC |
| | | AAAGC | | GCGTG |
| | | AAGGG | | TTATT |
| | | ACAGA | | GAAAC |
| | | A | | |
| rs232504 | 595 | TTCAG | 596 | CACAC |
| | | TGCTT | | ACACG |
| | | TCCGT | | CACTA |
| | | TGGA | | AGCAA |
| rs258679 | 597 | TCACC | 598 | AATAC |
| | | TCATA | | CTCAA |
| | | CATGT | | AGGAC |
| | | TTTCT | | TGTAA |
| | | TTT | | TG |
| rs260097 | 599 | TGCTG | 600 | GAACT |
| | | CATTC | | CTGGT |
| | | ATTTG | | GTTCC |
| | | TCAAC | | TAGTG |
| rs376293 | 601 | TGTAT | 602 | GGCAG |
| | | TTGCC | | AGTTC |
| | | TAAAA | | TCTTG |
| | | GTAAG | | ACGTG |
| | | AGG | | |
| rs390316 | 603 | AAGGA | 604 | AGGCT |
| | | AGTAA | | AACTC |
| | | AGGTA | | TAACA |
| | | TGTGC | | TCCTG |
| rs468141 | 605 | ACTTA | 606 | TTATT |
| | | AAACC | | GGGTG |
| | | AAACC | | TTGCA |
| | | CTCA | | AGTGT |
| rs500399 | 607 | GTTTA | 608 | GGGCA |
| | | TTGAT | | GAGTG |
| | | GAACT | | ATATC |
| | | GGTGC | | ACAG |
| rs522810 | 609 | TCCCC | 610 | CAGCA |
| | | TCTAC | | CTGAT |
| | | CCCTT | | GACAT |
| | | GAAGC | | CTGGG |
| rs534665 | 611 | ACGGG | 612 | GCCTG |
| | | GTCTT | | AGAAG |
| | | ATGGT | | CAATT |
| | | TCCTC | | AACCT |
| | | | | G |
| rs535468 | 613 | TGCTA | 614 | TTTAT |
| | | ACCTG | | TTGCA |
| | | TGAAG | | TTGGT |
| | | TCCAT | | CTTTG |
| | | TC | | C |
| rs535689 | 615 | GCATA | 616 | CGATT |
| | | ATTTG | | ATGCC |
| | | AAAGC | | CATTG |
| | | TCTGT | | ATATT |
| | | TTG | | TTT |
| rs535923 | 617 | TCAAG | 618 | CTCCA |
| | | GGATT | | AACCA |
| | | GCTCC | | ATACC |
| | | AATGT | | TAAAA |
| | | | | A |
| rs567681 | 619 | CCAGC | 620 | GGAGA |
| | | CCTGC | | AGATC |
| | | TCCTT | | CTACA |
| | | TAATC | | CTCAG |
| rs570626 | 621 | GCTTC | 622 | CCTAG |
| | | TCATC | | AATAT |
| | | TGTGT | | GATGC |
| | | GCATT | | CCAAA |
| | | T | | CA |
| rs580581 | 623 | CCTCC | 624 | TGTAG |
| | | TCTAC | | AATAA |
| | | TAGAC | | GAAGG |
| | | CTCTG | | CAGTC |
| | | ACG | | CAA |
| rs600810 | 625 | ACCTA | 626 | AAGCC |
| | | GGGAA | | AGGGT |
| | | GGGGT | | TCATC |
| | | CAC | | TGC |
| rs622994 | 627 | CATCC | 628 | GGTGT |
| | | TACCT | | CTTAG |
| | | CTAGG | | TTACA |
| | | TACAC | | TGTGC |
| rs698459 | 629 | TCCAA | 630 | TCAAC |
| | | AATTC | | CTCCT |
| | | CTTGA | | ACAGC |
| | | TGTGT | | AACAA |
| | | CA | | AA |
| rs707210 | 631 | GGTTC | 632 | ATGTA |
| | | ACTAC | | CCTTT |
| | | AGAGC | | TGGGC |
| | | GTCTC | | CTTGC |
| | | AA | | |
| rs729334 | 633 | CCACC | 634 | TGATT |
| | | AACCT | | TGTGA |
| | | GCCTC | | TCAGT |
| | | TGG | | CTTCC |
| | | | | TCTT |
| rs747190 | 635 | ATTCT | 636 | TTTGG |
| | | TCCTC | | AAGTC |
| | | CTGCA | | GGTGC |
| | | ATCCA | | TAACC |
| rs751137 | 637 | GGCTT | 638 | CAAAG |
| | | GCTTA | | ATTGC |
| | | ACATG | | AGATA |
| | | TGCTG | | AAGTG |
| | | | | CT |
| rs765772 | 639 | TTCCT | 640 | TCCCA |
| | | TGGCA | | TGTAA |
| | | TTTTA | | CACCT |
| | | GTTTC | | TTCAG |
| | | C | | A |
| rs810834 | 641 | TTTGC | 642 | GGAAC |
| | | ATTCT | | CACTA |
| | | CCTGT | | CAGGA |
| | | CTCTT | | AACGA |
| | | TTT | | A |
| rs827707 | 643 | TTTTG | 644 | CTCCA |
| | | CCAAG | | TCGAG |
| | | CTATT | | GGATT |
| | | CACAG | | ATCAG |
| | | | | A |
| rs876901 | 645 | GCACC | 646 | AGAAT |
| | | TATTC | | CTTCC |
| | | ACAGA | | GATTC |
| | | CAGTT | | TGCAT |
| | | TGA | | |
| rs895506 | 647 | GCCCC | 648 | GAGGA |
| | | TATAA | | GCCAA |
| | | TCCTT | | AGAGC |
| | | GGAGT | | TGAAA |
| | | C | | |
| rs930698 | 649 | GGTTT | 650 | AGGAG |
| | | CATTA | | ATGTG |
| | | CTCTA | | CATTT |
| | | TGCTT | | CAGCA |
| | | CTTC | | |
| rs937799 | 651 | CAGGA | 652 | TTTTA |
| | | CAGGA | | AATAC |
| | | ATTAG | | TACGG |
| | | TGTTG | | AGTCA |
| | | C | | AAC |
| rs955456 | 653 | GCCCT | 654 | GCAGG |
| | | TGAAA | | ATATT |
| | | AGAGG | | CTCTG |
| | | GCTTA | | ACTGC |
| | | | | AA |
| rs974807 | 655 | AAAGA | 656 | CGTGT |
| | | GTATA | | AGTAG |
| | | GGGAT | | TCACC |
| | | GGACA | | CGGTT |
| | | CTGA | | T |
| rs994770 | 657 | GAAAG | 658 | TTTTC |
| | | CCTAC | | AGTGT |
| | | ACGCC | | CCTCA |
| | | CAAG | | CCTCT |
| | | | | GA |
| rs1002142 | 659 | TCCAA | 660 | GAGCC |
| | | CTGGA | | ACCTT |
| | | AAACA | | CAAGA |
| | | CCTCA | | CTCTT |
| | | | | TC |
| rs1017972 | 661 | CAAAA | 662 | ACTGA |
| | | TTTCC | | TTCCT |
| | | AGCGC | | CGCAG |
| | | ATTCT | | CCTTG |
| rs1057501 | 663 | ACTGC | 664 | AAAAG |
| | | ATTGT | | TACAT |
| | | GGCGG | | GATGC |
| | | TATCT | | ATTTA |
| | | | | AGC |
| rs1145814 | 665 | AAAAC | 666 | AATAG |
| | | ATAAT | | GAGGC |
| | | TGAAC | | TGCTC |
| | | ACCTA | | TATGC |
| | | GCA | | |
| rs1278329 | 667 | CGCTG | 668 | ACATG |
| | | GTAAA | | TTCCC |
| | | TACTT | | CATTG |
| | | AGAGA | | CTCA |
| | | TAAA | | |
| rs1336661 | 669 | CAGTC | 670 | GCAAC |
| | | TTGTT | | TGAGA |
| | | GTATT | | GGATG |
| | | CCCTA | | AGGTT |
| | | AAGA | | G |
| rs1340562 | 671 | GACCT | 672 | GTGCA |
| | | AAGAC | | AAGGA |
| | | TAGTG | | AACCA |
| | | CCGTG | | GGAGA |
| | | AA | | |
| rs1356258 | 673 | GGAAT | 674 | TTACC |
| | | AATAT | | CTTAA |
| | | ATGTG | | AAATT |
| | | GACTG | | CCTTG |
| | | CTT | | G |
| rs1396798 | 675 | AAAGC | 676 | TTGGT |
| | | AAATG | | TCTTT |
| | | GTTAA | | CTCTT |
| | | ATAGC | | TAATT |
| | | AGA | | GTG |
| rs1406275 | 677 | CAGAG | 678 | CCAAG |
| | | AGAAA | | ATACC |
| | | GCAGT | | TTGCC |
| | | TTGAA | | TTCTG |
| | | TTTG | | A |
| rs1437753 | 679 | CATCA | 680 | TCCTT |
| | | TATTC | | GGTAA |
| | | CTAAC | | AGAGG |
| | | TGTGC | | GTAAA |
| | | TCAT | | GAAA |
| rs1442330 | 681 | TACTG | 682 | TTAGA |
| | | CCAAC | | CCGCA |
| | | AGACA | | GACCT |
| | | ACTCG | | TTAGA |
| | | | | A |
| rs1444647 | 683 | GTCTA | 684 | CTACA |
| | | CTTCA | | TGCAT |
| | | AATCA | | ATCTG |
| | | TGCCT | | GAGAC |
| | | C | | |
| rs1482873 | 685 | ACTGA | 686 | TGGTT |
| | | GGAGT | | TTACC |
| | | AATTC | | TTTCT |
| | | ATGAG | | GAAAA |
| | | G | | ACA |
| rs1512820 | 687 | CACCT | 688 | CCTAA |
| | | CCTAA | | TCCAG |
| | | GACAA | | CAGAC |
| | | AATGG | | CATGT |
| | | CTA | | |
| rs1517350 | 689 | GGAGG | 690 | GCATA |
| | | CAGAA | | GCCAG |
| | | ATTGC | | CCATT |
| | | ATCAG | | AGCAT |
| rs1566838 | 691 | TCTCA | 692 | GCCCA |
| | | GAGCA | | ATCAG |
| | | ACATG | | ACATC |
| | | TACCA | | AATCC |
| | | AAA | | |
| rs1584254 | 693 | CCTCA | 694 | GAAGA |
| | | AGGCC | | GTTTT |
| | | TCTCC | | GACTT |
| | | ATTG | | TTTCT |
| | | | | GAGG |
| rs1610367 | 695 | ATCCC | 696 | ACAGC |
| | | CAAGC | | CATGA |
| | | CCAAG | | ACGAA |
| | | AAG | | GCATT |
| rs1714521 | 697 | GGCTC | 698 | AAGAA |
| | | ATGAA | | AGATT |
| | | CTAAG | | GTGGG |
| | | ATAGT | | ATTAG |
| | | TTGG | | ACA |
| rs1769678 | 699 | CCATC | 700 | TTGGA |
| | | AGAGC | | GGAGA |
| | | TTAGG | | AAGGC |
| | | GTTGA | | ATCAG |
| | | A | | |
| rs1979581 | 701 | CCATC | 702 | CCATC |
| | | TTAGT | | TTCTT |
| | | TGGAA | | TTCCC |
| | | ATAGC | | AAGCA |
| | | AACC | | |
| rs1990103 | 703 | ACATG | 704 | TTCTT |
| | | CTCCT | | GACGG |
| | | AGGGT | | TGTTC |
| | | GCTTC | | TGTTT |
| | | | | TT |
| rs2004187 | 705 | CCCTT | 706 | CCCTA |
| | | GTTGG | | TTTCC |
| | | GGAAA | | TACTG |
| | | TAACA | | AACGC |
| | | | | TTA |
| rs2010151 | 707 | TTGGA | 708 | CAAAC |
| | | ATGTC | | CCATG |
| | | CATCC | | GCCTT |
| | | TTTGA | | GAA |
| | | G | | |
| rs2022962 | 709 | GGTAT | 710 | AAGGT |
| | | GTATG | | TATGT |
| | | TGGGA | | AAGAA |
| | | AGGGA | | AGATG |
| | | AT | | TCA |
| rs2038784 | 711 | AAGGA | 712 | TGGGG |
| | | AGAAT | | CTAAA |
| | | TCTCA | | AGTCA |
| | | ATGAC | | GACCA |
| | | CT | | |
| rs2040242 | 713 | TTTAA | 714 | CTATT |
| | | GATAT | | AGTTA |
| | | GCTCT | | GGTTT |
| | | CTCCT | | CCAGT |
| | | GACT | | TGA |
| rs2055451 | 715 | AGGAA | 716 | CCTAA |
| | | ATCTG | | TAGAC |
| | | TGAGT | | CTAAC |
| | | AACTA | | AAGGA |
| | | TCAT | | TGC |
| rs2183830 | 717 | GCAAT | 718 | TGGAG |
| | | GATAA | | CCAAA |
| | | CAAGA | | GGGAG |
| | | ACACA | | TAATA |
| | | GCA | | |
| rs2204903 | 719 | TCTCT | 720 | TGTGT |
| | | CCACC | | GAAAC |
| | | TTTCC | | CTGTG |
| | | ACACT | | ACTTG |
| | | G | | C |
| rs2244160 | 721 | CATAT | 722 | TGTGG |
| | | TCATA | | AAACA |
| | | CCTTC | | CAGCC |
| | | AAGCC | | CATT |
| | | AAC | | |
| rs2251381 | 723 | GAAAG | 724 | CCCAT |
| | | GGATG | | GAACA |
| | | ATGGT | | CATTC |
| | | TCCAA | | ACAGC |
| rs2252730 | 725 | CAGGA | 726 | CAGAG |
| | | ACTCG | | GAGCA |
| | | CTGAA | | CCAGC |
| | | TACCC | | CTATG |
| rs2270541 | 727 | GCCAT | 728 | CAATC |
| | | GAATT | | CAACG |
| | | AGGAG | | AAGAT |
| | | CCTTG | | GACCA |
| rs2291711 | 729 | ACCAT | 730 | GGACG |
| | | GACCT | | ATCAG |
| | | GGCTT | | GTTAC |
| | | GAAGT | | ACCTA |
| | | | | AAA |
| rs2300857 | 731 | TCCAC | 732 | CAGCT |
| | | CTCCT | | GAACA |
| | | AACCA | | CTGAG |
| | | AGGAC | | ATTTT |
| | | | | T |
| rs2328334 | 733 | AAGCC | 734 | CATCT |
| | | CTGTT | | GCAGA |
| | | TCCCT | | AGACA |
| | | GTTTT | | GACTC |
| rs2373068 | 735 | ATCAT | 736 | GACAC |
| | | TCCCG | | AATGT |
| | | GAGCT | | GCCTT |
| | | CACA | | GAAA |
| rs2407163 | 737 | GTACA | 738 | CCCAG |
| | | GCTGG | | TTTCC |
| | | AATGG | | ATCCT |
| | | CCAAG | | CAGTC |
| rs2418157 | 739 | AACAA | 740 | TCTTG |
| | | TTTGC | | GCCTT |
| | | TCTGA | | CAGGG |
| | | GAACC | | TTTC |
| | | TC | | |
| rs2469183 | 741 | CCTTT | 742 | TCGTT |
| | | GTTAC | | TCTTA |
| | | TAAGA | | TTGTC |
| | | ATTGA | | TTCTG |
| | | AGTG | | TT |
| rs2530730 | 743 | CTCCC | 744 | CCACC |
| | | AATAT | | TCAGG |
| | | CCGAC | | ACAGG |
| | | AGCTC | | AGAGT |
| rs2622244 | 745 | TGGAT | 746 | CTGAG |
| | | TGATG | | GGCTT |
| | | GCAGA | | TTTGG |
| | | ACATT | | CTAAC |
| rs2794251 | 747 | TTTTA | 748 | TCAGA |
| | | TTTTT | | GAGAT |
| | | CTCAC | | AAAGA |
| | | AAGCC | | AGGAA |
| | | TGA | | AGGA |
| rs2828829 | 749 | TCTAA | 750 | GGCTG |
| | | TTAAG | | TGGTA |
| | | CCATG | | TGGCT |
| | | ACTCC | | AGCAG |
| rs2959272 | 751 | CACAG | 752 | AGGCA |
| | | AGAAA | | GACAG |
| | | GAACA | | ATGGA |
| | | GAATC | | CACAT |
| | | TGAA | | |
| rs3102087 | 753 | GAGCT | 754 | CCCAG |
| | | TTGCA | | CCTCT |
| | | TGCAG | | CTGTC |
| | | TAGGG | | TATGG |
| rs3103810 | 755 | TGACT | 756 | GTGCA |
| | | TCTAT | | GGAGA |
| | | CACCC | | GGAAA |
| | | CTACC | | GCAGA |
| rs3107034 | 757 | GTTGA | 758 | GCACG |
| | | TGACA | | ACGTA |
| | | CCCAC | | CGAAT |
| | | ATTCA | | GAGTC |
| rs3128687 | 759 | AGCAC | 760 | GAAGG |
| | | CAGGC | | ATGTG |
| | | TTTGG | | AGAAA |
| | | CTAT | | AGACC |
| | | | | TG |
| rs3756508 | 761 | GCATG | 762 | CAAGC |
| | | GTCAC | | CACAA |
| | | TGAGT | | GAGGT |
| | | TTTGC | | GATGA |
| rs3786167 | 763 | CACAG | 764 | TGGTA |
| | | AACAG | | CTAAG |
| | | CTTGT | | ACCCA |
| | | GAAAA | | CCAAA |
| | | TCA | | A |
| rs3902843 | 765 | AAAAC | 766 | GCTTG |
| | | CCTCT | | CTCTT |
| | | AACTA | | ATTAT |
| | | GGCAT | | TTTGA |
| | | TGAA | | CGTT |
| rs4290724 | 767 | AGAAT | 768 | AAACA |
| | | TTGGA | | GATCC |
| | | ACTCA | | TATTG |
| | | CTTTG | | TGTCT |
| | | G | | GGAA |
| rs4305427 | 769 | ACCTC | 770 | AAGTG |
| | | ATGCA | | TTGCT |
| | | CCAGC | | CCCTG |
| | | CCTTA | | CTGTC |
| rs4497515 | 771 | AAAGG | 772 | AGGTG |
| | | TCTTT | | GCCAT |
| | | CAGGA | | ACACA |
| | | GAATT | | TGCTT |
| | | TG | | |
| rs4510132 | 773 | GGTTG | 774 | TTTGC |
| | | TCCAT | | AGTGT |
| | | GTCCC | | TTATG |
| | | CAAG | | CCACA |
| rs4568650 | 775 | TCATG | 776 | TTTAA |
| | | GCAAT | | ATGGT |
| | | TTAAA | | GCCTT |
| | | TGATG | | GTTTC |
| | | AG | | TT |
| rs4644241 | 777 | CAGGG | 778 | GGGAT |
| | | CACTA | | ATGGA |
| | | ACTGA | | TTATC |
| | | AAAAT | | TTTCT |
| | | | | CAT |
| rs4684044 | 779 | AGCCC | 780 | CCCAG |
| | | CAAAC | | AGCCA |
| | | TAAGT | | GTGCA |
| | | GCTGA | | TTTA |
| rs4705133 | 781 | TGATG | 782 | CCTGG |
| | | AGAAA | | CTGAA |
| | | ACACA | | TCAAG |
| | | GAAAT | | GAAGA |
| | | GC | | |
| rs4712565 | 783 | CAGTG | 784 | TAGGA |
| | | ACAGT | | ACAAT |
| | | TTTCT | | CCCCA |
| | | CATTA | | ATCCA |
| | | AGC | | |
| rs4816274 | 785 | TGAGA | 786 | TGACA |
| | | AACTC | | GCAAT |
| | | ACTTG | | TCTGG |
| | | GGGTC | | TCTGC |
| | | A | | |
| rs4846886 | 787 | AGGCT | 788 | CIIII |
| | | TGAAG | | ICATA |
| | | AAAAG | | TCCAG |
| | | CTTCA | | TATTT |
| | | T | | CAG |
| rs4910512 | 789 | CAGCT | 790 | GGATA |
| | | AGAAT | | CAACA |
| | | CTATA | | GGAAC |
| | | CAAGG | | TAGGA |
| | | AAGG | | TCAA |
| rs4937609 | 791 | CCCAT | 792 | TCTGA |
| | | TATTA | | GAGTT |
| | | TGCTG | | AAATC |
| | | TTATG | | CTTGG |
| | | CTG | | TGA |
| rs6022676 | 793 | CACCT | 794 | GGCCG |
| | | CTTAA | | ACAGC |
| | | CAGTT | | TTCTA |
| | | TCATT | | CTTTA |
| | | TT | | |
| rs6023939 | 795 | AAGGA | 796 | GCTCT |
| | | GGGCT | | TTCTC |
| | | TAGCT | | ATCTT |
| | | AGTTG | | AAGGC |
| | | | | TTC |
| rs6069767 | 797 | GTTAA | 798 | CAGGC |
| | | AATTA | | AACCA |
| | | CTGTT | | AATAA |
| | | CCAGT | | TAACA |
| | | TGT | | AAA |
| rs6102760 | 799 | GGATT | 800 | CACCT |
| | | CTGCA | | TGCCA |
| | | GACCC | | CTCAC |
| | | TCAGT | | TGTTG |
| rs6434981 | 801 | GGGTT | 802 | GGTAA |
| | | CCAGC | | TGAAG |
| | | AATAT | | AAAGA |
| | | TCTAC | | CAAAA |
| | | CTT | | CA |
| rs6489348 | 803 | CTGTG | 804 | GCACA |
| | | TGGCT | | TAACC |
| | | GGGGA | | TCAGA |
| | | AGC | | ACCAG |
| rs6496517 | 805 | GGAGC | 806 | ATCCT |
| | | CCCAA | | CATCC |
| | | CCCTA | | TCCGC |
| | | ATTT | | ACA |
| rs6550235 | 807 | CGGTA | 808 | GGGCA |
| | | GCTAA | | GGAAT |
| | | GTATC | | TATTA |
| | | TGCTT | | TGTTC |
| | | TTT | | CA |
| rs6720308 | 809 | GGATG | 810 | ACTTG |
| | | TTTTT | | CTCTG |
| | | GCAGT | | ATACC |
| | | TTATT | | TAAAT |
| | | | | GA |
| rs6723834 | 811 | CGGCT | 812 | GCATT |
| | | CTCTC | | GCCAC |
| | | CTCAT | | TGAGA |
| | | TCTGT | | CATGA |
| rs6755814 | 813 | AAGAG | 814 | TTTAG |
| | | GAGGG | | TAGAG |
| | | CTTTG | | CTACT |
| | | AGTCC | | GATCA |
| | | | | TTCC |
| rs6768883 | 815 | CAATT | 816 | AAGCC |
| | | AAGTC | | ATTCA |
| | | AGGTA | | TTTGG |
| | | ATAAT | | GTTTG |
| | | GCTG | | |
| rs6778616 | 817 | TTGAT | 818 | GGCCT |
| | | TCCTA | | CTGAC |
| | | TTGAG | | ATCAC |
| | | CTTTC | | TCTCA |
| | | A | | |
| rs6795216 | 819 | GGCAA | 820 | GGATT |
| | | GGGTT | | GCGCC |
| | | TAGGA | | TCAAA |
| | | CTTGG | | ATAAA |
| rs6834618 | 821 | CTTCC | 822 | CTGTT |
| | | CTGCA | | TAGGA |
| | | CATCC | | AGAGT |
| | | TTTTG | | CATGT |
| | | | | AACC |
| rs6840915 | 823 | TGGCC | 824 | CTGCA |
| | | TATTT | | AGGCA |
| | | CTCAA | | CGATC |
| | | ATGCA | | TATGA |
| | | G | | |
| rs6848817 | 825 | GTGAT | 826 | TGCAT |
| | | TCTAA | | GTTAA |
| | | CAGGT | | CACCA |
| | | ATGTA | | CATTG |
| | | ATGA | | AG |
| rs6872422 | 827 | GGAGA | 828 | TTTCG |
| | | CCATA | | AGTTG |
| | | CTGAA | | GTGGT |
| | | GTTAT | | AATTT |
| | | TTT | | |
| rs6902640 | 829 | TCGAA | 830 | GATAG |
| | | GGTAG | | TGACT |
| | | AATTA | | TATAA |
| | | AATGT | | CAACT |
| | | TTC | | CCAA |
| rs6979000 | 831 | TGAAT | 832 | GCACA |
| | | TGAAG | | CGTTA |
| | | GGTTT | | AGATG |
| | | TGGAC | | GTTTG |
| | | | | AA |
| rs7006018 | 833 | GGGGA | 834 | TCCAG |
| | | GGGAG | | ATTTT |
| | | ACGTA | | CCTGT |
| | | AAAAC | | TCATG |
| | | | | ATT |
| rs7045684 | 835 | GCACA | 836 | CCCCA |
| | | TCACA | | GTAGG |
| | | AGTTA | | GAACA |
| | | AGAGG | | CACTT |
| rs7176924 | 837 | CAGGA | 838 | GGCTT |
| | | TGCAC | | CTCCC |
| | | TTTTT | | AGAAA |
| | | GGATG | | ATCTC |
| rs7215016 | 839 | GGGGA | 840 | GAAGG |
| | | GGCCC | | GAGGG |
| | | TACAA | | GCATC |
| | | GTTAT | | TTTA |
| rs7321353 | 841 | AAAAT | 842 | TGGAC |
| | | CACAT | | GATAG |
| | | CTGCT | | AACTT |
| | | AAATA | | GTTAG |
| | | TCC | | TGC |
| rs7325480 | 843 | CCATT | 844 | CTCCT |
| | | AAGCA | | TTGAA |
| | | GACAC | | AGTGG |
| | | ACCTA | | ATCAA |
| | | CG | | A |
| rs7539855 | 845 | TCTGA | 846 | TCCTT |
| | | AAATG | | AAAGC |
| | | GGGCT | | AGCCC |
| | | AAAAC | | TAAAA |
| | | TT | | |
| rs7568190 | 847 | AGTTT | 848 | TGGAG |
| | | AGATT | | AATAG |
| | | TCAGT | | CTCCT |
| | | CTATG | | GCAGT |
| | | CAA | | T |
| rs7580218 | 849 | TCTTT | 850 | CTGGA |
| | | CTGGA | | ATCTA |
| | | GACAC | | GAAAG |
| | | TCAGG | | AAAAA |
| | | | | GAA |
| rs7609643 | 851 | CAAAG | 852 | CTGAC |
| | | ATAGA | | ATTGA |
| | | TGAGA | | AAACT |
| | | TGCTT | | TGAAA |
| | | TT | | GAA |
| rs7632519 | 853 | AGCCC | 854 | GCCCA |
| | | TCCTC | | GCTAC |
| | | CACCG | | GATTT |
| | | TTAG | | CTCCT |
| rs7660174 | 855 | TTTTA | 856 | CCCTT |
| | | TGCAG | | AGTTC |
| | | CCTGT | | AATCA |
| | | GATGG | | AGCCA |
| | | | | AC |
| rs7711188 | 857 | CACTC | 858 | CTGAC |
| | | TTGCA | | CCTTG |
| | | ATCTC | | TGGGA |
| | | CCTCA | | TTCAT |
| | | G | | |
| rs7765004 | 859 | CTTTT | 860 | TGGAT |
| | | ATGAT | | CATCT |
| | | ATCCA | | GTCCA |
| | | CCAAG | | AAGTC |
| | | ACT | | A |
| rs7816339 | 861 | CCAAA | 862 | AAGAC |
| | | ACCTG | | TACTG |
| | | CTCTC | | AGGTT |
| | | CAAGA | | GTGCA |
| | | | | AAGA |
| rs7829841 | 863 | TTCAA | 864 | AGTCA |
| | | CTTGG | | GTTAG |
| | | TACCC | | TATGC |
| | | TGAAA | | AGTAC |
| | | AA | | TTGG |
| rs7916063 | 865 | TCTTA | 866 | GGTCA |
| | | AAAGT | | ATGGC |
| | | GTCTT | | TAAAT |
| | | GACTG | | CATTC |
| | | AAA | | G |
| rs7932189 | 867 | GCAAT | 868 | TTATC |
| | | TCCAG | | TACCC |
| | | ATATC | | ATGCT |
| | | TCTTT | | TCTCT |
| | | AT | | C |
| rs7968311 | 869 | GCATA | 870 | TGTTT |
| | | AACAA | | TCGTA |
| | | ATGTG | | GTCTT |
| | | TAACG | | TATTG |
| | | TGGT | | CT |
| rs8006558 | 871 | TGCTA | 872 | CGTTA |
| | | GCTAT | | GTTCC |
| | | ATGTA | | CTGGA |
| | | GGTCA | | AAGAT |
| | | GTT | | CA |
| rs8054353 | 873 | TTGCA | 874 | GACTT |
| | | TAGAT | | TCTTA |
| | | GTAGC | | AAGCT |
| | | AGTAT | | GCACA |
| | | TTC | | ATCA |
| rs8084326 | 875 | GTTTG | 876 | TGTGA |
| | | CTTGC | | AGCAC |
| | | TTTTA | | CATTT |
| | | CTTTG | | CTGTT |
| | | | | T |
| rs8097843 | 877 | AACAG | 878 | CCCAT |
| | | TGAGG | | TGTCA |
| | | CTCTC | | CCGAG |
| | | CTGTA | | GATA |
| | | GC | | |
| rs9289086 | 879 | CAGAG | 880 | GCTAT |
| | | AGCTC | | CTTGG |
| | | ACTTC | | GTCAT |
| | | TAGTT | | GAATT |
| | | CTGC | | TG |
| rs9310863 | 881 | CCTCA | 882 | CATTT |
| | | TGCAA | | CCCCT |
| | | TTCAA | | AGGTT |
| | | AGGAA | | TGTGC |
| rs9311051 | 883 | GTGGG | 884 | CTTAG |
| | | GCACA | | ATTTG |
| | | CAGTG | | TTCAT |
| | | TCTT | | CTGAT |
| | | | | GGT |
| rs9356755 | 885 | TTGGG | 886 | AACCC |
| | | TAGAT | | ATATG |
| | | GCAAT | | ACTAA |
| | | GCAAG | | GGTGA |
| | | | | A |
| rs9544749 | 887 | GCTGA | 888 | TGTCA |
| | | AAATT | | TAATG |
| | | CACAC | | AAGAG |
| | | TGTGG | | CTAGT |
| | | TC | | TGC |
| rs9547452 | 889 | GAGAG | 890 | GAGTT |
| | | GTAAG | | ATTTC |
| | | AGAGA | | CCTTA |
| | | GTATC | | AAAAC |
| | | TTTG | | CAG |
| rs9814549 | 891 | GCTAC | 892 | GGATG |
| | | GCTTG | | CTGTG |
| | | ACACC | | AGTGC |
| | | CTTAC | | TAAAT |
| | | A | | GA |
| rs9861140 | 893 | GGCAC | 894 | CTGGC |
| | | TGCGT | | TCCTT |
| | | CAGCA | | GCCAT |
| | | TACTA | | CAT |
| rs9919234 | 895 | TAGGC | 896 | TGCTA |
| | | CTCAG | | GGCTT |
| | | AAAGA | | ACTTC |
| | | ACGAG | | GTTTT |
| | | | | C |
| rs9955796 | 897 | AAAAT | 898 | CATCA |
| | | AATTC | | TGAAT |
| | | CCTTT | | TCTCC |
| | | GGTAT | | CAATG |
| | | GC | | C |
| rs10073918 | 899 | TTGGG | 900 | TACCT |
| | | TAAAT | | GGGGC |
| | | GTGTG | | CCTGA |
| | | ACTAC | | TTTAT |
| | | GC | | |
| rs10096021 | 901 | GCACT | 902 | CCTTA |
| | | GAAAA | | GTGAG |
| | | TGTTA | | GTATT |
| | | GTGAT | | TAGGT |
| | | T | | TACA |
| rs10197959 | 903 | AGGGA | 904 | TGATC |
| | | GTTAT | | AGGGG |
| | | GATGC | | TAGAA |
| | | CAAGG | | GAGAT |
| | | | | TT |
| rs10233000 | 905 | CGGCT | 906 | GACAA |
| | | TCCAA | | GTCAG |
| | | TCGTA | | AGAAC |
| | | TCTTG | | AAGCT |
| | | | | G |
| rs10444584 | 907 | TCATC | 908 | TCAGG |
| | | TGTAA | | AAAGA |
| | | CTAAT | | ATGCT |
| | | GAACC | | ACTCA |
| | | TTG | | |
| rs10473372 | 909 | AATTG | 910 | TGCCA |
| | | GATGC | | CATGA |
| | | TGTTT | | CAAAT |
| | | TAACC | | TATCA |
| | | | | CA |
| 9rs1077730 | 911 | CCAAG | 912 | CTGAT |
| | | GTTTA | | AGAAA |
| | | GCTAC | | AATTT |
| | | ATGTA | | CTGTT |
| | | TAA | | GTG |
| rs10783507 | 913 | ATTCC | 914 | ATTCC |
| | | TTCCC | | TGCAC |
| | | GCCTT | | AGGCT |
| | | GCT | | CAGAC |
| rs10802949 | 915 | AAATG | 916 | AAAGG |
| | | TTCAG | | ACTAG |
| | | TGTAA | | CAGCA |
| | | AAGGC | | TGTAA |
| | | TACA | | CTC |
| rs10816273 | 917 | CACTA | 918 | AAGAT |
| | | CTTCC | | CTGGT |
| | | CCTTC | | AGAAA |
| | | CCAAA | | TAAAT |
| | | | | GGA |
| rs10817141 | 919 | GCTTC | 920 | AAAAA |
| | | CAGGC | | GAAAA |
| | | TAAAA | | GCTGG |
| | | GAAGG | | TTAGG |
| rs10892855 | 921 | CACCT | 922 | CCTGG |
| | | CTATG | | GATTG |
| | | GTTTA | | AAAGC |
| | | GTCCA | | ACCTA |
| | | CTCC | | |
| rs11098234 | 923 | GGAAT | 924 | AGTGG |
| | | TGCCA | | TCCCC |
| | | CTCTG | | AACAA |
| | | GAGAA | | CTTGA |
| rs11119883 | 925 | TCAGA | 926 | ACCCA |
| | | TAAAA | | CAGAG |
| | | CAATT | | GAAAG |
| | | CCAGT | | CCTTG |
| | | TAC | | |
| rs11157734 | 927 | CCTGC | 928 | CCATG |
| | | TGGCA | | GGAAT |
| | | CACGT | | TTGAA |
| | | AAGTT | | CCACT |
| rs11166916 | 929 | AACCA | 930 | GCCAA |
| | | CAATC | | GTCAT |
| | | CACCT | | TAACA |
| | | CTTGC | | CAAAG |
| | | | | TGA |
| rs11223738 | 931 | CCCAC | 932 | GAGAA |
| | | TCTTC | | GGGGA |
| | | TGCTT | | AAGAG |
| | | TACTC | | AACAA |
| | | CA | | A |
| rs11247709 | 933 | GGCTT | 934 | AGTGG |
| | | TTTCC | | GCAAT |
| | | ACCCA | | AATAA |
| | | GCTTA | | ACCTT |
| rs11611055 | 935 | GGTGG | 936 | AAAGA |
| | | CTGGA | | CAATT |
| | | GAAAT | | TGGCT |
| | | TGAGA | | GGTGT |
| | | | | TT |
| rs11627579 | 937 | GCTAA | 938 | TTCCC |
| | | GTTGC | | TATTT |
| | | CTCCA | | CTGCC |
| | | AGCTG | | AAAGC |
| rs11636944 | 939 | TTCAT | 940 | CAGAT |
| | | GGAGA | | ACTCC |
| | | TTTGA | | TTTTT |
| | | CCAGT | | GGAGA |
| | | G | | GTCA |
| rs11643312 | 941 | CAGCT | 942 | CCAGA |
| | | AATGC | | ACATT |
| | | ATAAG | | TCATC |
| | | GGAGA | | ACTCC |
| | | TG | | AA |
| rs11738080 | 943 | GTACA | 944 | CATGA |
| | | GAGTC | | TCTGT |
| | | CCTGT | | CTCTC |
| | | CTCAC | | TCACT |
| | | A | | GAA |
| rs11750742 | 945 | GTGGC | 946 | TGTGG |
| | | AGAAC | | GGGCA |
| | | TGACA | | GACAG |
| | | TGCAA | | ACT |
| rs11774235 | 947 | TCCAC | 948 | CCTCT |
| | | CAGAA | | GTGGA |
| | | ACCCT | | AAGGA |
| | | TTGG | | AGGAA |
| rs11785511 | 949 | CCCGC | 950 | AAGAA |
| | | TCCAG | | ATCTG |
| | | GTTAT | | AAAAG |
| | | TCTC | | CAGAG |
| | | | | G |
| rs11924422 | 951 | AACTG | 952 | TTTGA |
| | | ATTCA | | GAGGC |
| | | CATGA | | AACAT |
| | | GGTTG | | TAACA |
| | | C | | A |
| rs11928037 | 953 | AGTCT | 954 | TAAGG |
| | | GTACA | | CTCCT |
| | | AGGGG | | GTGGT |
| | | CCACA | | AGACG |
| rs11943670 | 955 | CATCA | 956 | CAAGA |
| | | TGGAA | | TCAAG |
| | | GGTCC | | GCATT |
| | | CTCAC | | GGTAG |
| rs12332664 | 957 | AGGTT | 958 | CCTTG |
| | | CAGAT | | CCTAA |
| | | TCTAT | | GATAA |
| | | TTCTG | | CACAA |
| | | TCA | | CCA |
| rs12470927 | 959 | TGTTT | 960 | CCTCA |
| | | TGTAA | | AATAC |
| | | TTCCT | | TGAAG |
| | | TTCAG | | ATAGC |
| | | TCA | | AAGC |
| rs12603144 | 961 | GACAA | 962 | GGGAG |
| | | GAACT | | GAACA |
| | | GAAGG | | GAACA |
| | | CAAAG | | ACCTT |
| | | G | | C |
| rs12635131 | 963 | TCGCA | 964 | TCCAA |
| | | GTCTT | | TAGCT |
| | | TTGCA | | ACCTT |
| | | TCATT | | CACCA |
| | | | | GAA |
| rs12669654 | 965 | GGTTA | 966 | GCAGT |
| | | AATTC | | GTAGT |
| | | TACTT | | CTAAC |
| | | CGCAA | | TAGCT |
| | | CCA | | GTGT |
| rs12825324 | 967 | CAGCT | 968 | AATTG |
| | | TCCCA | | CTACA |
| | | GTTTC | | TTCCT |
| | | TCACA | | GTCTA |
| | | | | TTG |
| rs12999390 | 969 | GCGGA | 970 | TGCAT |
| | | AAGAC | | CTCAA |
| | | ATTCC | | TGATA |
| | | ATGTT | | TTGCT |
| | | | | TTT |
| rs13125675 | 971 | TCTCT | 972 | TGTGC |
| | | GAGAG | | AATAG |
| | | CAAAG | | TAATA |
| | | ACACT | | ATGGG |
| | | | | TCT |
| rs13155942 | 973 | GAGGG | 974 | GCTCA |
| | | TACCT | | GTGTC |
| | | TTCTT | | TGACA |
| | | TCTCC | | AAAGC |
| rs17361576 | 975 | TGGCT | 976 | AAGCA |
| | | GCCTA | | AATAA |
| | | AAATT | | GGCCA |
| | | ATTTA | | TCTAA |
| | | CGA | | GAA |
| rs17648494 | 977 | TCAAA | 978 | GAAAA |
| | | CAAAA | | GTTAA |
| | | ACAGT | | GTCAG |
| | | GTAGG | | AGGCT |
| | | CATT | | ATCG |

TABLE 7
Excluded SNPs

| | SEQ | First | SEQ | Second | Reasons |
|---|---|---|---|---|---|
| | ID | Primer | ID | Primer | for |
| SNP | NO | Sequence | NO | Sequence | exclusion |
| rs31036 | 979 | AAGTC | 980 | AGACA | High |
| | | ACCTA | | CAGCA | Unmapped |
| | | AATGG | | AGATG | Reads |
| | | CATGA | | CAAA | |
| | | | | A | |
| rs42101 | 981 | CAGCA | 982 | TGTTT | High |
| | | ACCCT | | TCTCT | Unmapped |
| | | TTGAA | | TCAAA | Reads |
| | | GCAAT | | TGCAA | |
| rs164301 | 983 | TGACT | 984 | GCAGC | High |
| | | CAGTG | | CCATT | Unmapped |
| | | GTGAA | | AATAC | Reads |
| | | CTGTC | | TAGCA | |
| | | T | | CA | |
| rs232474 | 985 | TGCAT | 986 | TCAGG | Low |
| | | TCAAG | | ACGAA | Depth |
| | | AGGAA | | TTCAC | |
| | | GAAAG | | AGGAT | |
| | | G | | | |
| rs235854 | 987 | ATGAA | 988 | GAACA | High Off- |
| | | GGCCA | | TTCAC | Target |
| | | GGCTG | | TGCCT | Reads, |
| | | TAGG | | TACTC | Low |
| | | | | TCA | Depth, |
| | | | | | High |
| | | | | | Unmapped |
| | | | | | Reads |
| rs238925 | 989 | TTCAG | 990 | GGCCA | High |
| | | TGAAG | | CAGGA | Unmapped |
| | | GGATG | | TCTCC | Reads |
| | | GACCT | | TATCT | |
| rs242656 | 991 | CCAAG | 992 | GCTAG | High |
| | | TAATC | | CTACG | Unmapped |
| | | ACTTC | | CCCAC | Reads |
| | | AACCC | | GAGAT | |
| | | TCT | | | |
| rs243992 | 993 | AACTC | 994 | GGAAT | Low |
| | | AAACC | | GGAAT | Depth, |
| | | TAAGT | | AGTGT | High |
| | | GCCCC | | GTGGG | Unmapped |
| | | | | | Reads |
| rs251344 | 995 | ACACT | 996 | CACAC | High Off- |
| | | GGTCT | | CTGTA | Target |
| | | CAAGC | | ATTCT | Reads |
| | | TCCC | | AGCCC | |
| rs254264 | 997 | AGAAG | 998 | AGCTT | High Off- |
| | | GAAGG | | TCCTC | Target |
| | | ATCAG | | CCCAC | Reads |
| | | AGAAG | | ACTG | |
| rs265518 | 999 | TAACA | 1000 | AGAAG | High Off- |
| | | AATTT | | CCAGG | Target |
| | | GCATG | | TGCTG | Reads |
| | | TCATC | | AAGTG | |
| rs290387 | 1001 | GCTGT | 1002 | GAATG | High |
| | | GTGGA | | AAATG | Unmapped |
| | | GCCCT | | GAGTT | Reads |
| | | ATAAA | | TGCAG | |
| rs357678 | 1003 | GGCAG | 1004 | AGGTA | High |
| | | TGTTT | | GTGAT | Unmapped |
| | | AAGGT | | TTCTA | Reads |
| | | GTTGG | | GGCTT | |
| | | | | ATCA | |
| rs378331 | 1005 | CCTGG | 1006 | GGGAC | High Off- |
| | | AAGTA | | ATCTG | Target |
| | | TTCAT | | GGTAG | Reads |
| | | TCATG | | CACTG | |
| | | TGG | | | |
| rs425002 | 1007 | AAGAG | 1008 | AACTG | High Off- |
| | | TGTCT | | GAGGC | Target |
| | | CCTCC | | TGTGT | Reads |
| | | CTCTG | | TAGAC | |
| rs447247 | 1009 | AAAAA | 1010 | ATGTL | High Off- |
| | | CCCCA | | CAGUG | Target |
| | | GGCTC | | UTCTT | Reads |
| | | CATTG | | TTC | |
| rs499946 | 1011 | ATGGC | 1012 | TTCGG | High |
| | | TTGTA | | TGGAA | Unmapped |
| | | CTTCC | | TAGCA | Reads |
| | | TCCTC | | GCAAG | |
| rs516084 | 1013 | AGTAT | 1014 | CTTCT | High |
| | | GCCAT | | TTGAC | Unmapped |
| | | CATGA | | TAAGG | Reads |
| | | AAGCC | | CTGAC | |
| rs602182 | 1015 | GATCT | 1016 | TCATT | Low |
| | | TCCAG | | TTGGT | Depth |
| | | GGGGC | | TTCGT | |
| | | ACT | | TCATT | |
| rs621425 | 1017 | CCTTT | 1018 | GGCAT | High Off- |
| | | TGTGG | | TCCAA | Target |
| | | CTTTT | | CATGA | Reads |
| | | CCTCA | | AAAGG | |
| rs642449 | 1019 | CAGCT | 1020 | CCAAA | High |
| | | GCTGT | | AAACC | Unmapped |
| | | TCCCT | | ATGCC | Reads |
| | | CAGA | | CTCTG | |
| rs686106 | 1021 | GGTTC | 1022 | TGAGT | High |
| | | ACAGA | | CTCTT | Unmapped |
| | | GCCCA | | ACTGA | Reads |
| | | AGTTA | | TCCTG | |
| | | C | | TGAC | |
| rs751834 | 1023 | CTTCC | 1024 | CCAAA | High |
| | | CTCTG | | GAGCT | Unmapped |
| | | CCTCT | | CAGGT | Reads |
| | | TTTAG | | CTCCA | |
| | | A | | | |
| rs755467 | 1025 | AGGTG | 1026 | ACCTC | High |
| | | AGCAT | | TTCCT | Unmapped |
| | | GGGGT | | TCCTC | Reads |
| | | TGATA | | ACCAA | |
| rs842274 | 1027 | GGCAG | 1028 | TCATC | High Off- |
| | | CTCCA | | TTTTG | Target |
| | | CACAC | | GTTTT | Reads, |
| | | CTTAG | | AGATT | High |
| | | | | GTG | Unmapped |
| | | | | | Reads |
| rs893226 | 1029 | CAACT | 1030 | AAGAC | High Bias |
| | | GCCCG | | AGCTT | |
| | | CTTAT | | GAAGA | |
| | | CCTT | | TTCTG | |
| | | | | G | |
| rs898212 | 1031 | AAGGT | 1032 | ATGGC | High |
| | | CTAAG | | CACGC | Unmapped |
| | | GGGGC | | TCTTT | Reads |
| | | ACAAG | | GTC | |
| rs949771 | 1033 | CCAGA | 1034 | TGATT | High Off- |
| | | TTATC | | AGGGT | Target |
| | | TTCTT | | TGGGA | Reads |
| | | CGCCC | | AGTGG | |
| | | TA | | | |
| rs955105 | 1035 | TTCAG | 1036 | TGAAA | High |
| | | CTCTT | | CAAGA | Unmapped |
| | | CTACT | | GAAGA | Reads |
| | | CTGGA | | CTGGA | |
| | | CTG | | TTTG | |
| rs959964 | 1037 | CAAGT | 1038 | GGCCT | High Bias |
| | | TAGTG | | CTACT | |
| | | AGAAA | | CCAAG | |
| | | CAGAG | | AAAGC | |
| | | TCG | | | |
| rs967252 | 1039 | GTTAT | 1040 | TTGGA | High Bias |
| | | ATCTC | | TTGTT | |
| | | TTTTG | | AGAGA | |
| | | TTTCT | | ATAAC | |
| | | CTCC | | G | |
| rs1007433 | 1041 | GTCCA | 1042 | AGAGG | Low |
| | | GCTGT | | GAGAT | Depth |
| | | GTGAT | | GGAAT | |
| | | TATCT | | AAAAA | |
| rs1062004 | 1043 | AAAAA | 1044 | ACATA | High Off- |
| | | TAAAC | | GCCAC | Target |
| | | ATCCC | | CAGCC | Reads, |
| | | TGTGG | | ACACT | High |
| | | | | | Unmapped |
| | | | | | Reads |
| rs1080107 | 1045 | TGCTC | 1046 | ATATT | High Off- |
| | | TTTTT | | GGTCA | Target |
| | | CTCAC | | GTGGG | Reads |
| | | AAATG | | GCAAA | |
| | | A | | | |
| rs1242074 | 1047 | GCACA | 1048 | TGGCA | High Off- |
| | | TGAGC | | GTATT | Target |
| | | TGAGA | | ACCTG | Reads, |
| | | CTGGA | | AGCAA | High |
| | | | | | Unmapped |
| | | | | | Reads |
| rs1263548 | 1049 | GCAGC | 1050 | GCCCA | Low |
| | | GTCTT | | GCTCT | Depth |
| | | GCCTC | | TAACA | |
| | | CTT | | CAACA | |
| rs1286923 | 1051 | AAAAG | 1052 | TCAGA | High Off- |
| | | GCTGG | | AGGCA | Target |
| | | AGGAT | | CCTCT | Reads, |
| | | GAAGG | | GTCAC | High |
| | | | | | Unmapped |
| | | | | | Reads |
| rs1353618 | 1053 | TGCAA | 1054 | TCCCT | High |
| | | CCAAA | | TGCCT | Unmapped |
| | | ACTCA | | ATCAT | Reads |
| | | GTTAT | | TGCTT | |
| | | CTA | | | |
| rs1355414 | 1055 | TTCCC | 1056 | TACAA | Low |
| | | AGCCT | | TGGCT | Depth |
| | | TCCAG | | GACTG | |
| | | GAG | | AGCAC | |
| rs1418232 | 1057 | TGATT | 1058 | ATTCC | High Off- |
| | | TAAAC | | TGTCC | Target |
| | | CTGAT | | ACCCT | Reads, |
| | | CTTGG | | GGTC | High |
| | | TGA | | | Unmapped |
| | | | | | Reads |
| rs1474408 | 1059 | CCTTT | 1060 | TTACT | High |
| | | GATCA | | CTTGG | Unmapped |
| | | CAAGC | | GTCAG | Reads |
| | | AACCA | | GTGCA | |
| | | | | T | |
| rs1496133 | 1061 | ATGGC | 1062 | CGATG | High |
| | | AGAAG | | CTGAC | Unmapped |
| | | AGCCC | | CTTCT | Reads |
| | | AGAG | | GGAGT | |
| rs1500666 | 1063 | GCTGA | 1064 | GGAGT | High Bias |
| | | AAAAC | | TGAGG | |
| | | CCAGG | | GAGAG | |
| | | AATCA | | GGTCT | |
| rs1514644 | 1065 | GACAG | 1066 | CTTTC | High Off- |
| | | AATGA | | TAATC | Target |
| | | AATGC | | CAGCA | Reads, |
| | | TGTGT | | GCCTC | High |
| | | | | T | Unmapped |
| | | | | | Reads |
| rs1565441 | 1067 | CTGAT | 1068 | CAGGA | High Bias |
| | | CCCCG | | TGAAA | |
| | | TAAGA | | CGGTG | |
| | | TCAGC | | CAG | |
| rs1674729 | 1069 | TCTCT | 1070 | TAAGG | High Off- |
| | | GACCT | | CAATA | Target |
| | | GCTTC | | GGCAC | Reads |
| | | CTCGT | | CAAGC | |
| rs1858587 | 1071 | AGCAA | 1072 | AGCTG | High Off- |
| | | TGGGG | | ATTCC | Target |
| | | TCAGA | | TTCCC | Reads |
| | | GTCC | | TGGAT | |
| rs1884508 | 1073 | CCTGA | 1074 | CTGCA | High Off- |
| | | TGGAG | | AAGCT | Target |
| | | GATCC | | TCCCA | Reads |
| | | ACTTG | | TCCT | |
| rs1885968 | 1075 | GGGGA | 1076 | GACAC | High Off- |
| | | TCTTA | | TCCCA | Target |
| | | AAAGC | | CTTCT | Reads |
| | | ACCAA | | GCCTA | |
| rs1894642 | 1077 | ATTTC | 1078 | CAGGC | High Bias |
| | | TTCAA | | AAACA | |
| | | GTGTA | | TTCCC | |
| | | TACAG | | TTGTA | |
| | | AGC | | | |
| rs1915616 | 1079 | CACTG | 1080 | CTTCC | High Off- |
| | | TTGAC | | CACAA | Target |
| | | TCCAA | | CAATG | Reads, |
| | | AACAA | | AGCTG | High |
| | | | | | Unmapped |
| | | AAA | | | Reads |
| rs1998008 | 1081 | GCAGC | 1082 | TCTTT | High |
| | | TAAGA | | GCTCC | Unmapped |
| | | AAGAC | | CCACC | Reads |
| | | TCTCC | | TATT | |
| | | AA | | | |
| rs2056123 | 1083 | TGAAT | 1084 | AAGAT | High |
| | | TCAAC | | TTAAT | Unmapped |
| | | TGATG | | CCTTT | Reads |
| | | GCACA | | GAGAT | |
| | | | | GC | |
| rs2126800 | 1085 | TGAAA | 1086 | TTTTG | Common |
| | | GGACC | | TTGTG | Deletion |
| | | CACCA | | TGTTT | in Primer |
| | | AATGT | | GCTTT | Binding |
| | | | | | Region |
| rs2215006 | 1087 | TTGCT | 1088 | TACAG | High Off- |
| | | GGCTT | | CTCAG | Target |
| | | ACATT | | CCAGT | Reads |
| | | CATTC | | TCTGC | |
| | | C | | | |
| rs2226114 | 1089 | TGGTT | 1090 | GCCTT | Low |
| | | GGTAT | | AGTTT | Depth |
| | | GGTTA | | CTCTT | |
| | | TTATT | | TCTGT | |
| | | GG | | | |
| | | | | AAAA | |
| rs2241954 | 1091 | GGCCA | 1092 | TCCTA | High |
| | | GCACA | | GGACT | Unmapped |
| | | AACAC | | CTCCC | Reads |
| | | ACC | | TTTAG | |
| | | | | A | |
| rs2278441 | 1093 | AATGG | 1094 | CCAGT | High |
| | | GCAGA | | ACCTA | Unmapped |
| | | TGAGA | | CCCCA | Reads |
| | | GCAAG | | TGTCC | |
| rs2285545 | 1095 | TCTTT | 1096 | TGGCC | High |
| | | TTGAC | | CAATT | Unmapped |
| | | AGGTC | | TTCAG | Reads |
| | | CACAT | | TAACT | |
| | | C | | TC | |
| rs2288344 | 1097 | CACCA | 1098 | GAGTA | High Bias |
| | | GGGGT | | TCCAT | |
| | | AGAAG | | GCCCA | |
| | | TAAGA | | GAAC | |
| | | CG | | C | |
| rs2292467 | 1099 | TGCAT | 1100 | ATGCT | Low |
| | | GTCTG | | CCCAC | Depth, |
| | | TATGT | | TGCAT | High |
| | | GTGTT | | CCTTA | Unmapped |
| | | | | | Reads |
| | | GG | | | |
| rs2300669 | 1101 | AAATG | 1102 | CCCAC | High Off- |
| | | AAGAG | | CAACA | Target |
| | | CCAGC | | CTAAC | Reads |
| | | AGCA | | CTAGC | |
| | | T | | | |
| | | | | A | |
| rs2300855 | 1103 | ACATC | 1104 | TGTGC | High |
| | | TAGCT | | AGATT | Unmapped |
| | | GAGGT | | TATGC | Reads |
| | | CAGAA | | AAATC | |
| | | | | AA | |
| rs2362540 | 1105 | GGGAA | 1106 | AAACA | High Off- |
| | | TTTCT | | CAGCT | Target |
| | | CTGGT | | TCATG | Reads, |
| | | TGGAG | | ACAA | High |
| | | | | G | Unmapped |
| | | | | | Reads |
| rs2376382 | 1107 | GGACT | 1108 | CCTGA | High |
| | | GAGCA | | ATTTT | Unmapped |
| | | TATGT | | TACTT | Reads |
| | | GGAAA | | CTTTG | |
| | | | | C | |
| | | | | TT | |
| rs2430989 | 1109 | TTGCT | 1110 | TGCTA | High Bias |
| | | GAGTA | | AACCA | |
| | | ACAGG | | TTAAA | |
| | | AAAA | | TAATC | |
| | | CAA | | | |
| | | | | TGG | |
| rs2442572 | 1111 | GATGC | 1112 | AGGGT | High |
| | | TAAGC | | AGGAA | Unmapped |
| | | CCATC | | GGATG | Reads |
| | | TCCTG | | CAATG | |
| rs2509973 | 1113 | GGAGC | 1114 | CTGAA | High Off- |
| | | GACCA | | GGGCT | Target |
| | | CTCTT | | CCCAG | Reads |
| | | CATTT | | GCTA | |
| rs2518112 | 1115 | GAAGA | 1116 | CCACA | Low |
| | | TTTTG | | ATGGT | Depth |
| | | TAGCT | | TTGTA | |
| | | GGTCT | | AGATT | |
| | | TGG | | | |
| | | | | T | |
| rs2545450 | 1117 | TGCGT | 1118 | CACAT | Common |
| | | TCTTT | | TTCTC | SNPs in |
| | | GGAGA | | ACCCA | Primer |
| | | TAAGA | | TGTCA | Binding |
| | | | | | Region |
| | | CC | | A | |
| rs2569456 | 1119 | GTTCC | 1120 | TGTGA | Low |
| | | CTCAT | | GATGA | Depth |
| | | CTGCC | | GTGGA | |
| | | CTTC | | GAGC | |
| | | | | AA | |
| rs2632051 | 1121 | TAAAT | 1122 | CCCTT | High Off- |
| | | GTGCC | | TCCTT | Target |
| | | TGGCT | | CCTTG | Reads, |
| | | TGATG | | GATGT | High |
| | | | | | Unmapped |
| | | | | | Reads |
| rs2732954 | 1123 | TGCAA | 1124 | CATTT | High Bias |
| | | GGACA | | GCACA | |
| | | CCAGA | | GCATC | |
| | | ACAGA | | TGACC | |
| rs2786951 | 1125 | GGGTG | 1126 | TTCTA | High |
| | | AGATC | | ATATG | Unmapped |
| | | AAATT | | TATTT | Reads |
| | | CTTAG | | GGGAG | |
| | | GC | | | |
| | | | | AGAG | |
| rs2822493 | 1127 | GCCAT | 1128 | TCTGT | High |
| | | GTTTT | | AAAGG | Unmapped |
| | | CATCT | | ACTTC | Reads |
| | | TGTGG | | ATGTT | |
| | | | | TCAT | |
| rs2881380 | 1129 | TCCTG | 1130 | CTTGT | High |
| | | CCATC | | GGCCT | Unmapped |
| | | TTAAT | | CTCAT | Reads |
| | | AGTCT | | TCTCC | |
| | | C | | | |
| | | ACA | | | |
| rs2906967 | 1131 | TGTTA | 1132 | GAGCT | High Off- |
| | | ATGTA | | CTGGC | Target |
| | | AAATT | | ATTTC | Reads |
| | | GCCTC | | TCTGC | |
| | | GAT | | | |
| rs2920653 | 1133 | TGCTG | 1134 | TTGGC | High Bias |
| | | GAAAG | | ATTAT | |
| | | TCATT | | TTGTG | |
| | | TTGA | | ATCC | |
| rs2993998 | 1135 | CCACA | 1136 | GGGAA | Low depth |
| | | CTCCC | | GACCA | |
| | | CAGAC | | GAACT | |
| | | CAG | | TCAGA | |
| | | | | AA | |
| rs3736590 | 1137 | CTCTT | 1138 | CTTTC | High |
| | | GCCTT | | CTCCC | Unmapped |
| | | CTCAT | | TTTGG | Reads |
| | | TCACA | | GACTC | |
| | | A | | | |
| rs3750880 | 1139 | CCCAC | 1140 | TCAGG | High |
| | | GCACT | | GCGAG | Unmapped |
| | | GTACC | | ATACA | Reads |
| | | ACA | | CCTTT | |
| rs3778354 | 1141 | GCCAG | 1142 | GAGGG | High Off- |
| | | CTCAG | | AAATT | Target |
| | | CTCCT | | CGAGC | Reads, |
| | | CTCT | | ATCAG | High |
| | | | | | Unmapped |
| | | | | | Reads |
| rs3907130 | 1143 | GGCAC | 1144 | GGGAG | High |
| | | TCAAT | | AGAGG | Unmapped |
| | | AAACA | | TGTTC | Reads |
| | | TTGAC | | TCAGC | |
| | | ACA | | | |
| rs4075073 | 1145 | CGCAA | 1146 | GGTGG | High Off- |
| | | TACCT | | GCTGC | Target |
| | | TCAAC | | ATTCA | Reads |
| | | AGCAG | | TAAAG | |
| rs4313714 | 1147 | TGCCA | 1148 | GGGGA | High |
| | | AGAAT | | GGGAG | Unmapped |
| | | CCACT | | AATTG | Reads |
| | | CCAAG | | GACTA | |
| rs4502972 | 1149 | CAAAG | 1150 | CACCA | High |
| | | AAACA | | ACCTG | Unmapped |
| | | GAATG | | GAATG | Reads |
| | | AAAAA | | CTTAC | |
| | | GTGG | | T | |
| rs4642852 | 1151 | TGACT | 1152 | ATACG | High |
| | | GCTCT | | CCAAA | Unmapped |
| | | AAAAT | | CAGTG | Reads |
| | | CTTTG | | AGATG | |
| | | TCA | | | |
| rs4708055 | 1153 | TGACC | 1154 | TGGGA | High |
| | | TATCT | | ATTTT | Unmapped |
| | | ATAAC | | AGTTT | Reads |
| | | CTGTC | | CTCTG | |
| | | CAC | | TCT | |
| rs4717565 | 1155 | ATTGA | 1156 | AATTA | High Off- |
| | | TCTAT | | AGACA | Target |
| | | GTGTC | | GTGTG | Reads |
| | | TGTAG | | GTATT | |
| | | CTT | | GG | |
| rs4768760 | 1157 | TTCAG | 1158 | TTCTT | High Bias |
| | | AGAGG | | CGCAA | |
| | | GACAC | | CCACA | |
| | | CCTTG | | CTTTG | |
| rs4793426 | 1159 | GAGGC | 1160 | AGCCT | High |
| | | TCTCT | | TCCAC | Unmapped |
| | | GGGGC | | CTGAT | Reads |
| | | TTG | | TGAAA | |
| rs4845835 | 1161 | AGAGT | 1162 | TGGTG | High Off- |
| | | CATGC | | GAGAC | Target |
| | | ATCCT | | ACAGA | Reads |
| | | TCATT | | TCCAA | |
| rs4880544 | 1163 | GCAGC | 1164 | CACTT | High |
| | | AGGAA | | GTGTC | Unmapped |
| | | CCATT | | CTCCA | Reads |
| | | CACA | | ACATT | |
| rs4903401 | 1165 | CCCCT | 1166 | CTCCT | High |
| | | CAGAG | | GACCC | Unmapped |
| | | TGATG | | AGCCA | Reads |
| | | ACTGG | | CTTT | |
| rs4909472 | 1167 | GAAAA | 1168 | AGAGA | High |
| | | TCTTG | | GGAGA | Unmapped |
| | | TGGAG | | TGGGG | Reads |
| | | CCTGA | | GAAAG | |
| | | A | | | |
| rs4909666 | 1169 | TGAGC | 1170 | GCCCT | Low depth |
| | | CTACA | | AATGT | |
| | | CTAAC | | AAACT | |
| | | ACATC | | AAAGA | |
| | | A | | CGTT | |
| rs4927069 | 1171 | GGAAA | 1172 | TTTTC | Lowdepth, |
| | | TGTGA | | CATAC | High |
| | | CCCTC | | CTAAA | Unmapped |
| | | ACAGG | | GAACG | Reads |
| rs4945026 | 1173 | CATCA | 1174 | GGCCT | High Off- |
| | | TCTCT | | GGGGG | Target |
| | | TCCTT | | TGCTA | Reads |
| | | ATGTT | | ATG | |
| | | CTCC | | | |
| rs5009912 | 1175 | GGGTG | 1176 | GCTAT | Lowdepth |
| | | GTCTG | | GCCAA | |
| | | GTGAT | | GGGAA | |
| | | GTGTT | | CCTAG | |
| | | | | A | |
| rs6082979 | 1177 | GGGAG | 1178 | CCTCC | High Off- |
| | | TACTC | | TGTCA | Target |
| | | TCCAA | | CTTTC | Reads, |
| | | AGC | | CCTCA | High |
| | | | | | Unmapped |
| | | | | | Reads |
| rs6088301 | 1179 | TGCTC | 1180 | TGGAA | High Bias |
| | | CACAG | | TGTGA | |
| | | ATGAC | | TGGAT | |
| | | ACAGT | | GAGA | |
| rs6124059 | 1181 | AGCCC | 1182 | TTGAC | High |
| | | TGCTT | | TACTG | Unmapped |
| | | CAGCT | | GAACT | Reads |
| | | TCTG | | TGGAG | |
| | | | | AGG | |
| rs6134639 | 1183 | TGGAA | 1184 | GTGGG | High |
| | | ACTTC | | TGGAA | Unmapped |
| | | TTGTG | | GACTT | Reads |
| | | GACCT | | GCTCT | |
| rs6499618 | 1185 | TTTCT | 1186 | CCCAA | High Off- |
| | | GGGCC | | GGTTC | Target |
| | | ACCTA | | TGGGC | Reads |
| | | CAAGT | | TAAG | |
| rs6538276 | 1187 | CCTCC | 1188 | CCCTT | High Off- |
| | | TCCTC | | TCTTA | Target |
| | | ACACT | | GCTCC | Reads, |
| | | GCTTC | | TGACC | High |
| | | | | A | Unmapped |
| | | | | | Reads |
| rs6560430 | 1189 | GGTCT | 1190 | GAATG | High |
| | | AAAGG | | GTCTT | Unmapped |
| | | GAGAG | | TTCGT | Reads |
| | | TAGGA | | CATTC | |
| | | GGTC | | C | |
| rs6602240 | 1191 | TTTTC | 1192 | CACAC | High |
| | | CCAAA | | ACAAG | Unmapped |
| | | ACCCC | | GAAAA | Reads |
| | | ACACT | | ACAGG | |
| | | | | A | |
| rs6681073 | 1193 | GCTGG | 1194 | TGCCT | Lowdepth |
| | | ATGGA | | GCCTG | |
| | | GGGTG | | TTAGA | |
| | | AGG | | ACATC | |
| rs6682943 | 1195 | GGCAA | 1196 | TGGAA | High Bias |
| | | TCCGA | | CCAAC | |
| | | AGTCT | | AACCT | |
| | | AAGAG | | ATCAT | |
| | | A | | CA | |
| rs6700298 | 1197 | GACTG | 1198 | TGAAA | High |
| | | GTACT | | ATCCA | Unmapped |
| | | TCCCC | | TTTGG | Reads |
| | | AAGGA | | TAGTT | |
| | | | | GCT | |
| rs6714809 | 1199 | AAAAT | 1200 | TGGTA | High |
| | | GACTG | | AGTGG | Unmapped |
| | | TCCCC | | GATGA | Reads |
| | | TATCT | | TACTG | |
| | | | | AGC | |
| rs6728087 | 1201 | AAGCA | 1202 | CCCCT | High Bias |
| | | TAGAA | | GAATG | |
| | | GGAAA | | AAACT | |
| | | AACAG | | ATTGA | |
| | | ATTG | | GC | |
| rs6765108 | 1203 | AGCAA | 1204 | TTGTC | High Off- |
| | | GGGAG | | AATCC | Target |
| | | GGAAG | | TTGCT | Reads, |
| | | ACACC | | CTACC | High |
| | | | | C | Unmapped |
| | | | | | Reads |
| rs6788750 | 1205 | TGAAG | 1206 | TAATC | High Bias |
| | | GGTAG | | TTTGG | |
| | | ATATG | | ACTCC | |
| | | AAGTT | | TTGAA | |
| | | TTTC | | | |
| rs6863383 | 1207 | TGATC | 1208 | CCCCT | High Bias |
| | | CCATG | | GAAAT | |
| | | TATTT | | GAGAG | |
| | | AAACC | | TCACC | |
| | | T | | | |
| rs6893628 | 1209 | CAAAA | 1210 | CTTTA | High Bias |
| | | TAAAC | | ACAAA | |
| | | CCAGG | | TATAG | |
| | | CAAAA | | GGCGA | |
| | | A | | TTT | |
| rs6986644 | 1211 | AAGTA | 1212 | TCCCC | High |
| | | CCAAA | | CTAAG | Unmapped |
| | | AAGGC | | ATCAG | Reads |
| | | ACATC | | GAACA | |
| | | G | | | |
| rs6994806 | 1213 | TGGAA | 1214 | AAGAG | High |
| | | CAGCA | | TGTAA | Unmapped |
| | | ACTTG | | ATGGG | Reads |
| | | CAAAC | | TCCTG | |
| | | | | A | |
| rs7098657 | 1215 | CTCCC | 1216 | TGCTC | High Off- |
| | | CTGAA | | ACATT | Target |
| | | CCTGA | | TCATT | Reads |
| | | GTGAC | | GACCA | |
| | | | | G | |
| rs7133402 | 1217 | TGAGG | 1218 | TGCGA | High |
| | | TGGGA | | CTGGA | Unmapped |
| | | AGAAA | | TACTA | Reads |
| | | CACAA | | TTTTT | |
| | | | | GG | |
| rs7157032 | 1219 | AGTTG | 1220 | TGTTG | High |
| | | CATGG | | GTGCA | Unmapped |
| | | AGTGG | | TTCAG | Reads |
| | | CTGA | | AGAGC | |
| rs7195624 | 1221 | CAAGT | 1222 | AGGCT | High |
| | | AATTC | | ACAAA | Unmapped |
| | | TTACC | | AAGGC | Reads |
| | | AGCCT | | AGCAG | |
| | | TT | | | |
| rs7251148 | 1223 | AAGGA | 1224 | GACCC | High |
| | | AACGG | | TGTGG | Unmapped |
| | | CCCCA | | ACTGA | Reads |
| | | GAG | | GAACC | |
| rs7479857 | 1225 | TCAGA | 1226 | CTTTT | High |
| | | GCACT | | TAAAG | Unmapped |
| | | CTGCA | | CCAGA | Reads |
| | | TTCCA | | AAAAT | |
| | | | | GG | |
| rs7521976 | 1227 | AGAAT | 1228 | CAGCT | High |
| | | CATAT | | TATCT | Unmapped |
| | | GACAC | | TTATC | Reads |
| | | ATGGA | | TGTTT | |
| | | A | | GCTT | |
| rs7564063 | 1229 | CACTT | 1230 | CAGAT | High Bias |
| | | TGCAG | | CTGAT | |
| | | CCAAT | | TTCCT | |
| | | CCATA | | GGAG | |
| rs7608890 | 1231 | TCCAT | 1232 | GTGCA | Lowdepth |
| | | ACAGG | | GTTTG | |
| | | AAGAT | | GGCTA | |
| | | CCATT | | CAAGA | |
| | | AAGA | | | |
| rs7684457 | 1233 | TGCTG | 1234 | AGAAA | High Off- |
| | | CCAGA | | GTTGT | Target |
| | | AGCAA | | GCCAA | Reads, |
| | | CCTAC | | GTGCT | High |
| | | | | | Unmapped |
| | | | | | Reads |
| rs7745188 | 1235 | TGTCT | 1236 | CATAA | High Off- |
| | | GGAAA | | AGCTA | Target |
| | | TCATT | | AAAGA | Reads |
| | | GCTTC | | TTGGA | |
| | | A | | CA | |
| rs7763061 | 1237 | CAAAT | 1238 | GTTTT | High |
| | | CAGTG | | GCCCA | Unmapped |
| | | TGCCC | | GAGGT | Reads |
| | | CAAC | | CATGT | |
| rs7820286 | 1239 | GCTCT | 1240 | CTATC | High |
| | | TCCCT | | ATTTC | Unmapped |
| | | CAGTG | | TCCCC | Reads |
| | | GCTTA | | AACAC | |
| | | | | A | |
| rs7830700 | 1241 | CTGGA | 1242 | TCAAG | High Bias |
| | | TTTCA | | TATCT | |
| | | AATTG | | AGTTG | |
| | | TTTCA | | TGATA | |
| | | | | GCC | |
| rs7833328 | 1243 | TAGAG | 1244 | CGAGA | High Off- |
| | | CAGCT | | CTGTT | Target |
| | | AGGGG | | CACCC | Reads, |
| | | ACTGC | | TTTGG | High |
| | | | | | Unmapped |
| | | | | | Reads |
| rs7982170 | 1245 | ATGCC | 1246 | TTTCA | High Off- |
| | | AGACT | | GTTTT | Target |
| | | TCACC | | GTTAT | Reads |
| | | ACTGC | | GTGGC | |
| | | | | TA | |
| rs8053194 | 1247 | TTGAA | 1248 | ATCAA | High |
| | | GTTAG | | CTCCC | Unmapped |
| | | TTCTT | | CACCT | Reads |
| | | TGTGG | | GGAAG | |
| | | ATGG | | | |
| rs9300647 | 1249 | TTTTC | 1250 | TGATT | High |
| | | CCTCA | | CCAGT | Unmapped |
| | | TTAGC | | TCACA | Reads |
| | | TGCAT | | GTAGT | |
| | | T | | CCA | |
| rs9371705 | 1251 | CATTT | 1252 | ACCCT | High Bias |
| | | CCAGC | | GAGGA | |
| | | TGACT | | GGGGC | |
| | | GGTTA | | TAGT | |
| rs9377381 | 1253 | GCCCA | 1254 | AGATC | High Off- |
| | | GTAGC | | ACCAA | Target |
| | | ACTGC | | GGCAG | Reads |
| | | TCTTC | | AAACC | |
| rs9405991 | 1255 | CCGAG | 1256 | GGCAG | High Bias |
| | | AACGC | | CAACA | |
| | | TCTGA | | GGAAA | |
| | | GTTG | | TAGCA | |
| rs9522306 | 1257 | ACAGG | 1258 | CACTG | High |
| | | AGTGG | | CAGGA | Unmapped |
| | | CTCGG | | AATGC | Reads |
| | | TCA | | AGCTT | |
| rs9864296 | 1259 | CGAAA | 1260 | AGCTA | High |
| | | TCCAT | | CACTA | Unmapped |
| | | AGGAC | | TTTCC | Reads |
| | | CTACA | | ATGTG | |
| | | | | AC | |
| rs9881075 | 1261 | AACAA | 1262 | CTGGG | High |
| | | GAAAG | | TCACG | Unmapped |
| | | GCAGG | | CCTCT | Reads |
| | | GAAGG | | TGA | |
| rs10041720 | 1263 | TACAA | 1264 | GCCAG | High Bias |
| | | ACAGT | | GCATG | |
| | | GGGGC | | GGCTT | |
| | | AACAA | | AAT | |
| rs10106215 | 1265 | TTCGT | 1266 | AACAG | High Bias |
| | | CTTTC | | AAAGA | |
| | | AGCAA | | GAGTT | |
| | | TTTGA | | ACATC | |
| | | | | TACA | |
| rs10142058 | 1267 | CCTCA | 1268 | CCCCC | High Off- |
| | | TGACC | | AATGC | Target |
| | | TAACC | | AAGAG | Reads |
| | | ACCTC | | TGTT | |
| rs10444986 | 1269 | TTTCA | 1270 | GCCCA | High |
| | | CAGTG | | GGACA | Unmapped |
| | | GAATG | | CACAA | Reads |
| | | AATCG | | AAA | |
| rs10765992 | 1271 | CTGGT | 1272 | CACCG | Low Depth, |
| | | CCTCT | | AATCT | High |
| | | GTGAA | | ATATC | Unmapped |
| | | TTGAA | | TGTGA | Reads |
| | | | | GG | |
| rs10787889 | 1273 | TCTTT | 1274 | TATGC | High Off- |
| | | ATGTG | | TGAAG | Target |
| | | GCCTT | | CTGCC | Reads |
| | | CACTT | | ATCCT | |
| | | G | | | |
| rs10790395 | 1275 | GGGCA | 1276 | GCTGT | High |
| | | GGAAA | | CCTAT | Unmapped |
| | | CAGGG | | TTCAG | Reads |
| | | ACTA | | GTTGC | |
| | | | | AT | |
| rs10800542 | 1277 | TCCAC | 1278 | AGCAA | High |
| | | TGGAA | | TCATC | Unmapped |
| | | TTGGT | | CTAGG | Reads |
| | | AGACA | | AGGTC | |
| | | GA | | A | |
| rs10815682 | 1279 | TTCTG | 1280 | GGGCA | High |
| | | ACTTC | | AGTCA | Unmapped |
| | | ACAGA | | CTTAG | Reads |
| | | GGGTA | | CATTT | |
| rs10874506 | 1281 | TTCTC | 1282 | TGAAA | High |
| | | AGACT | | AGATA | Unmapped |
| | | TCAAA | | CCTAA | Reads |
| | | GCAAA | | AATCA | |
| | | GG | | AGG | |
| rs10906984 | 1283 | GAGAA | 1284 | ATTTC | High |
| | | GAACC | | TGCAG | Unmapped |
| | | AGACA | | CCCTG | Reads |
| | | GAACA | | TGACT | |
| | | CG | | | |
| rs10952780 | 1285 | CATGA | 1286 | TCCTA | High |
| | | AAAAT | | AGTTT | Unmapped |
| | | AAGGA | | TTCTG | Reads |
| | | AATGC | | ATCTG | |
| | | TGA | | TGG | |
| rs11058137 | 1287 | GCCTC | 1288 | CCTCT | High Bias |
| | | AGTTT | | CAACA | |
| | | CCTCC | | ACCCA | |
| | | TCAGA | | GGTAC | |
| | | | | T | |
| rs11153132 | 1289 | ACTGT | 1290 | AGTCC | High Off- |
| | | GGCTC | | AGGCA | Target |
| | | CAGCA | | CCACT | Reads |
| | | TGAA | | GCTAC | |
| rs11216096 | 1291 | GCTGG | 1292 | ATGGC | High Off- |
| | | AAGGA | | CACTA | Target |
| | | GAGAA | | GAGGG | Reads, |
| | | ACACG | | GAGTC | High |
| | | | | | Unmapped |
| | | | | | Reads |
| rs11705789 | 1293 | GCATC | 1294 | TGGTC | High Bias |
| | | CTGTG | | AATAA | |
| | | GTGGG | | GCCTG | |
| | | AAG | | TTCCA | |
| rs11714718 | 1295 | GGTCA | 1296 | TCAAT | High Off- |
| | | GGACC | | AACTG | Target |
| | | TGTTT | | CTGGA | Reads, |
| | | TCTCA | | GATGT | High |
| | | A | | GG | Unmapped |
| | | | | | Reads |
| rs11745637 | 1297 | GCCCA | 1298 | GCAGC | Low Depth, |
| | | ATCTA | | CAAGA | High |
| | | ATCAT | | AAGGC | Unmapped |
| | | GTGAG | | TGT | Reads |
| | | G | | | |
| rs11786747 | 1299 | GGAAA | 1300 | TCCTC | High |
| | | GCAGT | | TTCCC | Unmapped |
| | | GAAGA | | CAGAA | Reads |
| | | CAGCA | | CTTGA | |
| rs12210929 | 1301 | GTTGG | 1302 | TCCTT | Low Depth |
| | | GGCAG | | TACTA | |
| | | TACTC | | CATCA | |
| | | AGCAG | | TGGGT | |
| | | | | CA | |
| rs12287505 | 1303 | GGCCT | 1304 | TTGAA | High Off- |
| | | CCCCT | | CTAGT | Target |
| | | TCATT | | TTATA | Reads |
| | | CAA | | CACCC | |
| | | | | AGAA | |
| rs12321981 | 1305 | CACAC | 1306 | CAAAG | High |
| | | ATACA | | AAGAA | Unmapped |
| | | CAAAA | | GGAGC | Reads |
| | | TAAAG | | AAGG | |
| | | GT | | | |
| rs12349140 | 1307 | TTATC | 1308 | CCCGG | High Off- |
| | | CAGGA | | TGATA | Target |
| | | CAGGA | | ACAGA | Reads, |
| | | AGCTG | | ACGAT | High |
| | | | | | Unmapped |
| | | | | | Reads |
| rs12448708 | 1309 | CATGG | 1310 | TTTTA | Low Depth, |
| | | GACTC | | ATCTC | High |
| | | TAGAG | | TCTTG | Unmapped |
| | | GTAGA | | CTCTC | Reads |
| | | A | | C | |
| rs12500918 | 1311 | TCATA | 1312 | TTTAC | High Off- |
| | | GAGTA | | CAGCC | Target |
| | | AGCCA | | AGCTC | Reads, |
| | | GATAT | | AGTCC | High |
| | | AAGC | | | Unmapped |
| | | | | | Reads |
| rs12554667 | 1313 | TCCTG | 1314 | ACCAA | High Off- |
| | | AAGGG | | GGTCT | Target |
| | | TAAGC | | TCCCT | Reads, |
| | | AGGAA | | CTGC | Low |
| | | | | | Depth |
| rs12660563 | 1315 | AGGTC | 1316 | GCTCC | High |
| | | AGCTC | | ATTGA | Off- |
| | | AGGGT | | AGGGT | Target |
| | | GAAGT | | AAAGG | Reads |
| rs12711664 | 1317 | TGGAA | 1318 | AGCCC | High |
| | | TAGAA | | ACACA | Unmapped |
| | | TGCAA | | GGTTG | Reads |
| | | TCCTG | | GTAAG | |
| | | A | | | |
| rs12881798 | 1319 | CAGAT | 1320 | GTGGA | High Off- |
| | | GCTGC | | TCACA | Target |
| | | AGGAA | | GGGTC | Reads, |
| | | ACAGA | | ACCTC | High |
| | | | | | Unmapped |
| | | | | | Reads |
| rs12917529 | 1321 | CCTCA | 1322 | AAGGC | Low Depth, |
| | | AGCTG | | AGGCA | High |
| | | GCCTG | | AGACG | Unmapped |
| | | CAA | | TAGC | Reads |
| rs13019275 | 1323 | CAAAT | 1324 | TGATG | High |
| | | ATACT | | CATTG | Unmapped |
| | | GATTC | | AGATT | Reads |
| | | TGTGG | | TTGAT | |
| | | CAAA | | GA | |
| rs13042906 | 1325 | CGTCT | 1326 | GGTAG | High Bias |
| | | CCCAC | | GCTTT | |
| | | ATTCT | | GTAAC | |
| | | TTTGG | | TTGCA | |
| | | | | CTG | |
| rs13267077 | 1327 | TGAAT | 1328 | GCCTC | High |
| | | CCTGG | | ACCTA | Unmapped |
| | | CTGGG | | CAAAG | Reads |
| | | AAA | | CTTAT | |
| | | | | TCA | |
| rs13362486 | 1329 | TGCAG | 1330 | TGAAG | High |
| | | TTTGC | | CTACA | Unmapped |
| | | TATGC | | CAGAT | Reads |
| | | AGTCT | | AAGAA | |
| | | TT | | GC | |
| rs17077156 | 1331 | TCATT | 1332 | GCCAG | High |
| | | CTGGG | | GAAAA | Unmapped |
| | | TTACC | | GACAG | Reads |
| | | CTMTG | | TGCAT | |
| rs17382358 | 1333 | TCTCA | 1334 | GCACA | Low Depth |
| | | GCACA | | TTTAT | |
| | | GAGAA | | TCACT | |
| | | GGTGC | | CAGCA | |
| | | T | | AA | |
| rs17699274 | 1335 | TGTCC | 1336 | CAMTT | High |
| | | TCTGT | | CCAAG | Unmapped |
| | | AAACC | | GTTGT | Reads |
| | | AGACA | | TTCTG | |
| | | A | | T | |

### Example 3. Hematopoietic Stem Cell Transplantation Engraftment Testing by SNP Allele Frequency Measurement by Targeted Sequencing

47 genomic DNA samples were derived from remnant patient genomic DNAs previously tested for donor engraftment by standard of care short tandem repeat (STR) analysis using targeted PCR and detection by capillary electrophoresis. Sample information is shown in the table below.

| set | source | post type |
|---|---|---|
| S1 | PRE | |
| S1 | DON | |
| S1 | POST | blood |
| S2 | PRE | |
| S2 | DON | |
| S2 | POST | bone marrow |
| S3 | PRE | |
| S3 | DON | |
| S3 | POST | blood |
| S4 | PRE | |
| S4 | DON | |
| S4 | POST | CD56 |
| S5 | PRE | |
| S5 | DON | |
| S5 | POST | blood |
| S6 | PRE | |
| S6 | DON | |
| S6 | POST | blood |
| S7 | PRE | |
| S7 | DON | |
| S7 | POST1 | blood |
| S7 | POST2 | blood |
| S8 | PRE | |
| S8 | DON | |
| S8 | POST1 | CD3 |
| S8 | POST2 | CD3 |
| S9 | PRE | |
| S9 | DON | |
| S9 | POST | CD3 |
| S10 | PRE | |
| S10 | POST | blood |
| S11 | PRE | |
| S11 | DON | |
| S11 | POST | CD3 |
| S12 | PRE | |
| S12 | DON | |
| S12 | POST | CD3 |
| S13 | PRE | |
| S13 | DON | |
| S13 | POST1 | CD3 |
| S13 | POST2 | blood |
| S14 | PRE | |
| S14 | DON | |
| S14 | POST | blood |
| S15 | PRE | |
| S15 | DON | |
| S15 | POST | blood |

Note:
each sample set comprises three samples: “PRE” and “POST” samples were taken from the recipients before and after transplantation, and the “DON” sampels was taken from the donors.

The genomic DNA samples were purified and concentrations of the purified DNA were determined to have ranged from 0.035-95 ng/uL. Using 10 uL of genomic DNA per reaction (in cases of high concentration samples were diluted), the SNP targets as in Table 6 were amplified in a single-tube multiplexed PCR, essentially as illustrated in FIG. 13. In brief, all forward loci primers are designed to contain a common adapter sequence on the 5′ end of the adapter to enable subsequent incorporation of sequencing adapters. Similarly, all reverse loci primers are designed to contain a common adapter sequence (distinct from that on the forward primers) on the 5′ end of the adapter to enable subsequent incorporation of sequencing adapters. Loci PCR product was quantified by capillary electrophoresis and normalized to a standard concentration. Normalized loci PCR product was then amplified with dual-index barcoded universal PCR primers targeting the adapter sequences incorporated into the loci specific PCR primers. Universal PCR product was quantified by qPCR and samples were normalized to equimolar concentrations. Barcoded, normalized universal PCR product was then combined at equimolar concentrations and sequenced with 42 cycles of paired end reads to cover the genomic region and dual index sequencing.

Samples are sequenced and sequence data are demultiplexed using the dual-index sample barcodes. Sequence reads are aligned to hg19 reference genome. Of reads aligning to the expected loci SNP targets, paired-end insert sequence reads are checked for matching consensus reads at the SNP allele base position. Of the paired consensus matched reads, SNP base position counts are determined for the expected reference and alternate SNP alleles as well as unexpected non-reference and non-alternate alleles.

To determine the engraftment success or failure, the reference and alternate allele based SNP allele frequencies are used to determine the genotypes of the donor and pre-transplant recipient sample. As an initial guideline for donor and recipient genotyping, SNP allele frequencies from 0.9-1 would indicate homozygosity for the reference allele, SNP allele frequencies from 0-0.1 would indicate homozygosity for the alternate alleles, and allele frequencies from 0.4-0.6 would indicate heterozygosity.

SNPs for which the donor and recipient are opposing homozygous genotypes (i.e., AA versus aa) are most useful to determine both engraftment and relapse in the post-transplant recipient sample. For engraftment, donor alleles may be the major contributing factor in the post-transplant allele frequency measurement. With full, successful engraftment the contribution of the recipient allele frequency will be undetectable. Unlike the current technology with STRs and capillary measurement, the lower limits of detection are ˜5% STR allele frequencies, the SNP allele frequency measurement limit of detection can be much lower-down to a 1% range or so. In cases where a patient has successfully engrafted, monitoring for disease relapse can be useful. Patients are be determined to have a relapse if the SNP alleles from the recipient start to reappear over time and regain a significant fractional allele concentration.

SNPs for which the donor is homozygous and recipient is heterozygous will be most useful in determining re-population of the PBMC population or sub-populations with relapsing recipient cells. SNPs for which the donor is heterozygous and recipient is homozygous will be most useful in determining engraftment of the donor PBMC population or sub-populations.

The DNAs are sequenced and the SNP alleles are counted from the sequence reads. From the counts of SNP reference and alternate alleles, reference and alternate allele frequencies are determined. Based on genotypes of the donor and recipient and using the DF4 approach described above, the donor fraction, indicating the status of engraftment/relapse in each recipient, is then determined based on SNP allele frequencies. We expect the donor fractions to show a linear correlation to the results of the prior STR analysis.

1. A method of determining transplant status comprising:
(a) obtaining a sample from a hematopoietic stem cell transplant (HSCT) recipient who has received hematopoietic stem cells from an allogenic source;
(b) measuring the amount of one or more identified recipient-specific nucleic acids or donor-specific nucleic acids in the sample; and
(c) determining transplant status by monitoring the amount of the one or more identified recipient-specific nucleic acids or donor-specific nucleic acids after transplantation,
wherein said the one or more recipient-specific or the donor-specific nucleic acids are identified based on one or more polymorphic nucleic acid targets, and
wherein the nucleic acid is genomic DNA.

2. (canceled)

3. The method of claim 1, the method further comprising determining a donor-specific nucleic acid fraction based on the amount of the polymorphic nucleic acid targets that are specific for donor and the total amount of the polymorphic nucleic acid targets in total nucleic acids in the biological sample.

4. The method of claim 1, wherein the biological sample is blood or bone marrow.

5-6. (canceled)

7. The method of claim 1, wherein one or more polymorphic nucleic acid targets are one or more SNPS, and
wherein the one or more SNPs do not comprise a SNP for which the reference allele and alternate allele combination is selected from the group consisting of A_G, G_A, C_T, and T_C.

8. The method of claim 4, wherein the genomic DNA is isolated from one or more cell populations purified from the sample, or
the genomic DNA is isolated from peripheral white blood cells in the sample.

9. The method of claim 8, wherein the one or more cell populations are selected from a group consisting of B-cells, granulocytes, and T-cells.

10. (canceled)

11. The method of claim 8, wherein the purified cell population are peripheral blood mononuclear cells.

12. The method of claim 1, wherein the HSCT recipient has at least one hematological disorder from a group consisting of leukemias, lymphomas, immune-deficiency illnesses, hemoglobinopathy, congenital metabolic defects, and non-malignant marrow failures.

13. The method of claim 1, wherein the determining the transplant status step (c) comprises determining the transplant status as a graft failure if the one or more recipient-specific nucleic acids are increased during a time interval post-transplantation, or
if the one or more donor-specific nucleic acids are decreased during a time interval post-transplantation.

14. (canceled)

15. The method of claim 1 wherein the determining the transplant status step (c) comprises determining the transplant status as engraftment of the HSCT if:
i) the one or more recipient-specific nucleic acids in the peripheral blood cells is below a threshold post-transplantation,
ii) the one or more recipient-specific nucleic acids are decreased during a time interval post-transplantation,
iii) the one or more donor-specific nucleic acids in the peripheral blood cells is above a threshold post-transplantation, or
iv) the one or more donor-specific nucleic acids are increased during a time interval post-transplantation.

16. The method of claim 15 wherein the threshold is a percentage of recipient-specific nucleic acid relative to a total of recipient-specific and donor-specific nucleic acids.

17. (canceled)

18. The method of claim 1, wherein the recipient-specific nucleic acid or the donor-specific nucleic acid is determined by measuring the one or more polymorphic nucleic acid targets in at least one assay, and
wherein the at least one assay is high-throughput sequencing, capillary electrophoresis or digital polymerase chain reaction (dPCR).

19. The method of claim 1, wherein the recipient-specific nucleic acid or the donor-specific nucleic acid is determined by targeted amplification using a forward and a reverse primer designed specifically for a native genomic nucleic acid, and a variant synthetic oligo that contains a variant as compared to the native genomic sequence,
wherein the variant can be a substitution of single nucleotides or multiple nucleotides compared to the native sequence
wherein the variant oligo is added to the amplification reaction in a known amount
wherein the method further comprises:
determining the ratio of the amount of the amplified native genomic nucleic acid to the amount of the amplified variant oligo,
determining the total copy number of genomic DNA by multiplying the ratio with the amount of the variant oligo added to the amplification reaction.

20. The method of claim 19, wherein the method further comprises determining total copy number of genomic DNA in the biological sample, and determining the copy number of the recipient-specific or donor-specific nucleic acid by multiplying the recipient-specific or donor-specific nucleic acid fraction and the total copy number of genomic DNA.

21. The method of claim 1, wherein said polymorphic nucleic acid targets comprise one or more SNPs.

22. The method of claim 21, wherein each of the one or more SNPs has a minor allele frequency of 15%-49%, and/or
wherein the SNPs comprise at least one, two, three, four, or more SNPs in Table 1 or Table 6.

23. (canceled)

24. The method of claim 1, wherein the recipient and/or donor is genotyped prior to transplantation using one or more SNPs in Table 1 or Table 6, or
wherein the donor is not genotyped, the recipient is not genotyped, or neither the donor nor the recipient is genotyped for any one of the one or more polymorphic nucleic acid targets prior to transplantation.

25-27. (canceled)

28. The method of claim 18, wherein the high-throughput sequencing is targeted amplification using a forward and a reverse primer designed specifically for the one or more polymorphic nucleic acid targets or targeted hybridization using a probe sequence that contains the one or more polymorphic nucleic acid targets,
wherein the targeted amplification or targeted hybridization is a multiplex reaction.

29. (canceled)

30. The method of claim 1, wherein the allogenic source is from the group comprising bone marrow transplant, peripheral blood stem cell transplant, and umbilical cord blood.

31. (canceled)

32. The methods of claim 24, wherein the genotypes for at least one of the donor and the recipient is not known prior to the transplantation determination, wherein the one or more nucleic acids from said HSCT recipient are identified as recipient-specific nucleic acid or donor-specific nucleic acid using a computer algorithm based on measurements of one or more polymorphic nucleic acid target.

33. The method of claim 32, wherein the algorithm comprises one or more of the following: (i) a fixed cutoff, (ii) a dynamic clustering, and (iii) an individual polymorphic nucleic acid target threshold.

34. The method of claim 33, wherein the fixed cutoff algorithm detects donor-specific nucleic acids if the deviation between the measured frequency of a reference allele of the one or more polymorphic nucleic acid targets in the nucleic acids in the sample and the expected frequency of the reference allele in a reference population is greater than a fixed cutoff,
wherein the expected frequency for the reference allele is in the range of
0.00-0.03 if the recipient is homozygous for the alternate allele,
0.40-0.60 if the recipient is heterozygous for the alternate allele, or
0.97-1.00 if the recipient is homozygous for the reference allele.

35. The method of claim 33, wherein the recipient is homozygous for the reference allele and the fixed cutoff algorithm detects donor-specific nucleic acids if the measured allele frequency of the reference allele of the one or more polymorphic nucleic acid targets is greater than the fixed cutoff.

36. The method of any of claim 33, wherein the fixed cutoff is based on the homozygous allele frequency of the reference or alternate allele of the one or more polymorphic nucleic acid targets in a reference population.

37. The method of claim 33, wherein the fixed cutoff is based on a percentile value of distribution of the homozygous allele frequency of the reference or alternate allele of the one or more polymorphic nucleic acid targets in the reference population.

38. The method of claim 37, wherein the percentile value is at least 90.

39. The method of claim 33, wherein identifying one or more nucleic acids as donor-specific nucleic acids using the dynamic clustering algorithm comprises
(i) stratifying the one or more polymorphic nucleic acid targets in the nucleic acids into recipient homozygous group and recipient heterozygous group based on the measured allele frequency for a reference allele or an alternate allele of each of the polymorphic nucleic acid targets;
(ii) further stratifying recipient homozygous groups into non-informative and informative groups; and
(iii) measuring the amounts of one or more polymorphic nucleic acid targets in the informative groups.

40. The method of claim 33, wherein the dynamic clustering algorithm is a dynamic K-means algorithm, and
wherein the individual polymorphic nucleic acid target threshold algorithm identifies the one or more nucleic acids as donor-specific nucleic acids if the allele frequency of each of the one or more of the polymorphic nucleic acid targets is greater than a threshold.

41. (canceled)

42. The method of claim 40, wherein the threshold is based on the homozygous allele frequency of each of the one or more polymorphic nucleic acid targets in a reference population.

43. The method of claim 42, wherein the threshold is a percentile value of a distribution of the homozygous allele frequency of each of the one or more polymorphic nucleic acid targets in the reference population.

44. The method of claim 1, further comprises determining the patient as having mixed chimerism when the donor-specific nucleic acid faction in the post-transplant sample from a recipient ranges from 5% to 90%, and/or that the recipient fraction in the post-transplant sample ranges from 95% to 10%,

45. The method of claim 1, further comprises isolating DNA from individual cell populations from the patient and determining the patient as having split chimerism when the donor-specific nucleic acid fraction in one cell population is in the range of 91% to 100%, and wherein the donor fraction in another cell population is less than 91%.

46. A system for determining transplantation status comprising one or more processors; and memory coupled to one or more processors, the memory encoded with a set of instructions configured to perform a process comprising:
(a) obtaining measurements of one or more identified recipient-specific nucleic acids or donor-specific nucleic acids in the sample after transplantation
(b) determining the amount of the one or more identified recipient-specific nucleic acids or donor-specific nucleic acids in the sample after transplantation based on (a); and
(c) determining a transplantation status based on the amount of the identified recipient-specific nucleic acids or donor-specific nucleic acids.
